(19) United States
(12) Patent Application Publication
(10) Pub. No.: US 2016/0083391 A1
(43) Pub. Date: Mar. 24, 2016

(54) TRICYCLIC PIPERAZINE DERIVATIVE

(71) Applicants: Sunovion Pharmaceuticals Inc., Marlborough, MA (US); Sumitomo Dainippon Pharma Co., Ltd., Osaka (JP)

(72) Inventors: Douglas F. Burdi, Arlington, MA (US); Daisuke Tanaka, Osaka (JP)

(21) Appl. No.: 14/857,677

(22) Filed: Sep. 17, 2015

Related U.S. Application Data
(60) Provisional application No. 62/052,367, filed on Sep. 18, 2014.

(51) Int. Cl. C07D 487/14; C07D 498/14

(52) U.S. Cl. CPC C07D 487/14; C07D 498/14

(57) ABSTRACT

Disclosed are compounds useful as inhibitors of Phosphodiesterase 1 (PDE1), compositions thereof, and methods of using the same.

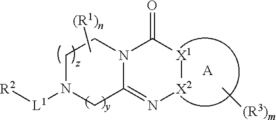

### BACKGROUND OF THE INVENTION

The prevalence of neurological and psychiatric disorders is increasing worldwide. Up to one billion people suffer from debilitating neurological conditions such as Alzheimer's disease and Parkinson's disease, with almost seven million people dying every year. “Neurological disorders: public health challenges” World Health Organization, 2006. Neurological and psychiatric disorders are prevalent in all countries, often without regard to age, sex, education or income. However, as many neurological disorders are correlated with increased age, as the global population ages, the impact of these disorders becomes more evident.

Despite the availability of treatments for some of these diseases, first line therapies (such as L-DOPA for Parkinson's) are often burdened by unfavorable side effects, or may lack efficacy. For instance, there is currently no approved treatment for the cognitive deficits in schizophrenia despite the high unmet medical need.

The continuing and increasing problem of neurological and psychiatric disorders, and the current lack of safe and effective drugs for treating them, highlight the overwhelming need for new drugs to treat these conditions and their underlying causes.

### SUMMARY OF THE INVENTION

It has now been found that compounds of this invention, and pharmaceutically acceptable compositions thereof, are effective as inhibitors of Phosphodiesterase 1 (PDE1) enzymes. Such compounds have the general formula I:

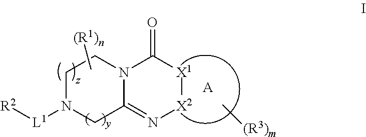

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of PDE1 enzymes. Such diseases, disorders, or conditions include those described herein.

Compounds provided by this invention are also useful for the study of PDE1 enzymes in biological and pathological phenomena; the study of intracellular signal transduction pathways occurring in PDE1-expressing tissues; and the comparative evaluation of new PDE1 inhibitors or other regulators neuronal activity in vitro or in vivo.

### DETAILED DESCRIPTION OF CERTAIN EMBODIMENTS

### 1. General Description of Compounds of the Invention

In certain embodiments, the present invention provides inhibitors of PDE1. In some embodiments, such compounds include those of formula I:

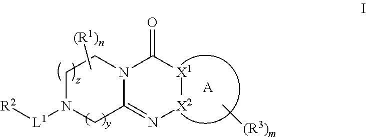

or a pharmaceutically acceptable salt thereof, wherein:
X¹ and X² are each independently C or N;
Ring A is a 5-membered heteroaryl ring having 1-4 heteroatoms independently selected from nitrogen, oxygen and sulfur;
L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —OC(O)—, —C(O)O—, —O—, —S—, —S(O)— or —S(O)₂—;
each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R, —S(O)R, or S(O)₂R;
R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy;
Cy is a ring, substituted with p instances of R⁵; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-1.0 membered bicyclic heteroaromatic ring having 1-5 heteroatoms independently selected from nitrogen, oxygen, and sulfur;
each R is independently hydrogen, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂;
each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂; or
two R⁵ are taken together with their intervening atoms to form a 5-6 membered saturated, partially unsaturated, or aromatic ring having 0-3 heteroatoms independently selected from nitrogen, oxygen, and sulfur;
wherein one or more of {two instances of R¹}, {R² and an R¹}, and {two instances of R³} may be taken together with their intervening atoms to form a ring substituted with q instances of R⁵; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur;
   y is 1-2;
   z is 1-2;
   m is 0-3;
   n is 0-3;
   p is 0-5; and
   q is 0-5.

### 2. Compounds and Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl alkynyl groups and hybrids thereof such as (cycloalkyl)alkyl, (cycloalkenyl)alkyl or (cycloalkyl)alkenyl.

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, boron, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆ or C₁₋₄) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is a carbocyclic aromatic ring and wherein each ring in the system contains 3 to 7 ring members. The term “aryl” may be used interchangeably with the term “aryl ring.” In certain embodiments of the present invention, “aryl” refers to a carbocyclic aromatic ring system which includes, but not limited to, phenyl, naphthyl, anthracyl and the like, which may be optionally substituted. Also included within the scope of the term. “aryl,” as it is used herein, is a group in which a carbocyclic aromatic ring is fused to one or more nonaromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 1.0 ring atoms, preferably 5, 6, 9 or 10 ring atoms; having 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7- to 10-membered bicyclic heterocyclic moiety that is either saturated or partially unsaturated, and having, in addition to carbon atoms, one or more, preferably one to four, heteroatoms. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —NO₂; —CN; —N₃; —(CH₂)₀₋₄N(R^{∘})₂; —(CH₂)₀₋₄N(R^{∘})C(O)R^{∘}; —(CH₂)₀₋₄N(R^{∘})C(S)R^{∘}; —(CH₂)₀₋₄N(R^{∘})C(O)NR^{∘}₂; —(CH₂)₀₋₄N(R^{∘})C(S)NR^{∘}₂; —(CH₂)₀₋₄N(R^{∘})C(O)OR^{∘}; —(CH₂)₀₋₄N(R^{∘})N(R^{∘})C(O)R^{∘}; —(CH₂)₀₋₄N(R^{∘})N(R^{∘})C(O)NR^{∘}₂; —(CH₂)₀₋₄N(R^{∘})N(R^{∘})C(O)OR^{∘}; —(CH₂)₀₋₄C(O)R^{∘}; —(CH₂)₀₋₄C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}—, —(CH₂)₀₋₄SC(S)SR^{∘}; —(CH₂)₀₋₄SC(O)R^{∘}; —(CH₂)₀₋₄C(O)NR^{∘}₂; —(CH₂)₀₋₄C(S)NR^{∘}₂; —(CH₂)₀₋₄C(S)SR^{∘}; —(CH₂)₀₋₄SC(S)SR^{∘}, —(CH₂)₀₋₄OC(O)NR^{∘}₂; —(CH₂)₀₋₄C(O)N(OR^{∘})R^{∘}; —(CH₂)₀₋₄C(O)C(O)R^{∘}; —(CH₂)₀₋₄C(O)CH₂C(O)R^{∘}; —(CH₂)₀₋₄C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —(CH₂)₀₋₄S(O)₂NR^{∘}₂; —(CH₂)₀₋₄S(O)R^{∘}; —(CH₂)₀₋₄N(R^{∘})S(O)₂NR^{∘}₂; —(CH₂)₀₋₄N(R^{∘})S(O)₂R^{∘}; —(CH₂)₀₋₄N(OR^{∘})R^{∘}; —(CH₂)₀₋₄C(NH)NR^{∘}₂; —(CH₂)₀₋₄P(O)₂R^{∘}—(CH₂)₀₋₄P(O)R^{∘}₂; —(CH₂)₀₋₄OP(O)R^{∘}₂; —(CH₂)₀₋₄OP(O)(OR^{∘)}₂; (CH₂)₀₋₄SiR^{∘}₃; —(CH₂)₁₋₄O—N(R^{∘})₂; or —(CH₂)₁₋₄C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂—(5-6 membered heteroaryl ring), or a 5- or 6-membered saturated, partially unsaturated, or aromatic ring having 0-4 heteroatoms independently selected from nitrogen, oxygen, or sulfur, or, notwithstanding the definition above, two independent occurrences of R^{∘}, taken together with their intervening atom(s), form a 3- to 12-membered saturated, partially unsaturated, or aromatic mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, —(CH₂)₀₋₂OH, —(CH₂)₀₋₂OR^{•}, —(CH₂)₀₋₂CH(OR^{•})₂, —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —OSiR^{•}₃, —C(O)SR^{•}, —(C₁₋₄ straight or branched alkylene)C(O)OR^{•}, or —SSW^{•} wherein each R^{•} is unsubstituted or substituted with one or more halogens, and is independently selected from C₁₋₄ aliphatic, —CH₂Ph, —O(CH₂)₀₋₁Ph, or a 5- or 6-membered saturated, partially unsaturated, or aromatic ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR^{*}₂, ═NNHC(O)R^{*}, ═NNHC(O)OR*, ═NNHS(O)₂R^{*}, ═NR^{*}, ═NOR^{*}, —O(C(R^{*}₂))₂₋₃O—, or —S(C(R^{*}₂))₂₋₃S—, wherein each independent occurrence of R^{*} is selected from hydrogen, C₁₋₆ aliphatic which may be substituted as defined below, or an unsubstituted 5- or 6-membered saturated, partially unsaturated, or aromatic ring having 0-4 heteroatoms independently selected from nitrogen, oxygen, or sulfur. Suitable divalent substituents that are bound to vicinal substitutable carbons of an “optionally substituted” group include: —O(CR^{*}₂)₂₋₃O—, wherein each independent occurrence of R^{*} is selected from hydrogen, C₁₋₆ aliphatic which may be substituted as defined below, or an unsubstituted 5- or 6-membered saturated, partially unsaturated, or aromatic ring having 0-4 heteroatoms independently selected from nitrogen, oxygen, or sulfur.

Suitable substituents on the aliphatic group of R^{*} include halogen, —R^{•}, —OH, —OR^{•}, —CN, —C(O)OH, —C(O)OR^{•}, —NH₂, —NHR^{•}, —NR^{•}₂, or —NO₂, wherein each R^{•} is unsubstituted or substituted with one or more halogens, and is independently C₁₋₄ aliphatic, —CH₂Ph, —O(CH₂)₀₋₁Ph, or a 5- or 6-membered saturated, partially unsaturated, or aromatic ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5- or 6-membered saturated, partially unsaturated, or aromatic ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aromatic mono- or bicyclic ring having 0-4 heteroatoms independently selected from nitrogen, oxygen, or sulfur.

Suitable substituents on the aliphatic group of R^{†} are independently halogen, —R^{•}, —OH, —OR^{•}, —CN, —C(O)OH, —C(O)OR^{•}, —NH₂, —NHR^{•}, —NR^{•}₂, or —NO₂, wherein each R^{•} is unsubstituted or substituted with one or more halogens, and is independently C₁₋₄ aliphatic, —CH₂Ph, —O(CH₂)₀₋₁Ph, or a 5- or 6-membered saturated, partially unsaturated, or aromatic ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecyl sulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

### 3. Description of Exemplary Embodiments

In certain embodiments, the present invention provides inhibitors of PDE1. In some embodiments, such compounds include those of formula I:

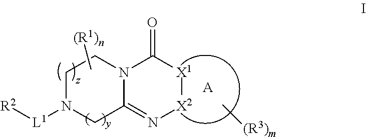

or a pharmaceutically acceptable salt thereof, wherein:
X¹ and X² are each independently C or N;
Ring A is a 5-membered heteroaryl ring having 1-4 heteroatoms independently selected from nitrogen, oxygen and sulfur;
L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —OC(O)—, —C(O)O—, —O—, —S—, —S(O)— or S(O)₂—;
each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R, —S(O)R, or —S(O)₂R;
R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy;
Cy is a ring, substituted with p instances of R⁵; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur;
each R is independently hydrogen, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂;
each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂; or
two R⁵ are taken together with their intervening atoms to form a 5-6 membered saturated, partially unsaturated, or aromatic ring having 0-3 heteroatoms independently selected from nitrogen, oxygen, and sulfur;
wherein one or more of {two instances of R¹}, {R² and an R¹}, and {two instances of R³} may be taken together with their intervening atoms to form a ring, substituted with q instances of R⁵; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur;
   y is 1-2;
   z is 1-2;
   m is 0-3;
   n is 0-3;
   p is 0-5; and
   q is 0-5.

As defined generally above, X¹ and X² are each independently C or N. In some embodiments, X¹ is C, and X² is N. In some embodiments, X¹ is N, and X² is C. In some embodiments both of X¹ and X² are C.

As defined generally above, Ring A is a 5-membered heteroaryl ring having 1-4 heteroatoms independently selected from nitrogen, oxygen and sulfur. In some embodiments, Ring A is pyrrolo. In some embodiments, Ring A is pyrazolo. In some embodiments, Ring A is imidazolo. In some embodiments, Ring A is oxazolo. In some embodiments, Ring A is isoxazolo. In some embodiments, Ring A is triazolo. In some embodiments, Ring A is tetrazolo. In some embodiments, Ring A is selected from pyrazolo and imidazolo. In some embodiments, Ring A is not pyrrolo, furan, thieno, or tetrazolo.

As defined generally above, each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R, —S(O)R, or —S(O)₂R. In certain embodiments, R¹ is hydrogen. In certain embodiments, R¹ is —R wherein —R is not hydrogen. In some embodiments, R¹ is C₁₋₆ aliphatic. In some embodiments, R¹ is methyl. In some embodiments, R¹ is trifluoromethyl. In some embodiments, R¹ is benzyl. In some embodiments, R¹ is phenyl.

As defined generally above, L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —C(O)O—, —OC(O)—, —O—, —S—, —S(O)— or —S(O)₂—. In some embodiments, L¹ is a covalent bond. In some embodiments, L¹ is a C₁₋₄ bivalent straight or branched hydrocarbon chain, wherein one or more hydrogen atoms of the chain are optionally and independently replaced by halogen. In some embodiments, L¹ is a C₁₋₄ bivalent straight or branched hydrocarbon chain, wherein one or two methylene units of the chain are optionally and independently replaced by —N(R)—, —N(R)C(O)—, —C(O)N(R)—, —N(R)S(O)₂—, —S(O)₂N(R)—, —C(O)O—, —OC(O)—, —C(O)—, —O—, —S—, —S(O)— or —S(O)₂—. In some embodiments, L¹ is methylene. In some embodiments, L¹ is —O—. In some embodiments, L¹ is —S—, —S(O)—, or —S(O)₂—.

As defined generally above, R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy. In some embodiments, R² is hydrogen. In some embodiments, R² is not hydrogen. In some embodiments, R² is Cy. In some embodiments, R² is C₁₋₆ aliphatic.

As defined generally above, Cy is a ring, substituted with p instances of R⁵; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur. In some embodiments, said ring of Cy is phenyl. In some embodiments, said ring of Cy is a 5-6 membered monocyclic heteroaromatic ring having 1-4 heteroatoms independently selected from nitrogen, oxygen, and sulfur. In some embodiments, said ring of Cy is a 6-membered monocyclic heteroaromatic ring having 1-2 nitrogens. In some embodiments, said ring of Cy is pyrimidinyl. In some embodiments, said ring of Cy is pyridyl.

As defined generally above, each, R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or —B(OR)₂. In some embodiments, each R³ is —R. In some embodiments, an R³ is an optionally substituted C₁₋₆ aliphatic group. In some embodiments, an R³ is methyl, ethyl, propyl, isopropyl, butyl, sec-buytl, isobutyl, tert-butyl, pentyl, tert-pentyl, neopentyl, isopentyl, sec-pentyl (pentan-2-yl), or 3-pentyl (pentan-3-yl). In some embodiments, an R³ is isopropyl. In some embodiments, an R³ is methyl. In some embodiments, an R³ is propyl. In some embodiments, an R³ is neopentyl. In some embodiments, an R³ is benzyl. In some embodiments, an R³ is an optionally substituted 3-8 membered saturated or partially unsaturated monocyclic carbocyclic ring. In some embodiments, an R³ is optionally substituted cyclopropyl, cyclobutyl, cyclopentyl or cyclohexyl. In some embodiments, an R³ is optionally substituted cyclobutyl or cyclopentyl. In some embodiments, an R³ is optionally substituted phenyl. In some embodiments, an R³ is an optionally substituted 4-8 membered saturated or partially unsaturated monocyclic heterocyclic ring having 1-2 heteroatoms independently selected from nitrogen, oxygen, and sulfur. In some embodiments, an R³ is optionally substituted tetrahydropyranyl or tetrahydrofuranyl. In some embodiments, an R³ is —N(R)₂. In some embodiments, each R³ is the same. In some embodiments, each R³ is different.

As defined generally above, each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)SO₂R, —SO₂N(R)₂, —C(O)R, —C(O)OR, —OC(O)R, —S(O)R, —SO₂R, or —B(OR)₂; or two R⁵ are taken together with their intervening atoms to form a 5-6 membered saturated, partially unsaturated, or aromatic ring having 0-3 heteroatoms independently selected from nitrogen, oxygen, and sulfur. In some embodiments, each R⁵ is independently halogen, —R, —CN, or —OR. In some embodiments, each R⁵ is independently halogen, phenyl, methyl, ethyl, trifluoromethyl, —CN, methoxy, ethoxy, propoxy, or isopropoxy. In some embodiments, p is 1-2 and each R⁵ is independently halogen, phenyl, methyl, ethyl, trifluoromethyl, —CN, methoxy, ethoxy, propoxy, or isopropoxy. In some embodiments p is 2, and one R⁵ is —OR, and the other is halogen. In some embodiments, each R⁵ is the same. In some embodiments, each R⁵ is different. In some embodiments, two R⁵ are taken together with their intervening atoms to form a 5-6 membered saturated, partially unsaturated, or aromatic ring having 0-3 heteroatoms independently selected from nitrogen, oxygen, and sulfur. In some embodiments, two R⁵ are taken together with their intervening atoms to form a dioxolo ring.

As defined generally above one or more of {two instances of R¹}, {R² and an R¹}, and {two instances of R³} may be taken together with their intervening atoms to form a ring, substituted with q instances of R⁵; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; or a 5-6 membered monocyclic heteroaromatic ring having 1-4 heteroatoms independently selected from nitrogen, oxygen, and sulfur. In some embodiments, none of {two instances of R¹}, {R² and an R¹}, and {two instances of R³} are taken together with their intervening atoms to form a ring. In some embodiments, one of {two instances of R¹}, {R² and an R¹}, and {two instances of R³} is taken together with their intervening atoms to form a ring substituted with q instances of R⁵. In some embodiments, two of {two instances of R¹}, {R² and an R¹}, and {two instances of R³} are taken together with their intervening atoms to form a ring substituted with q instances of R⁵. In some embodiments, two instances of R¹ are taken together with their intervening atoms to form a ring substituted with q instances of R⁵. In some embodiments, R² and an R¹ are taken together with their intervening atoms to form a ring substituted with q instances of R⁵. In some embodiments, two instances of R³ are taken together with their intervening atoms to form a ring substituted with q instances of R⁵.

As defined generally above, y is 1-2 and z is 1-2. In some embodiments, both y and z are not simultaneously 2. In some embodiments, y is 1 and z is 1. In some embodiments, y is 1 and z is 2. In some embodiments, y is 2 and z is 1.

As defined generally above, m is 0-3. In some embodiments, m is 0. In some embodiments, m is 1-2. In some embodiments, m is 1. In some embodiments, m is 2.

As defined generally above, n is 0-3. In some embodiments, n is 0. In some embodiments, n is 1-2. In some embodiments, n is 1. In some embodiments, n is 2.

As defined generally above, p is 0-5. In some embodiments, p is 0. In some embodiments, p is 1-5. In some embodiments, p is 1-2. In some embodiments, p is 1. In some embodiments, p is 2. In some embodiments, p is 3.

As defined generally above, q is 0-5. In some embodiments, q is 0. In some embodiments, q is 1-5. In some embodiments, q is 1-2. In some embodiments, q is 1. In some embodiments, q is 2. In some embodiments, q is 3.

In some embodiments, the present invention provides a compound of formula I selected from formulas I-a, I-b, and I-c:

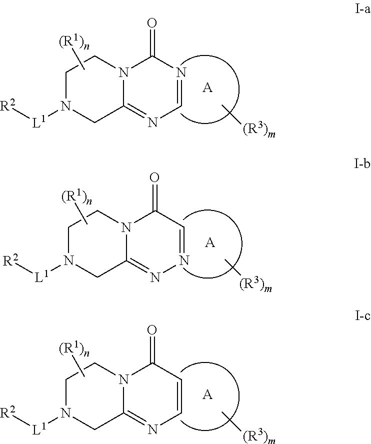

or a pharmaceutically acceptable salt thereof; wherein each of L¹, Ring A, R¹, R², R³, n, and m is as described in embodiments for formula I, supra, or described in embodiments herein, both singly and in combination.

In certain embodiments, the present invention provides a compound of formula I selected from formulas II-a, II-b, II-c, II-d, II-e, II-f, II-g, II-h, II-i, II-j and II-k:

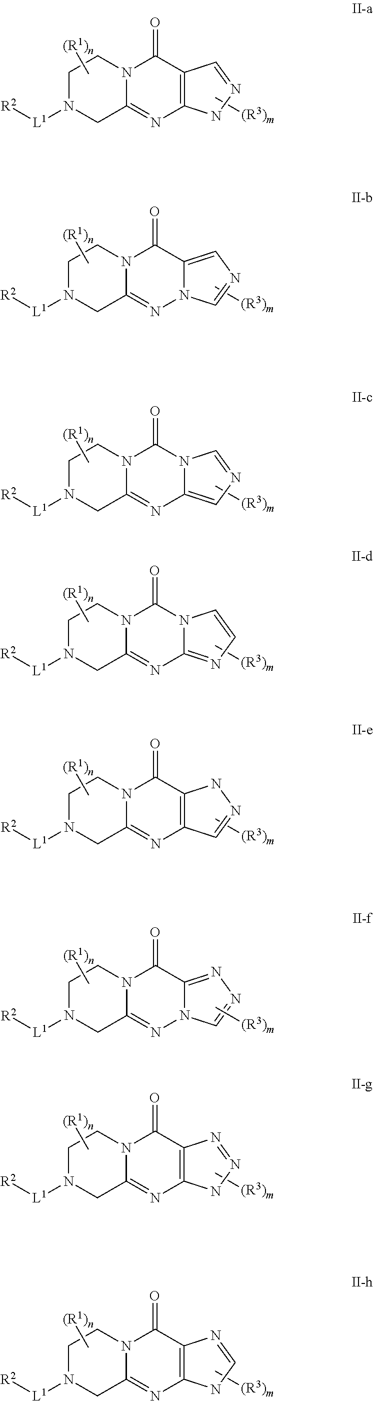

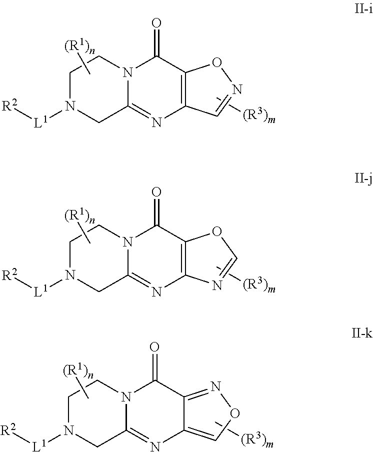

or a pharmaceutically acceptable salt thereof, wherein:
each of R¹, R², R³, n, and m is as described in embodiments for formula I, supra, or described in embodiments herein, both singly and in combination.

In certain embodiments, the present invention provides a compound selected from formulas II-a, II-b, II-c, II-d, II-e, II-f, II-g, II-h, II-i, II-j and II-k, wherein L¹ is a covalent bond, or a pharmaceutically acceptable salt thereof. In certain embodiments, the present invention provides a compound selected from formulas II-a, II-b, II-c, II-d, II-e, II-f, II-g, H-h, II-i, II-j, and II-k, wherein L¹ is a covalent bond, n is 1, and R¹ is not hydrogen, or a pharmaceutically acceptable salt thereof. In certain embodiments, the present invention provides a compound selected from formulas II-a, II-b, II-c, II-d, II-e, II-f, II-g, II-h, II-j, and II-k, wherein L¹ is a covalent bond, n is 1, and R¹ is optionally substituted C₁₋₆ aliphatic or optionally substituted phenyl, or a pharmaceutically acceptable salt thereof. In certain embodiments, the present invention provides a compound selected from formulas II-a, II-b, II-c, II-d, II-e, II-f, II-g, II-h, II-i, II-j, and II-k, wherein L¹ is a covalent bond, R² is hydrogen, n is 1, and R¹ is not hydrogen, or a pharmaceutically acceptable salt thereof. In certain embodiments, the present invention provides a compound selected from formulas II-a, II-b, II-c, II-d, II-e, II-f, II-g, II-h, II-i, II-j, and II-k, wherein L¹ is a covalent bond, R² is hydrogen, n is 1, and R¹ is optionally substituted C₁₋₆ aliphatic or optionally substituted phenyl, or a pharmaceutically acceptable salt thereof. In certain embodiments, the present invention provides a compound selected from formulas II-a, II-b, II-c, II-d, II-e, II-f, II-g, II-h, II-i, II-j and II-k, wherein L¹ is methylene, or a pharmaceutically acceptable salt thereof. In certain embodiments, the present invention provides a compound selected from formulas II-a, II-b, II-c, II-d, II-e, II-f, II-g, II-h, II-i, II-j and II-k, wherein L¹ is a covalent bond and R² is Cy, or a pharmaceutically acceptable salt thereof. In certain embodiments, the present invention provides a compound selected from formulas II-a, II-b, II-c, II-d, II-e, II-f, II-g, II-h, II-i, II-j and II-k, wherein L¹ is methylene and R² is Cy, or a pharmaceutically acceptable salt thereof.

In certain embodiments, the present invention provides a compound selected from formulas II-a, II-b, II-c, II-d, II-e, II-f, II-g, II-h, II-i, II-j and II-k, wherein L¹ is a covalent bond or methylene, R² is hydrogen, n is 0, and m is 1-2, or a pharmaceutically acceptable salt thereof. In certain embodiments, the present invention provides a compound selected from formulas II-a, II-b, II-c, II-d, II-e, II-f, II-g, II-h, II-i, II-j and II-k, wherein L¹ is a covalent bond or methylene, R² is Cy, n is 0, and m is 1-2, or a pharmaceutically acceptable salt thereof.

In certain embodiments, the present invention provides a compound of formula I selected from formulas III-a, III-b, III-c, III-d, III-e, III-f, III-g, III-h, III-i, III-j and III-k:

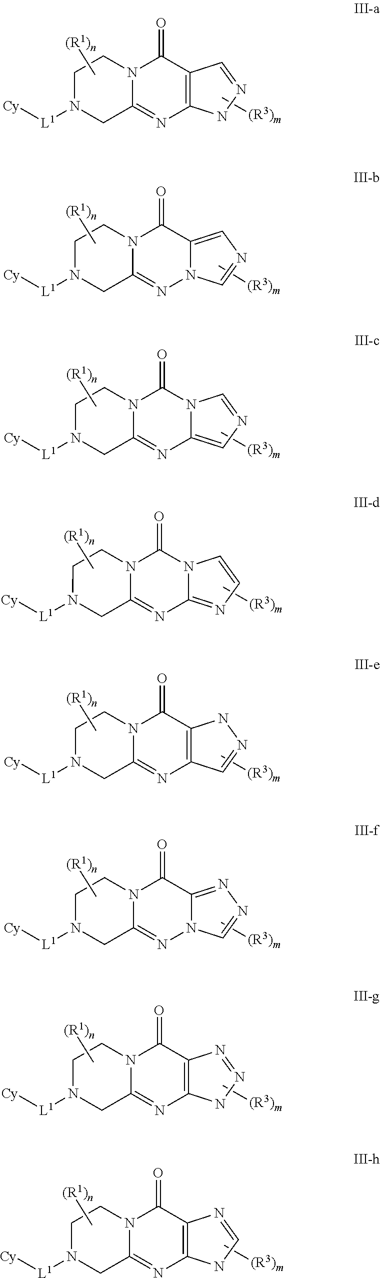

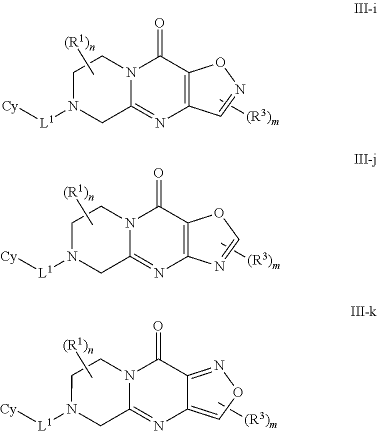

or a pharmaceutically acceptable salt thereof, wherein:
each of L¹, R¹, R³, Cy, n, and m is as described in embodiments for formula I, supra, or described in embodiments herein, both singly and in combination.

In certain embodiments, the present invention provides a compound of one of formulas III-a, III-b, III-c, III-d, III-e, III-f, III-g, III-h, III-i, III-j and III-k, wherein L¹ is a covalent bond, or a pharmaceutically acceptable salt thereof. In certain embodiments, the present invention provides a compound of one of formulas III-a, III-b, III-c, III-d, III-e, III-f, III-g, III-h, III-i, III-j and III-k, wherein L¹ is a covalent bond, and Cy is phenyl substituted with p instances of R⁵, or a pharmaceutically acceptable salt thereof. In certain embodiments, the present invention provides a compound of one of formulas III-a, III-b, III-c, III-d, III-e, III-f, III-g, III-h, III-i, III-j and III-k, wherein L¹ is a covalent bond, Cy is phenyl substituted with p instances of R³, and p is 0-2, or a pharmaceutically acceptable salt thereof. In certain embodiments, the present invention provides a compound of one of formulas III-a, III-b, III-c, III-d, III-e, III-f, III-g, III-h, III-i, III-j and III-k, wherein L¹ is a covalent bond, and m is 1, or a pharmaceutically acceptable salt thereof.

In certain embodiments, the present invention provides a compound of one of formulas III-a, III-b, III-c, III-d, III-e, III-f, III-g, III-h, III-i, III-j and III-k, wherein L¹ is methylene, or a pharmaceutically acceptable salt thereof. In certain embodiments, the present invention provides a compound of one of formulas III-a, III-b, III-c, III-d, III-e, III-f, III-g, III-h, III-i, III-j and III-k, wherein L¹ is methylene, and Cy is phenyl substituted with p instances of R⁵, or a pharmaceutically acceptable salt thereof. In certain embodiments, the present invention provides a compound of one of formulas III-a, III-b, III-c, III-d, III-e, III-f, III-g, III-h, III-i, III-j and III-k, wherein L¹ is methylene, Cy is phenyl substituted with p instances of R⁵, and p is 0-2, or a pharmaceutically acceptable salt thereof. In certain embodiments, the present invention provides a compound of one of formulas III-a, III-b, III-c, III-d, III-e, III-f, III-g, III-h, III-i, III-j and III-k, wherein L¹ is methylene, and m is 1, or a pharmaceutically acceptable salt thereof.

In certain embodiments, the present invention provides a compound of formula I of formula IV:

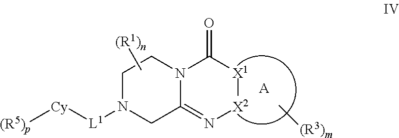

or a pharmaceutically acceptable salt thereof, wherein:
each of X¹, X², Ring A, L¹, R¹, R³, R⁵, Cy, n, m, and p is as described in embodiments for formula I, supra, or described in embodiments herein, both singly and in combination.

In certain embodiments, the present invention provides a compound of formula IV, wherein L¹ is a covalent bond or methylene, or a pharmaceutically acceptable salt thereof. In certain embodiments, the present invention provides a compound of formula IV, wherein L¹ is a covalent bond, or a pharmaceutically acceptable salt thereof. In certain embodiments, the present invention provides a compound of formula IV, wherein L¹ is methylene, or a pharmaceutically acceptable salt thereof.

In certain embodiments, the present invention provides a compound of formula IV, wherein X¹ is N, and X² is C, or a pharmaceutically acceptable salt thereof. In certain embodiments, the present invention provides a compound of formula IV, wherein X¹ is C, and X² is N, or a pharmaceutically acceptable salt thereof. In certain embodiments, the present invention provides a compound of formula IV, wherein both X¹ and X² are C, or a pharmaceutically acceptable salt thereof.

In certain embodiments, the present invention provides a compound of formula IV, wherein X¹ is N, X² is C, m is 1, and p is 1-5, or a pharmaceutically acceptable salt thereof. In certain embodiments, the present invention provides a compound of formula IV, wherein X¹ is C, X² is N, m is 1, and p is 1-5, or a pharmaceutically acceptable salt thereof. In certain embodiments, the present invention provides a compound of formula IV, wherein both X¹ and X² are C, m is 1, and p is 1-5, or a pharmaceutically acceptable salt thereof.

In certain embodiments, the present invention provides a compound of formula IV selected from formulas V-a, V-b, V-c, V-d, V-e, V-f, V-g, V-h, and V-i:

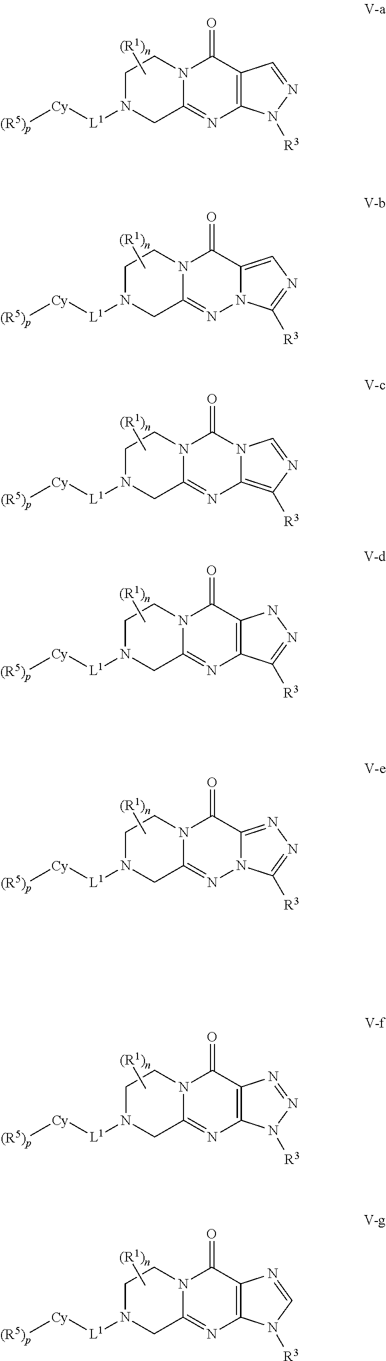

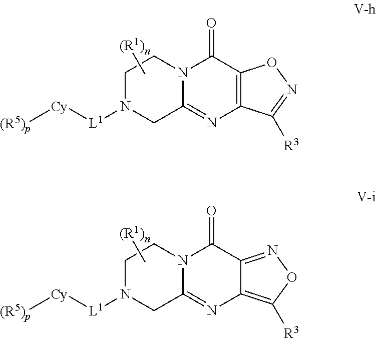

or a pharmaceutically acceptable salt thereof, wherein:
each of L¹, R¹, R³, R⁵, Cy, n, m, and p is as described in embodiments for formula I, supra, or described in embodiments herein, both singly and in combination. In certain embodiments, the present invention provides a compound of one of formulas V-a, V-b, V-c, V-d, V-e, V-f, V-g, V-h, and V-i, wherein L¹ is a covalent bond, or a pharmaceutically acceptable salt thereof. In certain embodiments, the present invention provides a compound of one of formulas V-a, V-b, V-c, V-d, V-e, V-f, V-g, V-h, and V-i, wherein L¹ is a covalent bond, Cy is phenyl, and p is 0-2, or a pharmaceutically acceptable salt thereof. In certain embodiments, the present invention provides a compound of one of formulas V-a, V-b, V-c, V-d, V-e, V-f, V-g, V-h, and V-i, wherein is a covalent bond, and R³ is optionally substituted C₁₋₆ aliphatic or optionally substituted phenyl, or a pharmaceutically acceptable salt thereof. In certain embodiments, the present invention provides a compound of one of formulas V-a, V-b, V-c, V-d, V-e, V-f, V-g, V-h, and V-i, wherein L¹ is methylene, or a pharmaceutically acceptable salt thereof. In certain embodiments, the present invention provides a compound of one of formulas V-a, V-b, V-c, V-d, V-e, V-f, V-g, V-h, and V-i, wherein L¹ is methylene, Cy is phenyl, and p is 0-2, or a pharmaceutically acceptable salt thereof. In certain embodiments, the present invention provides a compound of one of formulas V-a, V-b, V-c, V-d, V-e, V-f, V-g, V-h, and V-i, wherein L¹ is methylene, and R³ is optionally substituted C₁₋₆ aliphatic or optionally substituted phenyl, or a pharmaceutically acceptable salt thereof.

In certain embodiments, the present invention provides a compound of formula I of formulas VI-a or VI-b:

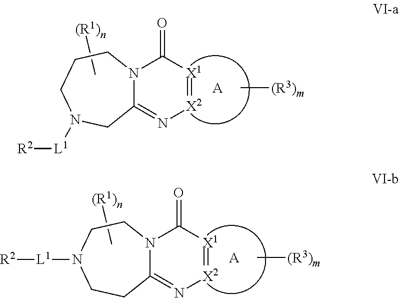

or a pharmaceutically acceptable salt thereof, wherein:
each of X¹, X², L¹, Ring A, R¹, R², R³, n, and m, is as described in embodiments for formula I, supra, or described in embodiments herein, both singly and in combination. In certain embodiments, the present invention provides a compound of one of formulas VI-a and VI-b, wherein L¹ is a covalent bond, or a pharmaceutically acceptable salt thereof. In certain embodiments, the present invention provides a compound of one of formulas VI-a and VI-b, wherein L¹ is methylene, or a pharmaceutically acceptable salt thereof.

In some embodiments, compounds of the invention are not selected from the following:

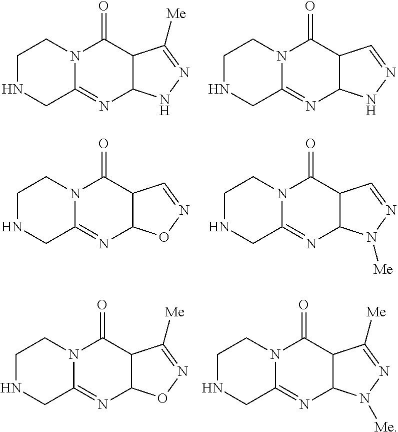

Exemplary compounds of formula I are set forth in Table 1, below:

TABLE 1
Exemplary Compounds of Formula I

| Compound | | Structure | |
|---|---|---|---|
| 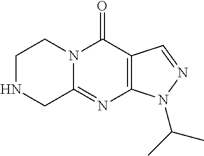 [SMILES: CC(C)n1ncc2c(=O)n3c(nc21)CNCC3] | I-1 | 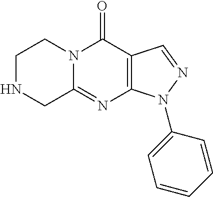 [SMILES: O=c1c2cnn(-c3ccccc3)c2nc2n1CCNC2] | I-2 |
| 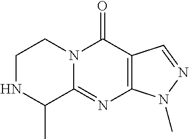 [SMILES: CC1NCCn2c1nc1c(cnn1C)c2=O] | I-3 | 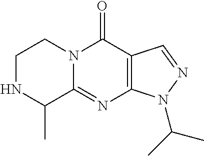 [SMILES: CC1NCCn2c1nc1c(cnn1C(C)C)c2=O] | I-4 |
| 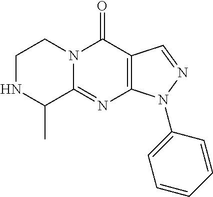 [SMILES: CC1NCCn2c1nc1c(cnn1-c1ccccc1)c2=O] | I-5 | 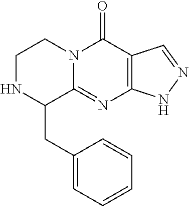 [SMILES: O=c1c2cn[nH]c2nc2n1CCNC2Cc1ccccc1] | I-6 |
| 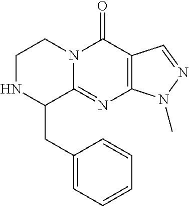 [SMILES: Cn1ncc2c(=O)n3c(nc21)C(Cc1ccccc1)NCC3] | I-7 | 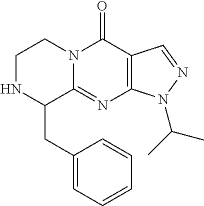 [SMILES: CC(C)n1ncc2c(=O)n3c(nc21)C(Cc1ccccc1)NCC3] | I-8 |
| 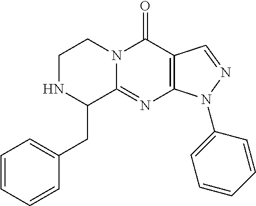 [SMILES: O=c1c2cnn(-c3ccccc3)c2nc2n1CCNC2Cc1ccccc1] | I-9 | 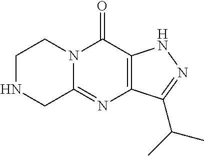 [SMILES: CC(C)c1n[nH]c2c(=O)n3c(nc12)CNCC3] | I-10 |
| 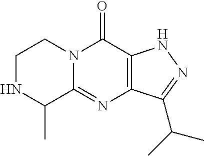 [SMILES: CC(C)c1n[nH]c2c(=O)n3c(nc12)C(C)NCC3] | I-11 | 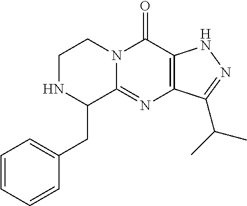 [SMILES: CC(C)c1n[nH]c2c(=O)n3c(nc12)C(Cc1ccccc1)NCC3] | I-12 |
| 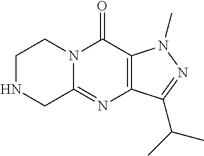 [SMILES: CC(C)c1nn(C)c2c(=O)n3c(nc12)CNCC3] | I-13 | 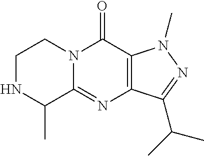 [SMILES: CC(C)c1nn(C)c2c(=O)n3c(nc12)C(C)NCC3] | I-14 |
| 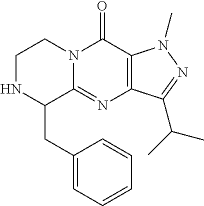 [SMILES: CC(C)c1nn(C)c2c(=O)n3c(nc12)C(Cc1ccccc1)NCC3] | I-15 | 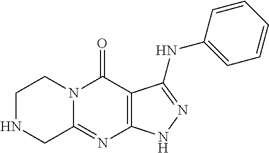 [SMILES: O=c1c2c(Nc3ccccc3)n[nH]c2nc2n1CCNC2] | I-16 |
| 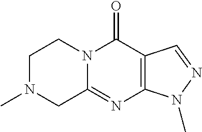 [SMILES: CN1CCn2c(nc3c(cnn3C)c2=O)C1] | I-17 | 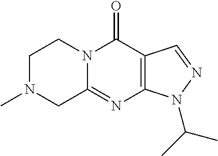 [SMILES: CC(C)n1ncc2c(=O)n3c(nc21)CN(C)CC3] | I-18 |
| 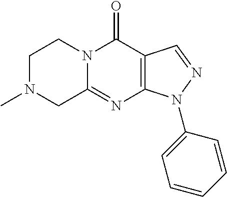 [SMILES: CN1CCn2c(nc3c(cnn3-c3ccccc3)c2=O)C1] | I-19 | 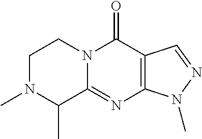 [SMILES: CC1c2nc3c(cnn3C)c(=O)n2CCN1C] | I-20 |
| 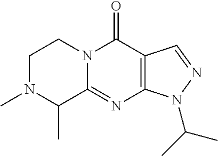 [SMILES: CC1c2nc3c(cnn3C(C)C)c(=O)n2CCN1C] | I-21 | 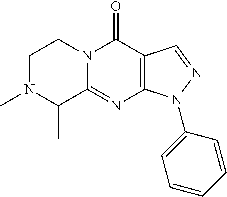 [SMILES: CC1c2nc3c(cnn3-c3ccccc3)c(=O)n2CCN1C] | I-22 |
| 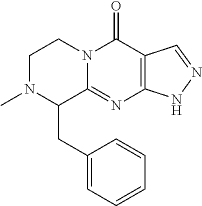 [SMILES: CN1CCn2c(nc3[nH]ncc3c2=O)C1Cc1ccccc1] | I-23 | 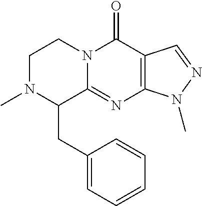 [SMILES: CN1CCn2c(nc3c(cnn3C)c2=O)C1Cc1ccccc1] | I-24 |
| 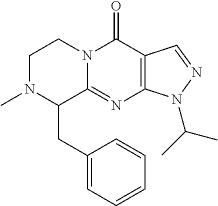 [SMILES: CC(C)n1ncc2c(=O)n3c(nc21)C(Cc1ccccc1)N(C)CC3] | I-25 | 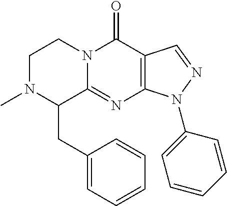 [SMILES: CN1CCn2c(nc3c(cnn3-c3ccccc3)c2=O)C1Cc1ccccc1] | I-26 |
| 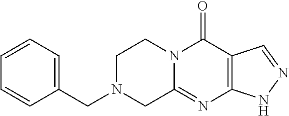 [SMILES: O=c1c2cn[nH]c2nc2n1CCN(Cc1ccccc1)C2] | I-27 | 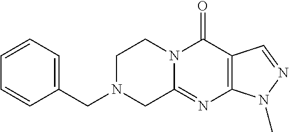 [SMILES: Cn1ncc2c(=O)n3c(nc21)CN(Cc1ccccc1)CC3] | I-28 |
| 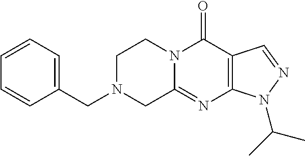 [SMILES: CC(C)n1ncc2c(=O)n3c(nc21)CN(Cc1ccccc1)CC3] | I-29 | 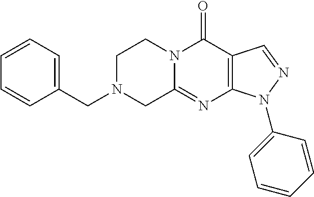 [SMILES: O=c1c2cnn(-c3ccccc3)c2nc2n1CCN(Cc1ccccc1)C2] | I-30 |
| 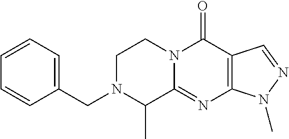 [SMILES: CC1c2nc3c(cnn3C)c(=O)n2CCN1Cc1ccccc1] | I-31 | 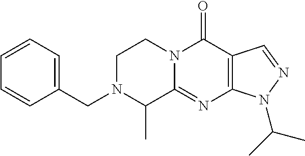 [SMILES: CC1c2nc3c(cnn3C(C)C)c(=O)n2CCN1Cc1ccccc1] | I-32 |
| 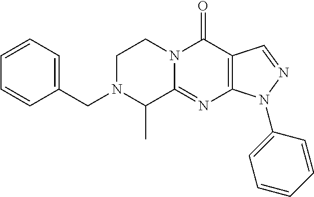 [SMILES: CC1c2nc3c(cnn3-c3ccccc3)c(=O)n2CCN1Cc1ccccc1] | I-33 | 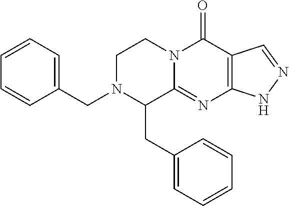 [SMILES: O=c1c2cn[nH]c2nc2n1CCN(Cc1ccccc1)C2Cc1ccccc1] | I-34 |
| 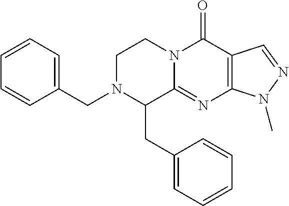 [SMILES: Cn1ncc2c(=O)n3c(nc21)C(Cc1ccccc1)N(Cc1ccccc1)CC3] | I-35 | 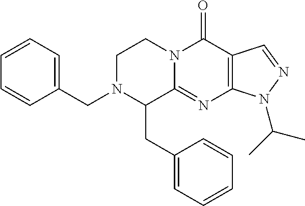 [SMILES: CC(C)n1ncc2c(=O)n3c(nc21)C(Cc1ccccc1)N(Cc1ccccc1)CC3] | I-36 |
| 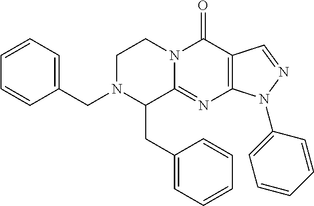 [SMILES: O=c1c2cnn(-c3ccccc3)c2nc2n1CCN(Cc1ccccc1)C2Cc1ccccc1] | I-37 | 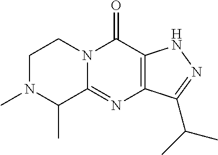 [SMILES: CC(C)c1n[nH]c2c(=O)n3c(nc12)C(C)N(C)CC3] | I-38 |
| 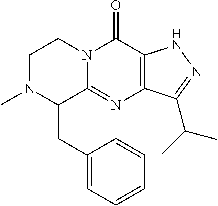 [SMILES: CC(C)c1n[nH]c2c(=O)n3c(nc12)C(Cc1ccccc1)N(C)CC3] | I-39 | 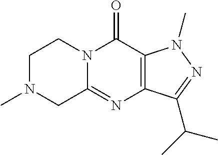 [SMILES: CC(C)c1nn(C)c2c(=O)n3c(nc12)CN(C)CC3] | I-40 |
| 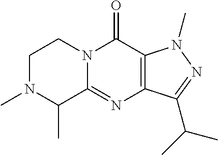 [SMILES: CC(C)c1nn(C)c2c(=O)n3c(nc12)C(C)N(C)CC3] | I-41 | 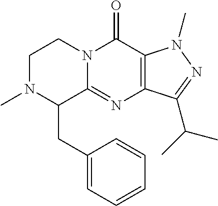 [SMILES: CC(C)c1nn(C)c2c(=O)n3c(nc12)C(Cc1ccccc1)N(C)CC3] | I-42 |
| 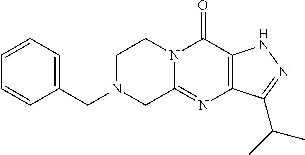 [SMILES: CC(C)c1n[nH]c2c(=O)n3c(nc12)CN(Cc1ccccc1)CC3] | I-43 | 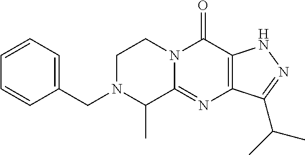 [SMILES: CC(C)c1n[nH]c2c(=O)n3c(nc12)C(C)N(Cc1ccccc1)CC3] | I-44 |
| 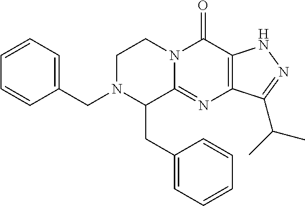 [SMILES: CC(C)c1n[nH]c2c(=O)n3c(nc12)C(Cc1ccccc1)N(Cc1ccccc1)CC3] | I-45 | 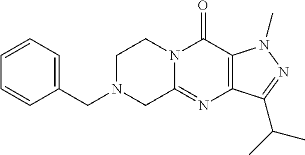 [SMILES: CC(C)c1nn(C)c2c(=O)n3c(nc12)CN(Cc1ccccc1)CC3] | I-46 |
| 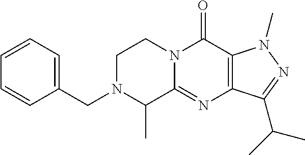 [SMILES: CC(C)c1nn(C)c2c(=O)n3c(nc12)C(C)N(Cc1ccccc1)CC3] | I-47 | 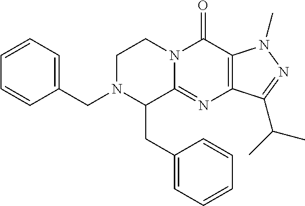 [SMILES: CC(C)c1nn(C)c2c(=O)n3c(nc12)C(Cc1ccccc1)N(Cc1ccccc1)CC3] | I-48 |
| 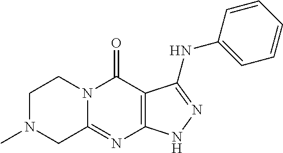 [SMILES: CN1CCn2c(nc3[nH]nc(Nc4ccccc4)c3c2=O)C1] | I-49 | 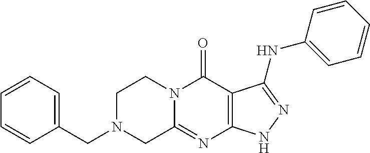 [SMILES: O=c1c2c(Nc3ccccc3)n[nH]c2nc2n1CCN(Cc1ccccc1)C2] | I-50 |
| 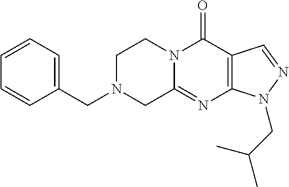 [SMILES: CC(C)Cn1ncc2c(=O)n3c(nc21)CN(Cc1ccccc1)CC3] | I-51 | 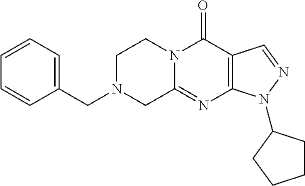 [SMILES: O=c1c2cnn(C3CCCC3)c2nc2n1CCN(Cc1ccccc1)C2] | I-52 |
| 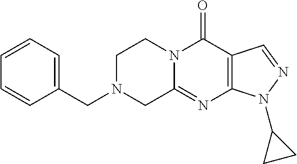 [SMILES: O=c1c2cnn(C3CC3)c2nc2n1CCN(Cc1ccccc1)C2] | I-53 | 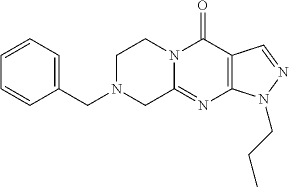 [SMILES: CCCn1ncc2c(=O)n3c(nc21)CN(Cc1ccccc1)CC3] | I-54 |
| 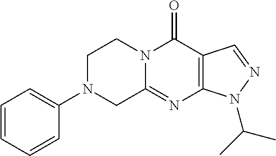 [SMILES: CC(C)n1ncc2c(=O)n3c(nc21)CN(c1ccccc1)CC3] | I-55 | 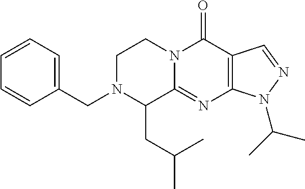 [SMILES: CC(C)CC1c2nc3c(cnn3C(C)C)c(=O)n2CCN1Cc1ccccc1] | I-56 |
| 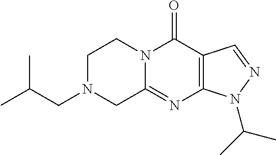 [SMILES: CC(C)CN1CCn2c(nc3c(cnn3C(C)C)c2=O)C1] | I-57 | 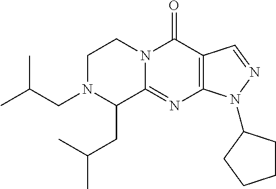 [SMILES: CC(C)CC1c2nc3c(cnn3C3CCCC3)c(=O)n2CCN1CC(C)C] | I-58 |
| 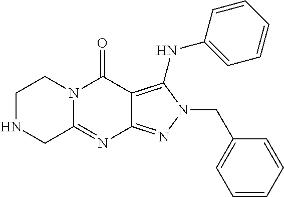 [SMILES: O=c1c2c(Nc3ccccc3)n(Cc3ccccc3)nc2nc2n1CCNC2] | I-59 | 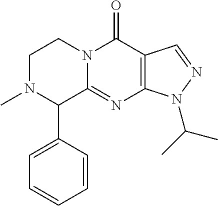 [SMILES: CC(C)n1ncc2c(=O)n3c(nc21)C(c1ccccc1)N(C)CC3] | I-60 |
| 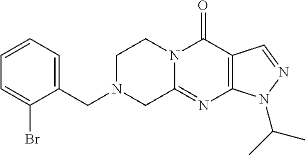 [SMILES: CC(C)n1ncc2c(=O)n3c(nc21)CN(Cc1ccccc1Br)CC3] | I-61 | 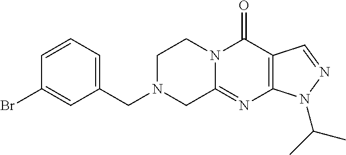 [SMILES: CC(C)n1ncc2c(=O)n3c(nc21)CN(Cc1cccc(Br)c1)CC3] | I-62 |
| 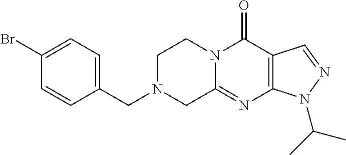 [SMILES: CC(C)n1ncc2c(=O)n3c(nc21)CN(Cc1ccc(Br)cc1)CC3] | I-63 | 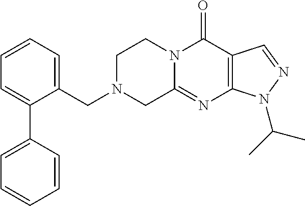 [SMILES: CC(C)n1ncc2c(=O)n3c(nc21)CN(Cc1ccccc1-c1ccccc1)CC3] | I-64 |
| 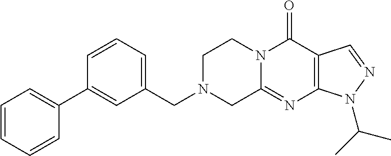 [SMILES: CC(C)n1ncc2c(=O)n3c(nc21)CN(Cc1cccc(-c2ccccc2)c1)CC3] | I-65 | 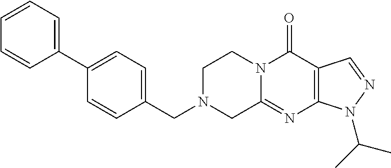 [SMILES: CC(C)n1ncc2c(=O)n3c(nc21)CN(Cc1ccc(-c2ccccc2)cc1)CC3] | I-66 |
| 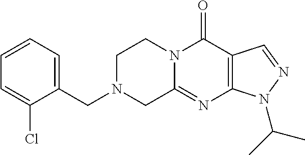 [SMILES: CC(C)n1ncc2c(=O)n3c(nc21)CN(Cc1ccccc1Cl)CC3] | I-67 | 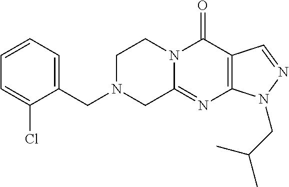 [SMILES: CC(C)Cn1ncc2c(=O)n3c(nc21)CN(Cc1ccccc1Cl)CC3] | I-68 |
| 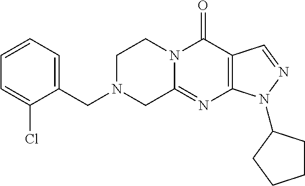 [SMILES: O=c1c2cnn(C3CCCC3)c2nc2n1CCN(Cc1ccccc1Cl)C2] | I-69 | 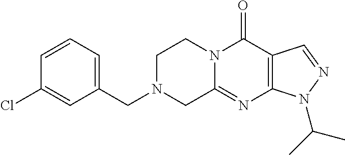 [SMILES: CC(C)n1ncc2c(=O)n3c(nc21)CN(Cc1cccc(Cl)c1)CC3] | I-70 |
| 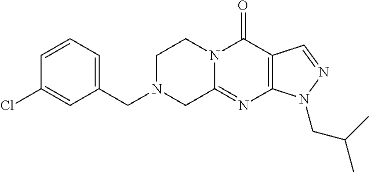 [SMILES: CC(C)Cn1ncc2c(=O)n3c(nc21)CN(Cc1cccc(Cl)c1)CC3] | I-71 | 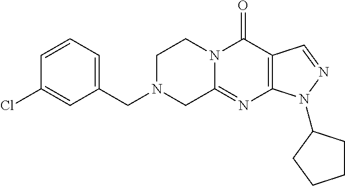 [SMILES: O=c1c2cnn(C3CCCC3)c2nc2n1CCN(Cc1cccc(Cl)c1)C2] | I-72 |
| 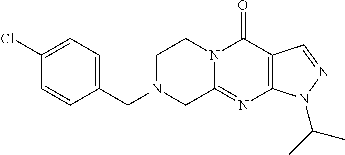 [SMILES: CC(C)n1ncc2c(=O)n3c(nc21)CN(Cc1ccc(Cl)cc1)CC3] | I-73 | 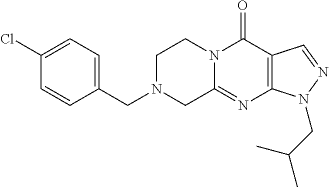 [SMILES: CC(C)Cn1ncc2c(=O)n3c(nc21)CN(Cc1ccc(Cl)cc1)CC3] | I-74 |
| 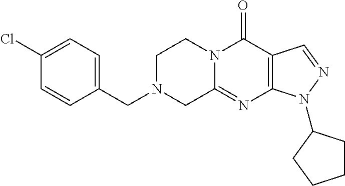 [SMILES: O=c1c2cnn(C3CCCC3)c2nc2n1CCN(Cc1ccc(Cl)cc1)C2] | I-75 | 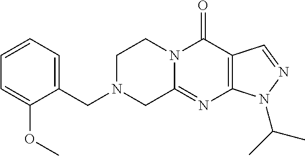 [SMILES: COc1ccccc1CN1CCn2c(nc3c(cnn3C(C)C)c2=O)C1] | I-76 |
| 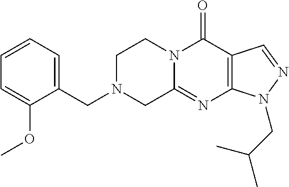 [SMILES: COc1ccccc1CN1CCn2c(nc3c(cnn3CC(C)C)c2=O)C1] | I-77 | 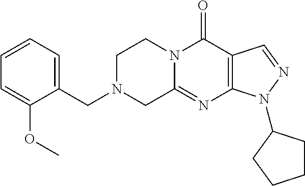 [SMILES: COc1ccccc1CN1CCn2c(nc3c(cnn3C3CCCC3)c2=O)C1] | I-78 |
| 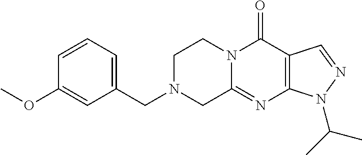 [SMILES: COc1cccc(CN2CCn3c(nc4c(cnn4C(C)C)c3=O)C2)c1] | I-79 | 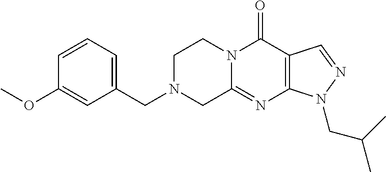 [SMILES: COc1cccc(CN2CCn3c(nc4c(cnn4CC(C)C)c3=O)C2)c1] | I-80 |
| 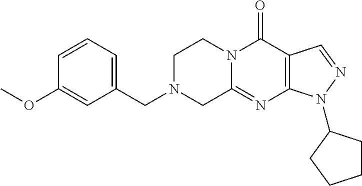 [SMILES: COc1cccc(CN2CCn3c(nc4c(cnn4C4CCCC4)c3=O)C2)c1] | I-81 | 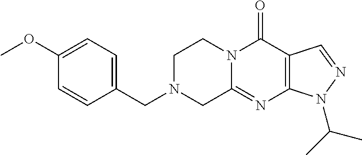 [SMILES: COc1ccc(CN2CCn3c(nc4c(cnn4C(C)C)c3=O)C2)cc1] | I-82 |
| 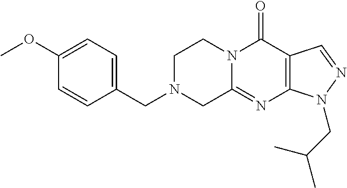 [SMILES: COc1ccc(CN2CCn3c(nc4c(cnn4CC(C)C)c3=O)C2)cc1] | I-83 | 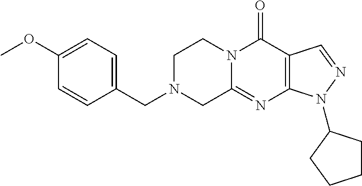 [SMILES: COc1ccc(CN2CCn3c(nc4c(cnn4C4CCCC4)c3=O)C2)cc1] | I-84 |
| 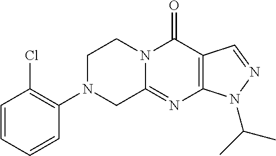 [SMILES: CC(C)n1ncc2c(=O)n3c(nc21)CN(c1ccccc1Cl)CC3] | I-85 | 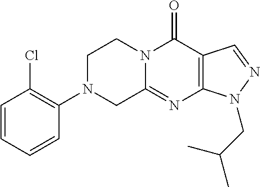 [SMILES: CC(C)Cn1ncc2c(=O)n3c(nc21)CN(c1ccccc1Cl)CC3] | I-86 |
| 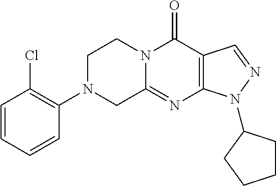 [SMILES: O=c1c2cnn(C3CCCC3)c2nc2n1CCN(c1ccccc1Cl)C2] | I-87 | 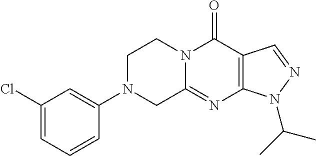 [SMILES: CC(C)n1ncc2c(=O)n3c(nc21)CN(c1cccc(Cl)c1)CC3] | I-88 |
| 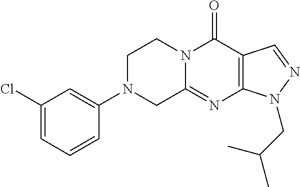 [SMILES: CC(C)Cn1ncc2c(=O)n3c(nc21)CN(c1cccc(Cl)c1)CC3] | I-89 | 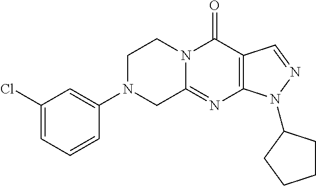 [SMILES: O=c1c2cnn(C3CCCC3)c2nc2n1CCN(c1cccc(Cl)c1)C2] | I-90 |
| 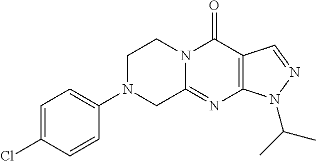 [SMILES: CC(C)n1ncc2c(=O)n3c(nc21)CN(c1ccc(Cl)cc1)CC3] | I-91 | 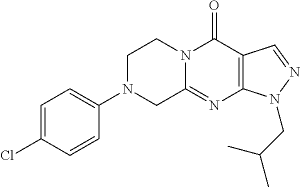 [SMILES: CC(C)Cn1ncc2c(=O)n3c(nc21)CN(c1ccc(Cl)cc1)CC3] | I-92 |
| 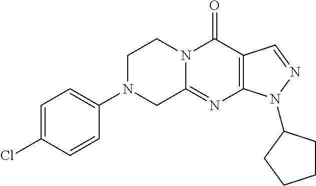 [SMILES: O=c1c2cnn(C3CCCC3)c2nc2n1CCN(c1ccc(Cl)cc1)C2] | I-93 | 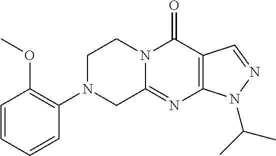 [SMILES: COc1ccccc1N1CCn2c(nc3c(cnn3C(C)C)c2=O)C1] | I-94 |
| 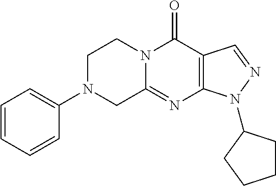 [SMILES: O=c1c2cnn(C3CCCC3)c2nc2n1CCN(c1ccccc1)C2] | I-95 | 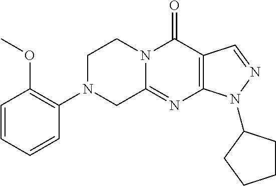 [SMILES: COc1ccccc1N1CCn2c(nc3c(cnn3C3CCCC3)c2=O)C1] | I-96 |
| 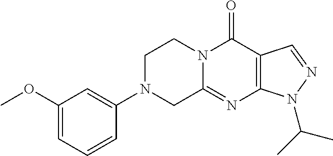 [SMILES: COc1cccc(N2CCn3c(nc4c(cnn4C(C)C)c3=O)C2)c1] | I-97 | 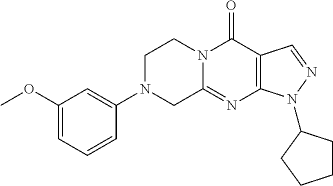 [SMILES: COc1cccc(N2CCn3c(nc4c(cnn4C4CCCC4)c3=O)C2)c1] | I-98 |
| 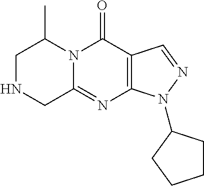 [SMILES: CC1CNCc2nc3c(cnn3C3CCCC3)c(=O)n21] | I-99 | 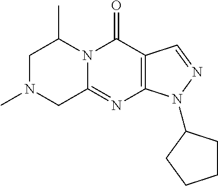 [SMILES: CC1CN(C)Cc2nc3c(cnn3C3CCCC3)c(=O)n21] | I-100 |
| 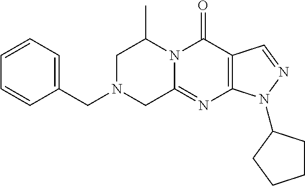 [SMILES: CC1CN(Cc2ccccc2)Cc2nc3c(cnn3C3CCCC3)c(=O)n21] | I-101 | 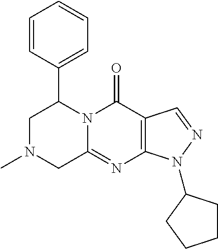 [SMILES: CN1Cc2nc3c(cnn3C3CCCC3)c(=O)n2C(c2ccccc2)C1] | I-102 |
| 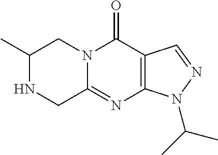 [SMILES: CC1Cn2c(nc3c(cnn3C(C)C)c2=O)CN1] | I-103 | 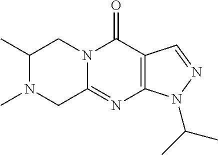 [SMILES: CC1Cn2c(nc3c(cnn3C(C)C)c2=O)CN1C] | I-104 |
| 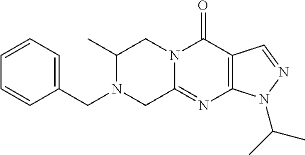 [SMILES: CC1Cn2c(nc3c(cnn3C(C)C)c2=O)CN1Cc1ccccc1] | I-105 | 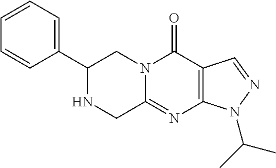 [SMILES: CC(C)n1ncc2c(=O)n3c(nc21)CNC(c1ccccc1)C3] | I-106 |
| 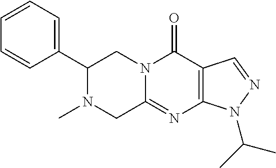 [SMILES: CC(C)n1ncc2c(=O)n3c(nc21)CN(C)C(c1ccccc1)C3] | I-107 | 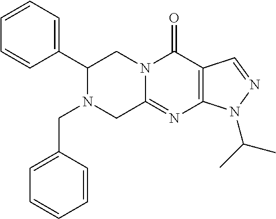 [SMILES: CC(C)n1ncc2c(=O)n3c(nc21)CN(Cc1ccccc1)C(c1ccccc1)C3] | I-108 |
| 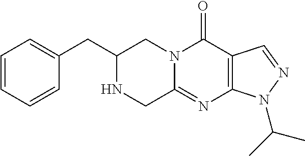 [SMILES: CC(C)n1ncc2c(=O)n3c(nc21)CNC(Cc1ccccc1)C3] | I-109 | 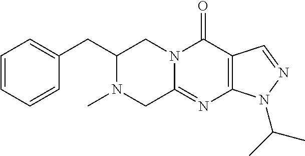 [SMILES: CC(C)n1ncc2c(=O)n3c(nc21)CN(C)C(Cc1ccccc1)C3] | I-110 |
| 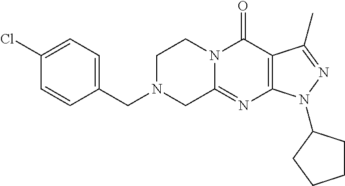 [SMILES: Cc1nn(C2CCCC2)c2nc3n(c(=O)c12)CCN(Cc1ccc(Cl)cc1)C3] | I-111 | 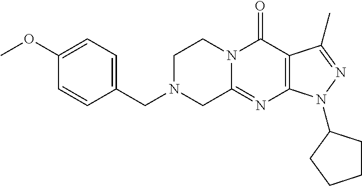 [SMILES: COc1ccc(CN2CCn3c(nc4c(c(C)nn4C4CCCC4)c3=O)C2)cc1] | I-112 |
| 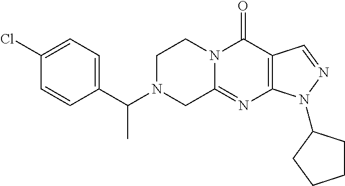 [SMILES: CC(c1ccc(Cl)cc1)N1CCn2c(nc3c(cnn3C3CCCC3)c2=O)C1] | I-113 | 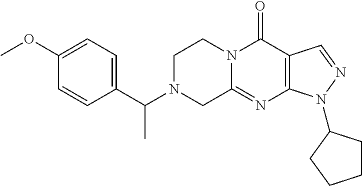 [SMILES: COc1ccc(C(C)N2CCn3c(nc4c(cnn4C4CCCC4)c3=O)C2)cc1] | I-114 |
| 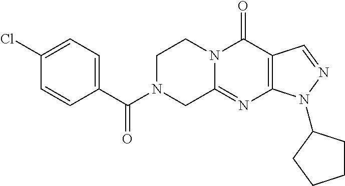 [SMILES: O=C(c1ccc(Cl)cc1)N1CCn2c(nc3c(cnn3C3CCCC3)c2=O)C1] | I-115 | 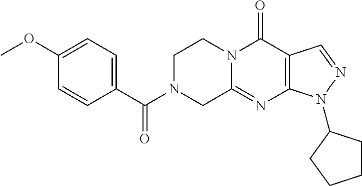 [SMILES: COc1ccc(C(=O)N2CCn3c(nc4c(cnn4C4CCCC4)c3=O)C2)cc1] | I-116 |
| 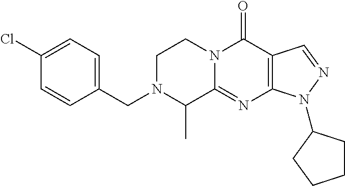 [SMILES: CC1c2nc3c(cnn3C3CCCC3)c(=O)n2CCN1Cc1ccc(Cl)cc1] | I-117 | 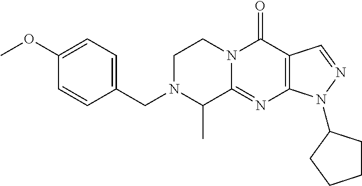 [SMILES: COc1ccc(CN2CCn3c(nc4c(cnn4C4CCCC4)c3=O)C2C)cc1] | I-118 |
| 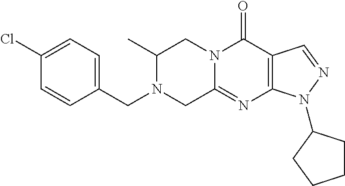 [SMILES: CC1Cn2c(nc3c(cnn3C3CCCC3)c2=O)CN1Cc1ccc(Cl)cc1] | I-119 | 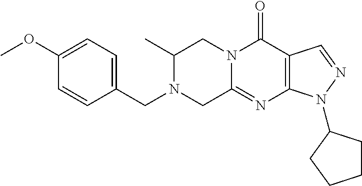 [SMILES: COc1ccc(CN2Cc3nc4c(cnn4C4CCCC4)c(=O)n3CC2C)cc1] | I-120 |
| 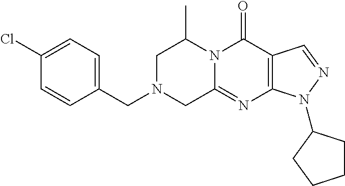 [SMILES: CC1CN(Cc2ccc(Cl)cc2)Cc2nc3c(cnn3C3CCCC3)c(=O)n21] | I-121 | 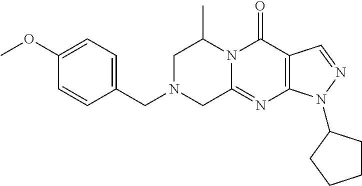 [SMILES: COc1ccc(CN2Cc3nc4c(cnn4C4CCCC4)c(=O)n3C(C)C2)cc1] | I-122 |
| 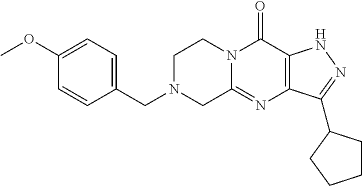 [SMILES: COc1ccc(CN2CCn3c(nc4c(C5CCCC5)n[nH]c4c3=O)C2)cc1] | I-123 | 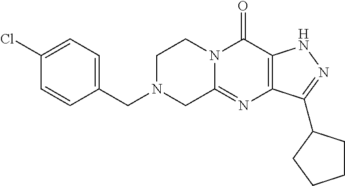 [SMILES: O=c1c2[nH]nc(C3CCCC3)c2nc2n1CCN(Cc1ccc(Cl)cc1)C2] | I-124 |
| 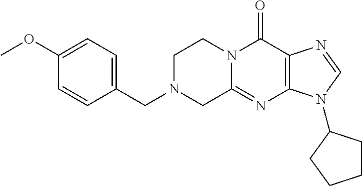 [SMILES: COc1ccc(CN2CCn3c(nc4c(ncn4C4CCCC4)c3=O)C2)cc1] | I-125 | 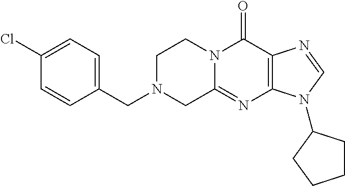 [SMILES: O=c1c2ncn(C3CCCC3)c2nc2n1CCN(Cc1ccc(Cl)cc1)C2] | I-126 |
| 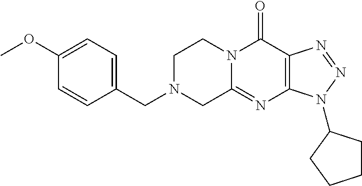 [SMILES: COc1ccc(CN2CCn3c(nc4c(nnn4C4CCCC4)c3=O)C2)cc1] | I-127 | 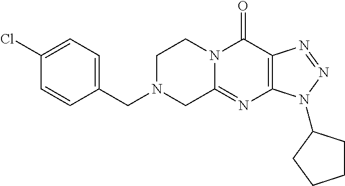 [SMILES: O=c1c2nnn(C3CCCC3)c2nc2n1CCN(Cc1ccc(Cl)cc1)C2] | I-128 |
| 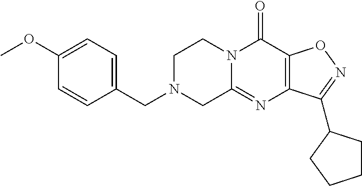 [SMILES: COc1ccc(CN2CCn3c(nc4c(C5CCCC5)noc4c3=O)C2)cc1] | I-129 | 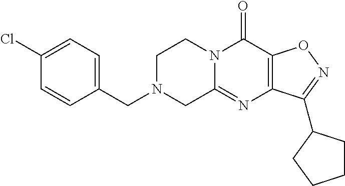 [SMILES: O=c1c2onc(C3CCCC3)c2nc2n1CCN(Cc1ccc(Cl)cc1)C2] | I-130 |
| 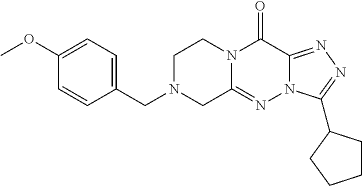 [SMILES: COc1ccc(CN2CCn3c(nn4c(C5CCCC5)nnc4c3=O)C2)cc1] | I-131 | 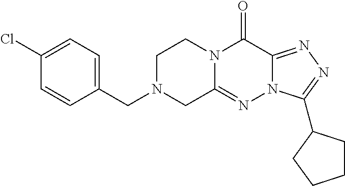 [SMILES: O=c1c2nnc(C3CCCC3)n2nc2n1CCN(Cc1ccc(Cl)cc1)C2] | I-132 |
| 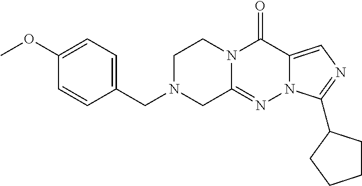 [SMILES: COc1ccc(CN2CCn3c(nn4c(C5CCCC5)ncc4c3=O)C2)cc1] | I-133 | 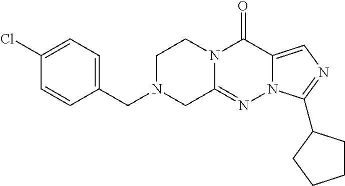 [SMILES: O=c1c2cnc(C3CCCC3)n2nc2n1CCN(Cc1ccc(Cl)cc1)C2] | I-134 |
| 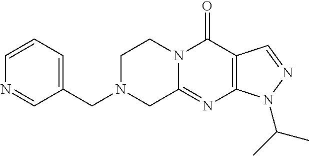 [SMILES: CC(C)n1ncc2c(=O)n3c(nc21)CN(Cc1cccnc1)CC3] | I-135 | 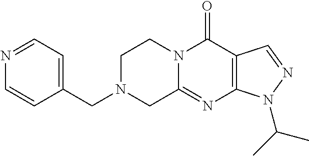 [SMILES: CC(C)n1ncc2c(=O)n3c(nc21)CN(Cc1ccncc1)CC3] | I-136 |
| 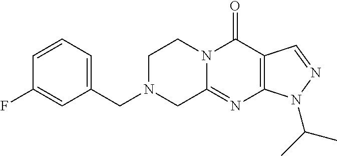 [SMILES: CC(C)n1ncc2c(=O)n3c(nc21)CN(Cc1cccc(F)c1)CC3] | I-137 | 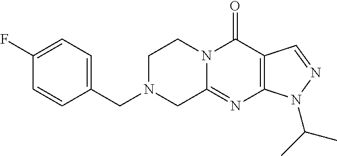 [SMILES: CC(C)n1ncc2c(=O)n3c(nc21)CN(Cc1ccc(F)cc1)CC3] | I-138 |
| 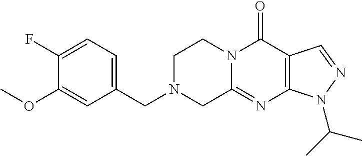 [SMILES: COc1cc(CN2CCn3c(nc4c(cnn4C(C)C)c3=O)C2)ccc1F] | I-139 | 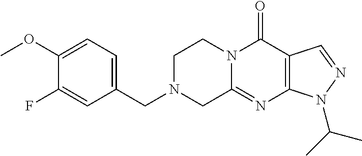 [SMILES: COc1ccc(CN2CCn3c(nc4c(cnn4C(C)C)c3=O)C2)cc1F] | I-140 |
| 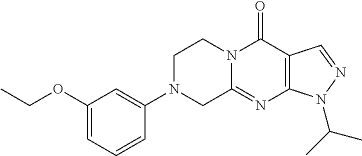 [SMILES: CCOc1cccc(N2CCn3c(nc4c(cnn4C(C)C)c3=O)C2)c1] | I-141 | 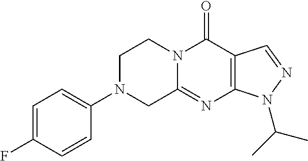 [SMILES: CC(C)n1ncc2c(=O)n3c(nc21)CN(c1ccc(F)cc1)CC3] | I-142 |
| 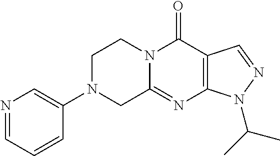 [SMILES: CC(C)n1ncc2c(=O)n3c(nc21)CN(c1cccnc1)CC3] | I-143 | 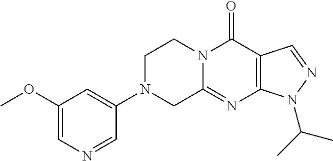 [SMILES: COc1cncc(N2CCn3c(nc4c(cnn4C(C)C)c3=O)C2)c1] | I-144 |
| 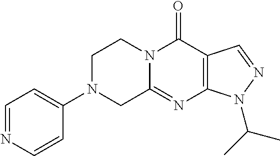 [SMILES: CC(C)n1ncc2c(=O)n3c(nc21)CN(c1ccncc1)CC3] | I-145 | 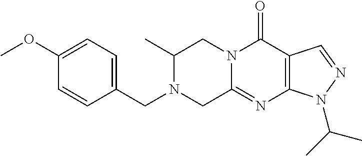 [SMILES: COc1ccc(CN2Cc3nc4c(cnn4C(C)C)c(=O)n3CC2C)cc1] | I-146 |
| 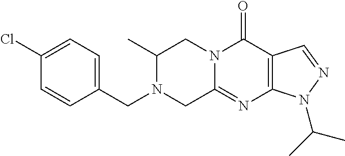 [SMILES: CC1Cn2c(nc3c(cnn3C(C)C)c2=O)CN1Cc1ccc(Cl)cc1] | I-147 | 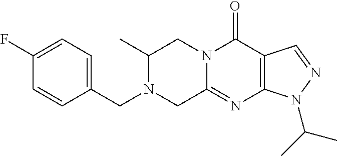 [SMILES: CC1Cn2c(nc3c(cnn3C(C)C)c2=O)CN1Cc1ccc(F)cc1] | I-148 |
| 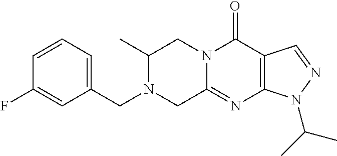 [SMILES: CC1Cn2c(nc3c(cnn3C(C)C)c2=O)CN1Cc1cccc(F)c1] | I-149 | 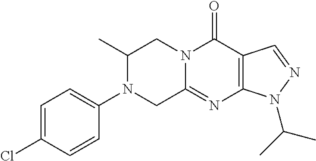 [SMILES: CC1Cn2c(nc3c(cnn3C(C)C)c2=O)CN1c1ccc(Cl)cc1] | I-150 |
| 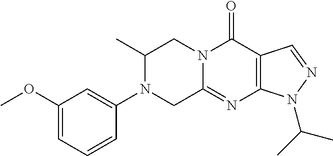 [SMILES: COc1cccc(N2Cc3nc4c(cnn4C(C)C)c(=O)n3CC2C)c1] | I-151 | 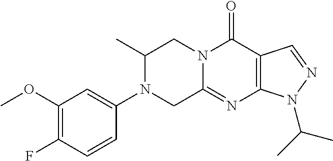 [SMILES: COc1cc(N2Cc3nc4c(cnn4C(C)C)c(=O)n3CC2C)ccc1F] | I-152 |
| 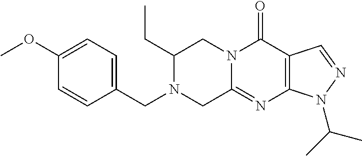 [SMILES: CCC1Cn2c(nc3c(cnn3C(C)C)c2=O)CN1Cc1ccc(OC)cc1] | I-153 | 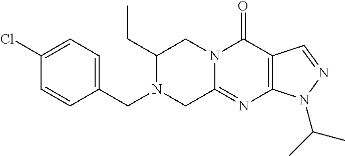 [SMILES: CCC1Cn2c(nc3c(cnn3C(C)C)c2=O)CN1Cc1ccc(Cl)cc1] | I-154 |
| 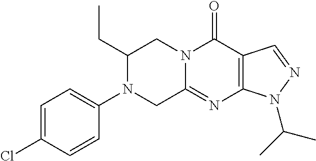 [SMILES: CCC1Cn2c(nc3c(cnn3C(C)C)c2=O)CN1c1ccc(Cl)cc1] | I-155 | 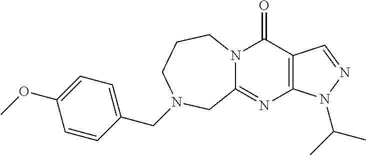 [SMILES: COc1ccc(CN2CCCn3c(nc4c(cnn4C(C)C)c3=O)C2)cc1] | I-156 |
| 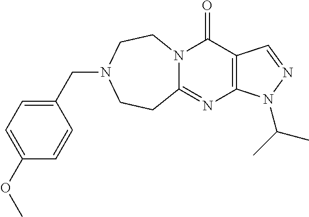 [SMILES: COc1ccc(CN2CCc3nc4c(cnn4C(C)C)c(=O)n3CC2)cc1] | I-157 | 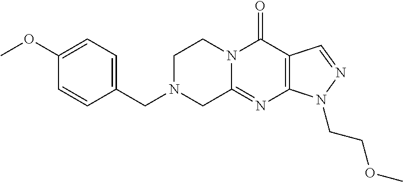 | I-158 |
| 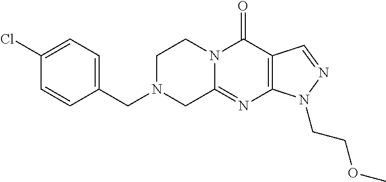 [SMILES: COCCn1ncc2c(=O)n3c(nc21)CN(Cc1ccc(Cl)cc1)CC3] | I-159 | 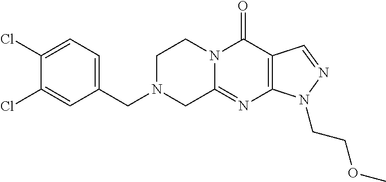 [SMILES: COCCn1ncc2c(=O)n3c(nc21)CN(Cc1ccc(Cl)c(Cl)c1)CC3] | I-160 |
| 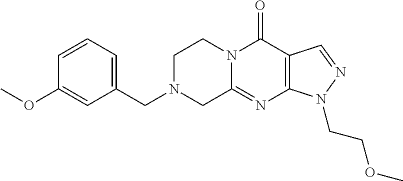 | I-161 | 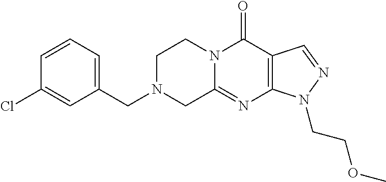 [SMILES: COCCn1ncc2c(=O)n3c(nc21)CN(Cc1cccc(Cl)c1)CC3] | I-162 |
| 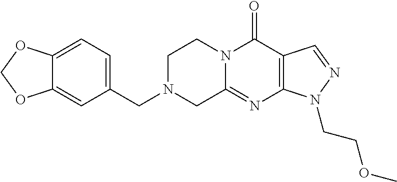 [SMILES: COCCn1ncc2c(=O)n3c(nc21)CN(Cc1ccc2c(c1)OCO2)CC3] | I-163 | 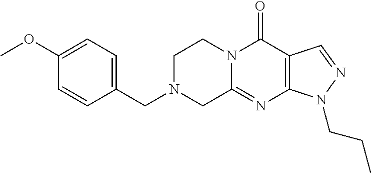 [SMILES: CCCn1ncc2c(=O)n3c(nc21)CN(Cc1ccc(OC)cc1)CC3] | I-164 |
| 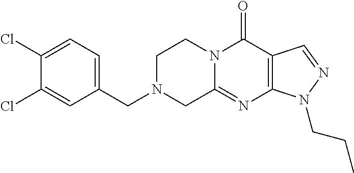 [SMILES: CCCn1ncc2c(=O)n3c(nc21)CN(Cc1ccc(Cl)c(Cl)c1)CC3] | I-165 | 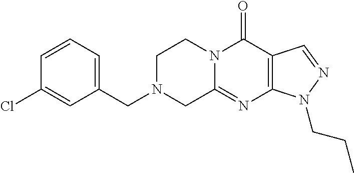 [SMILES: CCCn1ncc2c(=O)n3c(nc21)CN(Cc1cccc(Cl)c1)CC3] | I-166 |
| 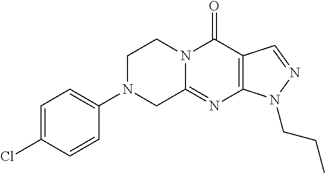 [SMILES: CCCn1ncc2c(=O)n3c(nc21)CN(c1ccc(Cl)cc1)CC3] | I-167 | 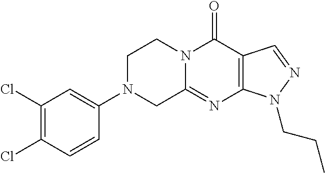 [SMILES: CCCn1ncc2c(=O)n3c(nc21)CN(c1ccc(Cl)c(Cl)c1)CC3] | I-168 |
| 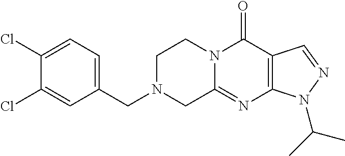 [SMILES: CC(C)n1ncc2c(=O)n3c(nc21)CN(Cc1ccc(Cl)c(Cl)c1)CC3] | I-169 | 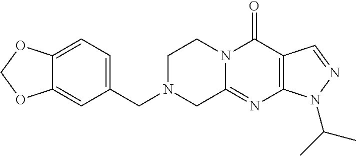 [SMILES: CC(C)n1ncc2c(=O)n3c(nc21)CN(Cc1ccc2c(c1)OCO2)CC3] | I-170 |
| 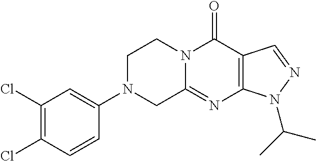 [SMILES: CC(C)n1ncc2c(=O)n3c(nc21)CN(c1ccc(Cl)c(Cl)c1)CC3] | I-171 | 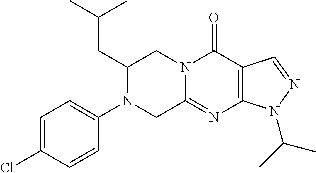 [SMILES: CC(C)CC1Cn2c(nc3c(cnn3C(C)C)c2=O)CN1c1ccc(Cl)cc1] | I-172 |
| 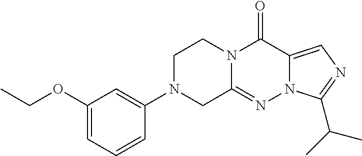 [SMILES: CCOc1cccc(N2CCn3c(nn4c(C(C)C)ncc4c3=O)C2)c1] | I-173 | 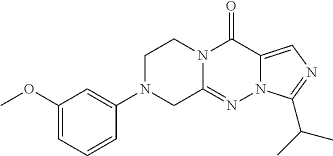 [SMILES: COc1cccc(N2CCn3c(nn4c(C(C)C)ncc4c3=O)C2)c1] | I-174 |
| 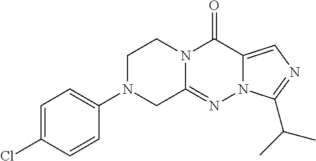 [SMILES: CC(C)c1ncc2c(=O)n3c(nn12)CN(c1ccc(Cl)cc1)CC3] | I-175 | 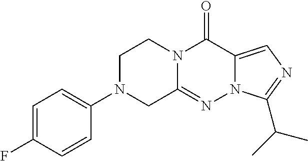 [SMILES: CC(C)c1ncc2c(=O)n3c(nn12)CN(c1ccc(F)cc1)CC3] | I-176 |
| 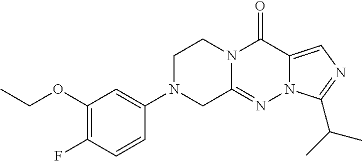 [SMILES: CCOc1cc(N2CCn3c(nn4c(C(C)C)ncc4c3=O)C2)ccc1F] | I-177 | 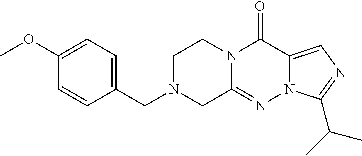 [SMILES: COc1ccc(CN2CCn3c(nn4c(C(C)C)ncc4c3=O)C2)cc1] | I-178 |
| 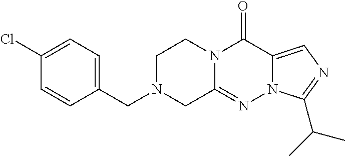 [SMILES: CC(C)c1ncc2c(=O)n3c(nn12)CN(Cc1ccc(Cl)cc1)CC3] | I-179 | 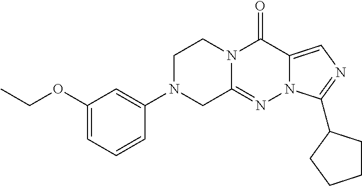 [SMILES: CCOc1cccc(N2CCn3c(nn4c(C5CCCC5)ncc4c3=O)C2)c1] | I-180 |
| 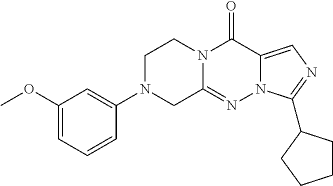 [SMILES: COc1cccc(N2CCn3c(nn4c(C5CCCC5)ncc4c3=O)C2)c1] | I-181 | 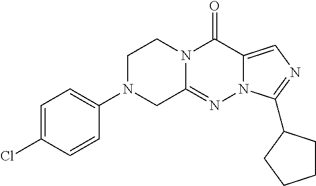 [SMILES: O=c1c2cnc(C3CCCC3)n2nc2n1CCN(c1ccc(Cl)cc1)C2] | I-182 |
| 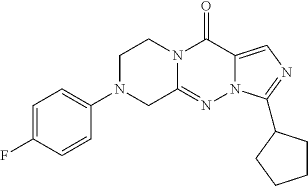 [SMILES: O=c1c2cnc(C3CCCC3)n2nc2n1CCN(c1ccc(F)cc1)C2] | I-183 | 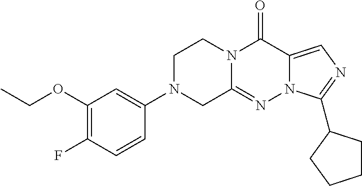 [SMILES: CCOc1cc(N2CCn3c(nn4c(C5CCCC5)ncc4c3=O)C2)ccc1F] | I-184 |
| 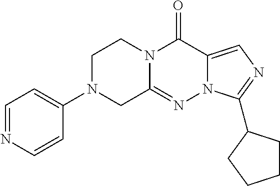 [SMILES: O=c1c2cnc(C3CCCC3)n2nc2n1CCN(c1ccncc1)C2] | I-185 | 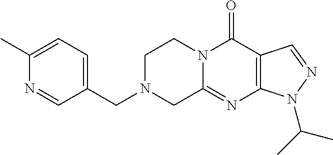 [SMILES: Cc1ccc(CN2CCn3c(nc4c(cnn4C(C)C)c3=O)C2)cn1] | I-186 |
| 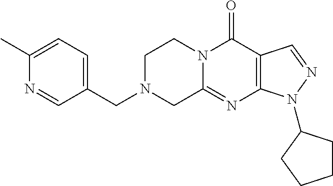 [SMILES: Cc1ccc(CN2CCn3c(nc4c(cnn4C4CCCC4)c3=O)C2)cn1] | I-187 | 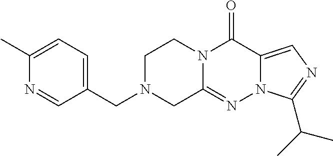 [SMILES: Cc1ccc(CN2CCn3c(nn4c(C(C)C)ncc4c3=O)C2)cn1] | I-188 |
| 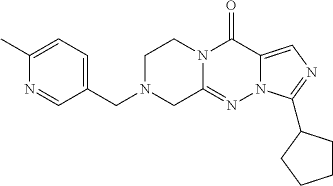 [SMILES: Cc1ccc(CN2CCn3c(nn4c(C5CCCC5)ncc4c3=O)C2)cn1] | I-189 | 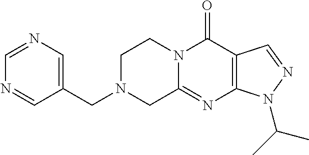 [SMILES: CC(C)n1ncc2c(=O)n3c(nc21)CN(Cc1cncnc1)CC3] | I-190 |
| 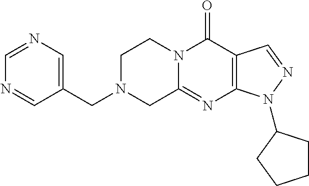 [SMILES: O=c1c2cnn(C3CCCC3)c2nc2n1CCN(Cc1cncnc1)C2] | I-191 | 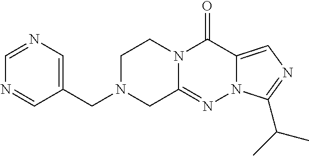 [SMILES: CC(C)c1ncc2c(=O)n3c(nn12)CN(Cc1cncnc1)CC3] | I-192 |
| 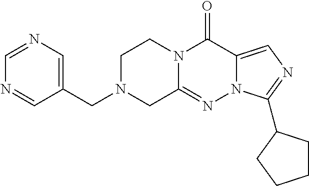 [SMILES: O=c1c2cnc(C3CCCC3)n2nc2n1CCN(Cc1cncnc1)C2] | I-193 | 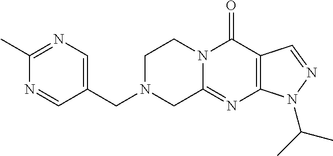 [SMILES: Cc1ncc(CN2CCn3c(nc4c(cnn4C(C)C)c3=O)C2)cn1] | I-194 |
| 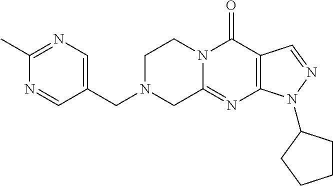 [SMILES: Cc1ncc(CN2CCn3c(nc4c(cnn4C4CCCC4)c3=O)C2)cn1] | I-195 | 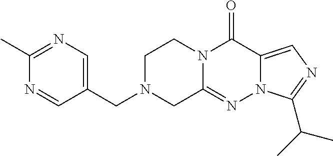 [SMILES: Cc1ncc(CN2CCn3c(nn4c(C(C)C)ncc4c3=O)C2)cn1] | I-196 |
| 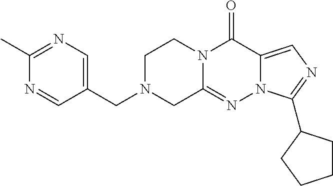 [SMILES: Cc1ncc(CN2CCn3c(nn4c(C5CCCC5)ncc4c3=O)C2)cn1] | I-197 | 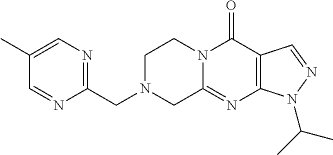 [SMILES: Cc1cnc(CN2CCn3c(nc4c(cnn4C(C)C)c3=O)C2)nc1] | I-198 |
| 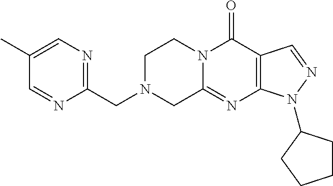 [SMILES: Cc1cnc(CN2CCn3c(nc4c(cnn4C4CCCC4)c3=O)C2)nc1] | I-199 | 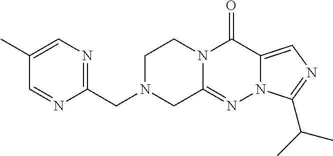 [SMILES: Cc1cnc(CN2CCn3c(nn4c(C(C)C)ncc4c3=O)C2)nc1] | I-200 |
| 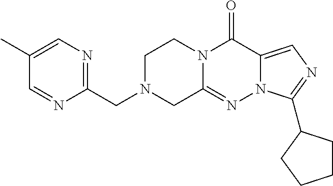 [SMILES: Cc1cnc(CN2CCn3c(nn4c(C5CCCC5)ncc4c3=O)C2)nc1] | I-201 | 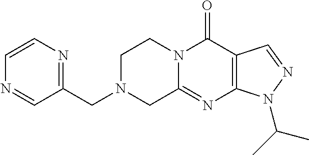 [SMILES: CC(C)n1ncc2c(=O)n3c(nc21)CN(Cc1cnccn1)CC3] | I-202 |
| 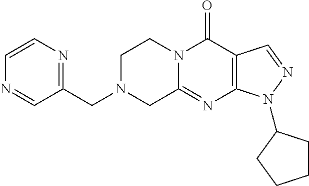 [SMILES: O=c1c2cnn(C3CCCC3)c2nc2n1CCN(Cc1cnccn1)C2] | I-203 | 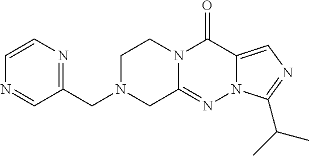 [SMILES: CC(C)c1ncc2c(=O)n3c(nn12)CN(Cc1cnccn1)CC3] | I-204 |
| 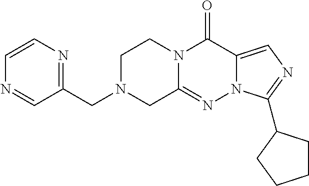 [SMILES: O=c1c2cnc(C3CCCC3)n2nc2n1CCN(Cc1cnccn1)C2] | I-205 | 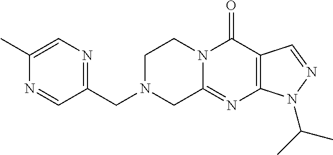 [SMILES: Cc1cnc(CN2CCn3c(nc4c(cnn4C(C)C)c3=O)C2)cn1] | I-206 |
| 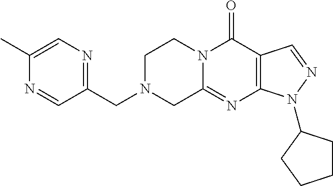 [SMILES: Cc1cnc(CN2CCn3c(nc4c(cnn4C4CCCC4)c3=O)C2)cn1] | I-207 | 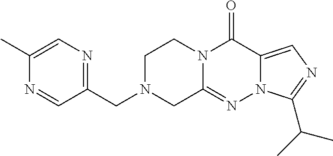 [SMILES: Cc1cnc(CN2CCn3c(nn4c(C(C)C)ncc4c3=O)C2)cn1] | I-208 |
| 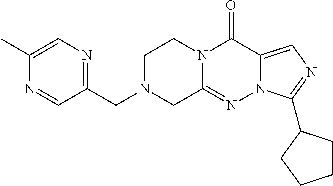 [SMILES: Cc1cnc(CN2CCn3c(nn4c(C5CCCC5)ncc4c3=O)C2)cn1] | I-209 | 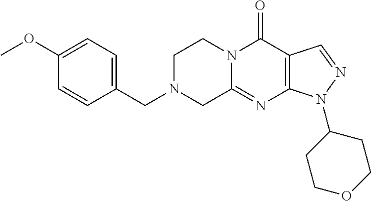 [SMILES: COc1ccc(CN2CCn3c(nc4c(cnn4C4CCOCC4)c3=O)C2)cc1] | I-210 |
| 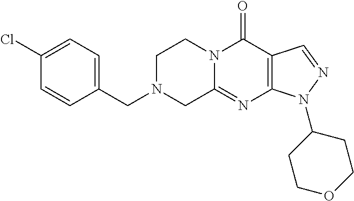 [SMILES: O=c1c2cnn(C3CCOCC3)c2nc2n1CCN(Cc1ccc(Cl)cc1)C2] | I-211 | 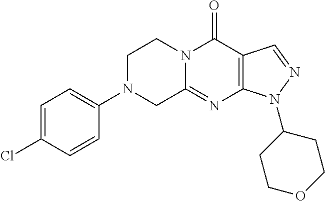 [SMILES: O=c1c2cnn(C3CCOCC3)c2nc2n1CCN(c1ccc(Cl)cc1)C2] | I-212 |
| 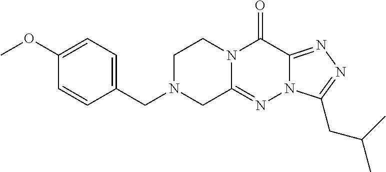 [SMILES: COc1ccc(CN2CCn3c(nn4c(CC(C)C)nnc4c3=O)C2)cc1] | I-213 | 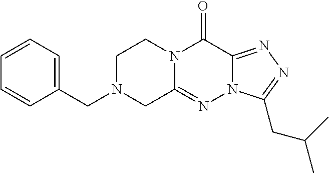 [SMILES: CC(C)Cc1nnc2c(=O)n3c(nn12)CN(Cc1ccccc1)CC3] | I-214 |
| 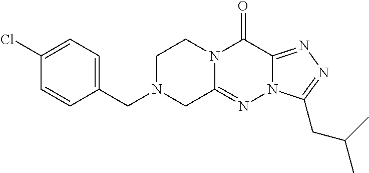 [SMILES: CC(C)Cc1nnc2c(=O)n3c(nn12)CN(Cc1ccc(Cl)cc1)CC3] | I-215 | 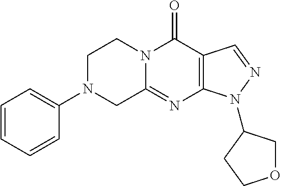 [SMILES: O=c1c2cnn(C3CCOC3)c2nc2n1CCN(c1ccccc1)C2] | I-216 |
| 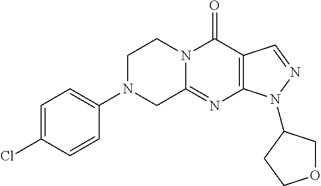 [SMILES: O=c1c2cnn(C3CCOC3)c2nc2n1CCN(c1ccc(Cl)cc1)C2] | I-217 | 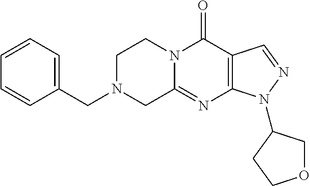 [SMILES: O=c1c2cnn(C3CCOC3)c2nc2n1CCN(Cc1ccccc1)C2] | I-218 |
| 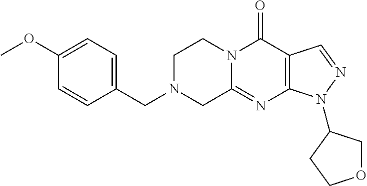 [SMILES: COc1ccc(CN2CCn3c(nc4c(cnn4C4CCOC4)c3=O)C2)cc1] | I-219 | 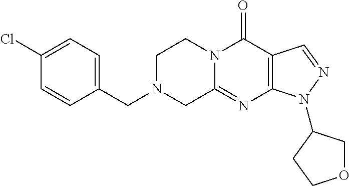 [SMILES: O=c1c2cnn(C3CCOC3)c2nc2n1CCN(Cc1ccc(Cl)cc1)C2] | I-220 |
| 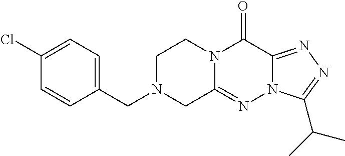 [SMILES: CC(C)c1nnc2c(=O)n3c(nn12)CN(Cc1ccc(Cl)cc1)CC3] | I-221 | 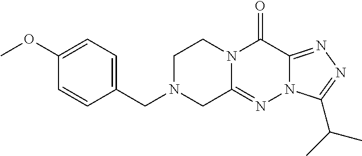 [SMILES: COc1ccc(CN2CCn3c(nn4c(C(C)C)nnc4c3=O)C2)cc1] | I-222 |
| 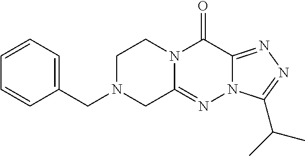 [SMILES: CC(C)c1nnc2c(=O)n3c(nn12)CN(Cc1ccccc1)CC3] | I-223 | 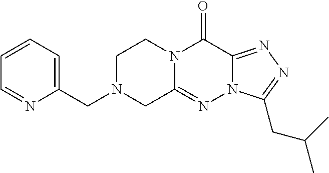 [SMILES: CC(C)Cc1nnc2c(=O)n3c(nn12)CN(Cc1ccccn1)CC3] | I-224 |
| 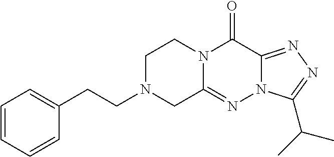 [SMILES: CC(C)c1nnc2c(=O)n3c(nn12)CN(CCc1ccccc1)CC3] | I-225 | 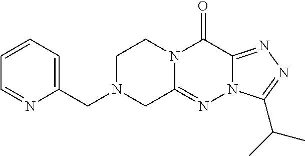 [SMILES: CC(C)c1nnc2c(=O)n3c(nn12)CN(Cc1ccccn1)CC3] | I-226 |
| 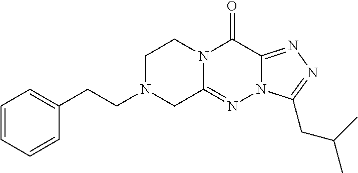 [SMILES: CC(C)Cc1nnc2c(=O)n3c(nn12)CN(CCc1ccccc1)CC3] | I-227 | 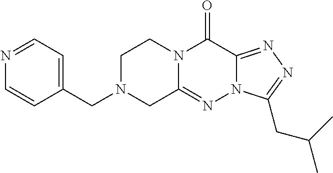 [SMILES: CC(C)Cc1nnc2c(=O)n3c(nn12)CN(Cc1ccncc1)CC3] | I-228 |
| 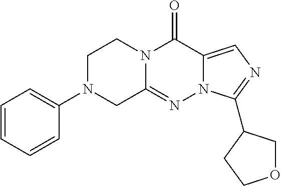 [SMILES: O=c1c2cnc(C3CCOC3)n2nc2n1CCN(c1ccccc1)C2] | I-229 | 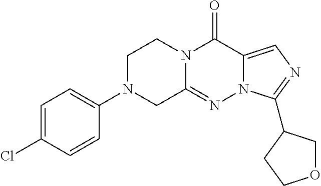 [SMILES: O=c1c2cnc(C3CCOC3)n2nc2n1CCN(c1ccc(Cl)cc1)C2] | I-230 |
| 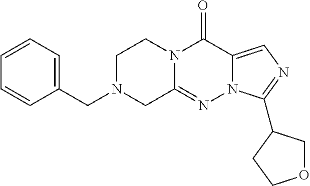 [SMILES: O=c1c2cnc(C3CCOC3)n2nc2n1CCN(Cc1ccccc1)C2] | I-231 | 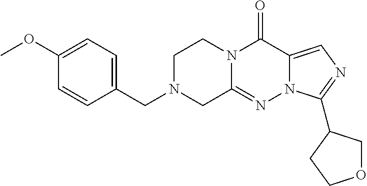 [SMILES: COc1ccc(CN2CCn3c(nn4c(C5CCOC5)ncc4c3=O)C2)cc1] | I-232 |
| 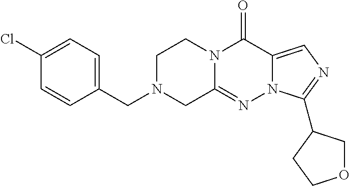 [SMILES: O=c1c2cnc(C3CCOC3)n2nc2n1CCN(Cc1ccc(Cl)cc1)C2] | I-233 | 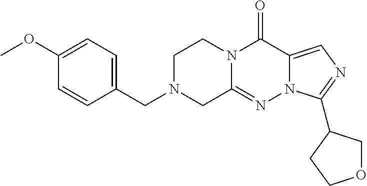 [SMILES: COc1ccc(CN2CCn3c(nn4c(C5CCOC5)ncc4c3=O)C2)cc1] | I-234 |
| 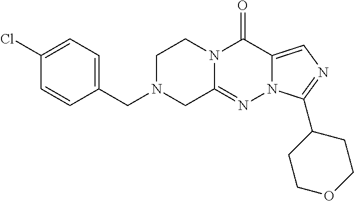 [SMILES: O=c1c2cnc(C3CCOCC3)n2nc2n1CCN(Cc1ccc(Cl)cc1)C2] | I-235 | 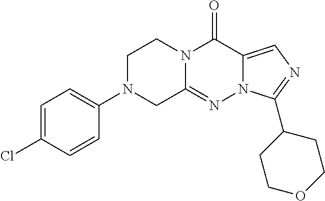 [SMILES: O=c1c2cnc(C3CCOCC3)n2nc2n1CCN(c1ccc(Cl)cc1)C2] | I-236 |
| 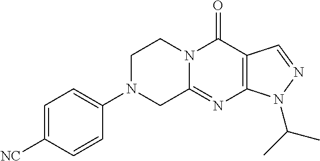 [SMILES: CC(C)n1ncc2c(=O)n3c(nc21)CN(c1ccc(C#N)cc1)CC3] | I-237 | 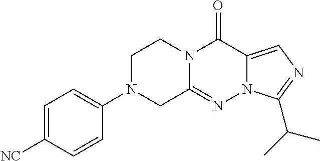 [SMILES: CC(C)c1ncc2c(=O)n3c(nn12)CN(c1ccc(C#N)cc1)CC3] | I-238 |
| 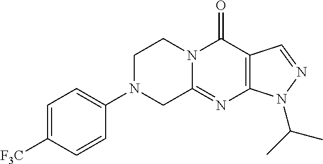 [SMILES: CC(C)n1ncc2c(=O)n3c(nc21)CN(c1ccc(C(F)(F)F)cc1)CC3] | I-239 | 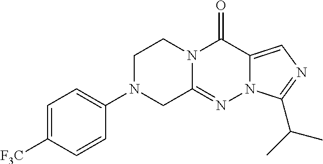 [SMILES: CC(C)c1ncc2c(=O)n3c(nn12)CN(c1ccc(C(F)(F)F)cc1)CC3] | I-240 |
| 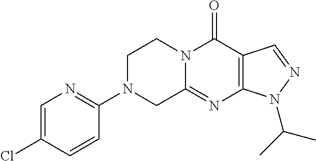 [SMILES: CC(C)n1ncc2c(=O)n3c(nc21)CN(c1ccc(Cl)cn1)CC3] | I-241 | 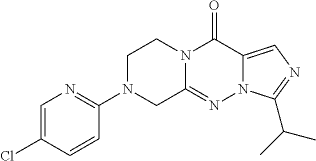 [SMILES: CC(C)c1ncc2c(=O)n3c(nn12)CN(c1ccc(Cl)cn1)CC3] | I-242 |
| 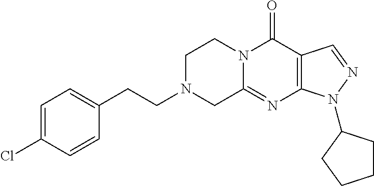 [SMILES: O=c1c2cnn(C3CCCC3)c2nc2n1CCN(CCc1ccc(Cl)cc1)C2] | I-243 | 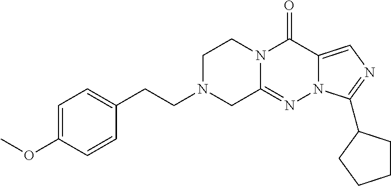 [SMILES: COc1ccc(CCN2CCn3c(nn4c(C5CCCC5)ncc4c3=O)C2)cc1] | I-244 |
| 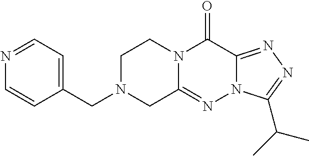 [SMILES: CC(C)c1nnc2c(=O)n3c(nn12)CN(Cc1ccncc1)CC3] | I-245 | 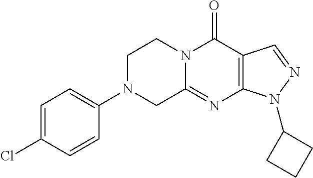 [SMILES: O=c1c2cnn(C3CCC3)c2nc2n1CCN(c1ccc(Cl)cc1)C2] | I-246 |
| 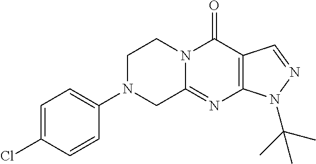 [SMILES: CC(C)(C)n1ncc2c(=O)n3c(nc21)CN(c1ccc(Cl)cc1)CC3] | I-247 | 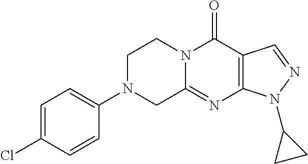 [SMILES: O=c1c2cnn(C3CC3)c2nc2n1CCN(c1ccc(Cl)cc1)C2] | I-248 |
| 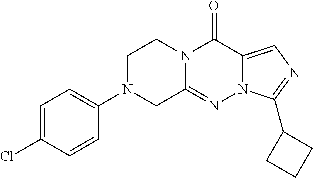 [SMILES: O=c1c2cnc(C3CCC3)n2nc2n1CCN(c1ccc(Cl)cc1)C2] | I-249 | 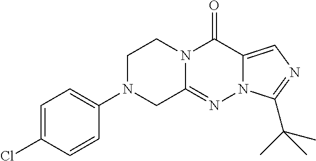 [SMILES: CC(C)(C)c1ncc2c(=O)n3c(nn12)CN(c1ccc(Cl)cc1)CC3] | I-250 |
| 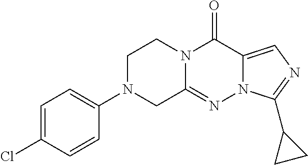 [SMILES: O=c1c2cnc(C3CC3)n2nc2n1CCN(c1ccc(Cl)cc1)C2] | I-251 | 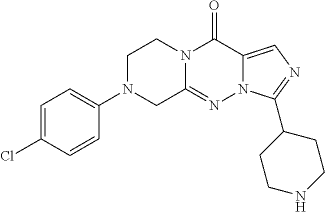 [SMILES: O=c1c2cnc(C3CCNCC3)n2nc2n1CCN(c1ccc(Cl)cc1)C2] | I-252 |
| 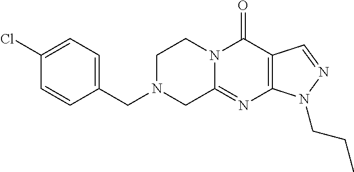 [SMILES: CCCn1ncc2c(=O)n3c(nc21)CN(Cc1ccc(Cl)cc1)CC3] | I-253 | 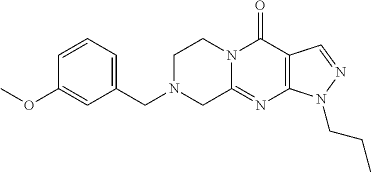 [SMILES: CCCn1ncc2c(=O)n3c(nc21)CN(Cc1cccc(OC)c1)CC3] | I-254 |
| 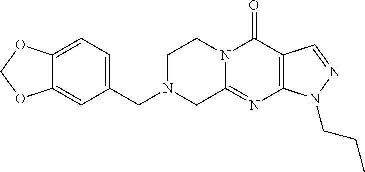 [SMILES: CCCn1ncc2c(=O)n3c(nc21)CN(Cc1ccc2c(c1)OCO2)CC3] | I-255 | 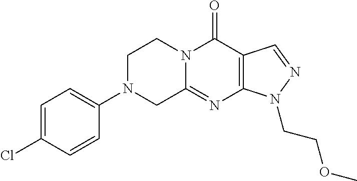 [SMILES: COCCn1ncc2c(=O)n3c(nc21)CN(c1ccc(Cl)cc1)CC3] | I-256 |
| 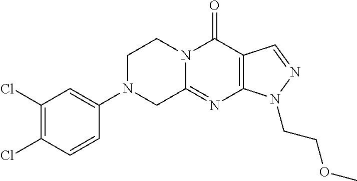 [SMILES: COCCn1ncc2c(=O)n3c(nc21)CN(c1ccc(Cl)c(Cl)c1)CC3] | I-257 | 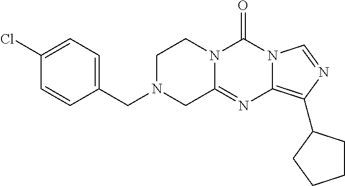 [SMILES: O=c1n2c(nc3c(C4CCCC4)ncn13)CN(Cc1ccc(Cl)cc1)CC2] | I-258 |

In certain embodiments, the present invention provides any compound selected from those depicted in Table 1, above, or a pharmaceutically acceptable salt thereof.

### 4. Uses, Formulation and Administration and Pharmaceutically Acceptable Compositions

According to another embodiment, the invention provides a composition comprising a compound of this invention or a pharmaceutically acceptable salt, ester, or salt of ester thereof and a pharmaceutically acceptable carrier, adjuvant, or vehicle. The amount of compound in compositions of this invention is such that is effective to measurably inhibit PDE1, in a biological sample or in a patient. In certain embodiments, the amount of compound in compositions of this invention is such that is effective to measurably inhibit PDE1, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily active metabolite or residue thereof.

As used herein, the term “inhibitorily active metabolite or residue thereof” means that a metabolite or residue thereof is also an inhibitor of PDE1.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon a variety of factors, including the host treated and the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Phosphodiesterases (PDE's) are enzymes that catalyze the hydrolysis of the cyclic phosphate bonds of cyclic guanosine monophosphate (cGMP) and/or cyclic adenosine monophosphate (cAMP). Lugnier, C., Pharmacology & Therapeutics (2006), 109, 366. The PDE superfamily can be grouped into 11 families (PDE1-11) based on their sequence, regulation and substrate specificity. Each family can contain multiple subtypes, each the product of individual genes. In particular, the PDE1 family, consisting of PDE1A, PDE1B and PDE1C, are so-called dual substrate enzymes that hydrolyze both cGMP and cAMP, and are regulated by Ca²⁺ and calmodulin. PDE1A is expressed throughout the brain, especially in the hippocampus and cerebellum, and at lower levels in the striatum, as well as in the peripheral vasculature. PDE1B, by contrast, is expressed primarily in the striatum and cerebellum, and is often found in regions of high dopaminergic tone and dopamine D1 receptor expression. PDE1C is primarily expressed in the heart, olfactory epithelium and striatum. Considering these expression patterns, a compound that is selective for PDE1B over PDE1A and/or PDE1C may have fewer effects on the cardiovascular system.

Due to the expression pattern of the PDE1 family, inhibition of PDE1 may be useful in the treatment of disorders involving learning and memory by enhancing neuronal plasticity. The increased levels of intracellular cAMP and cGMP caused by PDE1 inhibition trigger cascades that ultimately lead to the phosphorylation and activation of the transcription factors cAMP Responsive Element Binding Protein (CREB) and Serum Response Factor (SRF). Josselyn, S. A., Nguyen, P. V., Current Drug Targets—CNS & Neurological Disorders (2005) 4, 481. Activation of CREB and SRF can lead to the expression of plasticity-related genes which mediate the processes that are critical for neuronal plasticity such as the remodeling of dendritic spines. PDE1 inhibitors may therefore be useful in the treatment of cognitive symptoms of disorders such as Alzheimer's Disease, Parkinson's Disease, Stroke, Schizophrenia, Down Syndrome, Fetal Alcohol Syndrome and others.

Due to its location in the striatum and its role in modulating levels of secondary messengers such as cyclic nucleotides, PDE1 is also a regulator of locomotor activity. Reed, T. M. J., et al., Journal of Neuroscience (2002) 22, 5189). Due to their ability to increase levels of cyclic nucleotides in the striatum, PDE1 inhibitors are expected to potentiate the effects of D1 agonists by inhibiting the degradation of cAMP and cGMP. This potentiation of dopamine signaling may be useful in the treatment of diseases including, but not limited to Parkinson's Disease, depression and cognitive disorders including Cognitive Impairment Associated with. Schizophrenia.

The activity of a compound utilized in this invention as an inhibitor of PDE1 or a treatment for a neurological or psychiatric disorder, may be assayed in vitro or in vivo. An in vivo assessment of the efficacy of the compounds of the invention may be made using an animal model of a neurological or psychiatric disorder, e.g., a rodent or primate model. Cell-based assays may be performed using, e.g., a cell line isolated from a tissue that expresses PDE1, or a cell line that recombinantly expresses PDE1. Additionally, biochemical or mechanism-based assays, e.g., measuring cAMP or cGMP levels, Northern blot, RT-PCR, etc., may be performed. In vitro assays include assays that determine cell morphology, protein expression, and/or the cytotoxicity, enzyme inhibitory activity, and/or the subsequent functional consequences of treatment of cells with compounds of the invention. Alternate in vitro assays quantify the ability of the inhibitor to bind to protein or nucleic acid molecules within the cell. Inhibitor binding may be measured by radiolabelling the inhibitor prior to binding, isolating the inhibitor/target molecule complex and determining the amount of radiolabel bound. Alternatively, inhibitor binding may be determined by running a competition experiment where new inhibitors are incubated with purified proteins or nucleic acids bound to known radioligands. Detailed conditions for assaying a compound utilized in this invention as an inhibitor of PDE1 are set forth in the Examples below. The aforementioned assays are exemplary and not intended to limit the scope of the invention. The skilled practitioner can appreciate that modifications can be made to conventional assays to develop equivalent assays that obtain the same result.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The compounds and compositions, according to the method of the present invention, may be administered using any amount and any route of administration effective for treating or lessening the severity of a neurological or psychiatric disorder.

In some embodiments, the compounds and compositions, according to the method of the present invention, may be administered using any amount and any route of administration effective for treating or lessening the severity of a disease associated with PDE1.

In some embodiments, the compounds and compositions, according to the method of the present invention, may be administered using any amount and any route of administration effective for treating or lessening the severity of a neurological or psychiatric disorder.

In some embodiments, the neurological or psychiatric disorder is selected from schizophrenia or psychosis including schizophrenia (paranoid, disorganized, catatonic or undifferentiated), schizophreniform disorder, schizoaffective disorder, delusional disorder, brief psychotic disorder, shared psychotic disorder, psychotic disorder due to a general medical condition and substance-induced or drug-induced (phencyclidine, ketamine and other dissociative anesthetics, amphetamine and other psychostimulants and cocaine) psychosispsychotic disorder, psychosis associated with affective disorders, brief reactive psychosis, schizoaffective psychosis, “schizophrenia-spectrum” disorders such as schizoid or schizotypal personality disorders, or illness associated with psychosis (such as major depression, manic depressive (bipolar) disorder, Alzheimer's disease and post-traumatic stress syndrome), including both positive, negative, and cognitive symptoms of schizophrenia and other psychoses; cognitive disorders including dementia (associated with Alzheimer's disease, ischemia, multi-infarct dementia, trauma, vascular problems or stroke, HIV disease, Parkinson's disease, Huntington's disease, Down syndrome, Pick's disease, Creutzfeldt-Jacob disease, perinatal hypoxia, other general medical conditions or substance abuse); delirium, amnestic disorders or age related cognitive decline; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder; tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders; depression including unipolar depression, seasonal depression and post-partum depression, premenstrual syndrome (PMS) and premenstrual dysphoric disorder (PDD), mood disorders due to a general medical condition, and substance-induced mood disorders; learning disorders, pervasive developmental disorder including autistic disorder, attention disorders including attention-deficit hyperactivity disorder (ADHD) and conduct disorder; disorders such as autism, depression, benign forgetfulness, childhood learning disorders and closed head injury; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, Parkinsonism-ALS dementia complex and basal ganglia calcification), medication-induced Parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Gilles de la Tourette's syndrome, epilepsy, muscular spasms and disorders associated with muscular spasticity or weakness including tremors; dyskinesias {including drug e.g. L-DOPA induced dyskinesia tremor (such as rest tremor, postural tremor, intention tremor), chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), and dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)}; urinary incontinence; neuronal damage including ocular damage, retinopathy or macular degeneration of the eye, tinnitus, hearing impairment and loss, and brain edema; emesis; and sleep disorders including insomnia and narcolepsy

In some embodiments, the neurological or psychiatric disorder is selected from the group consisting of Alzheimer's Disease, Parkinson's Disease, depression, cognitive impairment, stroke, schizophrenia, Down Syndrome, and Fetal Alcohol Syndrome. In some embodiments, the neurological or psychiatric disorder is Alzheimer's Disease. In some embodiments, the neurological or psychiatric disorder is Parkinson's Disease. In some embodiments, the neurological or psychiatric disorder is depression. In some embodiments, the neurological or psychiatric disorder is cognitive impairment. In some embodiments, the neurological or psychiatric disorder is stroke. In some embodiments, the neurological or psychiatric disorder is schizophrenia. In some embodiments, the neurological or psychiatric disorder is Down Syndrome. In some embodiments, the neurological or psychiatric disorder is Fetal Alcohol Syndrome.

In some embodiments, the neurological or psychiatric disorder involves a deficit in cognition (cognitive domains as defined by the Diagnostic and Statistical Manual of Mental Disorders, 5th Ed., American Psychiatric Publishing (2013) (“DSM-5”) are: complex attention, executive function, learning and memory, language, perceptual-motor, social cognition). In some embodiments, the neurological or psychiatric disorder is associated with a deficit in dopamine signaling. In some embodiments, the neurological or psychiatric disorder is associated with basal ganglia dysfunction. In some embodiments, the neurological or psychiatric disorder is associated with dysregulated locomotor activity.

In some embodiments, the neurological or psychiatric disorder is associated with a deficit in cyclic nucleotide signaling molecules. In some embodiments, the neurological or psychiatric disorder is associated with a deficit in cAMP and/or cGMP. In some embodiments, the neurological or psychiatric disorder is associated with low activity of cAMP Responsive Element Binding Protein (CREB), Serum Response Factor (SRF), or both.

In some embodiments, the present invention provides a method of treating a neurological or psychiatric disorder described herein, comprising administering a compound of the invention in conjunction with one or more pharmaceutical agents. Suitable pharmaceutical agents that may be used in combination with the compounds of the present invention include anti-Parkinson's drugs, anti-Alzheimer's drugs, anti-depressants, anti-psychotics, anti-ischemics, CNS depressants, anti-cholinergics, and nootropics.

Suitable anti-Parkinson's drugs include, but are not limited to, dopamine replacement therapy (e.g. L-DOPA, carbidopa, COMT inhibitors such as entacapone), dopamine agonists (e.g. D1 agonists, D2 agonists, mixed D1/D2 agonists; bromocriptine, pergolide, cabergoline, ropinirole, pramipexole, or apomorphine in combination with domperidone), histamine H2 antagonists, and monoamine oxidase inhibitors such as selegiline and tranylcypromine.

In some embodiments, compounds of the invention may be used in combination with levodopa (with or without a selective extracerebral decarboxylase inhibitor such as carbidopa or benserazide), anticholinergics such as biperiden (optionally as its hydrochloride or lactate salt) and trihexyphenidyl(benzhexyl)hydrochloride, COMT inhibitors such as entacapone, MAO A/B inhibitors, antioxidants, Ata adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexole are commonly used in a non-salt form.

Suitable anti-Alzheimer's drugs include, but are not limited to, beta-secretase inhibitors, gamma-secretase inhibitors, HMG-CoA reductase inhibitors, NSAID's including ibuprofen, vitamin E, and anti-amyloid antibodies. In some embodiments, an anti-Alzheimer's drug is memantine.

Suitable anti-depressants and anti-anxiety agents include, but are not limited to norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists.

Specific suitable anti-depressant and anti-anxiety agents include, but are not limited to, amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The exact amount required will vary from subject to subject, depending on the species, age, and general condition of the subject, the severity of the infection, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the invention relates to a method of inhibiting PDE1 in a biological sample comprising the step of contacting said biological sample with a compound of this invention, or a composition comprising said compound. In some embodiments, the PDE1 is PDE1A. In some embodiments, the PDE1 is PDE1B. In some embodiments, the PDE1 is PDE1C. In some embodiments, the invention provides a method of inhibiting PDE1B selectively over PDE1A and/or PDE1C. In some embodiments, the invention provides a method of inhibiting PDE1B selectively over PDE1A. In some embodiments, the invention provides a method of inhibiting PDE1B selectively over PDE1C. In some embodiments, the invention provides a method of inhibiting PDE1B selectively over PDE1A and PDE1C. In some embodiments, the selectivity for PDE over PDE and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. Selectivity for one PDE1 isoform over another refers to the inverse ratio of IC₅₀ values against each respective isoform as determined using the HTRF PDF1 inhibition assay described in the Examples. For example, the selectivity of a compound of this invention for PDE1B over PDE1C refers to the ratio IC₅₀(PDE1C)/IC₅₀(PDE1B), wherein IC₅₀(PDE1C) is the IC₅₀ value of the compound against PDE1C as determined using the described HTRF PDE1 inhibition assay, and IC₅₀(PDE1B) is the IC₅₀ value of the compound against PDE1B as determined using the described HTRF PDE1 inhibition assay.

In certain embodiments, the invention relates to a method of modulating cyclic nucleotide levels in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of enzymes in a biological sample is useful for a variety of purposes that are known to one of skill in the art. Examples of such purposes include, but are not limited to biological assays, gene expression studies, and biological target identification.

Another embodiment of the present invention relates to a method of inhibiting PDE1 in a patient comprising the step of administering to said patient a compound of the present invention, or a composition comprising said compound. In some embodiments, the PDE1 is PDE1B. In some embodiments, the invention provides a method of inhibiting PDE1B in a patient selectively over PDE1A and/or PDE1C. In some embodiments, the invention provides a method of inhibiting PDE1B in a patient selectively over PDE1A. In some embodiments, the invention provides a method of inhibiting PDE1B in a patient selectively over PDE1C. In some embodiments, the invention provides a method of inhibiting PDE1B in a patient selectively over PDE1A and PDE1C. In some embodiments, the selectivity for PDE1B over PDE1A and/or PDE1C is up to and including five-fold. In some embodiments, the selectivity for PDE1B over PDE1A and/or PDE1C is up to and including ten-fold. In some embodiments, the selectivity for PDE1B over PDE1A and/or PDE1C is up to and including twenty-fold. In some embodiments, the selectivity for PDE1B over PDE1C is up to and including fifty-fold. In some embodiments, the selectivity for PDE1B over PDE1C is up to and including one hundred-fold. In some embodiments, the selectivity for PDE1B over PDE1C is up to and including two hundred-fold. Selectivity for one PDE1 isoform over another refers to the inverse ratio of IC₅₀ values against each respective isoform as determined using the HTRF PDE1 inhibition assay described in the Examples. For example, the selectivity of a compound of this invention for PDE1B over PDE1C refers to the ratio IC₅₀(PDE1C)/IC₅₀(PDE1B), wherein IC₅₀(PDE1C) is the IC₅₀ value of the compound against PDE1C as determined using the described HTRF PDE1 inhibition assay, and IC₅₀(PDE1B) is the IC₅₀ value of the compound against PDE1B as determined using the described HTRF PDE1 inhibition assay.

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated”.

In certain embodiments, a combination of 2 or more therapeutic agents may be administered together with compounds of the invention. In certain embodiments, a combination of 3 or more therapeutic agents may be administered with compounds of the invention.

Other examples of agents the inhibitors of this invention may also be combined with include, without limitation: vitamins and nutritional supplements, antiemetics (e.g. 5-HT; receptor antagonists, dopamine antagonists, NK1 receptor antagonists, histamine receptor antagonists, cannabinoids, benzodiazepines, or anticholinergics), agents for treating Multiple Sclerosis (MS) such as beta interferon (e.g., Avonex® and Rebif®), Copaxone®, and mitoxantrone; treatments for asthma such as albuterol and Singulair®; anti-inflammatory agents such as corticosteroids, TNF blockers, IL-1 RA, azathioprine, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, agents for treating cardiovascular disease such as beta-blockers, ACE inhibitors, diuretics, nitrates, calcium channel blockers, and statins, fibrates, cholesterol absorption inhibitors, bile acid sequestrants, and niacin; agents for treating liver disease such as corticosteroids, cholestyramine, interferons, and anti-viral agents; agents for treating blood disorders such as corticosteroids, anti-leukemic agents, and growth factors; agents for treating immunodeficiency disorders such as gamma globulin; and anti-diabetic agents such as biguanides (metformin, phenformin, buformin), thiazolidinediones (rosiglitazone, pioglitazone, troglitazone), sulfonylureas (tolbutamide, acetohexamide, tolazamide, chlorpropamide, glipizide, glyburide, glimepiride, gliclazide), meglitinides (repaglinide, nateglinide), alpha-glucosidase inhibitors (miglitol, acarbose), incretin mimetics (exenatide, liraglutide, taspogiutide), gastric inhibitory peptide analogs, DPP-4 inhibitors (vildagliptin, sitagliptin, saxagliptin, linagliptin, alogliptin), amylin analogs (pramlintide), and insulin and insulin analogs.

In certain embodiments, compounds of the present invention, or a pharmaceutically acceptable composition thereof, are administered in combination with antisense agents, a monoclonal or polyclonal antibody or an siRNA therapeutic.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another, normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of formula I, an additional therapeutic agent, and a pharmaceutically acceptable carrier, adjuvant, or vehicle.

The amount of both, an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-100 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In some embodiments, the present invention provides a medicament comprising at least one compound of formula I or a pharmaceutically acceptable salt thereof and a pharmaceutically acceptable carrier.

In some embodiments, the present invention provides the use of a compound of formula I in the manufacture of a medicament for the treatment of a neurological or psychiatric disorder.

### EXEMPLIFICATION

As depicted in the Examples below, in certain exemplary embodiments, compounds are prepared according to the following procedures. It will be appreciated that, although the general methods depict the synthesis of certain compounds of the present invention, the following methods, and other methods known to one of ordinary skill in the art, can be applied to all compounds and subclasses and species of each of these compounds, as described herein.

In the examples below, unless otherwise indicated, all temperatures are set forth in degrees Celsius and all parts and percentages are by weight. Reagents were purchased from commercial suppliers, such as Sigma-Aldrich Chemical Company, and were used without further purification unless otherwise indicated. Reagents were prepared following standard literature procedures known to those skilled in the art. Solvents were purchased from Aldrich in Sure-Seal bottles and used as received. All solvents requiring purification or drying were treated using standard methods known to those skilled in the art, unless otherwise indicated.

The reactions set forth below were done generally at ambient temperature, unless otherwise indicated. The reaction flasks were fitted with rubber septa for introduction of substrates and reagents via syringe. Analytical thin layer chromatography (TLC) was performed using glass-backed silica gel pre-coated plates (Merck Art 5719) and eluted with appropriate solvent ratios (v/v). Reactions were assayed by TLC or LCMS, and terminated as judged by the consumption of starting material. Visualization of the TLC plates was done with UV light (254 wavelength) or with an appropriate TLC visualizing solvent, such as basic aqueous KMnO₄ solution activated with heat. Flash column chromatography (See, e.g., Still et al., J. Org. Chem., 43: 2923 (1978)) was performed using silica gel 60 (Merck Art 9385) or various MPLC systems.

The compound structures in the examples below were confirmed by one or more of the following methods: proton magnetic resonance spectroscopy, mass spectroscopy, and melting point. Proton magnetic resonance (¹H NMR) spectra were determined using an NMR spectrometer operating at 400 MHz field strength. Chemical shifts are reported in the form of delta (δ) values given in parts per million (ppm) relative to an internal standard, such as tetramethylsilane (TMS). Alternatively, ¹H NMR spectra were referenced to signals from residual protons in deuterated solvents as follows: CDCl₃=7.25 ppm; DMSO-d₆=2.49 ppm; C₆D₆=7.16 ppm; CD₃OD=3.30 ppm. Peak multiplicities are designated as follows: s, singlet; d, doublet; dd, doublet of doublets; t, triplet; dt, doublet of triplets; q, quartet; quint, quintet; sept, septet; br, broadened; and m, multiplet. Coupling constants are given in Hertz (Hz). Mass spectra (MS) data were obtained using a mass spectrometer with APCI or ESI ionization.

As used herein, and unless otherwise specified, “Me” means methyl, “Et” means ethyl, “Ac” means acetyl, “BINAP” means 2,2′-bis(diphenylphosphino)-1,1′-binaphthyl, “Dess-Martin reagent” means 1,1,1-tris(acetyloxy)-1,1-dihydro-1,2-benziodoxol-3-(1H)-one, “DCM” means dichloromethane, “DIEA” means diisopropylethylamine, “DMF” means dimethylformamide, “EDCI” means N-(3-dimethylaminopropyl)-N′-ethylcarbodiimide hydrochloride, “EtOAc” means ethyl acetate, “EtOH” means ethanol, “HATU” means O-(7-azabenzotriazol-1-yl)-N,N,N′,N′-tetramethyluronium hexafluorophosphate, “HOBt” means hydroxybenzotriazole, “m-CPBA” means 3-chloro-perbenzoic acid, “MeCN” means acetonitrile, “MeOH” means methanol, “PE” means petroleum ether, “RT” or “rt” means room temperature, “t-BuOH” means tert-butanol, “t-BuONa” means sodium tert-butoxide, “TBDMSCl” means tert-butyldimethylsilyl chloride, “TEA” means triethylamine, “THF” means tetrahydrofuran, “TMSI” means iodotrimethyl silane, “Xantphos” means 4,5-bis(diphenylphosphino)-9,9-dimethylxanthene, “h” or “hr” means hour(s), “rt” means room temperature, “min” means minute(s), “cat.” means catalytic, “aq” means aqueous, “TMSI” means trimethylsilyl iodide, and “TFA” means trifluoroacetic acid.

### Example 1

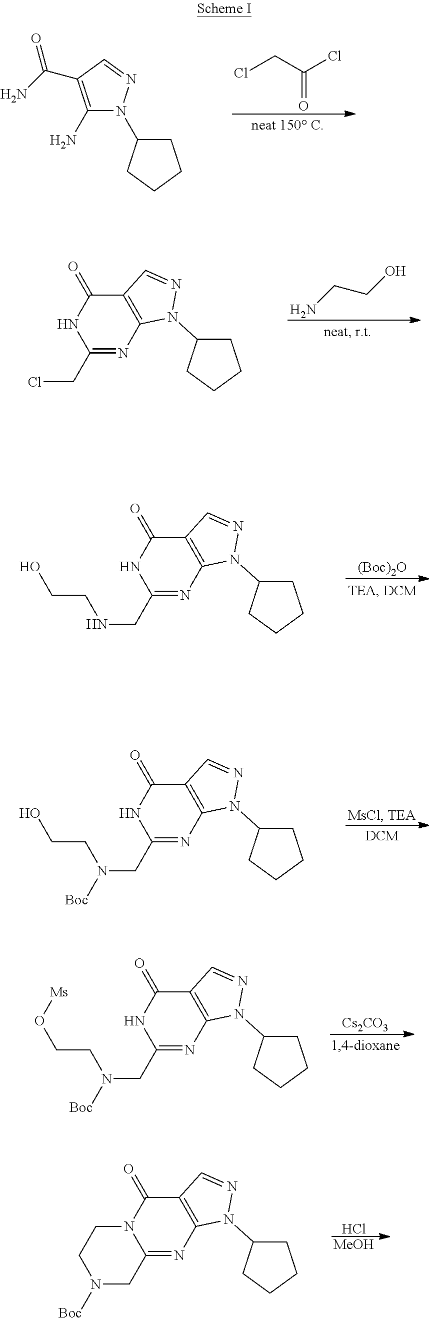

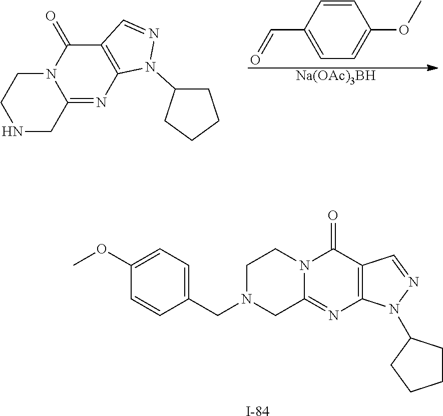

### Compound I-84

### 1-Cyclopentyl-8-(4-methoxybenzyl)-6,7,8,9-tetrahydropyrazino[1,2-a]pyrazolo[3,4-d]pyrimidin-4(1H)-one

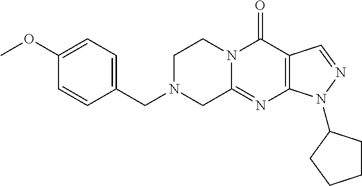 [SMILES: COc1ccc(CN2CCn3c(nc4c(cnn4C4CCCC4)c3=O)C2)cc1]

To a solution of 1-cyclopentyl-6,7,8,9-tetrahydropyrazino[1,2-a]pyrazolo[3,4-d]pyrimidin-4(1H)-one (30 mg, 0.1 mmol, 1.0 eq) in dichloromethane (5 mL) was added 4-methoxybenzaldehyde (30 mg, 0.2 mmol, 2.0 eq) and sodium triacetoxyhydroborate (86 mg, 0.4 mmol, 4.0 eq). The reaction mixture was stirred at room temperature overnight. Upon completion, the mixture was filtered and the filtrate was concentrated in vacuo to give a residue that was purified by Prep-HPLC in 0.01% aqueous ammonia to get 1-cyclopentyl-8-(4-methoxybenzyl)-6,7,8,9-tetrahydropyrazino[1,2-a]pyrazolo[3,4-d]pyrimidin-4(1H)-one (26 mg, yield: 68%) as a colorless oil. ¹H NMR (400 MHz, CDCl₃): δ 8.05 (s, 1H), 7.27 (m, 2H), 6.90 (m, 2H), 5.08 (quint, J=7.6 Hz, 1H), 4.02 (t, J=6.0 Hz, 2H), 3.82 (s, 3H), 3.70 (s, 2H), 3.64 (s, 2H), 2.88 (t, J=5.6 Hz, 2H), 2.10-2.02 (m, 4H), 1.99-1.90 (m, 2H), 1.74-1.66 (m, 2H), LC/MS: m/e=380 (M+H)⁺.

### 6-(Chloromethyl)-1-cyclopentyl-1H-pyrazolo[3,4-d]pyrimidin-4(5H)-one

A mixture of 5-amino-1-cyclopentyl-1H-pyrazole-4-carboxamide (prepared according to the procedure in Haning, H., et al., Bioorg. Med. Chem. Lett. 2005, 15, 3900) (11 g, 56 mmol) and 2-chloroacetyl chloride (12.8 g, 113 mmol) was heated to 150° C. with stirring for 2 hours. Upon completion, the solvent was removed and diluted with dichloromethane (100 mL), washed with sodium bicarbonate solution (30 mL×3), dried and concentrated. The crude product was washed with dichloromethane to get 6-(chloromethyl)-1-cyclopentyl-1H-pyrazolo[3, 4-d]pyrimidin-4(5H)-one (8.2 g, yield: 57%) as a white solid. LC/MS: m/e=253 (M+H)⁺.

### 1-Cyclopentyl-6-((2-hydroxyethylamino)methyl)-1H-pyrazolo[3,4-d]pyrimidin-4(5H)-one

A mixture of 6-(chloromethyl)-1-cyclopentyl-1H-pyrazolo[3,4-d]pyrimidin-4(5H)-one (8 g, 31.7 mmol) and 2-aminoethanol (6 mL) was stirring at room temperature for 2 hours. Upon the completion, the mixture was diluted with dichloromethane (20 mL), purified by silica gel (eluted from dichloromethane:methanol=100:1 to dichloromethane:methanol=60:1) to get 1-cyclopentyl-6-((2-hydroxyethylamino)methyl)-1H-pyrazolo[3,4-d]pyrimidin-4(5H)-one (5.3 g, yield: 61%). LC/MS: m/e=278 (M+H)⁺.

### tert-Butyl (1-cyclopentyl-4-oxo-4,5-dihydro-1H-pyrazolo[3,4-d]pyrimidin-6-yl)methyl(2-hydroxyethyl)carbamate

To a solution of 1-cyclopentyl-6-((2-hydroxyethylamino)methyl)-1H-pyrazolo[3,4-d]pyrimidin-4(5H)-one (5.2 g, 18.7 mmol) and di-tert-butyl dicarbonate (4.9 g, 22.5 mmol) in dichloromethane (30 mL) was added triethylamine (3.79 g, 37.5 mmol) The mixture was stirred at room temperature for overnight. Upon the completion, the reaction mixture was washed with water (20 mL×2), dried and concentrated in vacuo to give tert-butyl (1-cyclopentyl-4-oxo-4,5-dihydro-1H-pyrazolo[3,4-d]pyrimidin-6-yl)methyl(2-hydroxyethyl)carbamate, which was used without further purification. LC/MS: m/e=378 (M+H)⁺.

### 2-(tert-Butoxycarbonyl((1-cyclopentyl-4-oxo-4,5-dihydro-1H-pyrazolo[3,4-d]pyrimidin-6-yl)methyl)amino)ethylmethanesulfonate (I-1.4)

To a solution of tert-butyl(1-cyclopentyl-4-oxo-4,5-dihydro-1H-pyrazolo[3,4-d]pyrimidin-6-yl)methyl(2-hydroxyethyl)carbamate (4.14 g, 10.98 mmol) and triethylamine (2.2 g, 21.96 mmol) in dichloromethane (30 mL) was added methanesulfonyl chloride (1.52 g, 13.17 mmol) slowly at 0° C., The mixture was stirred for 1 hour at room temperature. Upon completion, the reaction mixture was washed with water (20 mL×2), dried and concentrated in vacuo to give 2-(tert-butoxycarbonyl((1-cyclopentyl-4-oxo-4,5-dihydro-1H-pyrazolo[3,4-d]pyrimidin-6-yl)methyl)amino)ethyl methanesulfonate, which was suitable for use without further purification. LC/MS: m/e=456 (M+H)⁺.

### tert-Butyl 1-cyclopentyl-4-oxo-4,6,7,9-tetrahydropyrazino[1,2-a]pyrazolo[3,4-d]pyrimidine-8(1H)-carboxylate

A mixture of 2-(tert-butoxycarbonyl((1-cyclopentyl-4-oxo-4,5-dihydro-1H-pyrazolo[3,4-d]pyrimidin-6-yl)methyl)amino)ethyl methanesulfonate (3.9 g, 8.57 mmol) and cesium carbonate (5.6 g, 17.1 mmol) in dioxane (20 mL) heated to reflux with stirring for 1 hour. Upon completion, the reaction mixture was filtered and the filtrate was concentrated. The crude product was purified by silica gel (eluted from petroleum ether:ethyl acetate=50:1 to petroleum ether:ethyl acetate=20:1) to afford tert-butyl 1-cyclopentyl-4-oxo-4,6,7,9-tetrahydropyrazino[1,2-a]pyrazolo[3,4-d]pyrimidine-8(1H)-carboxylate (3.3 g, yield: 48% based on 1-8) as a white solid. LC/MS: m/e=360 (M+H)⁺.

### 1-Cyclopentyl-6,7,8,9-tetrahydropyrazino[1,2-a]pyrazolo[3,4-d]pyrimidin-4(1H)-one

A mixture of tert-butyl 1-cyclopentyl-4-oxo-4,6,7,9-tetrahydropyrazino[1,2-a]pyrazolo[3,4-d]pyrimidine-8(1H)-carboxylate (3.3 g, 9.2 mmol) in hydrochloric acid in methanol (4 mol/L, 20 mL) was stirred at room temperature for 3 hours. Upon the completion, the solvent was removed and the residue was dissolved in dichloromethane (20 mL), washed with washed with sodium bicarbonate solution (20 mL×2), dried and concentrated to get 1-cyclopentyl-6,7,8,9-tetrahydropyrazino[1,2-a]pyrazolo[3,4-d]pyrimidin-4(1H)-one (1.9 g, yield: 80%) as a white solid. 1H-NMR (400 MHz, CDCl₃): 8.06 (s, 1H), 5.14-5.10 (m, 1H), 4.11 (s, 2H), 4.00-3.97 (t, J=5.6 Hz, 2H), 3.31-3.28 (t, J=5.6 Hz, 2H), 2.13-1.93 (m, 6H), 1.74-1.69 (m, 2H), LC/MS: m/e=260 (M+H)⁺.

### Compound I-81

### 1-Cyclopentyl-8-(3-methoxybenzyl)-6,7,8,9-tetrahydropyrazino[1,2-a]pyrazolo[3,4-d]pyrimidin-4(1H)-one

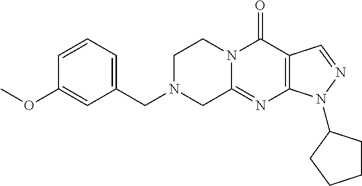 [SMILES: COc1cccc(CN2CCn3c(nc4c(cnn4C4CCCC4)c3=O)C2)c1]

The title compound was prepared via the procedure used for Compound I-84, substituting 3-methoxybenzaldehyde for 4-methoxybenzaldehyde. ¹H NMR (400 MHz, CDCl₃): δ 8.06 (s, 1H), 7.29 (m, 1H), 6.95 (m, 2H), 6.87 (m, 1H), 5.08 (quint, J=7.61 z, 1H), 4.07 (t, J=5.6 Hz, 2H), 3.83 (s, 3H), 3.78 (s, 2H), 3.74 (s, 2H), 2.96 (t, J=5.6 Hz, 2H), 2.11-2.05 (m, 4H), 1.95 (m, 2H), 1.70 (m, 2H), LC/MS: m/e=380 (M+H)⁺.

### Compound I-75

### 8-(4-Chlorobenzyl)-1-cyclopentyl-6,7,8,9-tetrahydropyrazino[1,2-a]pyrazolo[3,4-d]pyrimidin-4(1H)-one

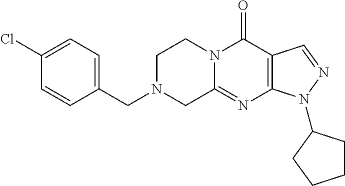 [SMILES: O=c1c2cnn(C3CCCC3)c2nc2n1CCN(Cc1ccc(Cl)cc1)C2]

The title compound was prepared via the procedure used for I-84 substituting 4-chlorobenzaldehyde for 4-methoxybenzaldehyde. ¹H NMR (400 MHz, CDCl₃): δ 8.05 (s, 1H), 7.36-7.27 (m, 4H), 5.07 (quint, J=7.6 Hz, 1H), 4.04 (t, J=6.0 Hz, 2H), 3.69 (s, 2H), 3.67 (s, 2H), 2.90 (t, J=5.6 Hz, 2H), 2.11-2.04 (m, 4H), 1.95 (m, 2H), 1.68 (m, 2H), LC/MS: m/e=384 (M+H)⁺.

### Compound I-78

### 1-Cyclopentyl-8-(2-methoxybenzyl)-6,7,8,9-tetrahydropyrazino[1,2-a]pyrazolo[3,4-d]pyrimidin-4(1H)-one

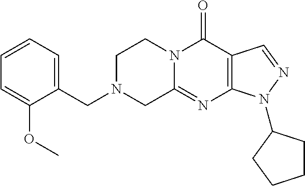 [SMILES: COc1ccccc1CN1CCn2c(nc3c(cnn3C3CCCC3)c2=O)C1]

The title compound was prepared via the procedure used for I-84 substituting 2-methoxybenzaldehyde for 4-methoxybenzaldehyde. ¹H NMR (400 MHz, CDCl₃): δ 8.05 (s, 1H), 7.37-7.27 (m, 2H), 6.99-6.91 (m, 2H), 5.09 (quint, J=7.6 Hz, 1H), 4.03 (t, J=6.0 Hz, 2H), 3.86 (s, 3H), 3.78 (s, 2H), 3.76 (s, 2H), 2.94 (t, J=5.6 Hz, 2H), 2.13-2.03 (m, 4H), 2.00-1.90 (m, 2H), 1.75-1.65 (m, 2H), LC/MS: m/e=380 (M+H)⁺.

### Compound I-52

### 8-Benzyl-1-cyclopentyl-6,7,8,9-tetrahydropyrazino[1,2-a]pyrazolo[3,4-d]pyrimidin-4(1H)-one

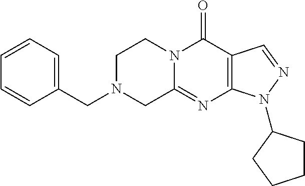 [SMILES: O=c1c2cnn(C3CCCC3)c2nc2n1CCN(Cc1ccccc1)C2]

The title compound was prepared via the procedure used for I-84 substituting benzaldehyde for 4-methoxybenzaldehyde. ¹H NMR (400 MHz, CDCl₃): δ 8.05 (s, 1H), 7.40-7.27 (m, 5H), 5.08 (sept, J=7.6 Hz, 1H), 4.04 (t, J=6.0 Hz, 2H), 3.72 (d, J=6.0 Hz, 4H), 2.91 (t, J=5.6 Hz, 2H), 2.12-2.01 (m, 4H), 2.00-1.91 (m, 2H), 1.74-1.65 (m, 2H), LC/MS: m/e=350 (M+H)⁺.

### Compound I-69

### 8-(2-Chlorobenzyl)-1-cyclopentyl-6,7,8,9-tetrahydropyrazino[1,2-a]pyrazolo[3,4-d]pyrimidin-4(1H)-one

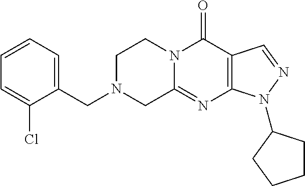 [SMILES: O=c1c2cnn(C3CCCC3)c2nc2n1CCN(Cc1ccccc1Cl)C2]

The title compound was prepared via the procedure used for I-84 substituting 2-chlorobenzaldehyde for 4-methoxybenzaldehyde. ¹H NMR (400 MHz, CDCl₃): δ 8.05 (s, 1H), 7.48 (m, 1H), 7.41 (m, 1H), 7.27 (m, 2H), 5.09 (quint, J=7.2 Hz, 1H), 4.05 (t, J=5.6 Hz, 2H), 3.83 (s, 2H), 3.80 (s, 2H), 2.97 (t, J=5.6 Hz, 2H), 2.10-2.05 (m, 4H), 2.00-1.90 (m, 2H), 1.76-1.63 (m, 2H), LC/MS: m/e=384 (M+H)⁺.

### Compound I-72

### 8-(3-Chlorobenzyl)-1-cyclopentyl-6,7,8,9-tetrahydropyrazino[1,2-a]pyrazolo[3,4-d]pyrimidin-4(1H)-one

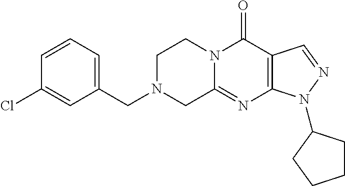 [SMILES: O=c1c2cnn(C3CCCC3)c2nc2n1CCN(Cc1cccc(Cl)c1)C2]

The title compound was prepared via the procedure used for I-84 substituting 3-chlorobenzaldehyde for 4-methoxybenzaldehyde. ¹H NMR (400 MHz, CDCl₃): δ 8.06 (s, 1H), 7.39 (s, 1H), 7.31-7.25 (m, 3H), 5.08 (quint, J=7.6 Hz, 1H), 4.06 (t, J=5.6 Hz, 2H), 3.71 (s, 2H), 3.69 (s, 2H), 2.93 (t, J=6.0 Hz, 2H), 2.06 (m, 4H), 1.97 (m, 2H), 1.70 (m, 2H), LC/MS: m/e=384 (M+H)⁺.

### Compound I-113

### (±)-8-(1-(4-Chlorophenyl)ethyl)-1-cyclopentyl-6,7,8,9-tetrahydropyrazino[1,2-a]pyrazolo[3,4-d]pyrimidin-4(1H)-one

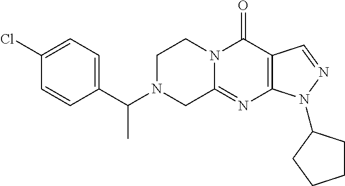 [SMILES: CC(c1ccc(Cl)cc1)N1CCn2c(nc3c(cnn3C3CCCC3)c2=O)C1]

The title compound was prepared via the procedure used for I-84 substituting 4-chloroacetophenone for 4-methoxybenzaldehyde. ¹H NMR (400 MHz, CDCl₃): δ 8.06 (s, 1H), 7.36 (d, J=6.8 Hz, 2H), 7.30 (d, J=6.8 Hz, 2H), 5.10 (quint, J=6.0 Hz, 1H), 4.03 (m, 1H), 3.97 (m, 1H), 3.84 (d, J=13.6 Hz, 1H), 3.68 (d, J=13.6 Hz, 1H), 3.54 (m, 1H), 2.86 (t, J=4.8 Hz, 2H), 2.13-2.05 (m, 4H), 1.98 (m, 2H), 1.73 (m, 2H), 1.48 (d, J=5.2 Hz, 3H), LC/MS: m/e=398 (M+H)⁻.

### Compound I-114

### (±)-1-Cyclopentyl-8-(1-(4-methoxyphenyl)ethyl)-6,7,8,9-tetrahydropyrazino[1,2-a]pyrazolo[3,4-d]pyrimidin-4(1H)-one

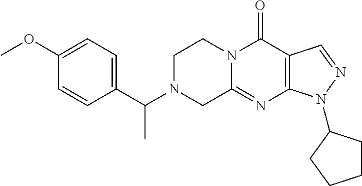 [SMILES: COc1ccc(C(C)N2CCn3c(nc4c(cnn4C4CCCC4)c3=O)C2)cc1]

The title compound was prepared via the procedure used for I-84 substituting 4-methoxyacetophenone for 4-methoxybenzaldehyde. ¹H NMR (400 MHz, CDCl₃): δ 8.04 (s, 1H), 7.25 (m, 2H), 6.90 (m, 2H), 5.08 (quint, J=7.6 Hz, 1H), 4.02 (m, 1H), 3.94 (m, 1H), 3.84 (d, J=16.8 Hz, 1H), 3.82 (s, 3H), 3.66 (d, J=16.8 Hz, 1H), 3.53 (dd, J=13.2 Hz, J₂=6.8 Hz, 1H), 2.81 (m, 2H), 2.11-2.03 (m, 4H), 1.96 (m, 2H), 1.72 (m, 2H), 1.46 (d, J=6.8 Hz, 3H), LC/MS: m/e=394 (M+H)⁺.

### Compound I-67

### 8-(2-Chlorobenzyl)-1-isopropyl-6,7,8,9-tetrahydropyrazino[1,2-a]pyrazolo[3,4-d]pyrimidin-4(1H)-one

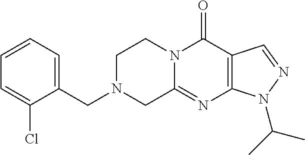 [SMILES: CC(C)n1ncc2c(=O)n3c(nc21)CN(Cc1ccccc1Cl)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 2-chlorobenzaldehyde for 4-methoxybenzaldehyde. ¹H NMR (400 MHz, CDCl₃): δ 8.06 (s, 1H), 7.48 (dd, J₁=6.8 Hz, J₂=2.0 Hz, 1H), 7.41 (dd, J₁=7.6 Hz, J₂=2.0 Hz, 1H), 7.31-7.24 (m, 2H), 4.95 (sept, J=6.4 Hz, 1H), 4.05 (t, J=5.6 Hz, 2H), 3.83 (s, 2H), 3.79 (s, 2H), 2.97 (t, J=6.0 Hz, 2H), 1.51 (d, J=6.8 Hz, 6H), LC/MS: m/e=358 (M+H)⁺.

### Compound I-68

### 8-(2-Chlorobenzyl)-1-isobutyl-6,7,8,9-tetrahydropyrazino[1,2-a]pyrazolo[3,4-d]pyrimidin-4(1H)-one

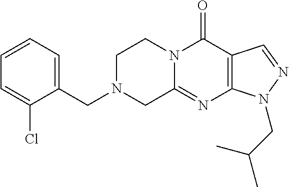 [SMILES: CC(C)Cn1ncc2c(=O)n3c(nc21)CN(Cc1ccccc1Cl)CC3]

The title compound was prepared via the procedure used for I-84 substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 2-chlorobenzaldehyde for 4-methoxybenzaldehyde. ¹H NMR (400 MHz, CDCl₃): δ 8.06 (s, 1H), 7.50 (dd, J₁=6.8 Hz, J₂=1.6 Hz, 1H), 7.42 (dd, J₁=7.2 Hz, J₂=2.0 Hz, 1H), 7.32-7.25 (m, 2H), 4.07 (m, 4H), 3.86 (s, 2H), 3.82 (s, 2H), 2.99 (t, J=5.6 Hz, 2H), 2.32 (m, 1H), 0.91 (d, J=6.8 Hz, 6H), LC/MS: m/e=372 (M+H)⁺.

### Compound I-70

### 8-(3-Chlorobenzyl)-1-isopropyl-6,7,8,9-tetrahydropyrazino[1,2-a]pyrazolo[3,4-d]pyrimidin-4(1H)-one

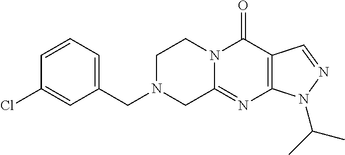 [SMILES: CC(C)n1ncc2c(=O)n3c(nc21)CN(Cc1cccc(Cl)c1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-chlorobenzaldehyde for 4-methoxybenzaldehyde. ¹H NMR (400 MHz, CDCl₃): δ 8.06 (s, 1H), 7.38 (s, 1H), 7.29 (m, 2H), 7.25 (m, 1H), 4.94 (sept, J=6.4 Hz, 1H), 4.05 (t, J=6.0 Hz, 2H), 3.71 (s, 2H), 3.68 (s, 2H), 2.91 (t, J=5.6 Hz, 2H), 1.50 (d, J=6.4 Hz, 6H), LC/MS: m/e=358 (M+H)⁺.

### Compound I-71

### 8-(3-Chlorobenzyl)-1-isobutyl-6,7,8,9-tetrahydropyrazino[1,2-a]pyrazolo[3,4-d]pyrimidin-4(1B)-one

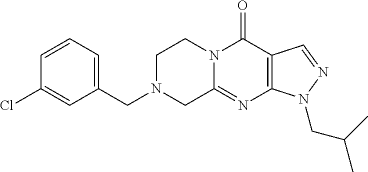 [SMILES: CC(C)Cn1ncc2c(=O)n3c(nc21)CN(Cc1cccc(Cl)c1)CC3]

The title compound was prepared via the procedure used for I-84 substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-chlorobenzaldehyde for 4-methoxybenzaldehyde. ¹H NMR (400 MHz, CDCl₃): δ 8.04 (s, 1H), 7.38 (s, 1H), 7.30-7.24 (m, 3H), 4.04 (m, 4H), 3.71 (s, 2H), 3.67 (s, 2H), 2.90 (t, J=5.6 Hz, 2H), 2.30 (m, 1H), 0.88 (d, J=6.8 Hz, 6H), LC/MS: m/e=372 (M+H)⁺.

### Compound I-73

### 8-(4-Chlorobenzyl)-1-isopropyl-6,7,8,9-tetrahydropyrazino[1,2-a]pyrazolo[3,4-d]pyrimidin-4(1H)-one

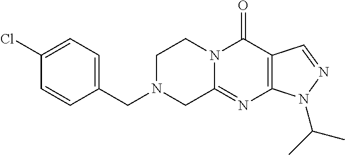 [SMILES: CC(C)n1ncc2c(=O)n3c(nc21)CN(Cc1ccc(Cl)cc1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-chlorobenzaldehyde for 4-methoxybenzaldehyde. ¹H NMR (400 MHz, CDCl₃): δ 8.06 (s, 1H), 7.35-7.30 (m, 4H), 4.05 (sept, J=6.8 Hz, 1H), 4.04 (t, J=5.6 Hz, 2H), 3.69 (s, 2H), 3.67 (s, 2H), 2.90 (t, J=5.6 Hz, 2H), 1.51 (d, J=6.8 Hz, 6H), LC/MS: m/e=358 (M+H)⁺.

### Compound I-74

### 8-(4-Chlorobenzyl)-1-isobutyl-6,7,8,9-tetrahydropyrazino[1,2-a]pyrazolo[3,4-d]pyrimidin-4(1H)-one

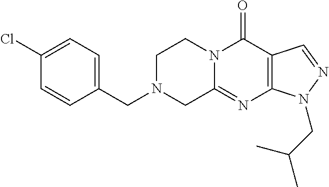 [SMILES: CC(C)Cn1ncc2c(=O)n3c(nc21)CN(Cc1ccc(Cl)cc1)CC3]

The title compound was prepared via the procedure used for I-84 substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-chlorobenzaldehyde for 4-methoxybenzaldehyde. ¹H NMR (400 MHz, CDCl₃): δ 8.05 (s, 1H), 7.36-7.27 (m, 4H), 4.04 (m, 4H), 3.70 (s, 2H), 3.67 (s, 2H), 2.90 (t, J=5.6 Hz, 2H), 2.31 (m, 1H), 0.90 (d, J=6.8 Hz, 6H), LC/MS: m/e=372 (M+H)⁺.

### Compound I-76

### 1-Isopropyl-8-(2-methoxybenzyl)-6,7,8,9-tetrahydropyrazino[1,2-a]pyrazolo[3,4-d]pyrimidin-4(1H)-one

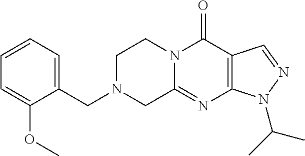 [SMILES: COc1ccccc1CN1CCn2c(nc3c(cnn3C(C)C)c2=O)C1]

The title compound was prepared via the procedure used for I-84 substituting 5-amino-1-isopropyl-1H-pyrazole-4-carboxamide (prepared according to the procedure in planing, H., et al., Bioorg. Med. Chem. Lett. 2005, 15, 3900) for 5-amino-1-cyclopentyl-1H-pyrazole-4-carboxamide, and substituting 2-methoxybenzaldehyde for 4-methoxybenzaldehyde. ¹H NMR (400 MHz, CDCl₃): δ 8.05 (s, 1H), 7.37-7.26 (m, 2H), 7.00-6.91 (m, 2H), 4.95 (sept, J=6.8 Hz, 1H), 4.03 (m, 2H), 3.86 (s, 3H), 3.77 (s, 2H), 3.76 (s, 2H), 2.94 (m, 2H), 1.51 (d; J=6.4 Hz, 6H), LC/MS: m/e=354 (M+H)⁺.

### Compound I-77

### 1-Isobutyl-8-(2-methoxybenzyl)-6,7,8,9-tetrahydropyrazino[1,2-a]pyrazolo[3,4-d]pyrimidin-4(1H)-one

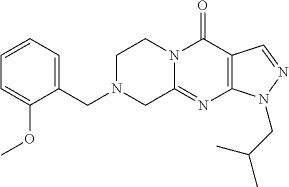 [SMILES: COc1ccccc1CN1CCn2c(nc3c(cnn3CC(C)C)c2=O)C1]

The title compound was prepared via the procedure used for I-84 substituting 5-amino-1-isobutyl-1H-pyrazole-4-carboxamide (prepared according to the procedure in Haning, H., et al., Bioorg. Med. Chem. Leu. 2005, 15, 3900) for 5-amino-1-cyclopentyl-1H-pyrazole-4-carboxamide, and substituting 2-methoxybenzaldehyde for 4-methoxybenzaldehyde. ¹H NMR (400 MHz, CDCl₃): δ 8.05 (s, 1H), 7.37 (d, J=7.2 Hz, 1H), 7.33 (m, 1H), 6.99 (t, J=7.6 Hz, 1H), 6.94 (d, J=8.4 Hz, 1H), 4.04 (m, 4H), 3.87 (s, 3H), 3.79 (m, 4H), 2.96 (m, 2H), 2.32 (m, 1H), 0.90 (d, J=6.8 Hz, 6H), LC/MS: m/e=368 (M+H)⁺.

### Compound I-79

### 1-Isopropyl-8-(3-methoxybenzyl)-6,7,8,9-tetrahydropyrazino[1,2-a]pyrazolo[3,4-d]pyrimidin-4(1H)-one

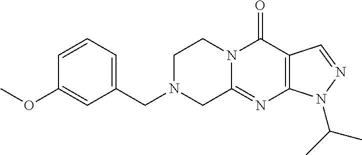 [SMILES: COc1cccc(CN2CCn3c(nc4c(cnn4C(C)C)c3=O)C2)c1]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-methoxybenzaldehyde for 4-methoxybenzaldehyde. ¹H NMR (400 MHz, CDCl₃): δ 8.05 (s, 1H), 7.27 (m, 1H), 6.94 (m, 2H), 6.85 (m, 1H), 4.94 (sept; J=6.8 Hz, 1H), 4.04 (t, J=5.6 Hz, 2H), 3.82 (s, 3H), 3.72 (s, 2H), 3.68 (s, 2H), 2.91 (t, J=5.6 Hz, 2H), 1.50 (d, J=6.8 Hz, 6H), LC/MS: m/e=354 (M+H)⁺.

### Compound I-80

### 1-Isobutyl-8-(3-methoxybenzyl)-6,7,8,9-tetrahydropyrazino[1,2-a]pyrazolo[3,4-d]pyrimidin-4(1H)-one

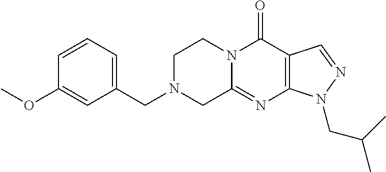 [SMILES: COc1cccc(CN2CCn3c(nc4c(cnn4CC(C)C)c3=O)C2)c1]

The title compound was prepared via the procedure used for I-84 substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-methoxybenzaldehyde for 4-methoxybenzaldehyde. ¹H NMR (400 MHz, CDCl₃): δ 8.05 (s, 1H), 7.29 (m, 1H), 6.94 (m, 2H), 6.86 (m, 1H), 4.05 (m, 4H), 3.83 (s, 3H), 3.73 (s, 2H), 3.68 (s, 2H), 2.91 (t, J=5.6 Hz, 2H), 2.31 (m, 1H), 0.89 (d, J=6.4 Hz, 6H), LC/MS: m/e=368 (M+H)⁺.

### Compound I-82

### 1-Isopropyl-8-(4-methoxybenzyl)-6,7,8,9-tetrahydropyrazino[1,2-a]pyrazolo[3,4-d]pyrimidin-4(1H)-one

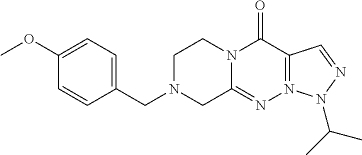

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 8.05 (s, 1H), 7.27 (m, 2H), 6.89 (m, 2H), 4.93 (sept, J=6.8 Hz, 1H), 4.02 (t, J=5.6 Hz, 2H), 3.82 (s, 3H), 3.69 (s, 2H), 3.64 (s, 2H), 2.88 (t, J=5.6 Hz, 2H), 1.50 (d, J=6.4 Hz, 6H), LC/MS: m/e=354 (M+H)⁺.

### Compound I-83

### 1-Isobutyl-8-(4-methoxybenzyl)-6,7,8,9-tetrahydropyrazino[1,2-a]pyrazolo[3,4-d]pyrimidin-4(1H)-one

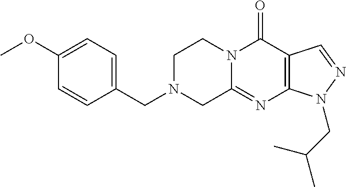 [SMILES: COc1ccc(CN2CCn3c(nc4c(cnn4CC(C)C)c3=O)C2)cc1]

The title compound was prepared via the procedure used for I-84 substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 8.05 (s, 1H), 7.28 (d, J=8.8 Hz, 2H), 6.91 (d, 8.8 Hz, 2H), 4.06 (d, J=7.6 Hz, 2H), 4.03 (t, J=6.0 Hz, 2H), 3.83 (s, 3H), 3.71 (s, 2H), 3.65 (s, 2H), 2.89 (t, J=5.6 Hz, 2H), 2.31 (m, 1H), 0.89 (d, J=6.8 Hz, 6H), LC/MS: m/e=368 (M+H)⁺.

### Compound I-135

### 1-Isopropyl-8-(pyridin-3-ylmethyl)-6,7,8,9-tetrahydropyrazino[1,2-a]pyrazolo[3,4-d]pyrimidin-4(1H)-one

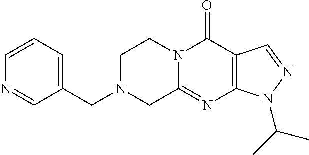 [SMILES: CC(C)n1ncc2c(=O)n3c(nc21)CN(Cc1cccnc1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-pyridinecarboxaldehyde for 4-methoxybenzaldehyde. ¹H NMR (400 MHz, CDCl₃): δ 8.60 (m, 1H), 8.57 (m, 1H), 8.05 (s, 1H), 7.72 (d, J=7.6 Hz, 1H), 7.31 (dd, J₁=8.0 Hz, J₂=4.8 Hz, 1H), 4.93 (sept, J=6.8 Hz, 1H), 4.04 (t, J=5.6 Hz, 2H), 3.72 (s, 4H), 2.92 (t, J=5.6 Hz, 2H), 1.49 (d, J=6.8 Hz, 6H), LC/MS: m/e=325 (M+H)⁺.

### Compound I-136

### 1-Isopropyl-8-(pyridin-4-ylmethyl)-6,7,8,9-tetrahydropyrazino[1,2-a]pyrazolo[3,4-d]pyrimidin-4(1H)-one

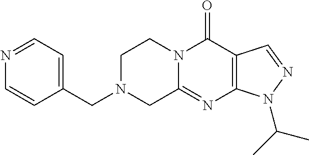 [SMILES: CC(C)n1ncc2c(=O)n3c(nc21)CN(Cc1ccncc1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-pyridinecarboxaldehyde for 4-methoxybenzaldehyde. ¹H NMR (400 MHz, CDCl₃): δ 8.61 (d, J=6.4 Hz, 2H), 8.06 (s, 1H), 7.33 (d, J=6.0 Hz, 2H), 4.93 (sept, J=6.8 Hz, 1H), 4.06 (t, J=5.6 Hz, 2H), 3.72 (s, 2H), 3.72 (s, 2H), 2.93 (t, J=5.6 Hz, 2H), 1.51 (d, J=6.8 Hz, 6H), LC/MS: m/e=325 (M+H)⁺.

### Compound I-137

### 8-(3-Fluorobenzyl)-1-isopropyl-6,7,8,9-tetrahydropyrazino[1,2-a]pyrazolo[3,4-d]pyrimidin-4(1H)-one

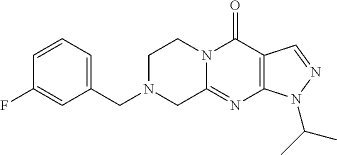 [SMILES: CC(C)n1ncc2c(=O)n3c(nc21)CN(Cc1cccc(F)c1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-fluorobenzaldehyde for 4-methoxybenzaldehyde. ¹H NMR (400 MHz, CDCl₃): δ 8.07 (s, 1H), 7.33 (m, 1H), 7.13 (m, 2H), 7.02 (m, 1H), 4.94 (sept, J=6.8 Hz, 1H), 4.05 (t, J=5.6 Hz, 2H), 3.72 (s, 2H), 3.70 (s, 2H), 2.92 (t, J=6.0 Hz, 2H), 1.51 (d, J=6.4 Hz, 6H), LC/MS: m/e=342 (M+H)⁺.

### Compound I-138

### 8-(4-Fluorobenzyl)-1-isopropyl-6,7,8,9-tetrahydropyrazino[1,2-a]pyrazolo[3,4-d]pyrimidin-4(1H)-one

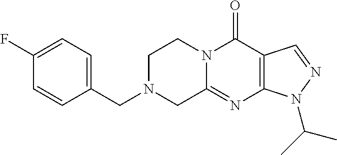 [SMILES: CC(C)n1ncc2c(=O)n3c(nc21)CN(Cc1ccc(F)cc1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-fluorobenzaldehyde for 4-methoxybenzaldehyde. ¹H NMR (400 MHz, CDCl₃): δ 8.06 (s, 1H), 7.33 (dd, J₁=8.4 Hz, J₂=5.6 Hz, 2H), 7.05 (m, 2H), 4.94 (sept, J=7.2 Hz, 1H), 4.03 (t, J=5.6 Hz, 2H), 3.69 (s, 2H), 3.67 (s, 2H), 2.90 (t, J=6.0 Hz, 2H), 1.50 (d, J=6.8 Hz, 6H), LC/MS: m/e=342 (M+H)⁺.

### Compound I-139

### 8-(4-Fluoro-3-methoxybenzyl)-1-isopropyl-6,7,8,9-tetrahydropyrazino[1,2-a]pyrazolo[3,4-d]pyrimidin-4(1H)-one

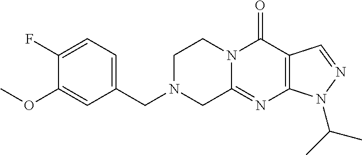 [SMILES: COc1cc(CN2CCn3c(nc4c(cnn4C(C)C)c3=O)C2)ccc1F]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-fluoro-3-methoxybenzaldehyde for 4-methoxybenzaldehyde. ¹H NMR (400 MHz, CDCl₃): δ 8.06 (s, 1H), 7.05 (m, 1H), 7.02 (m, 1H), 6.86 (m, 1H), 4.94 (sept, J=6.8 Hz, 1H), 4.04 (t, J=6.0 Hz, 2H), 3.90 (s, 3H), 3.70 (s, 2H), 3.65 (s, 2H), 2.90 (t, J=6.0 Hz, 2H), 1.50 (d, J=6.8 Hz, 6H). LC/MS: 117/C=372 (M+H)⁺.

### Compound I-140

### 8-(3-Fluoro-4-methoxybenzyl)-1-isopropyl-6,7,8,9-tetrahydropyrazino[1,2-a]pyrazolo[3,4-d]pyrimidin-4(1H)-one

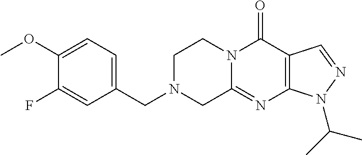 [SMILES: COc1ccc(CN2CCn3c(nc4c(cnn4C(C)C)c3=O)C2)cc1F]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-fluoro-4-methoxybenzaldehyde for 4-methoxybenzaldehyde. ¹H NMR (400 MHz, CDCl₃): δ 8.07 (s, 1H), 7.13 (dd, J₁=12.4 Hz, J₂=2.0 Hz, 1H), 7.06 (d, J=8.4 Hz, 1H), 6.95 (t, J=8.4 Hz, 1H), 4.93 (sept, J=6.8 Hz, 1H), 4.04 (t, J=6.0 Hz, 2H), 3.91 (s, 3H), 3.70 (s, 2H), 3.63 (s, 2H), 2.90 (t, J=5.6 Hz, 2H), 1.51 (d, J=6.8 Hz, 6H), LC/MS: m/e=372 (M+H)⁺.

### Compound I-158

### 8-(4-Methoxybenzyl)-1-(2-methoxyethyl)-6,7,8,9-tetrahydropyrazino[1,2-a]pyrazolo[3,4-d]pyrimidin-4(1H)-one

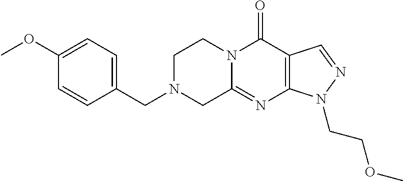

The title compound was prepared via the procedure used for I-84 substituting 5-amino-1-(2-methoxyethyl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 8.08 (s, 1H), 7.28 (d, J=6.8 Hz, 2H), 6.91 (d, J=6.8 Hz, 2H), 4.45 (t, J=4.4 Hz, 2H), 4.04 (t, J=4.4 Hz, 2H), 3.84 (s, 3H), 3.83 (t, J=4.0 Hz, 2H), 3.72 (s, 2H), 3.65 (s, 2H), 3.33 (s, 3H), 2.90 (t, J=4.8 Hz, 2H), LC/MS: m/e=370 (M+H)⁺.

### Compound I-159

### 8-(4-Chlorobenzyl)-1-(2-methoxyethyl)-6,7,8,9-tetrahydropyrazino[1,2-a]pyrazolo[3,4-d]pyrimidin-4(1H)-one

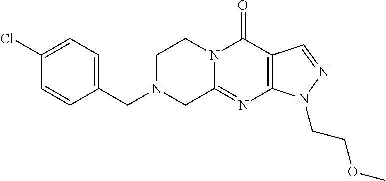 [SMILES: COCCn1ncc2c(=O)n3c(nc21)CN(Cc1ccc(Cl)cc1)CC3]

The title compound was prepared via the procedure used for I-84 substituting 5-amino-1-(2-methoxyethyl)-1H-pyrazole-4-carboxamide (prepared according to the procedure in Hailing, H., et al., Bioorg. Med. Chem. Lett. 2005, 15, 3900) for 5-amino-1-cyclopentyl-1H-pyrazole-4-carboxamide, and substituting 4-chlorobenzaldehyde for 4-methoxybenzaldehyde. ¹H NMR (400 MHz, CDCl₃): δ 8.07 (s, 1H), 7.35 (d, J=8.8 Hz, 2H), 7.31 (d, J=8.4 Hz, 2H), 4.44 (t, J=5.6 Hz, 2H), 4.03 (t, J=5.6 Hz, 2H), 3.82 (t, J=5.6 Hz, 2H), 3.71 (s, 2H), 3.67 (s, 2H), 3.31 (s, 3H), 2.90 (t, J=5.6 Hz, 2H), LC/MS: m/e=374 (M+H)⁺.

### Compound I-160

### 8-(3,4-Dichlorobenzyl)-1-(2-methoxyethyl)-6,7,8,9-tetrahydropyrazino[1,2-a]pyrazolo[3,4-d]pyrimidin-4(1H)-one

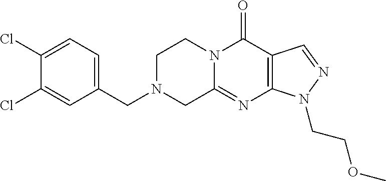 [SMILES: COCCn1ncc2c(=O)n3c(nc21)CN(Cc1ccc(Cl)c(Cl)c1)CC3]

The title compound was prepared via the procedure used for I-84 substituting 5-amino-1-(2-methoxyethyl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3,4-dichlorobenzaldehyde for 4-methoxybenzaldehyde. ¹H NMR (400 MHz, CDCl₃): δ 8.08 (s, 1H), 7.49 (d, J₁=2.0 Hz, 1H), 7.44 (d, J=8.0 Hz, 1H), 7.23 (dd, J₁=8.0 Hz, J₂=2.0 Hz, 1H), 4.44 (t, J=5.6 Hz, 2H), 4.05 (t, J=6.0 Hz, 2H), 3.82 (t, J=5.6 Hz, 2H), 3.71 (s, 2H), 3.65 (s, 2H), 3.32 (s, 3H), 2.92 (t, J=5.6 Hz, 2H), LC/MS: m/e=408 (M+H)⁺.

### Compound I-161

### 8-(3-Methoxybenzyl)-1-(2-methoxyethyl)-6,7,8,9-tetrahydropyrazino[1,2-a]pyrazolo[3,4-d]pyrimidin-4(1H)-one

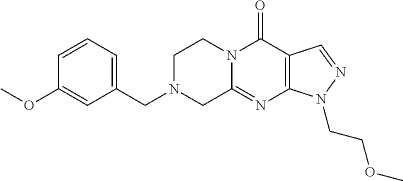

The title compound was prepared via the procedure used for I-84 substituting 5-amino-1-(2-methoxyethyl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-methoxybenzaldehyde for 4-methoxybenzaldehyde. ¹H NMR (400 MHz, CDCl₃): δ 8.07 (s, 1H), 7.27 (dd. J₁=7.6 Hz, J₂=5.6 Hz, 1H), 6.93 (m, 2H), 6.86 (m, 1H), 4.44 (t, J=5.6 Hz, 2H), 4.04 (t, J=5.6 Hz, 2H), 3.83 (s, 3H), 3.82 (t, J=5.6 Hz, 2H), 3.73 (s, 2H), 3.68 (s, 2H), 3.31 (s, 3H), 2.90 (t, J=5.6 Hz, 2H), LC/MS: m/e=370 (M+H)⁺.

### Compound I-162

### 8-(3-Chlorobenzyl)-1-(2-methoxyethyl)-6,7,8,9-tetrahydropyrazino[1,2-a]pyrazolo[3,4-d]pyrimidin-4(1H)-one

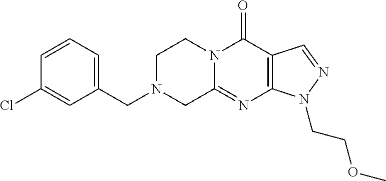 [SMILES: COCCn1ncc2c(=O)n3c(nc21)CN(Cc1cccc(Cl)c1)CC3]

The title compound was prepared via the procedure used for I-84 substituting 5-amino-1-(2-methoxyethyl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-chlorobenzaldehyde for 4-methoxybenzaldehyde. ¹H NMR (400 MHz, CDCl₃): δ 8.07 (s, 1H), 7.38 (s, 1H), 7.31-7.24 (m, 3H), 4.44 (t, J=5.6 Hz, 2H), 4.05 (t, J=5.6 Hz, 2H), 3.82 (t, J=5.2 Hz, 2H), 3.72 (s, 2H), 3.68 (s, 2H), 3.31 (s, 3H), 2.91 (t, J=5.6 Hz, 2H), LC/MS: m/e=374 (M+H)⁺.

### Compound I-163

### 8-(Benzo[d][1.3]dioxol-5-ylmethyl)-1-(2-methoxyethyl)-6,7,8,9-tetrahydropyrazino[1,2-a]pyrazolo[3,4-d]pyrimidin-4(1H)-one

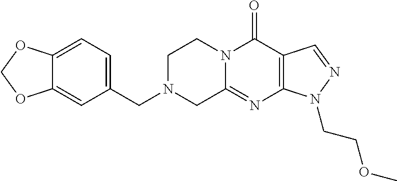 [SMILES: COCCn1ncc2c(=O)n3c(nc21)CN(Cc1ccc2c(c1)OCO2)CC3]

The title compound was prepared via the procedure used for I-84 substituting 5-amino-1-(2-methoxyethyl)-1H-pyrazole-4-carboxamide (prepared according to the procedure in Hailing, H., et al., Bioorg. Med. Chem. Lett. 2005, 15, 3900) for 5-amino-1-cyclopentyl-1H-pyrazole-4-carboxamide, and substituting benzo[d][1,3]dioxole-5-carbaldehyde for 4-methoxybenzaldehyde. ¹H NMR (400 MHz, CDCl₃): δ 8.07 (s, 1H), 6.88 (s, 1H), 6.79 (s, 2H), 6.00 (s, 2H), 4.44 (t, J=5.6 Hz, 2H), 4.03 (t, J=6.0 Hz, 2H), 3.82 (t, J=5.6 Hz, 2H), 3.71 (s, 2H), 3.61 (s, 2H), 3.32 (s, 3H), 2.89 (t, J=5.6 Hz, 2H), LC/MS: m/e=384 (M+H)⁺.

### Compound I-164

### 8-(4-Methoxybenzyl)-1-propyl-6,7,8,9-tetrahydropyrazino[1,2-a]pyrazolo[3,4-d]pyrimidin-4(1H)-one

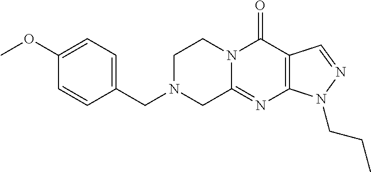 [SMILES: CCCn1ncc2c(=O)n3c(nc21)CN(Cc1ccc(OC)cc1)CC3]

The title compound was prepared via the procedure used for I-84 substituting 5-amino-1-propyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 8.05 (s, 1H), 7.29 (d, J=8.4 Hz, 2H), 6.91 (d, J=8.4 Hz, 2H), 4.23 (t, J=7.6 Hz, 2H), 4.04 (t, J=4.8 Hz, 2H), 3.84 (s, 3H), 3.71 (s, 2H), 3.66 (s, 2H), 2.90 (t, J=4.8 Hz, 2H), 1.90 (sext, J=7.6 Hz, 2H), 0.90 (t, J=7.2 Hz, 3H), LC/MS: m/e=354 (M+H)⁺.

### Compound I-253

### 8-(4-Chlorobenzyl)-1-propyl-6,7,8,9-tetrahydropyrazino[1,2-a]pyrazolo[3,4-d]pyrimidin-4(1H)-one

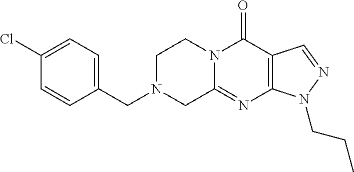 [SMILES: CCCn1ncc2c(=O)n3c(nc21)CN(Cc1ccc(Cl)cc1)CC3]

The title compound was prepared via the procedure used for I-84 substituting 5-amino-1-propyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-chlorobenzaldehyde for 4-methoxybenzaldehyde. NMR (400 MHz, CDCl₃): δ 8.05 (s, 1H), 7.36-7.29 (m, 4H), 4.23 (t, J=6.0 Hz, 2H), 4.04 (t, J=4.8 Hz, 2H), 3.71 (s, 2H), 3.68 (s, 2H), 2.91 (t, J=4.4 Hz, 2H), 1.92 (sext, J=6.0 Hz, 2H), 0.91 (t, J=6.0 Hz, 3H), LC/MS: m/e=358 (M+H)⁺.

### Compound I-165

### 8-(3,4-Dichlorobenzyl)-1-propyl-6,7,8,9-tetrahydropyrazino[1,2-a]pyrazolo[3,4-d]pyrimidin-4(1H)-one

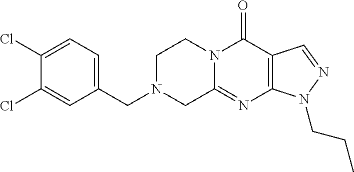 [SMILES: CCCn1ncc2c(=O)n3c(nc21)CN(Cc1ccc(Cl)c(Cl)c1)CC3]

The title compound was prepared via the procedure used for I-84 substituting 5-amino-1-propyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3,4-dichlorobenzaldehyde for 4-methoxybenzaldehyde. ¹H NMR (400 MHz, CDCl₃): δ 8.05 (s, 1H), 7.49 (d, J=2.0 Hz, 1H), 7.44 (d, J=8.4 Hz, 1H), 7.22 (dd, J₁=8.4 Hz, J₂=2.0 Hz, 1H), 4.22 (t, J=7.6 Hz, 2H), 4.05 (t, J=5.6 Hz, 2H), 3.70 (s, 2H), 3.66 (s, 2H), 2.92 (t, J=6.0 Hz, 2H), 1.91 (sext, J=7.6 Hz, 2H), 0.90 (t, J=7.2 Hz, 3H), LC/MS: m/e=392 (M+H)⁺.

### Compound I-254

### 8-(3-Methoxybenzyl)-1-propyl-6,7,8,9-tetrahydropyrazino[1,2-a]pyrazolo[3,4-d]pyrimidin-4(1H)-one

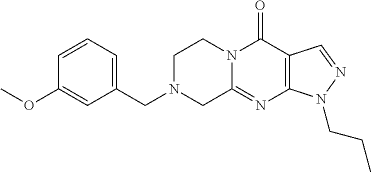 [SMILES: CCCn1ncc2c(=O)n3c(nc21)CN(Cc1cccc(OC)c1)CC3]

The title compound was prepared via the procedure used for I-84 substituting 5-amino-1-propyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-methoxybenzaldehyde for 4-methoxybenzaldehyde. ¹H NMR (400 MHz, CDCl₃): δ 8.06 (s, 1H), 7.30 (m, 1H), 6.97 (m, 2H), 6.89 (m, 1H), 4.24 (t, J=5.6 Hz, 2H), 4.06 (t, J=4.8 Hz, 2H), 3.84 (s, 3H), 3.75 (s, 2H), 330 (s, 2H), 2.93 (t, J=4.4 Hz, 2H), 1.92 (sext, J=5.6 Hz, 2H), 0.92 (t, J=6.4 Hz, 3H), LC/MS: m/e=354 (M+H)⁺.

### Compound I-166

### 8-(3-Chlorobenzyl)-1-propyl-6,7,8,9-tetrahydropyrazino[1,2-a]pyrazolo[3,4-d]pyrimidin-4(1H)-one

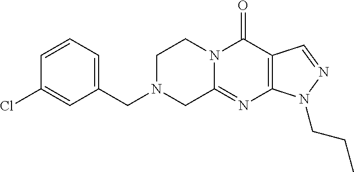 [SMILES: CCCn1ncc2c(=O)n3c(nc21)CN(Cc1cccc(Cl)c1)CC3]

The title compound was prepared via the procedure used for I-84 substituting 5-amino-1-propyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 3-chlorobenzaldehyde for 4-methoxybenzaldehyde. ¹H NMR (400 MHz, CDCl₃): δ 8.04 (s, 1H), 7.38 (s, 1H), 7.30-7.24 (m, 3H), 4.22 (t, J=7.6 Hz, 2H), 4.04 (t, J=5.6 Hz, 2H), 3.71 (s, 2H), 3.68 (s, 2H), 2.91 (t, J=5.6 Hz, 2H), 1.91 (sext, J=7.6 Hz, 2H), 0.89 (t, J=7.6 Hz, 3H), LC/MS: m/e=358 (M+H)⁺.

### Compound I-255

### 8-(Benzo[d][1,3]dioxol-5-ylmethyl)-1-propyl-6,7,8,9-tetrahydropyrazino[1,2-a]pyrazolo[3,4-d]pyrimidin-4(1H)-one

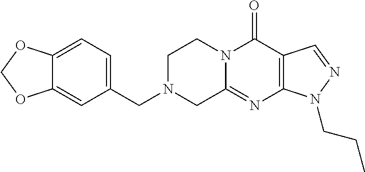 [SMILES: CCCn1ncc2c(=O)n3c(nc21)CN(Cc1ccc2c(c1)OCO2)CC3]

The title compound was prepared via the procedure used for I-84 substituting 5-amino-1-propyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting benzo[d][1,3]dioxole-5-carbaldehyde for 4-methoxybenzaldehyde. ¹H NMR (400 MHz, CDCl₃): δ 8.05 (s, 1H), 6.88 (s, 1H), 6.80 (s, 2H), 5.98 (s, 2H), 4.23 (t, J=7.2 Hz, 2H), 4.04 (t, J=5.6 Hz, 2H), 3.70 (s, 2H), 3.61 (s, 2H), 2.89 (t, J=5.6 Hz, 2H), 1.92 (sext, J=7.2 Hz, 2H), 0.90 (t, J=7.2 Hz, 3H), LC/MS: m/e=368 (M+H)⁺.

### Compound I-169

### 8-(3,4-Dichlorobenzyl)-1-isopropyl-6,7,8,9-tetrahydropyrazino[1,2-a]pyrazolo[3,4-d]pyrimidin-4(1H)-one

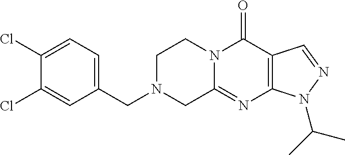 [SMILES: CC(C)n1ncc2c(=O)n3c(nc21)CN(Cc1ccc(Cl)c(Cl)c1)CC3]

The title compound was prepared via the procedure used for I-84 substituting 5-amino-1-isopropyl-1H-pyrazole-4-carboxamide (prepared according to the procedure in Haning, H., et al., Bioorg Med. Chem. Lett. 2005, 15, 3900) for 5-amino-1-cyclopentyl-1H-pyrazole-4-carboxamide, and substituting 3,4-dichlorobenzaldehyde for 4-methoxybenzaldehyde. ¹H NMR (400 MHz, CDCl₃): δ 8.07 (s, 1H), 7.50 (d, J=1.6 Hz, 1H), 7.45 (d, J=8.4 Hz, 1H), 7.23 (dd, J₁=8.4 Hz, J₂=1.6 Hz, 1H), 4.95 (sept, J=6.8 Hz, 1H), 4.06 (t, J=5.6 Hz, 2H), 3.70 (s, 2H), 3.66 (s, 2H), 2.93 (t, J=6.0 Hz, 2H), 1.50 (d, J=6.8 Hz, 6H), LC/MS: m/e=392 (M+H)⁺.

### Compound I-170

### 8-(Benzo[d][1,3]dioxol-5-ylmethyl)-1-isopropyl-6,7,8,9-tetrahydropyrazino[1,2-a]pyrazolo[3,4-d]pyrimidin-4(1H)-one

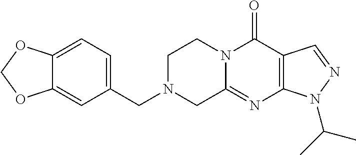 [SMILES: CC(C)n1ncc2c(=O)n3c(nc21)CN(Cc1ccc2c(c1)OCO2)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting benzo[d][1,3]dioxole-5-carbaldehyde for 4-methoxybenzaldehyde. H NMR (400 MHz, CDCl₃): δ 8.06 (s, 1H), 6.88 (s, 1H), 6.79 (s, 2H), 5.97 (s, 2H), 4.95 (sept, J=6.4 Hz, 1H), 4.03 (t, J=5.6 Hz, 2H), 3.70 (s, 2H), 3.61 (s, 2H), 2.89 (t, J=5.6 Hz, 2H), 1.51 (d, J=6.4 Hz, 6H), LC/MS: m/e=368 (M+H)⁺.

### Compound I-186

### 1-Isopropyl-8-((6-methylpyridin-3-yl)methyl)-6,7,8,9-tetrahydropyrazino[1,2-a]pyrazolo[3,4-d]pyrimidin-4(1H)-one

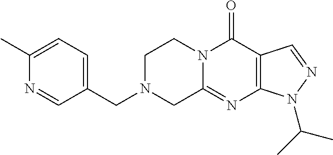 [SMILES: Cc1ccc(CN2CCn3c(nc4c(cnn4C(C)C)c3=O)C2)cn1]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 6-methylnicotinaldehyde for 4-methoxybenzaldehyde. ¹H NMR (400 MHz, CDCl₃): δ 8.48 (s, 1H), 8.07 (s, 1H), 7.63 (d, J=8.0 Hz, 1H), 7.19 (d, J=8.0 Hz, 1H), 4.96 (sept, J=6.4 Hz, 1H), 4.04 (t, J=5.6 Hz, 2H), 3.72 (s, 2H), 3.69 (s, 2H), 2.91 (t, J=6.0 Hz, 2H), 2.59 (s, 3H), 1.51 (d, J=6.8 Hz, 6H), LC/MS: m/e=339 (M+H)⁺.

### Compound I-187

### 1-Cyclopentyl-8-((6-methylpyridin-3-yl)methyl)-6,7,8,9-tetrahydropyrazino[1,2-a]pyrazolo[3,4-d]pyrimidin-4(1H)-one

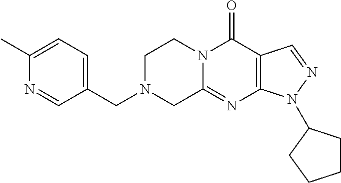 [SMILES: Cc1ccc(CN2CCn3c(nc4c(cnn4C4CCCC4)c3=O)C2)cn1]

The title compound was prepared via the procedure used for I-84 substituting 6-methylnicotinaldehyde for 4-methoxybenzaldehyde. ¹H NMR (400 MHz, CDCl₃): δ 8.43 (s, 1H), 8.01 (s, 1H), 7.58 (d, J=8.0 Hz, 1H), 7.14 (d, J=8.0 Hz, 1H), 5.04 (quint, J=7.6 Hz, 1H), 3.99 (t, J=5.6 Hz, 2H), 3.68 (s, 2H), 3.65 (s, 2H), 2.87 (t, J=6.0 Hz, 2H), 2.54 (s, 3H), 2.10-1.84 (m, 4H), 1.91 (m, 2H), 1.66 (m, 2H), LC/MS: m/e=365 (M+H)⁺.

### Compound I-190

### 1-Isopropyl-8-(pyrimidin-5-ylmethyl)-6,7,8,9-tetrahydropyrazino[1,2-a]pyrazolo[3,4-d]pyrimidin-4(1H)-one

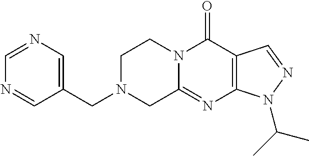 [SMILES: CC(C)n1ncc2c(=O)n3c(nc21)CN(Cc1cncnc1)CC3]

The title compound was prepared via the procedure used for I-84 substituting 5-amino-1-isopropyl-1H-pyrazole-4-carboxamide (prepared according to the procedure in Flailing, H., et al., Bioorg. Med. Chem. Lett. 2005, 15, 3900) for 5-amino-1-cyclopentyl-1H-pyrazole-4-carboxamide, and substituting pyrimidine-5-carbaldehyde for 4-methoxybenzaldehyde. ¹H NMR (400 MHz, CDCl₃): δ 9.22 (s, 1H), 8.79 (s, 2H), 8.08 (s, 1H), 4.94 (sept, J=6.8 Hz, 1H), 4.07 (t, J=5.6 Hz, 2H), 3.75 (s, 4H), 2.97 (t, J=6.0 Hz, 2H), 1.51 (d, J=6.0 Hz, 6H), LC/MS: m/e=326 (M+H)⁺.

### Compound I-191

### 1-Cyclopentyl-8-(pyrimidin-5-ylmethyl)-6,7,8,9-tetrahydropyrazino[1,2-a]pyrazolo[3,4-d]pyrimidin-4(1H)-one

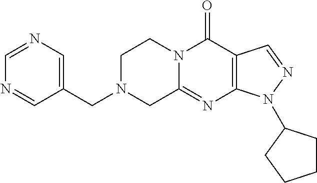 [SMILES: O=c1c2cnn(C3CCCC3)c2nc2n1CCN(Cc1cncnc1)C2]

The title compound was prepared via the procedure used for I-84 substituting pyrimidine-5-carbaldehyde for 4-methoxybenzaldehyde. ¹H NMR (400 MHz, CDCl₃): δ 9.21 (s, 1H), 8.78 (s, 2H), 8.06 (s, 1H), 5.08 (quint, J=7.6 Hz, 1H), 4.07 (t, J=5.6 Hz, 2H), 3.74 (s, 4H), 2.97 (t, J=5.6 Hz, 2H), 2.15-2.00 (m, 4H), 1.97 (m, 2H), 1.72 (m, 2H), LC/MS: m/e=352 (M±H)⁺.

### Compound I-194

### 1-Isopropyl-8-((2-methylpyrimidin-5-yl)methyl)-6,7,8,9-tetrahydropyrazino[1,2-a]pyrazolo[3,4-d]pyrimidin-4(1H)-one

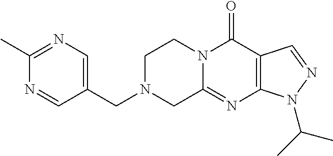 [SMILES: Cc1ncc(CN2CCn3c(nc4c(cnn4C(C)C)c3=O)C2)cn1]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 2-methylpyrimidine-5-carbaldehyde for 4-methoxybenzaldehyde. ¹H NMR (400 MHz, CDCl₃): δ 8.73 (s, 2H), 8.05 (s, 1H), 5.00 (sept, J=6.8 Hz, 1H), 4.05 (t, J=5.6 Hz, 2H), 3.81 (s, 2H), 3.80 (s, 2H), 3.02 (t, J=5.6 Hz, 2H), 2.75 (s, 3H), 1.51 (d, J=6.4 Hz, 6H), LC/MS: m/e=340 (M+H)⁺.

### Compound I-195

### 1-Cyclopentyl-8-((2-methylpyrimidin-5-yl)methyl)-6,7,8,9-tetrahydropyrazino[1,2-a]pyrazolo[3,4-d]pyrimidin-4(1H)-one

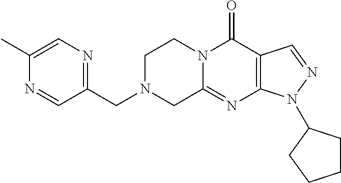 [SMILES: Cc1cnc(CN2CCn3c(nc4c(cnn4C4CCCC4)c3=O)C2)cn1]

The title compound was prepared via the procedure used for I-84 substituting 2-methylpyrimidine-5-carbaldehyde for 4-methoxybenzaldehyde. ¹H NMR (400 MHz, CDCl₃): δ 8.65 (s, 2H), 8.05 (s, 1H), 5.07 (quint, J=7.6 Hz, 1H), 4.04 (t, J=5.6 Hz, 2H), 3.72 (s, 2H), 3.69 (s, 2H), 2.93 (t, J=6.0 Hz, 2H), 2.76 (s, 3H), 2.14-2.00 (m, 4H), 1.95 (m, 2H), 1.69 (m, 2H), LC/MS: m/e=366 (M+H)⁺.

### Compound I-198

### 1-Isopropyl-8-((5-methylpyrimidin-2-yl)methyl)-6,7,8,9-tetrahydropyrazino[1,2-a]pyrazolo[3,4-d]pyrimidin-4(1H)-one

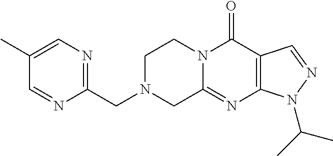 [SMILES: Cc1cnc(CN2CCn3c(nc4c(cnn4C(C)C)c3=O)C2)nc1]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 5-methylpyrimidine-2-carbaldehyde for 4-methoxybenzaldehyde. ¹H NMR (400 MHz, CDCl₃): δ 8.62 (s, 2H), 8.07 (s, 1H), 4.96 (sept, J=6.8 Hz, 1H), 4.12 (t, J=5.6 Hz, 2H), 4.02 (s, 2H), 3.92 (s, 2H), 3.08 (t, J=5.6 Hz, 2H), 2.33 (s, 3H), 1.52 (d, J=6.8 Hz, 6H), LC/MS: m/e=340 (M+H)⁺.

### Compound I-199

### 1-Cyclopentyl-8-((5-methylpyrimidin-2-yl)methyl)-6,7,8,9-tetrahydropyrazino[1,2-a]pyrazolo[3,4-d]pyrimidin-4(1H)-one

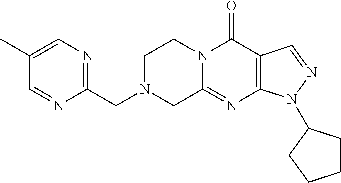 [SMILES: Cc1cnc(CN2CCn3c(nc4c(cnn4C4CCCC4)c3=O)C2)nc1]

The title compound was prepared via the procedure used for I-84 substituting 5-methylpyrimidine-2-carbaldehyde for 4-methoxybenzaldehyde. ¹H NMR (400 MHz, CDCl₃): δ 8.62 (s, 2H), 8.06 (s, 1H), 5.09 (quint, J=7.2 Hz, 1H), 4.11 (t, J=5.6 Hz, 2H), 4.02 (s, 2H), 3.91 (s, 2H), 3.08 (t, J=5.6 Hz, 2H), 2.35 (s, 3H), 2.08 (m, 4H), 1.96 (m, 2H), 1.71 (m, 2H), LC/MS: m/e=366 (M+H)⁺.

### Compound I-202

### 1-Isopropyl-8-(pyrazin-2-ylmethyl)-6,7,8,9-tetrahydropyrazino[1,2-a]pyrazolo[3,4-d]pyrimidin-4(1H)-one

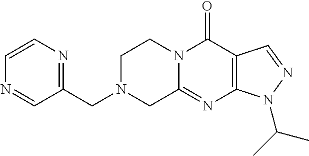 [SMILES: CC(C)n1ncc2c(=O)n3c(nc21)CN(Cc1cnccn1)CC3]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting pyrazine-2-carbaldehyde for 4-methoxybenzaldehyde. ¹H NMR (400 MHz, CDCl₃): δ 8.74 (s, 1H), 8.60 (s, 1H), 8.56 (m, 1H), 8.07 (s, 1H), 4.94 (sept, J=6.8 Hz, 1H), 4.09 (t, J=5.6 Hz, 2H), 3.93 (s, 2H), 3.83 (s, 2H), 3.03 (t, J=6.0 Hz, 2H), 1.51 (d, J=6.4 Hz, 6H), LC/MS: m/e=326 (M+H)⁺.

### Compound I-203

### 1-Cyclopentyl-8-(pyrazin-2-ylmethyl)-6,7,8,9-tetrahydropyrazino[1,2-a]pyrazolo[3,4-d]pyrimidin-4(1H)-one

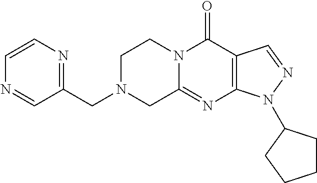 [SMILES: O=c1c2cnn(C3CCCC3)c2nc2n1CCN(Cc1cnccn1)C2]

The title compound was prepared via the procedure used for I-84 substituting pyrazine-2-carbaldehyde for 4-methoxybenzaldehyde. ¹H NMR (400 MHz, CDCl₃): δ 8.74 (s, 1H), 8.60 (m, 1H), 8.56 (m, 1H), 8.06 (s, 1H), 5.08 (quint, J=7.2 Hz, 1H), 4.08 (t, J=5.6 Hz, 2H), 3.93 (s, 2H), 3.82 (s, 2H), 3.02 (t, J=5.6 Hz, 2H), 2.11-2.00 (m, 4H), 1.97 (m, 2H), 1.72 (m, 2H), LC/MS: m/e=352 (M+H)⁺.

### Compound I-206

### 1-Isopropyl-8-((5-methylpyrazin-2-yl)methyl)-6,7,8,9-tetrahydropyrazino[1,2-a]pyrazolo[3,4-d]pyrimidin-4(1H)-one

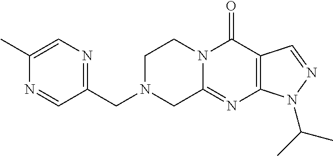 [SMILES: Cc1cnc(CN2CCn3c(nc4c(cnn4C(C)C)c3=O)C2)cn1]

The title compound was prepared via the procedure used for I-84 substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 5-methylpyrazine-2-carbaldehyde for 4-methoxybenzaldehyde. ¹H NMR (400 MHz, CDCl₃): δ 8.59 (s, 1H), 8.48 (s, 1H), 8.07 (s, 1H), 4.95 (sept, J=6.8 Hz, 1H), 4.08 (t, J=5.6 Hz, 2H), 3.88 (s, 2H), 3.82 (s, 2H), 3.01 (t, J=5.2 Hz, 2H), 2.61 (s, 3H), 1.51 (d, J=6.4 Hz, 6H), LC/MS: m/e=340 (M+H)⁺.

### Compound I-207

### 1-Cyclopentyl-8-((5-methylpyrazin-2-yl)methyl)-6,7,8,9-tetrahydropyrazino[1,2-a]pyrazolo[3,4-d]pyrimidin-4(1H)-one

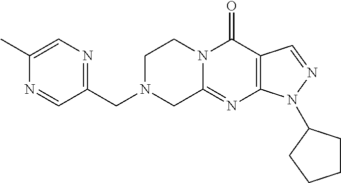 [SMILES: Cc1cnc(CN2CCn3c(nc4c(cnn4C4CCCC4)c3=O)C2)cn1]

The title compound was prepared via the procedure used for I-84 substituting 5-methylpyrazine-2-carbaldehyde for 4-methoxybenzaldehyde. ¹H NMR (400 MHz, CDCl₃): δ 8.58 (s, 1H), 8.47 (s, 1H), 8.05 (s, 1H), 5.08 (quint, J=7.2 Hz, 1H), 4.07 (t, J=6.0 Hz, 2H), 3.88 (s, 2H), 3.81 (s, 2H), 3.00 (t, J=5.6 Hz, 2H), 2.60 (s, 3H), 2.15-2.00 (m, 4H), 1.95 (m, 2H), 1.70 (m, 2H), LC/MS: m/e=366 (M+H)⁺.

### Compound I-210

### 8-(4-Methoxybenzyl)-1-(tetrahydro-2H-pyran-4-yl)-6,7,8,9-tetrahydropyrazino[1,2-a]pyrazolo[3,4-d]pyrimidin-4(1H)-one

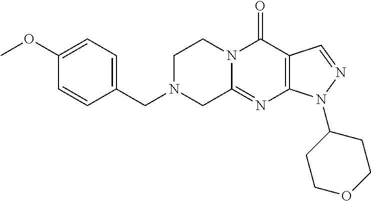 [SMILES: COc1ccc(CN2CCn3c(nc4c(cnn4C4CCOCC4)c3=O)C2)cc1]

The title compound was prepared via the procedure used for I-84 substituting 5-amino-1-(tetrahydro-2H-pyran-4-yl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 8.06 (s, 1H), 7.28 (d, J=8.8 Hz, 2H), 6.91 (d, J=8.4 Hz, 2H), 4.75 (m, 1H), 4.12 (m, 2H), 4.03 (t, J=6.0 Hz, 2H), 3.83 (s, 3H), 3.69 (s, 2H), 3.65 (s, 2H), 3.58 (m, 2H), 2.90 (t, J=6.0 Hz, 2H), 2.35 (m, 2H), 1.86 (m, 2H), LC/MS: m/e=396 (M+H)⁺.

### Compound I-211

### 8-(4-Chlorobenzyl)-1-(tetrahydro-2H-pyran-4-yl)-6,7,8,9-tetrahydropyrazino[1,2-a]pyrazolo[3,4-d]pyrimidin-4(1H)-one

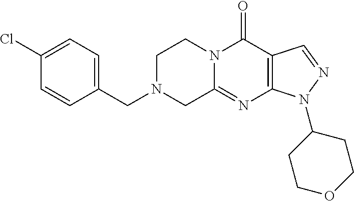 [SMILES: O=c1c2cnn(C3CCOCC3)c2nc2n1CCN(Cc1ccc(Cl)cc1)C2]

The title compound was prepared via the procedure used for I-84 substituting 5-amino-1-(tetrahydro-2H-pyran-4-yl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-chlorobenzaldehyde for 4-methoxybenzaldehyde. ¹H NMR (400 MHz, CDCl₃): δ 8.07 (s, 1H), 7.38 (d, J=8.4 Hz, 2H), 7.33 (d, J=8.8 Hz, 2H), 4.77 (m, 1H), 4.14 (dd, J₁=11.2 Hz, J₂=4.0 Hz, 2H), 4.06 (t, J=5.6 Hz, 2H), 3.70 (s, 2H), 3.69 (s, 2H), 3.59 (dd, J₁=11.2 Hz, J₂=1.6 Hz, 2H), 2.93 (t, J=5.6 Hz, 2H), 2.37 (m, 2H), 1.88 (dd, J₁=12.0 Hz, J₂=2.4 Hz, 2H), LC/MS: m/e=400 (M+H)⁺.

### Compound I-218

### (±)-8-Benzyl-1-(tetrahydrofuran-3-yl)-6,7,8,9-tetrahydropyrazino[1,2-a]pyrazolo[3,4-d]pyrimidin-4(1H)-one

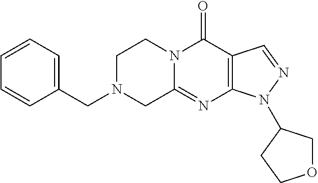 [SMILES: O=c1c2cnn(C3CCOC3)c2nc2n1CCN(Cc1ccccc1)C2]

The title compound was prepared via the procedure used for I-84 substituting racemic 5-amino-1-(tetrahydrofuran-3-yl)-1H-pyrazole-4-carboxamide (prepared according to the procedure in Halting, H., et al., Bioorg. Med. Chem. Lett. 2005, 15, 3900) for 5-amino-1-cyclopentyl-1H-pyrazole-4-carboxamide, and substituting benzaldehyde for 4-methoxybenzaldehyde. ¹H NMR (400 MHz, CDCl₃): δ 8.09 (s, 1H), 7.36 (m, 5H), 5.37 (m, 1H), 4.21 (dd, J₁=16.0 Hz, J₂=8.4 Hz, 1H), 4.15 (m, 1H), 4.04 (m, 4H), 3.74 (s, 2H), 3.73 (s, 2H), 2.93 (t, J=5.6 Hz, 2H), 2.43 (m, 2H), LC/MS: m/e=352 (M+H)⁺.

### Compound I-219

### (±)-8-(4-methoxybenzyl)-1-(tetrahydrofuran-3-yl)-6,7,8,9-tetrahydropyrazino[1,2-a]pyrazolo[3,4-d]pyrimidin-4(1H)-one

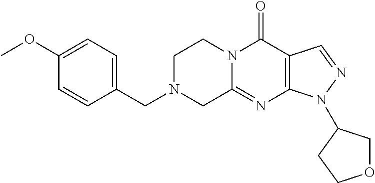 [SMILES: COc1ccc(CN2CCn3c(nc4c(cnn4C4CCOC4)c3=O)C2)cc1]

The title compound was prepared via the procedure used for I-84 substituting racemic 5-amino-1-(tetrahydrofuran-3-yl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 8.08 (s, 1H), 7.29 (d, J=6.0 Hz, 2H), 6.91 (d, J=8.0 Hz, 2H), 5.36 (m, 1H), 4.23 (dd, J₁=16.0 Hz, J₂=8.4 Hz, 1H), 4.15 (m, 1H), 4.03 (m, 4H), 3.84 (s, 3H), 3.71 (s, 2H), 3.66 (s, 2H), 2.90 (t, J=5.6 Hz, 2H), 2.44 (m, 2H), LC/MS: m/e=382 (M+H)⁺.

### Compound I-220

### (±)-8-(4-Chlorobenzyl)-1-(tetrahydrofuran-3-yl)-6,7,8,9-tetrahydropyrazino[1,2-a]pyrazolo[3,4-d]pyrimidin-4(1H)-one

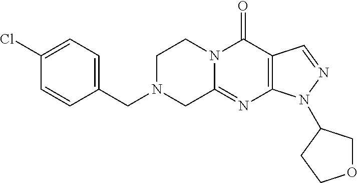 [SMILES: O=c1c2cnn(C3CCOC3)c2nc2n1CCN(Cc1ccc(Cl)cc1)C2]

The title compound was prepared via the procedure used for I-84 substituting racemic 5-amino-1-(tetrahydrofuran-3-yl)-1H-pyrazole-4-carboxamide (prepared according to the procedure in Haning, H., et al., Bioorg. Med. Chem. Lett. 2005, 15, 3900) for 5-amino-1-cyclopentyl-1H-pyrazole-4-carboxamide, and substituting 4-chlorobenzaldehyde for 4-methoxybenzaldehyde. ¹H NMR (400 MHz, CDCl₃): δ 8.09 (s, 1H), 7.36 (d, J=8.0 Hz, 2H), 7.32 (d, J=8.4 Hz, 2H), 5.36 (m, 1H), 4.23 (dd, J₁=16.0 Hz, J₂=7.6 Hz, 1H), 4.15 (m, 1H), 4.07 (m, 4H), 3.71 (s, 2H), 3.69 (s, 2H), 2.92 (t, J=5.6 Hz, 2H), 2.44 (m, 2H), LC/MS: m/e=386 (M+H)⁺.

### Compound I-243

### 8-(4-Chlorophenethyl)-1-cyclopentyl-6,7,8,9-tetrahydropyrazino[1,2-a]pyrazolo[3,4-d]pyrimidin-4(1H)-one

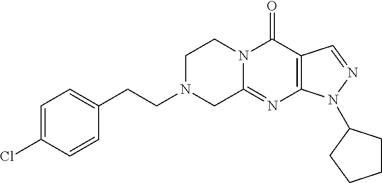 [SMILES: O=c1c2cnn(C3CCCC3)c2nc2n1CCN(CCc1ccc(Cl)cc1)C2]

The title compound was prepared via the procedure used for I-84 substituting 4-phenylacetaldehyde for 4-methoxybenzaldehyde. ¹H NMR (400 MHz, d⁴-MeOH): δ 8.00 (s, 1H), 7.26 (m, 4H), 5.15 (quint, J=7.2 Hz, 1H), 3.97 (t, J=5.6 Hz, 2H), 3.96 (s, 2H), 2.99 (t, J=5.6 Hz, 2H), 2.86 (m, 2H), 2.78 (m, 2H), 2.13-2.00 (m, 4H), 1.96 (m, 2H), 1.73 (m, 2H), LC/MS: m/e=398 (M+H)⁺.

### Example 2

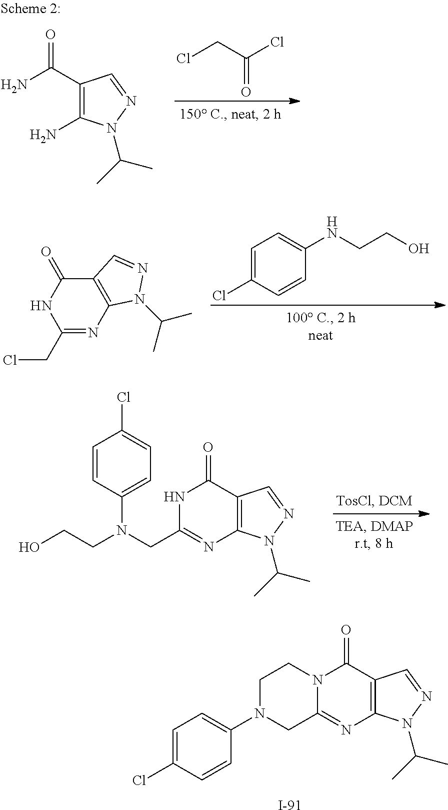

### Compound I-91

### 8-(4-Chlorophenyl)-1-isopropyl-6,7,8,9-tetrahydropyrazino[1,2-a]pyrazolo[3,4-d]pyrimidin-4(1H)-one

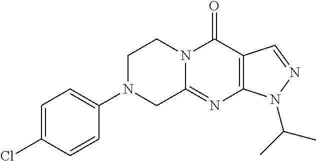 [SMILES: CC(C)n1ncc2c(=O)n3c(nc21)CN(c1ccc(Cl)cc1)CC3]

To a solution of 6-(((4-chlorophenyl)(2-hydroxyethyl)-amino)methyl)-1-isobutyl-1H-pyrazolo[3,4-d]pyrimidin-4(5H)-one (165 mg, 0.46 mmol) in dichloromethane (10 mL) was added 4-methylbenzene-1-sulfonyl chloride (176 mg, 0.92 mmol), triethylamine (139 mg, 1.38 mmol) and N,N-dimethylpyridin-4-amine (18 mg, cat) in one portion. The mixture was stirred at room temperature for 8 hours. Upon completion, the reaction mixture was washed with water (10 mL×2), dried and concentrated in vacuo to give the crude product which was purified by reverse column (acetonnitrile/water=60/40, 0.1% ammonia) to give 8-(4-Chlorophenyl)-1-isopropyl-6,7,8,9-tetrahydropyrazino[1,2-a]pyrazolo[3,4-a]pyrimidin-4(1H)-one (20 mg, yield: 12%) as a light yellow solid. ¹H NMR (400 MHz, CDCl₃): δ 8.09 (s, 1H), 7.26 (m, 2H), 6.84 (m, 2H), 6.77 (m, 1H), 5.00 (sept, J=6.8 Hz, 1H), 4.49 (s, 2H), 4.28 (t, J=5.6 Hz, 2H), 3.63 (t, 5.6 Hz, 2H), 1.54 (d, J=6.8 Hz, 6H), LC/MS: m/e=344 (M+H)⁺.

### 6-(Chloromethyl)-1-isopropyl-1H-pyrazolo[3,4-d]pyrimidin-4(5H)-one

A mixture of 5-amino-1-isopropyl-1H-pyrazole-4-carboxamide (prepared according to the procedure in Haning, H., et al., Bioorg. Med. Chem. Lett. 2005, 15, 3900) (10 g, 59.52 mmol) and 2-chloroacetyl chloride (13.5 g, 119 mmol) was heated to 150° C. and stirred for 2 h. The reaction mixture was dissolved in dichloromethane (100 mL) and washed with saturated sodium bicarbonate (50 mL×3). The organic layer was dried over anhydrous sodium sulfate and then concentrated in vacuo. A residue precipitated which was dissolved in dichloromethane (50 mL), filtered and washed with dichloromethane (10 mL×3) to get 6-(chloromethyl)-1-isopropyl-1H-pyrazolo[3,4-d]pyrimidin-4(5H)-one (6.5 g, yield: 48%) as a pale-yellow solid. LC/MS: m/e=227 (M+H)⁺.

### 2-(4-Chlorophenylamino)ethanol

To a mixture of 1-chloro-4-iodobenzene (3.3 g, 14 mmol) in dimethyl sulfoxide (4 mL) and water (2 mL) was added 2-aminoethanol (710 mg, 11.6 mmol), sodium hydroxide (1.1 g, 28 mmol) and cuprous iodide (315 mg, 1.6 mmol). The reaction was purged with nitrogen and stirred at 90° C. overnight. The reaction mixture was charged directly to a reverse phase column and purified (acetonitrile/water=31/69, 0.1% ammonia) to afford 2-(4-chlorophenylamino)ethanol (1.4 g, yield: 58%) as a white oil. LC/MS: m/e=172 (M+H)⁺.

### 6-(((4-Chlorophenyl)(2-hydroxyethyl)amino)methyl)-1-isobutyl-1H-pyrazolo[3,4-d]pyrimidin-4(5H)-one

A mixture of 6-(chloromethyl)-1-isobutyl-1H-pyrazolo[3,4-d]pyrimidin-4(5H)-one (100 mg, 0.44 mmol) and 2-(4-chlorophenylamino)ethanol (150 mg, 0.88 mmol) was heated to 100° C. with stirring for 2 h. Upon completion, the reaction mixture was cooled to room temperature, diluted with dichloromethane (20 mL), washed with 0.5 N aqueous hydrochloride (30 mL×2), dried over sodium sulfate and concentrated in vacuo to give 6-(((4-chlorophenyl)(2-hydroxyethyl)amino)-methyl)-1-isobutyl-1H-pyrazolo[3,4-d]pyrimidin-4(5H)-one (165 mg, purity: 79%) as a brown solid. LC/MS: m/e=362 (M+H)⁺.

### Compound I-55

### 1-Isopropyl-8-phenyl-6,7,8,9-tetrahydropyrazino[1,2-a]pyrazolo[3,4-d]pyrimidin-4(1H)-one

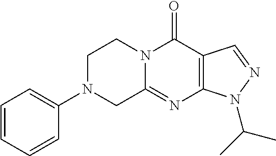 [SMILES: CC(C)n1ncc2c(=O)n3c(nc21)CN(c1ccccc1)CC3]

The title compound was prepared via the procedure used for I-91, substituting 2-(phenylamino)ethanol) for 2-(4-chlorophenylamino)ethanol. ¹H NMR (400 MHz, CDCl₃): δ 8.10 (s, 1H), 7.35 (m, 2H), 6.94 (m, 3H), 5.02 (sept, J=6.4 Hz, 1H), 4.52 (s, 2H), 4.27 (t, J=5.6 Hz, 2H), 3.67 (t, J=5.6 Hz, 2H), 1.55 (d, J=6.4 Hz, 6H), LC/MS: m/e=310 (M+H)⁻.

### Compound I-85

### 8-(2-Chlorophenyl)-1-isopropyl-6,7,8,9-tetrahydropyrazino[1,2-a]pyrazolo[3,4-d]pyrimidin-4(1H)-one

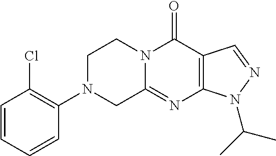 [SMILES: CC(C)n1ncc2c(=O)n3c(nc21)CN(c1ccccc1Cl)CC3]

The title compound was prepared via the procedure used for I-91, substituting 2-(2-chlorophenylamino)ethanol) for 2-(4-chlorophenylamino)ethanol. ¹H NMR (400 MHz, CDCl₃): δ 8.09 (s, 1H), 7.44 (dd, J₁=8.0 Hz, J₂=1.6 Hz, 1H), 7.28 (m, 1H), 7.09 (m, 2H), 5.00 (sept, J=6.8 Hz, 1H), 4.38 (s, 2H), 4.19 (t, J=5.6 Hz, 2H), 3.56 (t, J=5.6 Hz, 2H), 1.54 (d, J=6.8 Hz, 6H), LC/MS: m/e=344 (M+H)⁺.

### Compound I-86

### 8-(2-Chlorophenyl)-1-isobutyl-6,7,8,9-tetrahydropyrazino[1,2-a]pyrazolo[3,4-d]pyrimidin-4(1H)-one

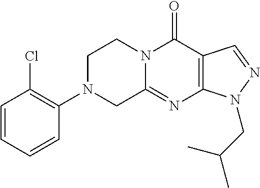 [SMILES: CC(C)Cn1ncc2c(=O)n3c(nc21)CN(c1ccccc1Cl)CC3]

The title compound was prepared via the procedure used for I-91, substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(2-chlorophenylamino)ethanol) for chlorophenylamino)ethanol. ¹H NMR (400 MHz, CDCl₃): δ 8.09 (s, 1H), 7.45 (dd, J₁=8.8 Hz, J₂=1.6 Hz, 1H), 7.29 (m, 1H), 7.10 (m, 2H), 4.38 (s, 2H), 4.20 (t, J=6.0 Hz, 2H), 4.12 (d, J=7.2 Hz, 2H), 3.57 (t, J=5.6 Hz, 2H), 2.34 (m, 1H), 0.92 (d, J=6.8 Hz, 6H), LC/MS: m/e=358 (M+H)⁺.

### Compound I-87

### 8-(2-Chlorophenyl)-1-cyclopentyl-6,7,8,9-tetrahydropyrazino[1,2-a]pyrazolo[3,4-d]pyrimidin-4(1H)-one

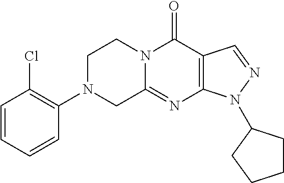 [SMILES: O=c1c2cnn(C3CCCC3)c2nc2n1CCN(c1ccccc1Cl)C2]

The title compound was prepared via the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(2-chlorophenylamino)ethanol) for 2-(4-chlorophenylamino)ethanol. ¹H NMR (400 MHz, CDCl₃): δ 8.09 (s, 1H), 7.45 (m, 1H), 7.29 (m, 1H), 7.11 (m, 2H), 5.15 (quint, J=7.6 Hz, 1H), 4.38 (s, 2H), 4.18 (t, J=5.6 Hz, 2H), 3.57 (t, J=5.6 Hz, 2H), 2.15-2.05 (m, 4H), 1.99 (m, 2H), 1.73 (m, 2H), LC/MS: m/e=370 (M+H)⁺.

### Compound I-88

### 8-(3-Chlorophenyl)-1-isopropyl-6,7,8,9-tetrahydropyrazino[1,2-a]pyrazolo[3,4-d]pyrimidin-4(1H)-one

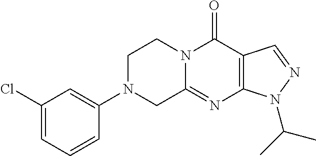 [SMILES: CC(C)n1ncc2c(=O)n3c(nc21)CN(c1cccc(Cl)c1)CC3]

The title compound was prepared via the procedure used for I-91, substituting 2-(3-chlorophenylamino)ethanol) for 2-(4-chlorophenylamino)ethanol. ¹H NMR (400 MHz, CDCl₃): δ 8.10 (s, 1H), 7.24 (m, 1H), 6.87 (m, 2H), 6.77 (m, 1H), 5.01 (sept, J=7.2 Hz, 1H), 4.53 (s, 2H), 4.31 (t, J=5.6 Hz, 2H), 3.65 (t, J=5.6 Hz, 2H), 1.54 (d, J=6.8 Hz, 6H), LC/MS: m/e=344 (M+H)⁺.

### Compound I-89

### 8-(3-Chlorophenyl)-1-isobutyl-6,7,8,9-tetrahydropyrazino[1,2-a]pyrazolo[3,4-d]pyrimidin-4(1H)-one

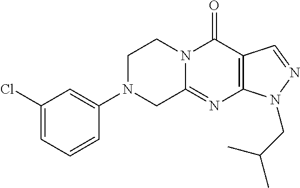 [SMILES: CC(C)Cn1ncc2c(=O)n3c(nc21)CN(c1cccc(Cl)c1)CC3]

The title compound was prepared via the procedure used for I-91, substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(3-chlorophenylamino)ethanol) for 2-(4-chlorophenylamino)ethanol. ¹H NMR (400 MHz, CDCl₃): δ 8.10 (s, 1H), 7.25 (m, 1H), 6.89 (m, 2H), 6.79 (m, 1H), 4.53 (s, 2H), 4.32 (t, J=5.6 Hz, 2H), 4.13 (d, J=7.2 Hz, 2H), 3.67 (t, J=5.6 Hz, 2H), 2.35 (m, 1H), 0.93 (d, J=6.4 Hz, 6H), LC/MS: m/e=358 (M+H)⁺.

### Compound I-90

### 8-(3-Chlorophenyl)-1-cyclopentyl-6,7,8,9-tetrahydropyrazino[1,2-a]pyrazolo[3,4-d]pyrimidin-4(1H)-one

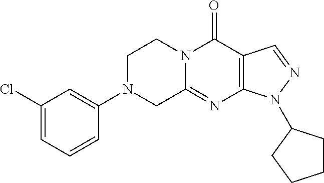 [SMILES: O=c1c2cnn(C3CCCC3)c2nc2n1CCN(c1cccc(Cl)c1)C2]

The title compound was prepared via the procedure used for I-91, substituting 5-amino-1-cyclopentyl-1H-pyrazole-4-carboxamide (prepared according to the procedure in Haning, H., et al., Bioorg Med. Chem. Lett. 2005, 15, 3900) for 5-amino-1-isopropyl-1H-pyrazole-4-carboxamide, and substituting 2-(3-chlorophenylamino)ethanol) for 2-(4-chlorophenylamino)ethanol. ¹H NMR (400 MHz, CDCl₃): δ 8.10 (s, 1H), 7.24 (m, 1H), 6.88 (m, 2H), 6.78 (dd, J₁=9.2 Hz, J₂=2.4 Hz, 1H), 5.15 (quint, J=7.6 Hz, 1H), 4.53 (s, 2H), 4.31 (t, J=5.6 Hz, 2H), 3.66 (t, J=5.6 Hz, 2H), 2.16-2.05 (m, 4H), 1.99 (m, 2H), 1.73 (m, 2H), LC/MS: m/e=370 (M+H)⁺.

### Compound I-92

### 8-(4-Chlorophenyl)-1-isobutyl-6,7,8,9-tetrahydropyrazino[1,2-a]pyrazolo[3,4-d]pyrimidin-4(1H)-one

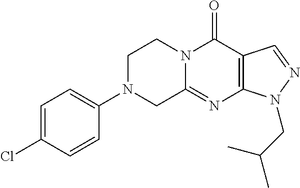 [SMILES: CC(C)Cn1ncc2c(=O)n3c(nc21)CN(c1ccc(Cl)cc1)CC3]

The title compound was prepared via the procedure used for I-91, substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide. ¹H NMR (400 MHz, CDCl₃): δ 8.09 (s, 1H), 7.29 (m, 2H), 6.86 (m, 2H), 4.49 (s, 2H), 4.29 (t, J=6.0 Hz, 2H), 4.12 (d, J=7.2 Hz, 2H), 3.65 (t, J=5.6 Hz, 2H), 2.35 (m, 1H), 0.93 (d, J=6.4 Hz, 6H), LC/MS: m/e=358 (M+H)⁺.

### Compound I-93

### 8-(4-Chlorophenyl)-1-cyclopentyl-6,7,8,9-tetrahydropyrazino[1,2-a]pyrazolo[3,4-d]pyrimidin-4(1H)-one

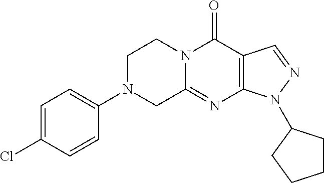 [SMILES: O=c1c2cnn(C3CCCC3)c2nc2n1CCN(c1ccc(Cl)cc1)C2]

The title compound was prepared via the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide. ¹H NMR (400 MHz, CDCl₃): δ 8.10 (s, 1H), 7.29 (dd, J₁=11.6 Hz, J₂=2.0 Hz, 2H), 6.87 (d, J=12.4 Hz, 2H), 5.15 (quint, 1=7.2 Hz, 1H), 4.50 (s, 2H), 4.29 (t, J=5.6 Hz, 2H), 3.64 (t, J=5.6 Hz, 2H), 2.18-2.07 (m, 4H), 1.99 (m, 2H), 1.74 (m, 2H), LC/MS: m/e=370 (M+H)⁺.

### Compound I-94

### 1-Isopropyl-8-(2-methoxyphenyl)-6,7,8,9-tetrahydropyrazino[1,2-a]pyrazolo[3,4-d]pyrimidin-4(1H)-one

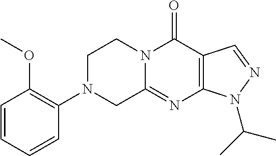 [SMILES: COc1ccccc1N1CCn2c(nc3c(cnn3C(C)C)c2=O)C1]

The title compound was prepared via the procedure used for I-91, substituting 2-(2-methoxyphenylamino)ethanol) for 2-(4-chlorophenylamino)ethanol. ¹H NMR (400 MHz, CDCl₃): δ 8.08 (s, 1H), 7.10 (m, 1H), 6.95 (m, 3H), 5.01 (sept, J=6.8 Hz, 1H), 4.39 (s, 2H), 4.13 (t, J=5.6 Hz, 2H), 3.91 (s, 3H), 3.57 (t, J=5.6 Hz, 2H), 1.53 (d, J=6.8 Hz, 6H), LC/MS: m/e=340 (M+H)⁺.

### Compound I-96

### 1-Cyclopentyl-8-(2-methoxyphenyl)-6,7,8,9-tetrahydropyrazino[1,2-a]pyrazolo[3,4-d]pyrimidin-4(1H)-one

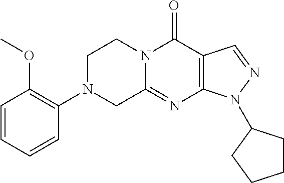 [SMILES: COc1ccccc1N1CCn2c(nc3c(cnn3C3CCCC3)c2=O)C1]

The title compound was prepared via the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(2-methoxyphenylamino)ethanol) for 2-(4-chlorophenylamino)ethanol. ¹H NMR (400 MHz, CDCl₃): δ 8.09 (s, 1H), 7.11 (m, 1H), 6.96 (m, 3H), 5.15 (quint, J=7.2 Hz, 1H), 4.41 (s, 2H), 4.14 (t, J=5.6 Hz, 2H), 3.92 (s, 3H), 3.58 (t, J=5.6 Hz, 2H), 2.16-2.05 (m, 4H), 1.98 (m, 2H), 1.74 (m, 2H), LC/MS: m/e=366 (M+H)⁺.

### Compound I-97

### 1-Isopropyl-8-(3-methoxyphenyl)-6,7,8,9-tetrahydropyrazino[1,2-a]pyrazolo[3,4-d]pyrimidin-4(1H)-one

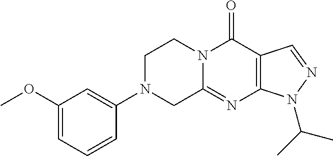 [SMILES: COc1cccc(N2CCn3c(nc4c(cnn4C(C)C)c3=O)C2)c1]

The title compound was prepared via the procedure used for I-91, substituting 2-(3-methoxyphenylamino)ethanol) for 2-(4-chlorophenylamino)ethanol. ¹H NMR (400 MHz, CDCl₃): δ 8.09 (s, 1H), 7.24 (m, 1H), 6.54 (dd, J₁=7.6 Hz, J₂=2.4 Hz, 1H), 6.48 (m, 2H), 5.01 (sept, J=6.4 Hz, 1H), 4.52 (s, 2H), 4.27 (t, J=5.6 Hz, 2H), 3.82 (s, 3H), 3.66 (t, J=5.6 Hz, 2H), 1.54 (d, J=6.8 Hz, 6H), LC/MS: m/e=340 (M+H)⁺.

### Compound I-98

### 1-Cyclopentyl-8-(3-methoxyphenyl)-6,7,8,9-tetrahydropyrazino[1,2-a]pyrazolo[3,4-d]pyrimidin-4(1H)-one

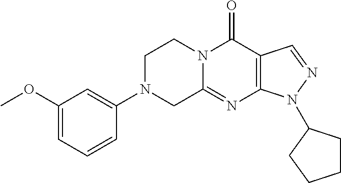 [SMILES: COc1cccc(N2CCn3c(nc4c(cnn4C4CCCC4)c3=O)C2)c1]

The title compound was prepared via the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(3-methoxyphenylamino)ethanol) for 2-(4-chlorophenylamino)ethanol. ¹H NMR (400 MHz, CDCl₃): δ 8.09 (s, 1H), 7.25 (t, J=8.0 Hz, 1H), 6.55 (dd, J₁=8.0 Hz, J₂=1.6 Hz, 1H), 6.47 (m, 2H), 5.16 (quint, J=7.2 Hz, 1H), 4.53 (s, 2H), 4.27 (t, J=6.0 Hz, 2H), 3.83 (s, 3H), 3.66 (t, J=5.6 Hz, 2H), 2.16-2.07 (m, 4H), 1.99 (m, 2H), 1.73 (m, 2H), LC/MS: m/e=366 (M+H)⁺.

### Compound I-95

### 1-Cyclopentyl-8-phenyl-6,7,8,9-tetrahydropyrazino[1,2-a]pyrazolo[3,4-d]pyrimidin-4(1H)-one

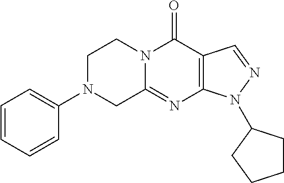 [SMILES: O=c1c2cnn(C3CCCC3)c2nc2n1CCN(c1ccccc1)C2]

The title compound was prepared via the procedure used for I-91, substituting 5-amino-1-cyclopentyl-1H-pyrazole-4-carboxamide (prepared according to the procedure in Haning, H., et al., Bioorg. Med. Chem. Leu. 2005, 15, 3900) for 5-amino-1-isopropyl-1H-pyrazole-4-carboxamide, and substituting 2-(phenylamino)ethanol) for 2-(4-chlorophenylamino)ethanol. ¹H NMR (400 MHz, d⁴-MeOH): δ 8.04 (s, 1H), 7.30 (m, 2H), 7.02 (d, J=8.0 Hz, 2H), 6.89 (t, J=7.6 Hz, 1H), 5.23 (quint, J=7.2 Hz, 1H), 4.54 (s, 2H), 4.21 (t, J=6.0 Hz, 2H), 3.71 (t, J=5.6 Hz, 2H), 2.17-2.00 (m, 4H), 1.98 (m, 2H), 1.76 (m, 2H), LC/MS: m/e=336 (M+H)⁺.

### Compound I-141

### 8-(3-Ethoxyphenyl)-1-isopropyl-6,7,8,9-tetrahydropyrazino[1,2-a]pyrazolo[3,4-d]pyrimidin-4(1H)-one

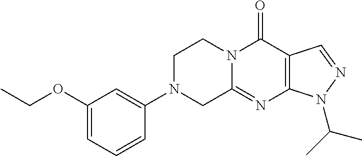 [SMILES: CCOc1cccc(N2CCn3c(nc4c(cnn4C(C)C)c3=O)C2)c1]

The title compound was prepared via the procedure used for I-91, substituting 2-(3-ethoxyphenylamino)ethanol) for 2-(4-chlorophenylamino)ethanol. ¹H NMR (400 MHz, CDCl₃): δ 8.10 (s, 1H), 7.23 (m, 1H), 6.53 (dd, J₁=8.0 Hz, J₂=1.6 Hz, 1H), 6.47 (m, 2H), 5.01 (sept, J=6.4 Hz, 1H), 4.52 (s, 2H), 4.27 (t, J=6.0 Hz, 2H), 4.06 (q, J=7.2 Hz, 2H), 3.66 (t, J=5.6 Hz, 2H), 1.55 (d, J=6.8 Hz, 6H), 1.43 (t, J=7.2 Hz, 3H), LC/MS: m/e=354 (M+H)⁺.

### Compound I-142

### 8-(4-Fluorophenyl)-1-isopropyl-6,7,8,9-tetrahydropyrazino[1,2-a]pyrazolo[3,4-d]pyrimidin-4(1H)-one

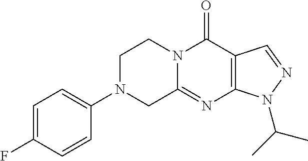 [SMILES: CC(C)n1ncc2c(=O)n3c(nc21)CN(c1ccc(F)cc1)CC3]

The title compound was prepared via the procedure used for I-91, substituting 2-(4-fluorophenylamino)ethanol) for 2-(4-chlorophenylamino)ethanol. ¹H NMR (400 MHz, CDCl₃): δ 8.10 (s, 1H), 7.04 (m, 2H), 6.93 (m, 2H), 5.01 (sept, J=6.8 Hz, 1H), 4.45 (s, 2H), 4.25 (t, J=5.6 Hz, 2H), 3.61 (t, J=5.6 Hz, 2H), 1.55 (d, J=5.6 Hz, 6H), LC/MS: m/e=328 (M+H)⁺.

### Compound I-143

### 1-Isopropyl-8-(pyridin-3-yl)-6,7,8,9-tetrahydropyrazino[1,2-a]pyrazolo[3,4-d]pyrimidin-4(1H)-one

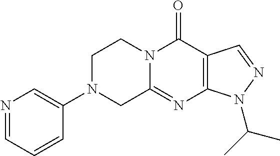 [SMILES: CC(C)n1ncc2c(=O)n3c(nc21)CN(c1cccnc1)CC3]

The title compound was prepared via the procedure used for I-91, substituting 2-(pyridin-3-ylamino)ethanol for 2-(4-chlorophenylamino)ethanol. ¹H NMR (400 MHz, d⁴-MeOH): δ 8.31 (m, 1H), 8.10 (s, 1H), 8.04 (m, 1H), 7.48 (m, 1H), 7.37 (m, 1H), 5.08 (sept, J=7.2 Hz, 1H), 4.66 (s, 2H), 4.33 (t, J=5.6 Hz, 2H), 3.77 (t, J=5.6 Hz, 2H), 1.52 (d, J=7.2 Hz, 6H), LC/MS: m/e=311 (M+H)⁺.

### Compound I-144

### 1-Isopropyl-8-(5-methoxypyridin-3-yl)-6,7,8,9-tetrahydropyrazino[1,2-a]pyrazolo[3,4-d]pyrimidin-4(1H)-one

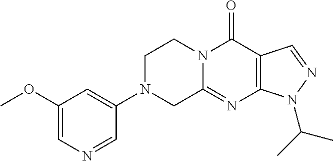 [SMILES: COc1cncc(N2CCn3c(nc4c(cnn4C(C)C)c3=O)C2)c1]

The title compound was prepared via the procedure used for I-91, substituting 2-((5-methoxypyridin-3-yl)amino)ethanol for 2-(4-chlorophenylamino)ethanol. ¹H NMR (400 MHz, CDCl₃): 8.11 (s, 1H), 8.07-7.85 (m, 2H), 6.72 (s, 1H), 5.02 (sept, J=6.4 Hz, 1H), 4.57 (s, 2H), 4.35 (t, J=6.0 Hz, 2H), 3.90 (s, 3H), 3.70 (t, J=5.6 Hz, 2H), 1.55 (d, J=6.8 Hz, 6H), LC/MS: m/e=341 (M+H)⁺.

### Compound I-145

### 1-Isopropyl-8-(pyridin-4-yl)-6,7,8,9-tetrahydropyrazino[1,2-a]pyrazolo[3,4-d]pyrimidin-4(1H)-one

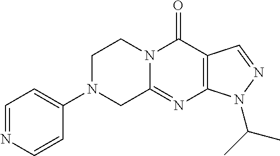 [SMILES: CC(C)n1ncc2c(=O)n3c(nc21)CN(c1ccncc1)CC3]

The title compound was prepared via the procedure used for I-91, substituting 2-(pyridin-4-ylamino)ethanol for 2-(4-chlorophenylamino)ethanol. ¹H NMR (400 MHz, CDCl₃): δ 8.38 (m, 2H), 8.12 (s, 1H), 6.72 (m, 2H), 5.02 (sept, J=7.2 Hz, 1H), 4.65 (s, 2H), 4.45 (t, J=5.6 Hz, 2H), 3.73 (t, J=5.6 Hz, 2H), 1.56 (d, J=6.8 Hz, 6H), LC/MS: m/e=311 (M+H)⁺.

### Compound I-256

### 8-(4-Chlorophenyl)-1-(2-methoxyethyl)-6,7,8,9-tetrahydropyrazino[1,2-a]pyrazolo[3,4-d]pyrimidin-4(1H)-one

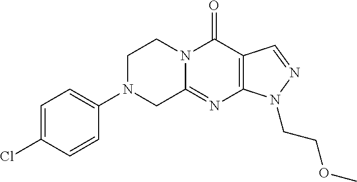 [SMILES: COCCn1ncc2c(=O)n3c(nc21)CN(c1ccc(Cl)cc1)CC3]

The title compound was prepared via the procedure used for I-91, substituting 5-amino-1-(2-methoxyethyl)-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide. ¹H NMR (400 MHz, CDCl₃): δ 8.12 (s, 1H), 7.28 (m, 2H), 6.85 (m, 2H), 4.51 (s, 2H), 4.50 (t, J=7.2 Hz, 2H), 4.29 (t, J=6.0 Hz, 2H), 3.86 (t, J=5.2 Hz, 2H), 3.64 (t, J=5.6 Hz, 2H), 3.34 (s, 3H), LC/MS: m/e=360 (M+H)⁺.

### Compound I-257

### 8-(3,4-Dichlorophenyl)-1-(2-methoxyethyl)-6,7,8,9-tetrahydropyrazino[1,2-a]pyrazolo[3,4-d]pyrimidin-4(1H)-one

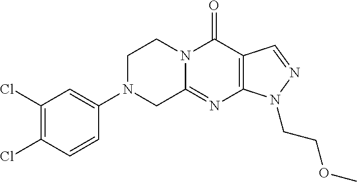 [SMILES: COCCn1ncc2c(=O)n3c(nc21)CN(c1ccc(Cl)c(Cl)c1)CC3]

The title compound was prepared via the procedure used for I-91, substituting 5-amino-1-(2-methoxyethyl)-1H-pyrazole-4-carboxamide (prepared according to the procedure in Hailing, H., et al., Bioorg. Med. Chem. Lett. 2005, 15, 3900) for 5-amino-1-isopropyl-1H-pyrazole-4-carboxamide, and substituting 2-(3,4-dichlorophenylamino)ethanol for 2-(4-chlorophenylamino)ethanol. ¹H NMR (400 MHz, CDCl₃): δ 8.12 (s, 1H), 7.36 (d, J=9.2 Hz, 1H), 6.97 (d, J=3.2 Hz, 1H), 6.74 (dd, J₁=9.2 Hz, J₂=2.8 Hz, 1H), 4.52 (s, 2H), 4.50 (t, J=5.2 Hz, 2H), 4.33 (t, J=6.0 Hz, 2H), 3.86 (t, J=5.6 Hz, 2H), 3.64 (t, J=5.6 Hz, 2H), 3.34 (s, 3H), LC/MS: m/e=394, 396 (M+H)⁺.

### Compound I-167

### 8-(4-Chlorophenyl)-1-propyl-6,7,8,9-tetrahydropyrazino[1,2-a]pyrazolo[3,4-d]pyrimidin-4(1H)-one

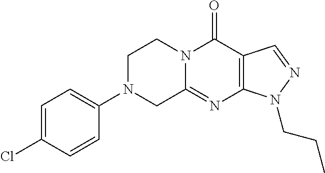 [SMILES: CCCn1ncc2c(=O)n3c(nc21)CN(c1ccc(Cl)cc1)CC3]

The title compound was prepared via the procedure used for I-91, substituting 5-amino-1-n-propyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide. ¹H NMR (400 MHz, CDCl₃): δ 8.09 (s, 1H), 7.29 (d, J=9.2 Hz, 2H), 6.86 (d, J=8.8 Hz, 2H), 4.50 (s, 2H), 4.29 (m, 4H), 4.65 (t, J=6.0 Hz, 2H), 1.95 (seat, J=7.2 Hz, 2H), 0.94 (t, J=7.6 Hz, 3H), LC/MS: m/e=344 (M+H)⁺.

### Compound I-168

### 8-(3,4-Dichlorophenyl)-1-propyl-6,7,8,9-tetrahydropyrazino[1,2-a]pyrazolo[3,4-d]pyrimidin-4(1H)-one

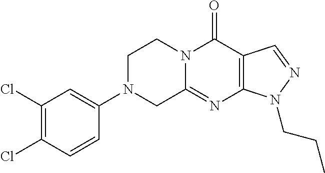 [SMILES: CCCn1ncc2c(=O)n3c(nc21)CN(c1ccc(Cl)c(Cl)c1)CC3]

The title compound was prepared via the procedure used for I-91, substituting 5-amino-1-n-propyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(3,4-dichlorophenylamino)ethanol for 2-(4-chlorophenylamino)ethanol. ¹H NMR (400 MHz, CDCl₃): δ 8.11 (s, 1H), 7.37 (d, J=6.8 Hz, 1H), 6.99 (d, J=2.0 Hz, 1H), 6.76 (dd, J₁=7.6 Hz, J₂=2.0 Hz, 1H), 4.52 (s, 2H), 4.34 (t, J=4.4 Hz, 2H), 4.30 (t, J=5.6 Hz, 2H), 3.66 (t, J=4.4 Hz, 2H), 1.96 (sext, J=5.6 Hz, 2H), 0.95 (t, J=5.6 Hz, 3H), LC/MS: m/e=378 (M+H)⁺.

### Compound I-171

### 8-(3,4-Dichlorophenyl)-1-isopropyl-6,7,8,9-tetrahydropyrazino[1,2-a]pyrazolo[3,4-d]pyrimidin-4(1H)-one

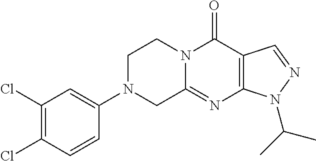 [SMILES: CC(C)n1ncc2c(=O)n3c(nc21)CN(c1ccc(Cl)c(Cl)c1)CC3]

The title compound was prepared via the procedure used for I-91, substituting 2-(3,4-dichlorophenylamino)ethanol for 2-(4-chlorophenylamino)ethanol, ¹H NMR (400 MHz, CDCl₃): δ 8.10 (s, 1H), 7.36 (d, J=8.8 Hz, 1H), 6.97 (d, J=2.8 Hz, 1H), 6.74 (dd, J₁=9.6 Hz, J₂=2.8 Hz, 1H), 5.01 (sept, J=7.2 Hz, 1H), 4.51 (s, 2H), 4.33 (t, J=6.0 Hz, 2H), 3.64 (t, J=5.6 Hz, 2H), 1.55 (d, J=6.4 Hz, 6H), LC/MS: m/e=378 (M+H)⁺.

### Compound I-212

### 8-(4-Chlorophenyl)-1-(tetrahydro-2H-pyran-4-yl)-6,7,8,9-tetrahydropyrazino[1,2-a]pyrazolo[3,4-d]pyrimidin-4(1H)-one

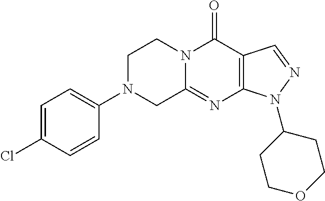 [SMILES: O=c1c2cnn(C3CCOCC3)c2nc2n1CCN(c1ccc(Cl)cc1)C2]

The title compound was prepared via the procedure used for I-91, substituting 5-amino-1-(tetrahydro-2H-pyran-4-yl)-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide. ¹H NMR (400 MHz, CDCl₃): δ 8.11 (s, 1H), 7.30 (m, 2H), 6.87 (m, 2H), 4.82 (m, 1H), 4.51 (s, 2H), 4.30 (t, J=5.6 Hz, 2H), 4.17 (dd, J₁=11.6 Hz, J₂=4.4 Hz, 2H), 3.65 (t, J=5.6 Hz, 2H), 3.60 (m, 2H), 2.40 (m, 2H), 1.93 (dd, J₁=12.8 Hz, J₂=2.0 Hz, 2H), LC/MS: m/e=386 (M+H)⁺.

### Compound I-216

### (±)-8-Phenyl-1-(tetrahydrofuran-3-yl)-6,7,8,9-tetrahydropyrazino[1,2-a]pyrazolo[3,4-d]pyrimidin-4(1H)-one

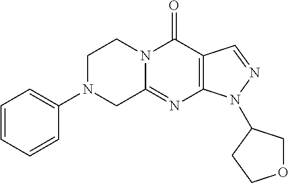 [SMILES: O=c1c2cnn(C3CCOC3)c2nc2n1CCN(c1ccccc1)C2]

The title compound was prepared via the procedure used for I-91, substituting racemic 5-amino-1-(tetrahydrofuran-3-yl)-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide, and substituting 2-(phenylamino)ethanol for 2-(4-chlorophenylamino)ethanol. ¹H NMR (400 MHz, CDCl₃): δ 8.12 (s, 1H), 7.35 (t, J=8.4 Hz, 2H), 6.95 (m, 3H), 5.44 (m, 1H), 4.53 (s, 2H), 4.28 (m, 3H), 4.20 (m, 1H), 4.04 (m, 2H), 3.68 (t, 5.6 Hz, 2H), 2.46 (m, 2H), LC/MS: m/e=338 (M+H)⁺.

### Compound I-217

### (±)-8-(4-Chlorophenyl)-1-(tetrahydrofuran-3-yl)-6,7,8,9-tetrahydropyrazino[1,2-a]pyrazolo[3,4-d]pyrimidin-4(1H)-one

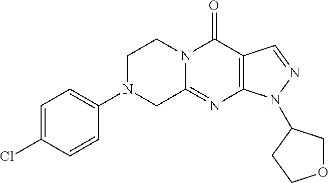 [SMILES: O=c1c2cnn(C3CCOC3)c2nc2n1CCN(c1ccc(Cl)cc1)C2]

The title compound was prepared via the procedure used for I-91, substituting (+/−)-5-amino-1-(tetrahydrofuran-3-yl)-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide. ¹H NMR (400 MHz, CDCl₃): δ 8.13 (s, 1H), 7.30 (d, J=8.8 Hz, 2H), 6.87 (d, J=8.8 Hz, 2H), 5.43 (m, 1H), 4.50 (s, 2H), 4.28 (m, 3H), 4.20 (m, 1H), 4.05 (m, 2H), 3.65 (t, J=5.6 Hz, 2H), 2.47 (m, 2H), LC/MS: m/e=372 (M+H)⁺.

### Compound I-237

### 4-(1-Isopropyl-4-oxo-6,7-dihydropyrazino[1,2-a]pyrazolo[3,4-d]pyrimidin-8(1H,4H,9H)-yl)benzonitrile

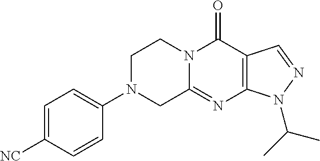 [SMILES: CC(C)n1ncc2c(=O)n3c(nc21)CN(c1ccc(C#N)cc1)CC3]

The title compound was prepared via the procedure used for I-91, substituting 4-((2-hydroxyethyl)amino)benzonitrile for 2-(4-chlorophenylamino)ethanol. ¹H NMR (400 MHz, d⁶-DMSO): δ 8.13 (s, 1H), 7.65 (d, J=8.8 Hz, 2H), 7.03 (d, J=8.4 Hz, 2H), 4.96 (sept, J=6.8 Hz, 1H), 4.70 (s, 2H), 4.27 (t, J=5.6 Hz, 2H), 3.73 (t, J=5.6 Hz, 2H), 1.45 (d, J=6.8 Hz, 6H), LC/MS: m/e=335 (M+H)⁺.

### Compound I-239

### 1-Isopropyl-8-(4-(trifluoromethyl)phenyl)-6,7,8,9-tetrahydropyrazino[1,2-a]pyrazolo[3,4-d]pyrimidin-4(1H)-one

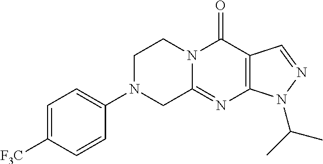 [SMILES: CC(C)n1ncc2c(=O)n3c(nc21)CN(c1ccc(C(F)(F)F)cc1)CC3]

The title compound was prepared via the procedure used for I-91, substituting 2-((4-(trifluoromethyl)phenyl)amino)ethanol for 2-(4-chlorophenylamino)ethanol. ¹H NMR (400 MHz, CDCl₃): δ 8.12 (s, 1H), 7.58 (d, J=8.4 Hz, 2H), 6.93 (d, J=8.4 Hz, 2H), 5.03 (sept, J=6.4 Hz, 1H), 4.63 (s, 2H), 4.39 (t, J=5.6 Hz, 2H), 3.74 (t, J=5.6 Hz, 2H), 1.58 (d, J=6.8 Hz, 6H), LC/MS: m/e=378 (M+H)⁺.

### Compound I-247

### 1-(tert-Butyl)-8-(4-chlorophenyl)-6,7,8,9-tetrahydropyrazino[1,2-a]pyrazolo[3,4-d]pyrimidin-4(1H)-one

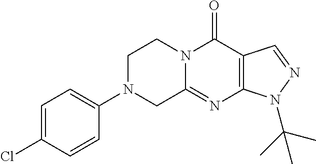 [SMILES: CC(C)(C)n1ncc2c(=O)n3c(nc21)CN(c1ccc(Cl)cc1)CC3]

The title compound was prepared via the procedure used for I-91, substituting 5-amino-1-t-butyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide. ¹H NMR (400 MHz, CDCl₃): δ 8.06 (s, 1H), 7.31 (d, J=8.8 Hz, 2H), 6.88 (d, J=9.2 Hz, 2H), 4.51 (s, 2H), 4.31 (t, J=5.6 Hz, 2H), 3.65 (t, J=5.6 Hz, 2H), 1.78 (s, 9H), LC/MS: m/e=358 (M+H)⁺.

### Compound I-99

### (±)-1-cyclopentyl-6-methyl-6,7,8,9-tetrahydropyrazino[1,2-a]pyrazolo[3,4-d]pyrimidin-4(1H)-one

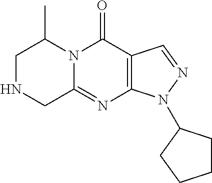 [SMILES: CC1CNCc2nc3c(cnn3C3CCCC3)c(=O)n21]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett, 2005, 15, 3900) for 5-amino-1-isopropyl-1H-pyrazole-4-carboxamide for 5-amino-1-isopropyl-1H-pyrazole-4-carboxamide, and substituting 1-aminopropan-2-ol for 2-(4-chlorophenylamino)ethanol. ¹H NMR (400 MHz, CDCl₃): δ 8.06 (s, 1H), 5.11 (quint, J=7.6 Hz, 1H), 4.80 (m, 1H), 4.17 (d, J=18.0 Hz, 1H), 4.06 (d, J=18.0 Hz, 1H), 3.18 (d, J=2.4 Hz, 2H), 2.15-2.05 (m, 4H), 1.98 (m, 2H), 1.85 (s, 1H), 1.73 (m, 2H), 1.43 (d, J=6.4 Hz, 3H), LC/MS: m/e=274 (M+H)⁺.

### Compound I-100

### (±)-1-Cyclopentyl-6,8-dimethyl-6,7,8,9-tetrahydropyrazino[1,2-a]pyrazolo[3,4-d]pyrimidin-4(1H)-one

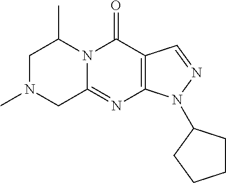 [SMILES: CC1CN(C)Cc2nc3c(cnn3C3CCCC3)c(=O)n21]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide, and substituting 1-(methylamino)propan-2-ol for 2-(4-chlorophenylamino)ethanol. ¹H NMR (400 MHz, CDCl₃): δ 8.05 (s, 1H), 5.11 (quint, J=7.6 Hz, 1H), 4.81 (m, 1H), 4.04 (dd, J₁=17.6 Hz, J₂=2.0 Hz, 1H), 3.28 (d, J=17.2 Hz, 1H), 2.91 (dt, J₁=11.6 Hz, J₂=2.0 Hz, 1H), 2.54 (dd, J₁=12.0 Hz, J₂=4.0 Hz, 1H), 2.45 (s, 3H), 2.15-2.07 (m, 4H), 1.96 (m, 2H), 1.73 (m, 2H), 1.47 (d, J=6.4 Hz, 3H), LC/MS: m/e=288 (M+H)⁺.

### Compound I-101

### (±)-8-Benzyl-1-cyclopentyl-6-methyl-6,7,8,9-tetrahydropyrazino[1,2-a]pyrazolo[3,4-d]pyrimidin-4(1H)-one

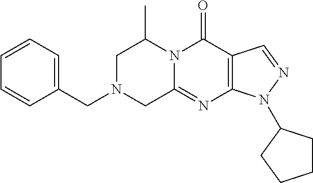 [SMILES: CC1CN(Cc2ccccc2)Cc2nc3c(cnn3C3CCCC3)c(=O)n21]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide, and substituting 1-(benzylamino)propan-2-ol for 2-(4-chlorophenylamino)ethanol. ¹H NMR (400 MHz, CDCl₃): δ 8.05 (s, 1H), 7.41-7.29 (m, 5H), 5.08 (quint, J=7.2 Hz, 1H), 4.80 (m, 1H), 4.08 (d, J=16.0 Hz, 1H), 3.69 (d, J=13.2 Hz, 1H), 6.45 (d, J=13.2 Hz, 1H), 3.40 (d, J=16.8 Hz, 1H), 2.99 (d, J=12.4 Hz, 1H), 2.57 (dd, J₁=12.0 Hz, J₂=3.2 Hz, 1H), 2.11-2.01 (m, 4H) 1.96 (m, 2H), 1.70 (m, 2H), 1.48 (d, J=6.4 Hz, 3H), LC/MS: m/e=364 (M+H)⁺.

### Compound I-102

### (±)-1-cyclopentyl-8-methyl-6-phenyl-6,7,8,9-tetrahydropyrazino[1,2-a]pyrazolo[3,4-d]pyrimidin-4(1H)-one

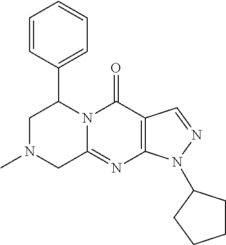 [SMILES: CN1Cc2nc3c(cnn3C3CCCC3)c(=O)n2C(c2ccccc2)C1]

The title compound was prepared in a manner similar to the procedure used for I-91, substituting 5-amino-1-cyclopentyl-1H-pyrazole-4-carboxamide (prepared according to the procedure in liming, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide, and substituting 2-(methylamino)-1-phenylethanol for 2-(4-chlorophenylamino)ethanol. ¹H NMR (400 MHz, CDCl₃): δ 7.97 (s, 1H), 7.31-7.17 (m, 5H), 5.74 (m, 1H), 5.15 (quint, J=7.6 Hz, 1H), 4.17-4.12 (m, 1H), 3.45 (d, J=16.8 Hz, 1H), 3.14 (d, J=12.0 Hz, 1H), 2.87 (dd, J₁=12.0 Hz, J₂=4.0 Hz, 1H), 2.36 (s, 3H), 2.19-2.07 (m, 4H), 1.98 (m, 2H), 1.73 (m, 2H), LC/MS: m/e=350 (M+H)⁺.

### Compound I-103

### (±)-1-Isopropyl-7-methyl-6,7,8,9-tetrahydropyrazino[1,2-a]pyrazolo[3,4-d]pyrimidin-4(1H)-one

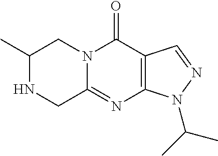 [SMILES: CC1Cn2c(nc3c(cnn3C(C)C)c2=O)CN1]

The title compound was prepared in a manner similar to the procedure used for I-91, substituting 2-aminopropan-1-ol for 2-(4-chlorophenylamino)ethanol. ¹H NMR (400 MHz, CDCl₃): δ 8.07 (s, 1H), 4.98 (sept, J=6.4 Hz, 1H), 4.37 (d, J=10.0 Hz, 1H), 4.21 (d, J=17.6 Hz, 1H), 4.11 (d, J=17.6 Hz, 1H), 3.29-3.20 (m, 2H), 2.19 (bs, 1H), 1.52 (d, J=6.8 Hz, 6H), 1.34 (d, J=6.0 Hz, 3H), LC/MS: =248 (M+H)⁺.

### Compound I-104

### (±)-1-Isopropyl-7,8-dimethyl-6,7,8,9-tetrahydropyrazino[1,2-a]pyrazolo[3,4-d]pyrimidin-4(1H)-one

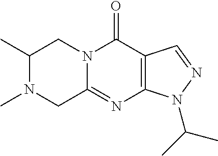 [SMILES: CC1Cn2c(nc3c(cnn3C(C)C)c2=O)CN1C]

The title compound was prepared in a manner similar to the procedure used for I-91, substituting 2-(methylamino)propan-1-ol for 2-(4-chlorophenylamino)ethanol. ¹H NMR (400 MHz, CDCl₃): δ 8.07 (s, 1H), 4.98 (sept, J=6.8 Hz, 1H), 4.24 (dd, J₁=14.0 Hz, J₂=4.0 Hz, 1H), 4.00 (d, J=17.2 Hz, 1H), 3.55 (d, J=17.2 Hz, 1H), 3.45 (dd, J₁=14.0 Hz, J₂ 10.4 Hz, 1H), 2.68 (m, 1H), 2.42 (s, 3H), 1.53 (dd, J₁=6.4 Hz, J₂=1.6 Hz, 6H), 1.25 (d, J=7.2 Hz, 3H), LC/MS: m/e=262 (M+H)⁺.

### Compound I-105

### (±)-8-Benzyl-1-isopropyl-7-methyl-6,7,8,9-tetrahydropyrazino[1,2-a]pyrazolo[3,4-d]pyrimidin-4(1H)-one

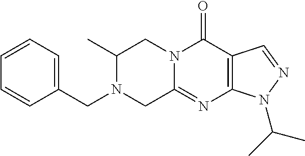 [SMILES: CC1Cn2c(nc3c(cnn3C(C)C)c2=O)CN1Cc1ccccc1]

The title compound was prepared in a manner similar to the procedure used for I-91, substituting 2-(benzylamino)propan-1-ol for 2-(4-chlorophenylamino)ethanol. ¹H NMR (400 MHz, CDCl₃): δ 8.07 (s, 1H), 7.37-7.29 (m, 5H), 4.92 (sept, J=6.8 Hz, 1H), 4.20 (dd, J₁=13.6 Hz, J₂=4.4 Hz, 1H), 4.01 (d, J=13.2 Hz, 1H), 3.86 (d, J=17.6 Hz, 1H), 3.70 (dd, J₁=13.6 Hz, J₂=8.0 Hz, 1H), 4.57 (d, J=17.6 Hz, 1H), 3.44 (d, J=13.2 Hz, 1H), 3.10 (m, 1H), 1.49 (d, J=6.4 Hz, 6H), 1.31 (d, J=6.4 Hz, 3H), LC/MS: m/e=338 (M+H)⁺.

### Compound I-106

### (±)-1-isopropyl-7-phenyl-6,7,8,9-tetrahydropyrazino[1,2-a]pyrazolo[3,4-d]pyrimidin-4(1H)-one

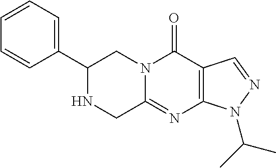 [SMILES: CC(C)n1ncc2c(=O)n3c(nc21)CNC(c1ccccc1)C3]

The title compound was prepared in a manner similar to the procedure used for I-91, substituting 2-amino-2-phenylethanol for 2-(4-chlorophenylamino)ethanol. ¹H NMR (400 MHz, CDCl₃): δ 8.08 (s, 1H), 7.47-7.34 (m, 5H), 5.01 (quint, J=6.4 Hz, 1H), 4.59 (dd, J₁=14.4 Hz, J₂=4.0 Hz, 1H), 4.34 (d, J=17.2 Hz, 1H), 4.25 (d, J=17.2 Hz, 1H), 4.18 (dd, J₁=11.2 Hz, J₂=4.4 Hz, 1H), 3.57 (dd, J₁=14.0 Hz, J₂=11.2 Hz, 1H), 1.76 (bs, 1H), 1.54 (d, J=6.4 Hz, 6H), LC/MS: m/e=310 (M+H)⁺.

### Compound I-107

### (±)-1-Isopropyl-8-methyl-7-phenyl-6,7,8,9-tetrahydropyrazino[1,2-a]pyrazolo[3,4-d]pyrimidin-4(1H)-one

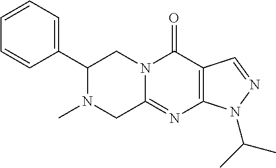 [SMILES: CC(C)n1ncc2c(=O)n3c(nc21)CN(C)C(c1ccccc1)C3]

The title compound was prepared in a manner similar to the procedure used for I-91, substituting 2-(methylamino)-2-phenylethanol for 2-(4-chlorophenylamino)ethanol. ¹H NMR (400 MHz, CDCl₃): δ 8.07 (s, 1H), 7.40-7.36 (m, 5H), 5.00 (sept, J=6.8 Hz, 1H), 4.45 (dd, J₁=14.4 Hz, J₂=4.4 Hz, 1H), 4.21 (d, J=17.2 Hz, 1H), 3.69 (m, 1H), 3.61 (d, J=17.2 Hz, 1H), 3.46 (dd, J₁=10.8 Hz, J₂=4.0 Hz, 1H), 2.19 (s, 3H), 1.54 (dd, J₁=6.4 Hz, J₂=0.8 Hz, 6H), LC/MS: m/e=324 (M+H)⁺.

### Compound I-108

### (±)-8-Benzyl-1-isopropyl-7-phenyl-6,7,8,9-tetrahydropyrazino[1,2-a]pyrazolo[3,4-d]pyrimidin-4(1H)-one

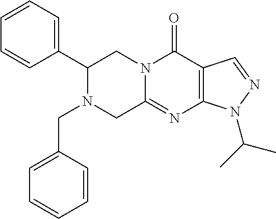 [SMILES: CC(C)n1ncc2c(=O)n3c(nc21)CN(Cc1ccccc1)C(c1ccccc1)C3]

The title compound was prepared in a manner similar to the procedure used for I-91, substituting 2-(benzylamino)-2-phenylethanol for 2-(4-chlorophenylamino)ethanol. ¹H NMR (400 MHz, CDCl₃): δ 8.07 (s, 1H), 7.54-7.27 (m, 10H), 4.94 (sept, J=6.4 Hz, 1H), 4.56 (m, 1H), 4.11 (d, J=17.2 Hz, 1H), 3.98 (d, J=12.8 Hz, 1H), 3.83 (m, 2H), 3.55 (d, J=17.6 Hz, 1H), 3.03 (d, J=12.4 Hz, 1H), 1.51 (dd, J₁=6.4 Hz, J₂=4.8 Hz, 6H), LC/MS: m/e=400 (M+H)⁺.

### Compound I-109

### (±)-7-Benzyl-1-isopropyl-6,7,8,9-tetrahydropyrazino[1,2-a]pyrazolo[3,4-d]pyrimidin-4(1H)-one

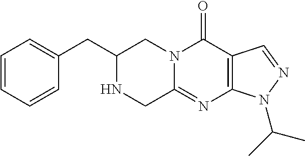 [SMILES: CC(C)n1ncc2c(=O)n3c(nc21)CNC(Cc1ccccc1)C3]

The title compound was prepared in a manner similar to the procedure used for I-91, substituting 2-amino-2-phenylethanol for 2-(4-chlorophenylamino)ethanol. ¹H NMR (400 MHz, CDCl₃): δ 8.07 (s, 1H), 7.39-7.27 (m, 5H), 4.98 (sept, J=6.8 Hz, 1H), 4.41 (dd, J₁=13.6 Hz, J₂=3.6 Hz, 1H), 4.17 (d, J=17.2 Hz, 1H), 3.99 (d, J=17.2 Hz, 1H), 3.41 (dd, J₁=13.6 Hz, J₂=10.8 Hz, 1H), 3.30 (m, 1H), 3.03 (dd, J₁=13.2 Hz, J₂=4.8 Hz, 1H), 2.80 (dd, J₁=14.0 Hz, J₂=8.4 Hz, 1H), 1.70 (bs, 1H), 1.52 (d, J=6.8 Hz, 6H), LC/MS: m/e=324 (M+H)⁺.

### Compound I-110

### (±)-7-Benzyl-1-isopropyl-8-methyl-6,7,8,9-tetrahydropyrazino[1,2-a]pyrazolo[3,4-d]pyrimidin-4(1H)-one

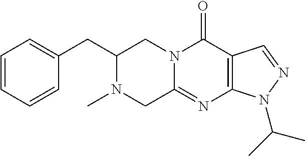 [SMILES: CC(C)n1ncc2c(=O)n3c(nc21)CN(C)C(Cc1ccccc1)C3]

The title compound was prepared in a manner similar to the procedure used for I-91, substituting 2-(methylamino)-2-phenylethanol for 2-(4-chlorophenylamino)ethanol. ¹H NMR (400 MHz, CDCl₃): δ 8.06 (s, 1H), 7.34 (m, 2H), 7.25 (m, 3H), 4.99 (sept, J=6.4 Hz, 1H), 4.01 (m, 2H), 3.76 (m, 2H), 3.10 (m, 2H), 2.67 (m, 1H), 2.54 (s, 3H), 1.54 (dd, J₁=6.4 Hz, J₂=2.4 Hz, 6H), LC/MS: m/e=338 (M+H)⁺.

### Compound I-119

### (±)-8-(4-Chlorobenzyl)-1-cyclopentyl-7-methyl-6,7,8,9-tetrahydropyrazino[1,2-a]pyrazolo[3,4-d]pyrimidin-4(1H)-one

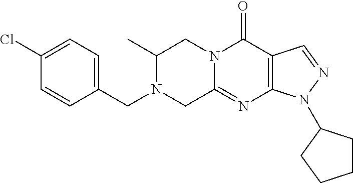 [SMILES: CC1Cn2c(nc3c(cnn3C3CCCC3)c2=O)CN1Cc1ccc(Cl)cc1]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide de for 5-amino-1-isopropyl-1H-pyrazole-4-carboxamide, and substituting 2-((4-chlorobenzyl)amino)propan-1-ol for 2-(4-chlorophenylamino)ethanol. ¹H NMR (400 MHz, CDCl₃): δ 8.06 (s, 1H), 7.33 (m, 4H), 5.06 (quint, J=7.6 Hz, 1H), 4.20 (dd, J₁=13.6 Hz, J₂=3.6 Hz, 1H), 3.98 (d, J=12.8 Hz, 1H), 3.83 (d, J=17.2 Hz, 1H), 3.70 (dd, J₁=14.0 Hz, J₂=8.4 Hz, 1H), 3.55 (d, J=17.2 Hz, 1H), 3.41 (d, J=12.8 Hz, 1H), 3.10 (m, 1H), 2.11-2.00 (m, 4H), 1.94 (m, 2H), 1.68 (m, 2H), 1.30 (d, J=6.4 Hz, 3H), LC/MS: m/e=398 (M+H)⁺.

### Compound I-120

### (±)-1-Cyclopentyl-8-(4-methoxybenzyl)-7-methyl-6,7,8,9-tetrahydropyrazino[1,2-a]pyrazolo[3,4-d]pyrimidin-4(1H)-one

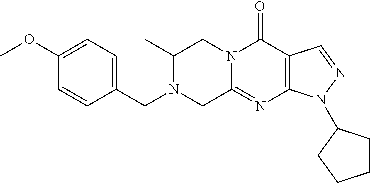 [SMILES: COc1ccc(CN2Cc3nc4c(cnn4C4CCCC4)c(=O)n3CC2C)cc1]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide, and substituting 2-((4-methoxybenzyl)amino)propan-1-ol for 2-(4-chlorophenylamino)ethanol. ¹H NMR (400 MHz, CDCl₃): δ 8.06 (s, 1H), 7.28 (d, J=8.0 Hz, 2H), 6.90 (d, J=8.0 Hz, 2H), 5.06 (quint, J=7.6 Hz, 1H), 4.18 (dd, J₁=13.6 Hz, J₂=3.6 Hz, 1H), 3.95 (d, J=12.4 Hz, 1H), 3.84 (d, J=18.0 Hz, 1H), 3.83 (s, 3H), 3.70 (dd, J₁=14.0 Hz, J₂=7.6 Hz, 1H), 3.55 (d, J=17.6 Hz, 1H), 3.38 (d, J=12.4 Hz, 1H), 3.07 (m, 1H), 2.11-2.02 (m, 4H), 1.94 (m, 2H), 1.68 (m, 2H), 1.30 (d, J=6.0 Hz, 3H), LC/MS: m/e=394 (M+H)⁺.

### Compound I-121

### (±)-8-(4-Chlorobenzyl)-1-cyclopentyl-6-methyl-6,7,8,9-tetrahydropyrazino[1,2-a]pyrazolo[3,4-d]pyrimidin-4(1H)-one

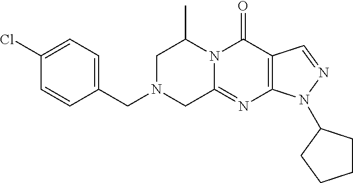 [SMILES: CC1CN(Cc2ccc(Cl)cc2)Cc2nc3c(cnn3C3CCCC3)c(=O)n21]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide, and substituting 1-((4-chlorobenzyl)amino)propan-2-ol for 2-(4-chlorophenylamino)ethanol. ¹H NMR (400 MHz, CDCl₃): δ 8.06 (s, 1H), 7.35 (m, 4H), 5.08 (quint, J=7.6 Hz, 1H), 4.81 (m, 1H), 4.05 (dd, J₁=16.8 Hz, J₂=1.6 Hz, 1H), 3.71 (d, J=13.2 Hz, 1H), 3.63 (d, J=13.2 Hz, 1H), 3.39 (d, J=16.8 Hz, 1H), 2.96 (d, J=12.0 Hz, 1H), 2.59 (dd, J₁=12.0 Hz, J₂=3.2 Hz, 1H), 2.13-2.05 (m, 4H), 1.95 (m, 2H), 1.70 (m, 2H), 1.48 (d, J=6.4 Hz, 3H), LC/MS: m/e=398 (M+H)⁺.

### Compound I-122

### (±)-1-Cyclopentyl-8-(4-methoxybenzyl)-6-methyl-6,7,8,9-tetrahydropyrazino[1,2-a]pyrazolo[3,4-d]pyrimidin-4(1H)-one

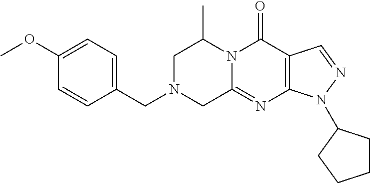 [SMILES: COc1ccc(CN2Cc3nc4c(cnn4C4CCCC4)c(=O)n3C(C)C2)cc1]

The title compound was prepared in a manner similar to the procedure used for I-91, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide, and substituting 1-((4-methoxybenzyl)amino)propan-2-ol for 2-(4-chlorophenylamino)ethanol. ¹H NMR (400 MHz, CDCl₃): δ 8.05 (s, 1H), 7.30 (d, J=8.4 Hz, 2H), 6.90 (d, J=8.8 Hz, 2H), 5.08 (quint, J=8.0 Hz, 1H), 4.79 (m, 1H), 4.06 (d, J=17.6 Hz, 1H), 3.83 (s, 3H), 3.69 (d, J=13.2 Hz, 1H), 3.59 (d, J=12.8 Hz, 1H), 3.37 (d, J=17.2 Hz, 1H), 2.97 (d, J=12.0 Hz, 1H), 2.54 (dd, J₁=12.0 Hz, J₂=3.2 Hz, 1H), 2.11-2.04 (m, 4H), 1.95 (m, 2H), 1.70 (m, 2H), 1.47 (d, J=6.4 Hz, 3H), LC/MS: m/e=394 (M+H)⁺.

### Compound I-146

### (±)-1-Isopropyl-8-(4-methoxybenzyl)-7-methyl-6,7,8,9-tetrahydropyrazino[1,2-a]pyrazolo[3,4-d]pyrimidin-4(1H)-one

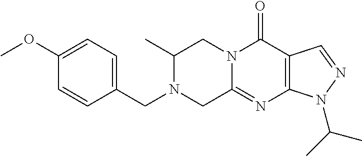 [SMILES: COc1ccc(CN2Cc3nc4c(cnn4C(C)C)c(=O)n3CC2C)cc1]

The title compound was prepared in a manner similar to the procedure used for I-91, substituting 2-((4-methoxybenzyl)amino)propan-1-ol for 2-(4-chlorophenylamino)ethanol. ¹H NMR (400 MHz, CDCl₃): δ 8.07 (s, 1H), 7.28 (m, 2H), 6.90 (m, 2H), 4.92 (sept, J=6.4 Hz, 1H), 4.18 (dd, J₁=13.6 Hz, J₂=4.4 Hz, 1H), 3.95 (d, J=12.8 Hz, 1H), 3.84 (d, J=17.6 Hz, 1H), 3.83 (s, 3H), 3.70 (dd, J₁=14.0 Hz, J₂=8.0 Hz, 1H), 3.54 (d, J=17.6 Hz, 1H), 3.38 (d, J=12.4 Hz, 1H), 3.07 (m, 1H), 1.51 (d, J=6.8 Hz, 6H), 1.30 (d, J=6.8 Hz, 3H), LC/MS: m/e=368 (M+H)⁺.

### Compound I-147

### (±)-8-(4-Chlorobenzyl)-1-isopropyl-7-methyl-6,7,8,9-tetrahydropyrazino[1,2-a]pyrazolo[3,4-d]pyrimidin-4(1H)-one

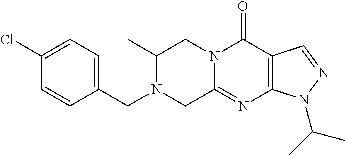 [SMILES: CC1Cn2c(nc3c(cnn3C(C)C)c2=O)CN1Cc1ccc(Cl)cc1]

The title compound was prepared in a manner similar to the procedure used for I-91, substituting 2-((4-chlorobenzyl)amino)propan-1-ol for 2-(4-chlorophenylamino)ethanol. ¹H NMR (400 MHz, CDCl₃): δ 8.06 (s, 1H), 7.32 (m, 4H), 4.92 (sept, J=6.8 Hz, 1H), 4.19 (dd, J₁=14.0 Hz, J₂=4.4 Hz, 1H), 3.97 (d, J=13.2 Hz, 1H), 3.81 (d, J=17.2 Hz, 1H), 3.68 (dd, J₁=14.2 Hz, J₂=7.6 Hz, 1H), 3.53 (d, J=17.2 Hz, 1H), 3.40 (d, J=13.2 Hz, 1H), 3.08 (m, 1H), 1.48 (dd, J₁=6.8 Hz, J₂=0.8 Hz, 6H), 1.29 (d. J=6.4 Hz, 3H), LC/MS: m/e=372 (M+H)⁺.

### Compound I-148

### (±)-8-(4-Fluorobenzyl)-1-isopropyl-7-methyl-6,7,8,9-tetrahydropyrazino[1,2-d]pyrazolo[3,4-d]pyrimidin-4(1H)-one

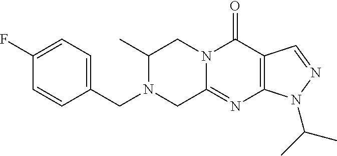 [SMILES: CC1Cn2c(nc3c(cnn3C(C)C)c2=O)CN1Cc1ccc(F)cc1]

The title compound was prepared in a manner similar to the procedure used for I-91, substituting 2-((4-fluorobenzyl)amino)propan-1-ol for 2-(4-chlorophenylamino)ethanol. ¹H NMR (400 MHz, CDCl₃): δ 8.07 (s, 1H), 7.34 (m, 2H), 7.05 (m, 2H), 4.92 (sept, J=6.8 Hz, 1H), 4.19 (dd, J₁=13.6 Hz, J₂=4.4 Hz, 1H), 3.97 (d, J=12.8 Hz, 1H), 3.82 (d, J=17.2 Hz, 1H), 3.69 (dd, J₁=13.2 Hz, J₂=8.0 Hz, 1H), 3.54 (d, J=17.2 Hz, 1H), 3.41 (d, J=13.2 Hz, 1H), 3.09 (m, 1H), 1.49 (dd, J₁=6.8 Hz, J₂=1.6 Hz, 6H), 1.30 (d, J=6.8 Hz, 3H), LC/MS: m/e=356 (M+H)⁺.

### Compound I-149

### (±)-8-(3-Fluorobenzyl)-1-isopropyl-7-methyl-6,7,8,9-tetrahydropyrazino[1,2-a]pyrazolo[3,4-d]pyrimidin-4(1H)-one

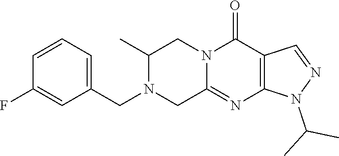 [SMILES: CC1Cn2c(nc3c(cnn3C(C)C)c2=O)CN1Cc1cccc(F)c1]

The title compound was prepared in a manner similar to the procedure used for I-91, substituting 2-((3-fluorobenzyl)amino)propan-1-ol for 2-(4-chlorophenylamino)ethanol. ¹H NMR (400 MHz, CDCl₃): δ 8.07 (s, 1H), 7.31 (m, 1H), 7.13 (m, 2H), 6.99 (m, 1H), 4.92 (sept, J=6.4 Hz, 1H), 4.21 (dd, J₁=14.0 Hz, J₂=4.4 Hz, 1H), 3.99 (d, J=13.2 Hz, 1H), 3.85 (d, J=17.2 Hz, 1H), 3.70 (dd, J₁=14.0 Hz, J₂=8.4 Hz, 1H), 3.57 (d, J=17.2 Hz, 1H), 3.45 (d, J=13.2 Hz, 1H), 3.10 (m, 1H), 1.49 (dd, J₁=6.8 Hz, J₂=1.6 Hz, 6H), 1.33 (d, J=6.8 Hz, 3H), LC/MS: m/e=356 (M+H)⁺.

### Compound I-150

### (±)-8-(4-Chlorophenyl)-1-isopropyl-7-methyl-6,7,8,9-tetrahydropyrazino[1,2-a]pyrazolo[3,4-d]pyrimidin-4(1H)-one

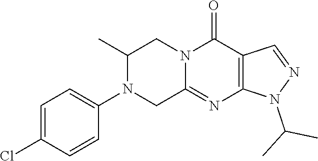 [SMILES: CC1Cn2c(nc3c(cnn3C(C)C)c2=O)CN1c1ccc(Cl)cc1]

The title compound was prepared in a manner similar to the procedure used for I-91, substituting 2-((4-chlorophenyl)amino)propan-1-ol for 2-(4-chlorophenylamino)ethanol. ¹H NMR (400 MHz, CDCl₃): δ 8.12 (s, 1H), 7.28 (m, 2H), 6.82 (m, 2H), 5.03 (sept, J=6.8 Hz, 1H), 4.50 (d, J=16.8 Hz, 1H), 4.45 (m, 1H), 4.40 (d, J=16.4 Hz, 1H), 4.23 (m, 1H), 4.09 (dd, J₁=14.0 Hz, J₂=4.4 Hz, 1H), 1.56 (m, 6H), 1.20 (d, J=6.4 Hz, 3H), LC/MS: m/e=358 (M+H)⁺.

### Compound I-151

### (±)-1-Isopropyl-8-(3-methoxyphenyl)-7-methyl-6,7,8,9-tetrahydropyrazino[1,2-a]pyrazolo[3,4-d]pyrimidin-4(1H)-one

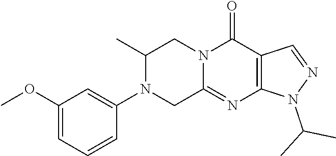 [SMILES: COc1cccc(N2Cc3nc4c(cnn4C(C)C)c(=O)n3CC2C)c1]

The title compound was prepared in a manner similar to the procedure used for I-91, substituting 2-((3-methoxyphenyl)amino)propan-1-ol for 2-(4-chlorophenylamino)ethanol. NMR (400 MHz, CDCl₃): δ 8.11 (s, 1H), 7.24 (t, J=8.0 Hz, 1H), 6.53 (m, 1H), 6.47 (m, 2H), 5.04 (sept, J=6.4 Hz, 1H), 4.54 (d, I=16.4 Hz, 1H), 4.50 (m, 1H), 4.44 (d, J=17.6 Hz, 1H), 4.07 (dd, J₁=13.6 Hz, J₂=4.4 Hz, 1H), 3.83 (s, 3H), 1.56 (t, J=6.8 Hz, 6H), 1.21 (d, J=6.4 Hz, 3H), LC/MS: m/e=354 (M+H)⁺.

### Compound I-152

### (±)-8-(4-Fluoro-3-methoxyphenyl)-1-isopropyl-7-methyl-6,7,8,9-tetrahydropyrazino[1,2-a]pyrazolo[3,4-d]pyrimidin-4(1H)-one

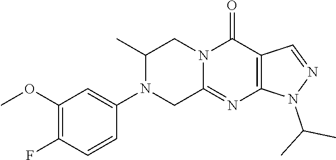 [SMILES: COc1cc(N2Cc3nc4c(cnn4C(C)C)c(=O)n3CC2C)ccc1F]

The title compound was prepared in a manner similar to the procedure used for I-91, substituting 2-((4-fluoro-3-methoxyphenyl)amino)propan-1-ol for 2-(4-chlorophenylamino)ethanol. ¹H NMR (400 MHz, CDCl₃): δ 8.11 (s, 1H), 7.03 (dd, J₁=11.2 Hz, J₂=8.4 Hz, 1H), 6.55 (dd, J₁=7.6 Hz, J₂=3.2 Hz, 1H), 6.42 (dt, J₁=8.8 Hz, J₂=3.2 Hz, 1H), 5.02 (sept, J=6.4 Hz, 1H), 4.46 (d, J=16.8 Hz, 1H), 4.39 (d, J=16.8 Hz, 1H), 4.31 (dd, J₁=13.2 Hz, J₂=4.0 Hz, 1H), 4.13 (m, 2H), 3.92 (s, 3H), 1.56 (m, 6H), 1.20 (d, J=6.0 Hz, 3H), LC/MS: m/e=372 (M+H)⁺.

### Compound I-152

### (±)-7-Ethyl-1-isopropyl-8-(4-methoxybenzyl)-6,7,8,9-tetrahydropyrazino[1,2-a]pyrazolo[3,4-a]pyrimidin-4(1H)-one

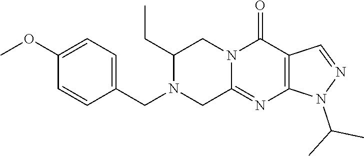 [SMILES: CCC1Cn2c(nc3c(cnn3C(C)C)c2=O)CN1Cc1ccc(OC)cc1]

The title compound was prepared in a manner similar to the procedure used for I-91, substituting 2-((4-methoxybenzyl)amino)butan-1-ol for 2-(4-chlorophenylamino)ethanol. ¹H NMR (400 MHz, CDCl₃): δ 8.08 (s, 1H), 7.27 (d, J=8.8 Hz, 2H), 6.90 (d, J=8.8 Hz, 2H), 4.95 (sept, J=6.8 Hz, 1H), 4.19 (dd, J₁=13.6 Hz, J₂=4.8 Hz, 1H), 3.86-3.78 (m, 3H), 3.83 (s, 3H), 3.65 (d, J=16.8 Hz, 1H), 3.55 (d, J=12.4 Hz, 1H), 2.96 (m, 1H), 1.75 (m, 1H), 1.59 (m, 1H), 1.51 (d, J=6.8 Hz, 6H), 1.05 (d, J=7.6 Hz, 3H), LC/MS: m/e=382 (M+H)⁺.

### Compound I-154

### (±)-8-(4-Chlorobenzyl)-7-ethyl-1-isopropyl-6,7,8,9-tetrahydropyrazino[1,2-a]pyrazolo[3,4-d]pyrimidin-4(1H)-one

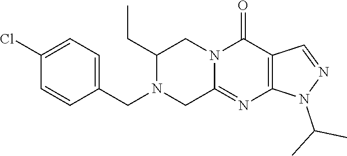 [SMILES: CCC1Cn2c(nc3c(cnn3C(C)C)c2=O)CN1Cc1ccc(Cl)cc1]

The title compound was prepared in a manner similar to the procedure used for I-91, substituting 2-((4-chlorobenzyl)amino)butan-1-ol for 2-(4-chlorophenylamino)ethanol. ¹H NMR (400 MHz, CDCl₃): δ 8.08 (s, 1H), 7.35-7.29 (m, 4H), 4.95 (sept, J=6.8 Hz, 1H), 4.21 (dd, J₁=14.0 Hz, J₂=4.8 Hz, 1H), 3.85-3.77 (m, 3H), 3.64 (d, J=17.2 Hz, 1H), 3.58 (d, J=14.2 Hz, 1H), 2.97 (m, 1H), 1.74 (m, 1H), 1.65 (m, 1H), 1.51 (d, J=7.2 Hz, 6H), 1.06 (d, J=7.2 Hz, 3H), LC/MS: m/e=386 (M+H)⁺.

### Compound I-155

### (±)-8-(4-Chlorophenyl)-7-ethyl-1-isopropyl-6,7,8,9-tetrahydropyrazino[1,2-a]pyrazolo[3,4-d]pyrimidin-4(1H)-one

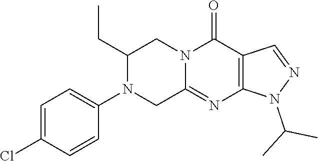 [SMILES: CCC1Cn2c(nc3c(cnn3C(C)C)c2=O)CN1c1ccc(Cl)cc1]

The title compound was prepared in a manner similar to the procedure used for I-91, substituting 2-((4-chlorophenyl)amino)butan-1-ol for 2-(4-chlorophenylamino)ethanol. ¹H NMR (400 MHz, CDCl₃): δ 8.11 (s, 1H), 7.27 (m, 2H), 6.78 (m, 2H), 5.02 (sept, J=6.4 Hz, 1H), 4.69 (dd, J₁=14.8 Hz, J₂=4.8 Hz, 1H), 4.50 (d, J=16.4 Hz, 1H), 4.42 (d, J=16.4 Hz, 1H), 3.97 (m, 2H), 1.69 (m, 1H), 1.56 (m, 6H), 1.42 (m, 1H), 0.99 (t, J=7.2 Hz, 3H), LC/MS: m/e=372 (M+H)⁺.

### Compound I-172

### (±)-8-(4-Chlorophenyl)-7-isobutyl-1-isopropyl-6,7,8,9-tetrahydropyrazino[1,2-a]pyrazolo[3,4-d]pyrimidin-4(1H)-one

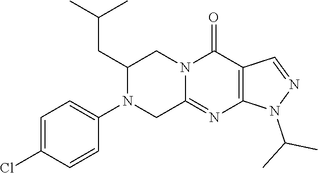 [SMILES: CC(C)CC1Cn2c(nc3c(cnn3C(C)C)c2=O)CN1c1ccc(Cl)cc1]

The title compound was prepared in a manner similar to the procedure used for I-91, substituting 2-((4-chlorophenyl)amino)-4-methylpentan-1-ol for 2-(4-chlorophenylamino)ethanol. ¹H NMR (400 MHz, CDCl₃): δ 8.10 (s, 1H), 7.26 (m, 2H), 6.79 (d, J=7.2 Hz, 2H), 5.02 (sept, J=5.2 Hz, 1H), 4.61 (dd, J₁=11.2 Hz, J₂=2.8 Hz, 1H), 4.49 (d, J=13.2 Hz, 1H), 4.39 (d, J=14.0 Hz, 1H), 4.17 (m, 1H), 3.91 (dd, J₁=10.8 Hz, J₂=3.2 Hz, 1H), 1.68 (m, 1H), 1.57 (m, 6H), 1.42 (m, 1H), 1.31 (m, 1H), 1.00 (d, J=5.2 Hz, 3H), 0.89 (d, J=5.2 Hz, 3H), LC/MS: m/e=400 (M+H)⁺.

### Compound I-241

### 8-(5-Chloropyridin-2-yl)-1-isopropyl-6,7,8,9-tetrahydropyrazino[1,2-a]pyrazolo[3,4-d]pyrimidin-4(1H)-one

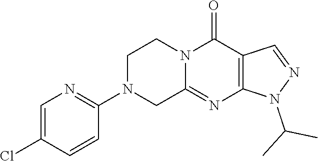 [SMILES: CC(C)n1ncc2c(=O)n3c(nc21)CN(c1ccc(Cl)cn1)CC3]

The title compound was prepared in a manner similar to the procedure used for I-91, substituting 2-((5-chloropyridin-2-yl)amino)ethanol for 2-(4-chlorophenylamino)ethanol. ¹H NMR (400 MHz, CDCl₃): δ 8.20 (d, J=2.0 Hz, 1H), 8.11 (s, 1H), 7.54 (dd, J₁=9.2 Hz, J₂=2.4 Hz, 1H), 6.61 (d, J=8.4 Hz, 1H), 5.04 (sept, J=6.8 Hz, 1H), 4.85 (s, 2H), 4.28 (t, J=5.6 Hz, 2H), 3.88 (t, J=6.0 Hz, 2H), 1.56 (d, J=6.8 Hz, 6H), LC/MS: m/e=345 (M+H)⁺.

### Compound I-246

### 8-(4-Chlorophenyl)-1-cyclobutyl-6,7,8,9-tetrahydropyrazino[1,2-a]pyrazolo[3,4-d]pyrimidin-4(1H)-one

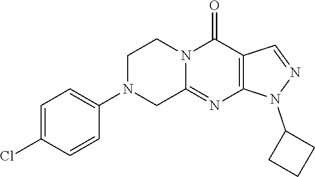 [SMILES: O=c1c2cnn(C3CCC3)c2nc2n1CCN(c1ccc(Cl)cc1)C2]

The title compound was prepared in a manner similar to the procedure used for I-91, substituting 5-amino-1-cyclobutyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide. ¹H NMR (400 MHz, CDCl₃): δ 8.12 (s, 1H), 7.29 (d, J=8.8 Hz, 2H), 6.85 (d, J=9.2 Hz, 2H), 5.25 (m, 1H), 4.49 (s, 2H), 4.27 (t, J=5.2 Hz, 2H), 3.63 (t, J=5.6 Hz, 2H), 2.79 (m, 2H), 2.45 (m, 2H), 1.90 (m, 2H), LC/MS: m/e=356 (M+H)⁺.

### Compound I-248

### 8-(4-Chlorophenyl)-1-cyclopropyl-6,7,8,9-tetrahydropyrazino[1,2-a]pyrazolo[3,4-d]pyrimidin-4(1H)-one

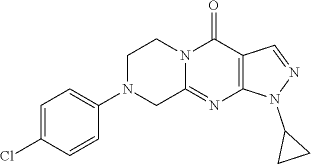 [SMILES: O=c1c2cnn(C3CC3)c2nc2n1CCN(c1ccc(Cl)cc1)C2]

The title compound was prepared in a manner similar to the procedure used for I-91, substituting 5-amino-1-cyclopropyl-1H-pyrazole-4-carboxamide (prepared according to the procedure in Haning, H., et al., Bioorg. Med. Chem. Lett. 2005, 15, 3900) for 5-amino-1-isopropyl-1H-pyrazole-4-carboxamide for 5-amino-1-isopropyl-1H-pyrazole-4-carboxamide. ¹H NMR (400 MHz, CDCl₃): δ 8.03 (s, 1H), 7.32 (m, 2H), 6.88 (m, 2H), 4.54 (s, 2H), 4.30 (t, J=5.6 Hz, 2H), 3.79 (m, 1H), 3.66 (t, J=5.6 Hz, 2H), 1.33 (m, 2H), 1.15 (m, 2H), LC/MS: m/e=342 (M+H)⁺.

### Example 3

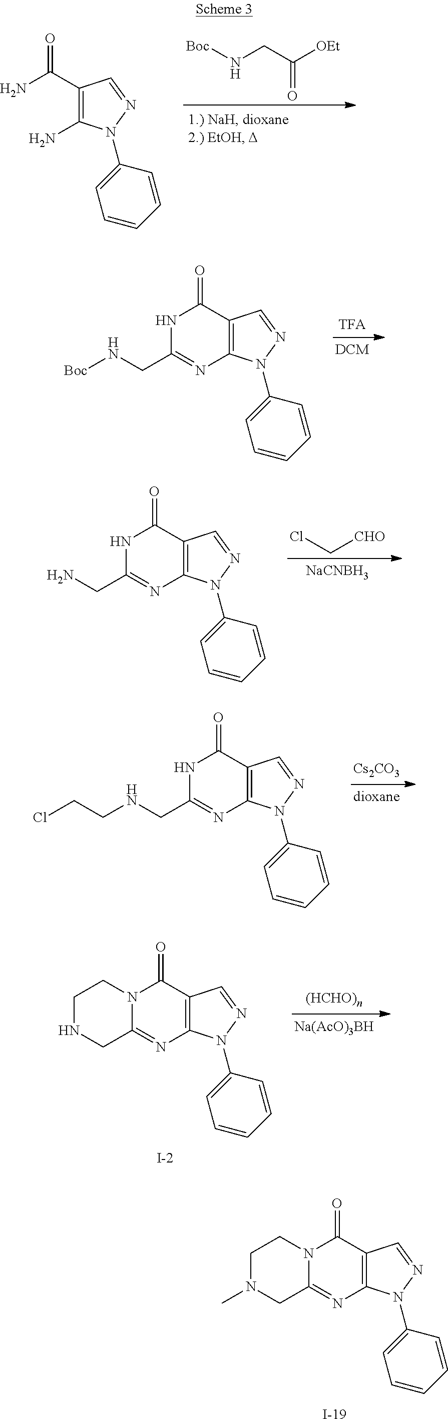

### Compound I-2

### 1-Phenyl-6,7,8,9-tetrahydropyrazino[1,2-a]pyrazolo[3,4-d]pyrimidin-4(1H)-one

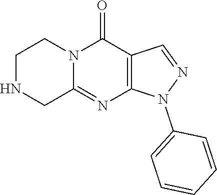 [SMILES: O=c1c2cnn(-c3ccccc3)c2nc2n1CCNC2]

To a solution of 6-((2-chloroethylamino)methyl)-1-phenyl-1H-pyrazolo[3,4-d]pyrimidin-4(5H)-one (0.33 g, 1.1 mmol) in dioxane (15 mL) was added Cs₂CO₃ (0.69 g, 2.2 mmol). The mixture was heated to reflux with stirring for 1 hour. Upon completion, the reaction mixture was filtered and the filtrate was concentrated in vacuo to afford the crude material. Purification by silica gel chromatography (eluted with dichloromethane:methanol=50:1) gave the title compound (0.19 g, 64%). ¹H NMR (400 MHz, CDCl₃): δ 8.24 (s, 1H), 8.07 (m, 2H), 7.51 (m, 2H), 7.35 (m, 1H), 4.16 (s, 2H), 4.03 (t, J=6.0 Hz, 2H), 3.32 (t, J=5.6 Hz, 2H), 1.78 (bs, 1H), LC/MS: m/e=268 (M+H)⁺.

### tert-Butyl (4-oxo-1-phenyl-4,5-dihydro-1H-pyrazolo[3,4-d]pyrimidin-6-yl)methylcarbamate

To a solution of 5-amino-1-phenyl-1H-pyrazole-4-carboxamide (prepared from phenylhydrazine according to the procedure in Haning, H., et al., Bioorg. Med. Chem. Lett. 2005, 15, 3900) (2.02 g, 10 mmol) and ethyl 2-(tert-butoxycarbonylamino)acetate (5.67 g, 30 mmol) in dioxane (50 mL) was added 60% NaH in mineral oil (4 g, 100 mmol, 10.0 eq) portionwise. The mixture was stirred at room temperature overnight, 50 mL of ethanol was quenched into the mixture and the reaction heated to reflux for 2 h. Upon completion, the mixture was evaporated in vacuo, diluted in 200 mL of water, extracted with ethyl acetate (100 mL×2), dried over sodium sulfate and concentrated in vacuo to give the title compound (3.5 g, 100%). LC/MS: m/e=342 (M+H)⁺.

### 6-(Aminomethyl)-1-phenyl-1H-pyrazolo[3,4-d]pyrimidin-4(5H)-one

To a solution of compound tert-butyl (4-oxo-1-phenyl-4,5-dihydro-1H-pyrazolo[3,4-d]pyrimidin-6-yl)methylcarbamate (3.5 g, 10.2 mmol) in dichloromethane (50 mL) was added trifluoroacetic acid (10 mL). The mixture was heated to 50° C. with stirring for 2 h. Upon the completion, the mixture was concentrated in vacuo to give the title compound as the trifluoroacetate salt (4.1 g, 100%). LC/MS: m/e=242 (M+H)⁺.

### 6-((2-Chloroethylamino)methyl)-1-phenyl-1H-pyrazolo[3,4-d]pyrimidin-4(5H)-one

To a solution of 6-(aminomethyl)-1-phenyl-1H-pyrazolo[3,4-d]pyrimidin-4(5H)-one trifluoroacetate (4.1 g, 11.8 mmol) in methanol (20 mL) was added 50% aqueous 2-chloroacetaldehyde (0.97 g, 6 mmol) and NaCNBH₃ (2.97 g, 48 mmol) sequentially. The mixture was stirred at room temperature for 4 hours. The reaction mixture was concentrated in vacuo and water (100 mL) was added. The resulting suspension was extracted with dichloromethane (50 mL). The organic layer was dried over sodium sulfate, filtered and the filtrate was concentrated in vacuo to afford the crude product. Purification by silica gel chromatography (eluting with dichloromethane:methanol=50:1) gave the title compound (0.7 g, 38%). LC/MS: m/e=304 (M+H)⁺.

### Compound I-19

### 8-Methyl-1-phenyl-6,7,8,9-tetrahydropyrazino[1,2-a]pyrazolo[3,4-d]pyrimidin-4(1H)-one

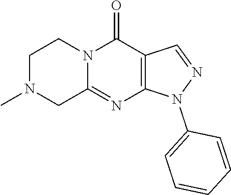 [SMILES: CN1CCn2c(nc3c(cnn3-c3ccccc3)c2=O)C1]

To a solution of 1-phenyl-6,7,8,9-tetrahydropyrazino[1,2-a]pyrazolo[3,4-d]pyrimidin-4(1H)-one (54 mg, 0.2 mmol) in dichloromethane (5 mL) was added paraformaldehyde (60 mg, 2.0 mmol) and sodium triacetoxyhydroborate (212 mg, 5.0 eq). The reaction mixture was stirred at room temperature overnight. Upon completion, the reaction was filtered and the filtrate was concentrated in vacuo to give the crude product. Purification by prep. HPLC in 0.01% aqueous ammonia gave the title compound (20 mg, 34%) as a white solid. ¹H NMR (400 MHz, CDCl₃): δ 8.24 (s, 1H), 8.09 (m, 2H), 7.51 (m, 2H), 7.35 (m, 1H), 4.10 (t, J=6.0 Hz, 2H), 3.76 (s, 2H), 2.89 (t, J=5.6 Hz, 2H), 2.50 (s, 3H), LC/MS: m/e=282 (M+H)⁺.

### Compound I-1

### 1-Isopropyl-6,7,8,9-tetrahydropyrazino[1,2-a]pyrazolo[3,4-d]pyrimidin-4(1H)-one

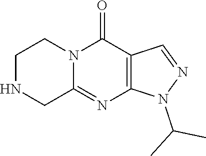 [SMILES: CC(C)n1ncc2c(=O)n3c(nc21)CNCC3]

The title compound was prepared via the procedure used for I-2, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide. ¹H NMR (400 MHz, CDCl₃): δ 8.07 (s, 1H), 4.98 (sept, J=7.2 Hz, 1H), 4.10 (s, 2H), 3.99 (t, J=5.6 Hz, 2H), 3.29 (t, J=5.6 Hz, 2H), 1.76 (bs, 1H), 1.53 (d, J=5.2 Hz, 6H), LC/MS: m/e=234 (M+H)⁺.

### Compound I-17

### 1,8-Dimethyl-6,7,8,9-tetrahydropyrazino[1,2-a]pyrazolo[3,4-d]pyrimidin-4(1H)-one

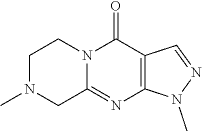 [SMILES: CN1CCn2c(nc3c(cnn3C)c2=O)C1]

The title compound was prepared via the procedure used for I-19, substituting 5-amino-1-methyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide. ¹H NMR (400 MHz, CDCl₃): δ 8.03 (s, 1H), 4.03 (t, J=5.6 Hz, 2H), 3.94 (s, 3H), 3.68 (s, 2H), 2.84 (t, J=5.6 Hz, 2H), 2.47 (s, 3H), LC/MS: m/e=220 (M+H)⁺.

### Compound I-18

### 1-Isopropyl-8-methyl-6,7,8,9-tetrahydropyrazino[1,2-a]pyrazolo[3,4-d]pyrimidin-4(1H)-one

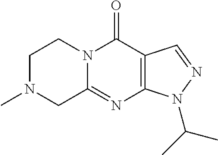 [SMILES: CC(C)n1ncc2c(=O)n3c(nc21)CN(C)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide. ¹H NMR (400 MHz, CDCl₃): δ 8.05 (s, 1H), 4.96 (sept, J=6.8 Hz, 1H), 4.03 (t, J=6.0 Hz, 2H), 3.67 (s, 2H), 2.83 (t, J=6.4 Hz, 2H), 2.46 (s, 2H), 1.51 (d, J=6.8 Hz, 6H), LC/MS: m/e=248 (M+H)⁺.

### Compound I-27

### 8-Benzyl-6,7,8,9-tetrahydropyrazino[1,2-a]pyrazolo[3,4-d]pyrimidin-4(1H)-one

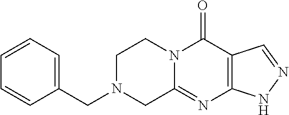 [SMILES: O=c1c2cn[nH]c2nc2n1CCN(Cc1ccccc1)C2]

The title compound was prepared via the procedure used for I-19, substituting 6,7,8,9-tetrahydropyrazino[1,2-a]pyrazolo[3,4-d]pyrimidin-4(1H)-one (I-158.1) for 1-phenyl-6,7,8,9-tetrahydropyrazino[1,2-a]pyrazolo[3,4-d]pyrimidin-4(1H)-one, and substituting benzaldehyde for paraformaldehyde. ¹H NMR (400 MHz, CDCl₃): δ 11.12 (bs, 1H), 8.14 (s, 1H), 7.38-7.32 (m, 5H), 4.06 (m, 2H), 3.78 (s, 2H), 3.72 (s, 2H), 2.92 (m, 2H), LC/MS: m/e=282 (M+H)⁺.

### 6,7,8,9-Tetrahydropyrazino[1,2-a]pyrazolo[3,4-d]pyrimidin-4(1H)-one

1-(4-Methoxybenzyl)-6,7,8,9-tetrahydropyrazino[1,2-a]pyrazolo[3,4-d]pyrimidin-4(1H)-one (prepared from para-methoxybenzylhydrazine according to the procedure used for I-2, substituting 5-amino-1-(4-methoxybenzyl)-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide) (100 mg, 0.32 mmol) was dissolved in trifluoroacetic acid (4 mL). The mixture was stirred in at 120° C. for 30 mins. Upon completion, the trifluoroacetic acid solution was evaporated in vacuo, and the residue was purified by prep. HPLC in 0.01% aqueous ammonia to afford the title compound (30 mg, 50%) as a white solid. ¹H NMR (400 MHz, CDCl₃): δ 8.11 (s, 1H), 4.03 (s, 2H), 3.96 (t, J=5.2 Hz, 2H), 3.35 (t, J=5.2 Hz, 2H), LC/MS: m/e=192 (M+H)⁺.

### Compound I-28

### 8-Benzyl-1-methyl-6,7,8,9-tetrahydropyrazino[1,2-a]pyrazolo[3,4-d]pyrimidin-4(1H)-one

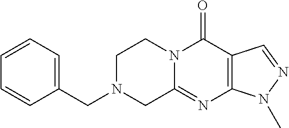 [SMILES: Cn1ncc2c(=O)n3c(nc21)CN(Cc1ccccc1)CC3]

The title compound was prepared via the procedure used for I-19, substituting 5-amino-1-methyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting benzaldehyde for paraformaldehyde. ¹H NMR (400 MHz, CDCl₃): δ 8.04 (s, 1H), 7.40-7.27 (m, 5H), 4.05 (t, J=6.4 Hz, 2H), 3.93 (s, 3H), 3.73 (d, J=7.2 Hz, 4H), 2.92 (t, J=6.4 Hz, 2H), LC/MS: m/e=296 (M+H)⁺.

### Compound I-29

### 8-Benzyl-1-isopropyl-6,7,8,9-tetrahydropyrazino[1,2-a]pyrazolo[3,4-d]pyrimidin-4(1H)-one

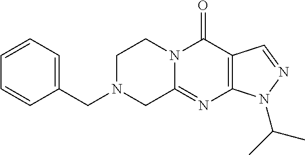 [SMILES: CC(C)n1ncc2c(=O)n3c(nc21)CN(Cc1ccccc1)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting benzaldehyde for paraformaldehyde. ¹H NMR (400 MHz, CDCl₃): δ 8.07 (s, 1H), 7.39-7.27 (m, 5H), 4.94 (sept, J=6.8 Hz, 1H), 4.06 (m, 2H), 3.74 (m, 4H), 2.93 (m, 2H), 1.51 (d, J=6.4 Hz, 6H), LC/MS: m/e=324 (M+H)⁺.

### Compound I-30

### 8-Benzyl-1-phenyl-6,7,8,9-tetrahydropyrazino[1,2-a]pyrazolo[3,4-d]pyrimidin-4(1H)-one

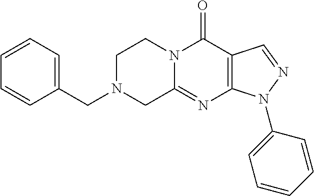 [SMILES: O=c1c2cnn(-c3ccccc3)c2nc2n1CCN(Cc1ccccc1)C2]

The title compound was prepared via the procedure used for I-19, substituting benzaldehyde for paraformaldehyde. ¹H NMR (400 MHz, CDCl₃): δ 8.23 (s, 1H), 8.05 (m, 2H), 7.49 (m, 2H), 7.39-7.32 (m, 6H), 4.09 (t, J=5.6 Hz, 2H), 3.79 (s, 2H), 3.73 (s, 2H), 2.94 (t, J=5.2 Hz, 2H). LC/MS: m/e=358 (M+H)⁺.

### Compound I-51

### 8-Benzyl-1-isobutyl-6,7,8,9-tetrahydropyrazino[1,2-a]pyrazolo[3,4-d]pyrimidin-4(1H)-one

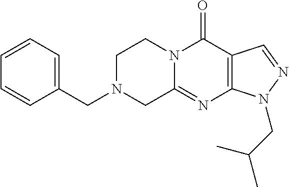 [SMILES: CC(C)Cn1ncc2c(=O)n3c(nc21)CN(Cc1ccccc1)CC3]

The title compound was prepared via the procedure used for I-19, substituting 5-amino-1-isobutyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting benzaldehyde for paraformaldehyde. ¹H NMR (400 MHz, CDCl₃): δ 8.05 (s, 1H), 7.41-7.31 (m, 5H), 4.04 (m, 4H), 3.73 (s, 2H), 3.71 (s, 2H), 2.91 (t, J=5.2 Hz, 2H), 2.35-2.28 (m, 1H), 0.89 (d, J=6.8 Hz, 6H), LC/MS: m/e=338 (M+H)⁺.

### Compound I-53

### 8-Benzyl-1-cyclopropyl-6,7,8,9-tetrahydropyrazino[1,2-a]pyrazolo[3,4-d]pyrimidin-4(1H)-one

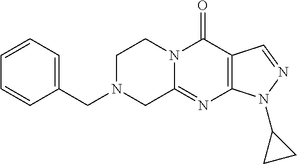 [SMILES: O=c1c2cnn(C3CC3)c2nc2n1CCN(Cc1ccccc1)C2]

The title compound was prepared via the procedure used for I-19, substituting 5-amino-1-cyclopropyl-1H-pyrazole-4-carboxamide (prepared according to the procedure in Planing, H., et al., Bioorg. Med. Chem. Lett. 2005, 15, 3900) for 5-amino-1-phenyl-1H-pyrazole-4-carboxamide, and substituting benzaldehyde for paraformaldehyde. ¹H NMR (400 MHz, CDCl₃): δ 7.98 (s, 1H), 7.39-7.31 (m, 5H), 4.06 (m, 2H), 3.78-3.69 (m, 5H), 2.93 (m, 2H), 1.27 (m, 2H), 1.09 (m, 2H), LC/MS: m/e=322 (M+H)⁺.

### Compound I-54

### 8-Benzyl-1-propyl-6,7,8,9-tetrahydropyrazino[1,2-a]pyrazolo[3,4-d]pyrimidin-4(1H)-one

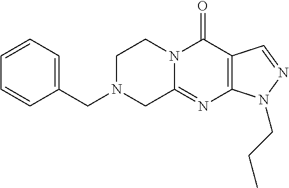 [SMILES: CCCn1ncc2c(=O)n3c(nc21)CN(Cc1ccccc1)CC3]

The title compound was prepared via the procedure used for I-19, substituting 5-amino-1-n-propyl-1H-pyrazole-4-carboxamide (prepared according to the procedure in Haning, H., et al., Bioorg. Med. Chem. Len. 2005, 15, 3900) for 5-amino-1-phenyl-1H-pyrazole-4-carboxamide, and substituting benzaldehyde for paraformaldehyde. ¹H NMR (400 MHz, CDCl₃): δ 8.04 (s, 1H), 7.40-7.27 (m, 5H), 4.22 (t, J=7.2 Hz, 2H), 4.04 (t, J=5.6 Hz, 2H), 3.72 (d, J=7.2 Hz, 4H), 2.91 (t, J=5.6 Hz, 2H), 1.91 (m, 2H), 0.89 (t, J=7.6 Hz, 3H), LC/MS: m/e=324 (M+H)⁺.

### Compound I-61

### 8-(2-Bromobenzyl)-1-isopropyl-6,7,8,9-tetrahydropyrazino[1,2-a]pyrazolo[3,4-d]pyrimidin-4(1H)-one

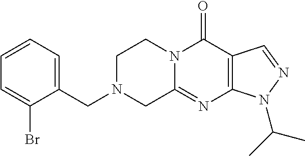 [SMILES: CC(C)n1ncc2c(=O)n3c(nc21)CN(Cc1ccccc1Br)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting 2-bromobenzaldehyde for paraformaldehyde. ¹H NMR (400 MHz, CDCl₃): δ 8.07 (s, 1H), 7.61 (dd, J₁=8.0 Hz, J₂=0.8 Hz, 1H), 7.48 (dd, J₁=7.6 Hz, J₂=1.2 Hz, 1H), 7.34 (td, J₁=8.0 Hz, J₂=1.2 Hz, 1H), 7.19 (td, J₁=7.2 Hz, J₂=2.0 Hz, 1H), 4.96 (sept, J=6.8 Hz, 1H), 4.06 (t, J=5.6 Hz, 2H), 3.82 (d, J=5.2 Hz, 4H), 2.98 (t, J=5.2 Hz, 2H), 1.52 (d, J=6.8 Hz, 6H), LC/MS: m/e=403 (M+H)⁺.

### Compound I-62

### 8-(3-Bromobenzyl)-1-isopropyl-6,7,8,9-tetrahydropyrazino[1,2-a]pyrazolo[3,4-d]pyrimidin-4(1H)-one

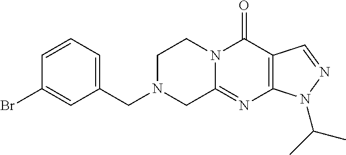 [SMILES: CC(C)n1ncc2c(=O)n3c(nc21)CN(Cc1cccc(Br)c1)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting 3-bromobenzaldehyde for paraformaldehyde. ¹H NMR (400 MHz, CDCl₃): δ 8.07 (s, 1H), 7.55 (m, 1H), 7.46 (m, 1H), 7.31 (m, 1H), 7.26 (m, 1H), 4.95 (sept, J=6.8 Hz, 1H), 4.06 (t, J=5.6 Hz, 2H), 3.71 (s, 2H), 3.68 (s, 2H), 2.92 (t, J=5.6 Hz, 2H), 1.51 (d, J=6.4 Hz, 6H), LC/MS: m/e=403 (M+H)⁺.

### Compound I-63

### 8-(4-Bromobenzyl)-1-isopropyl-6,7,8,9-tetrahydropyrazino[1,2-a]pyrazolo[3,4-d]pyrimidin-4(1H)-one

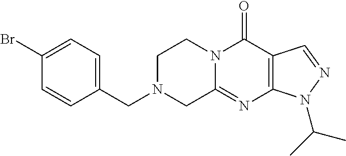 [SMILES: CC(C)n1ncc2c(=O)n3c(nc21)CN(Cc1ccc(Br)cc1)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting 4-bromobenzaldehyde for paraformaldehyde. ¹H NMR (400 MHz, CDCl₃): δ 8.07 (s, 1H), 7.51 (d, J=8.8 Hz, 2H), 7.26 (d, J=8.8 Hz, 2H), 4.94 (sept, J=6.4 Hz, 1H), 4.04 (t, J=5.6 Hz, 2H), 3.70 (s, 2H), 3.66 (s, 2H), 2.91 (t, J=6.0 Hz, 2H), 1.51 (d, J=6.8 Hz, 6H), LC/MS: m/e=403 (M+H)⁺.

### Compound I-64

### 8-([1,1′-Biphenyl]-2-ylmethyl)-1-isopropyl-6,7,8,9-tetrahydropyrazino[1,2-a]pyrazolo[3,4-d]pyrimidin-4(1H)-one

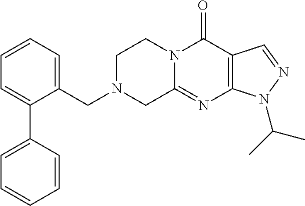 [SMILES: CC(C)n1ncc2c(=O)n3c(nc21)CN(Cc1ccccc1-c1ccccc1)CC3]

The title compound was prepared via the procedure used for I-19, substituting 5-amino-1-isopropyl-1H-pyrazole-4-carboxamide (prepared according to the procedure in timing, H., et al., Bioorg. Med. Chem. Lett. 2005, 15, 3900) for 5-amino-1-phenyl-1H-pyrazole-4-carboxamide, and substituting [1,1′-biphenyl]-2-carbaldehyde for paraformaldehyde. ¹H NMR (400 MHz, CDCl₃): δ 8.06 (s, 1H), 7.58 (m, 1H), 7.44-7.35 (m, 7H), 7.32 (m, 1H), 4.94 (sept, J=6.4 Hz, 1H), 3.96 (t, J=5.6 Hz, 2H), 3.64 (s, 2H), 3.63 (s, 2H), 2.79 (t, J=5.6 Hz, 2H), 1.51 (d, J=6.8 Hz, 6H), LC/MS: m/e=400 (M+H)⁺.

### Compound I-65

### 8-([1,1′-Biphenyl]-3-ylmethyl)-1-isopropyl-6,7,8,9-tetrahydropyrazino[1,2-a]pyrazolo[3,4-d]pyrimidin-4(1H)-one

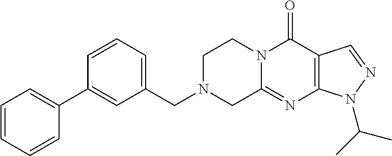 [SMILES: CC(C)n1ncc2c(=O)n3c(nc21)CN(Cc1cccc(-c2ccccc2)c1)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett, 2005, 15, 3900) for 5-amino-1-phenyl-1H-pyrazole-4-carboxamide, and substituting [1,1′-biphenyl]-3-carbaldehyde for paraformaldehyde. ¹H NMR (400 MHz, CDCl₃): δ 8.07 (s, 1H), 7.62 (m, 3H), 7.56 (d, J=7.6 Hz, 1H), 7.48-7.43 (m, 3H), 7.37 (m, 2H), 4.94 (sept, J=6.8 Hz, 1H), 4.06 (t, J=6.0 Hz, 2H), 3.78 (s, 2H), 3.76 (s, 2H), 2.95 (t, J=6.0 Hz, 2H), 1.51 (d, J=6.8 Hz, 6H), LC/MS: m/e=400 (M+H)⁺.

### Compound I-66

### 8-([1,1′-Biphenyl]-4-ylmethyl)-1-isopropyl-6,7,8,9-tetrahydropyrazino[1,2-a]pyrazolo[3,4-d]pyrimidin-4(1H)-one

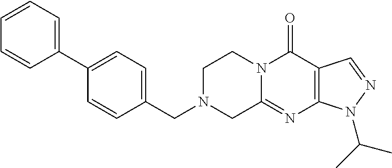 [SMILES: CC(C)n1ncc2c(=O)n3c(nc21)CN(Cc1ccc(-c2ccccc2)cc1)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting [1,1′-biphenyl]-4-carbaldehyde for paraformaldehyde. ¹H NMR (400 MHz, CDCl₃): δ 8.07 (s, 1H), 7.61 (m, 4H), 7.45 (m, 4H), 7.36 (m, 1H), 4.95 (sept, J=6.8 Hz, 1H), 4.06 (t, J=5.6 Hz, 2H), 3.75 (m, 4H), 2.94 (t, J=6.0 Hz, 2H), 1.52 (d, J=7.2 Hz, 6H), LC/MS: m/e 400 (M+H)⁺.

### Compound I-57

### 8-Isobutyl-1-isopropyl-6,7,8,9-tetrahydropyrazino[1,2-a]pyrazolo[3,4-d]pyrimidin-4(1H)-one

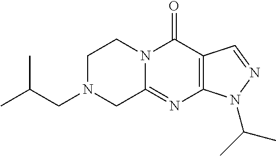 [SMILES: CC(C)CN1CCn2c(nc3c(cnn3C(C)C)c2=O)C1]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting isobutyraldehyde for paraformaldehyde. ¹H NMR (400 MHz, CDCl₃): δ 8.06 (s, 1H), 4.97 (sept, J=6.8 Hz, 1H), 4.03 (t, J=6.0 Hz, 2H), 3.70 (s, 2H), 2.85 (t, J=5.2 Hz, 2H), 2.28 (d, J=7.2 Hz, 2H), 1.88 (m, 1H), 1.52 (d, J=5.6 Hz, 6H), 0.95 (d, J=6.4 Hz, 6H), LC/MS: m/e=290 (M+H)⁺.

### Compound I-3

### (±)-1,9-Dimethyl-6,7,8,9-tetrahydropyrazino[1,2-a]pyrazolo[3,4-d]pyrimidin-4(1H)-one

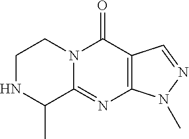 [SMILES: CC1NCCn2c1nc1c(cnn1C)c2=O]

The title compound was prepared via the procedure used for I-2, substituting 5-amino-1-methyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)propanoate for ethyl 2-(tert-butoxycarbonylamino)acetate. ¹H NMR (400 MHz, CDCl₃): δ 8.05 (s, 1H), 4.18-4.08 (m, 2H), 3.97 (s, 3H), 3.96-3.87 (m, 1H), 3.40 (dt, J₁=13.2 Hz, J₂=4.4 Hz, 1H), 3.22-3.16 (m, 1H), 1.65 (m, 3H), LC/MS: m/e=220 (M+H)⁺.

### Compound I-4

### (±)-1-Isopropyl-9-methyl-6,7,8,9-tetrahydropyrazino[1,2-a]pyrazolo[3,4-d]pyrimidin-4(1H)-one

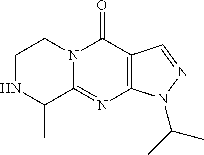 [SMILES: CC1NCCn2c1nc1c(cnn1C(C)C)c2=O]

The title compound was prepared via the procedure used for I-2, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)propanoate for ethyl 2-(tert-butoxycarbonylamino)acetate. ¹H NMR (400 MHz, CDCl₃): δ 8.03 (s, 1H), 4.98 (sept, J=7.2 Hz, 1H), 4.12 (m, 1H), 4.07 (m, 1H), 3.92-3.85 (m, 1H), 3.37 (dt, J₁=13.2 Hz, J₂=4.8 Hz, 1H), 3.20-3.13 (m, 1H), 1.85 (bs, 1H), 1.63 (d, J=7.2 Hz, 3H), 1.52 (dd, J₁=7.2 Hz, J₂=2.4 Hz, 6H), LC/MS: m/e=248 (M+H)⁺.

### Compound I-5

### (±)-9-Methyl-1-phenyl-6,7,8,9-tetrahydropyrazino[1,2-a]pyrazolo[3,4-d]pyrimidin-4(1H)-one

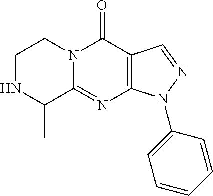 [SMILES: CC1NCCn2c1nc1c(cnn1-c1ccccc1)c2=O]

The title compound was prepared via the procedure used for I-2, substituting (+/−)-ethyl 2-((tert-butoxycarbonyl)amino)propanoate for ethyl 2-(tert-butoxycarbonylamino)acetate. ¹H NMR (400 MHz, CDCl₃): δ 8.24 (s, 1H), 8.13 (dd, J₁=8.8 Hz, J₂=1.2 Hz, 2H), 7.51 (m, 2H), 7.35 (m, 1H), 4.20 (m, 1H), 4.15 (m, 1H), 3.94 (m, 1H), 3.44 (m, 1H), 3.25-3.19 (m, 1H), 1.70 (bs, 1H), 1.69 (d, J=6.8 Hz, 3H), LC/MS: m/e=282 (M+H)⁺.

### Compound I-6

### (±)-9-Benzyl-6,7,8,9-tetrahydropyrazino[1,2-a]pyrazolo[3,4-d]pyrimidin-4(1H)-one

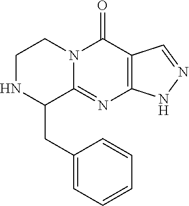 [SMILES: O=c1c2cn[nH]c2nc2n1CCNC2Cc1ccccc1]

The title compound was prepared via the procedure used for I-2, substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.18 (s, 1H), 7.37-7.27 (m, 5H), 4.23 (dd, J₁=10.4 Hz, J₂=3.6 Hz, 1H), 4.18 (dt, J₁=14.0 Hz, J₂=4.0 Hz, 1H), 3.86-3.79 (m, 1H), 3.72 (dd, J₁=14.0 Hz, J₂=3.6 Hz, 1H), 3.37 (dt, J₁=12.8 Hz, J₂=4.4 Hz, 1H), 3.08-3.02 (m, 2H), 1.65 (bs, 2H), LC/MS: m/e=282 (M+H)⁺.

### Compound I-7

### (±)-9-Benzyl-1-methyl-6,7,8,9-tetrahydropyrazino[1,2-a]pyrazolo[3,4-d]pyrimidin-4(1H)-one

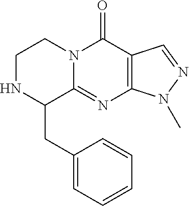 [SMILES: Cn1ncc2c(=O)n3c(nc21)C(Cc1ccccc1)NCC3]

The title compound was prepared via the procedure used for I-2, substituting 5-amino-1-methyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.07 (s, 1H), 7.38-7.27 (m, 5H), 4.19 (m, 2H), 4.01 (s, 3H), 3.82-3.75 (m, 2H), 3.36 (dt, J₁=12.8 Hz, J₂=4.4 Hz, 1H), 3.08-3.01 (m, 2H), 1.70 (bs, 1H) LC/MS: m/e=296 (M+H)⁺.

### Compound I-8

### (±)-9-Benzyl-1-isopropyl-6,7,8,9-tetrahydropyrazino[1,2-a]pyrazolo[3,4-d]pyrimidin-4(1H)-one

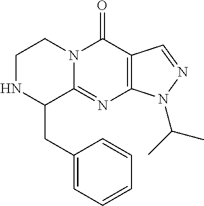 [SMILES: CC(C)n1ncc2c(=O)n3c(nc21)C(Cc1ccccc1)NCC3]

The title compound was prepared via the procedure used for I-2, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((test-butoxycarbonyl)amino)-3-phenylpropanoate for ethyl 2-(tert-butoxycarbonylamino)acetate. ¹H NMR (400 MHz, CDCl₃): δ 8.08 (s, 1H), 7.37-7.27 (m, 5H), 5.06 (sept, J=6.8 Hz, 1H), 4.22-4.14 (m, 2H), 3.81-3.74 (m, 2H), 3.36 (dt, J₁=12.4 Hz, J₂=4.4 Hz, 1H), 3.08-3.00 (m, 2H), 1.79 (bs, 1H), 1.57 (dd, J₁=6.4 Hz, J₂=1.2 Hz, 6H), LC/MS: m/e=324 (M+H)⁺.

### Compound I-9

### (±)-9-Benzyl-1-phenyl-6,7,8,9-tetrahydropyrazino[1,2-a]pyrazolo[3,4-d]pyrimidin-4(1H)-one

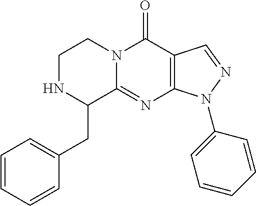 [SMILES: O=c1c2cnn(-c3ccccc3)c2nc2n1CCNC2Cc1ccccc1]

The title compound was prepared via the procedure used for I-2, substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.25 (s, 1H), 8.14 (m, 2H), 7.51 (m, 2H), 7.35 (m, 3H), 7.37-7.33 (m, 3H), 4.27-4.20 (m, 2H), 3.84-3.78 (m, 2H), 3.38 (m, 2H), 3.11-3.02 (m, 2H), 1.79 (s, 1H), LC/MS: m/e=358 (M+H)⁺.

### Compound I-20

### (±)-1,8,9-Trimethyl-6,7,8,9-tetrahydropyrazino[1,2-a]pyrazolo[3,4-d]pyrimidin-4(1H)-one

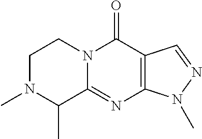 [SMILES: CC1c2nc3c(cnn3C)c(=O)n2CCN1C]

The title compound was prepared via the procedure used for I-19, substituting 5-amino-1-methyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)propanoate for ethyl 2-(tert-butoxycarbonylamino)acetate. ¹H NMR (400 MHz, CDCl₃): δ 8.04 (s, 1H), 4.25 (dt, J₁=14.0 Hz, J₂=4.0 Hz, 1H), 3.96 (s, 3H), 3.86-3.79 (m, 1H), 3.53 (dd, J₁=13.2 Hz, J₂=6.8 Hz, 1H), 3.19 (dt, J₁=12.8 Hz, J₂=4.4 Hz, 1H), 2.72-2.66 (m, 1H), 2.50 (s, 3H), 1.62 (d, J=6.8 Hz, 3H), LC/MS: m/e=234 (M+H)⁺.

### Compound I-21

### (±)-1-Isopropyl-8,9-dimethyl-6,7,8,9-tetrahydropyrazino[1,2-a]pyrazolo[3,4-d]pyrimidin-4(1H)-one

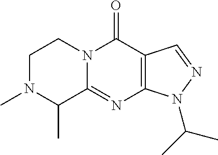 [SMILES: CC1c2nc3c(cnn3C(C)C)c(=O)n2CCN1C]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15 3900) for 5-amino-1-phenyl-1H-pyrazole-4-carboxamide, and substituting (+/−)-ethyl 2-((tert-butoxycarbonyl)amino)propanoate for ethyl 2-(tert-butoxycarbonylamino)acetate. ¹H NMR (400 MHz, CDCl₃): δ 8.05 (s, 1H), 4.99 (sept, J=6.8 Hz, 1H), 4.25 (dt, J₁=13.6 Hz, J₂=4.0 Hz, 1H), 3.85-3.78 (m, 1H), 3.51 (dd, J₁=13.2 Hz, J₂=6.4 Hz, 1H), 3.18 (dt, J₁=12.4 Hz, J₂=4.4 Hz, 1H), 2.72-2.66 (m, 1H), 2.50 (s, 3H), 1.62 (d, J=7.2 Hz, 3H), 1.54 (dd, J₁=6.8 Hz, J₂=2.8 Hz, 6H), LC/MS: m/e=262 (M+H)⁺.

### Compound I-22

### (±)-8,9-Dimethyl-1-phenyl-6,7,8,9-tetrahydropyrazino[1,2-a]pyrazolo[3,4-d]pyrimidin-4(1H)-one

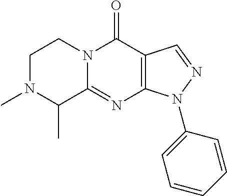 [SMILES: CC1c2nc3c(cnn3-c3ccccc3)c(=O)n2CCN1C]

The title compound was prepared via the procedure used for I-19, substituting (+/−)-ethyl 2-((tert-butoxycarbonyl)amino)propanoate for ethyl 2-(tert-butoxycarbonylamino)acetate. ¹H NMR (400 MHz, CDCl₃): δ 8.23 (s, 1H), 8.11 (m, 2H), 7.50 (m, 2H), 7.34 (m, 1H), 4.29 (dt, J₁=14.0 Hz, J₂=4.0 Hz, 1H), 3.88 (m, 1H), 3.58 (dd, J₁=13.2 Hz, J₂=6.4 Hz, 1H), 3.21 (dt, J₁=12.4 Hz, J₂=4.4 Hz, 1H), 2.73 (m, 1H), 2.51 (s, 3H), 1.62 (d, J=6.4 Hz, 3H), LC/MS: m/e=296 (M+H)⁺.

### Compound I-23

### (±)-9-Benzyl-8-methyl-6,7,8,9-tetrahydropyrazino[1,2-a]pyrazolo[3,4-d]pyrimidin-4(1H)-one

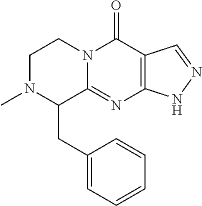 [SMILES: CN1CCn2c(nc3[nH]ncc3c2=O)C1Cc1ccccc1]

The title compound was prepared via the procedure used for I-2, substituting (+/−)-ethyl 2-((tert-butoxycarbonyl)amino)-3-phenylpropanoate for ethyl 2-(tert-butoxycarbonylamino)acetate, and substituting paraformaldehyde for benzaldehyde. ¹H NMR (400 MHz, CDCl₃): δ 8.17 (s, 1H), 7.18-7.08 (m, 5H), 4.27 (dt, J₁=14.0 Hz, J₂=3.6 Hz, 1H), 3.95 (t, J=4.8 Hz, 1H), 3.41-3.29 (m, 3H), 3.20 (dt, J₁=12.4 Hz, J₂=4.0 Hz, 1H), 2.67 (m, 1H), 2.56 (s, 3H), LC/MS: m/e=296 (M+H)⁺.

### Compound I-24

### (±)-9-Benzyl-1,8-dimethyl-6,7,8,9-tetrahydropyrazino[1,2-a]pyrazolo[3,4-d]pyrimidin-4(1H)-one

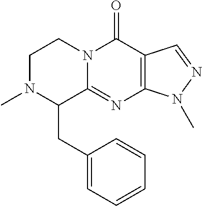 [SMILES: CN1CCn2c(nc3c(cnn3C)c2=O)C1Cc1ccccc1]

The title compound was prepared via the procedure used for I-19, substituting 5-amino-1-methyl-1H-pyrazole-4-carboxamide (prepared according to the procedure in Halting, H. et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.03 (s, 1H), 7.18 (m, 3H), 7.09 (m, 2H), 4.24 (dt, J₁=14.0 Hz, J₂=4.0 Hz, 1H), 3.97 (s, 3H), 3.87 (t, J=4.8 Hz, 1H), 3.43-3.26 (m, 3H), 3.17 (dt, J₁=12.4 Hz, J₂=4.0 Hz, 1H), 2.65 (m, 1H), 2.54 (s, 3H), LC/MS: m/e=310 (M+H)⁺.

### Compound I-25

### (±)-9-Benzyl-1-isopropyl-8-methyl-6,7,8,9-tetrahydropyrazino[1,2-a]pyrazolo[3,4-d]pyrimidin-4(1H)-one

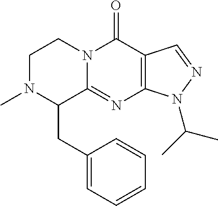 [SMILES: CC(C)n1ncc2c(=O)n3c(nc21)C(Cc1ccccc1)N(C)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.05 (s, 1H), 7.17 (m, 3H), 7.10 (m, 2H), 4.99 (sept, J=6.8 Hz, 1H), 4.25 (dt, J₁=13.6 Hz, J₂=4.0 Hz, 1H), 3.87 (m, 1H), 3.43-3.36 (m, 2H), 3.29 (dd, J₁=14.0 Hz, J₂=4.8 Hz, 1H), 3.18 (dt, J₁=12.4 Hz, J₂=4.0 Hz, 1H), 2.67 (m, 1H), 2.56 (s, 3H), 1.56 (dd, r=6.8 Hz, J₂=1.6 Hz, 6H), LC/MS: m/e=338 (M+H)⁺.

### Compound I-26

### (±)-9-Benzyl-8-methyl-1-phenyl-6,7,8,9-tetrahydropyrazino[1,2-a]pyrazolo[3,4-d]pyrimidin-4(1H)-one

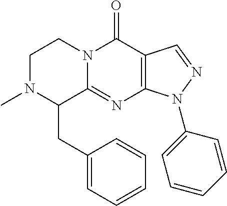 [SMILES: CN1CCn2c(nc3c(cnn3-c3ccccc3)c2=O)C1Cc1ccccc1]

The title compound was prepared via the procedure used for I-19, substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.23 (s, 1H), 8.05 (d, J=8.0 Hz, 2H), 7.49 (t, J=8.0 Hz, 2H), 7.35 (m, 1H), 7.20 (m, 3H), 7.12 (m, 2H), 4.29 (dt, J₁=14.0 Hz, J₂=4.0 Hz, 1H), 3.95 (t, J=4.8 Hz, 1H), 3.48-3.41 (m, 2H), 3.31 (m, 1H), 3.22 (dt, J₁=12.8 Hz, J₂=4.4 Hz, 1H), 2.70 (m, 1H), 2.58 (s, 3H), LC/MS: m/e=372 (M+H)⁺.

### Compound I-31

### (±)-8-Benzyl-1,9-dimethyl-6,7,8,9-tetrahydropyrazino[1,2-a]pyrazolo[3,4-d]pyrimidin-4(1H)-one

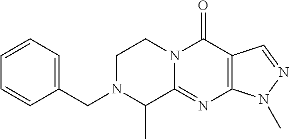 [SMILES: CC1c2nc3c(cnn3C)c(=O)n2CCN1Cc1ccccc1]

The title compound was prepared via the procedure used for I-19, substituting 5-amino-1-methyl-1H-pyrazole-4-carboxamide (prepared according to the procedure in Haning, H., et al., Bioorg. Med. Chem. Lett, 2005, 15, 3900) for 5-amino-1-phenyl-1H-pyrazole-4-carboxamide, substituting (+/−)-ethyl 2-((tert-butoxycarbonyl)amino)propanoate for ethyl 2-(tert-butoxycarbonylamino)acetate, and substituting benzaldehyde for paraformaldehyde. ¹H NMR (400 MHz, CDCl₃): δ 8.04 (s, 1H), 7.38-7.27 (m, 5H), 4.05 (m, 1H), 3.96 (s, 3H), 3.95-3.88 (m, 3H), 3.64 (d, J=13.2 Hz, 1H), 3.18 (m, 1H), 2.75 (m, 1H), 1.63 (d, J=6.8 Hz, 3H), LC/MS: m/e=310 (M+H)⁺.

### Compound I-32

### (±)-8-Benzyl-1-isopropyl-9-methyl-6,7,8,9-tetrahydropyrazino[1,2-a]pyrazolo[3,4-d]pyrimidin-4(1H)-one

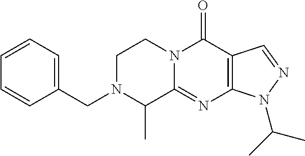 [SMILES: CC1c2nc3c(cnn3C(C)C)c(=O)n2CCN1Cc1ccccc1]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, substituting (+/−)-ethyl 2-((tert-butoxycarbonyl)amino)propanoate for ethyl 2-(tert-butoxycarbonylamino)acetate, and substituting benzaldehyde for paraformaldehyde. ¹H NMR (400 MHz, CDCl₃): δ 8.06 (s, 1H), 7.39-7.27 (m, 5H), 4.99 (sept, J=6.8 Hz, 1H), 4.09-4.03 (m, 1H), 3.98-3.87 (m, 3H), 3.63 (d, J=12.8 Hz, 1H), 3.20-3.14 (m, 1H), 2.77-2.71 (m, 1H), 1.63 (d, J=6.8 Hz, 3H), 1.54 (dd, J₁=6.8 Hz, J₂=2.8 Hz, 6H), LC/MS: m/e=338 (M+H)⁺.

### Compound I-33

### (±)-8-Benzyl-9-methyl-1-phenyl-6,7,8,9-tetrahydropyrazino[1,2-a]pyrazolo[3,4-d]pyrimidin-4(1H)-one

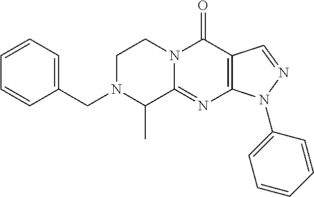 [SMILES: CC1c2nc3c(cnn3-c3ccccc3)c(=O)n2CCN1Cc1ccccc1]

The title compound was prepared via the procedure used for I-19, substituting (+/−)-ethyl 2-((tert-butoxycarbonyl)amino)propanoate for ethyl 2-(tert-butoxycarbonylamino)acetate, and substituting benzaldehyde for paraformaldehyde. ¹H NMR (400 MHz, CDCl₃): δ 8.24 (s, 1H), 8.11 (dd, J₁=6.8 Hz, J₂=0.4 Hz, 2H), 7.51 (m, 2H), 7.40-7.30 (m, 6H), 4.11 (m, 1H), 4.02-3.92 (m, 3H), 3.65 (d, J=10.8 Hz, 1H), 3.22 (m, 1H), 2.78 (m, 1H), 1.68 (d, J=5.2 Hz, 3H), LC/MS: m/e=372 (M+H)⁺.

### Compound I-34

### (±)-8,9-Dibenzyl-6,7,8,9-tetrahydropyrazino[1,2-a]pyrazolo[3,4-d]pyrimidin-4(1H)-one

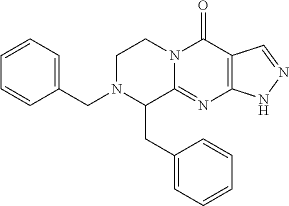 [SMILES: O=c1c2cn[nH]c2nc2n1CCN(Cc1ccccc1)C2Cc1ccccc1]

The title compound was prepared via the procedure used for I-2, substituting (+/−)-ethyl 2-((tert-butoxycarbonyl)amino)-3-phenylpropanoate for ethyl 2-(tert-butoxycarbonylamino)acetate. ¹H NMR (400 MHz, CDCl₃): δ 8.18 (s, 1H), 7.29 (m, 3H), 7.22-7.12 (m, 7H), 4.19-4.13 (m, 2H), 3.97 (d, J=13.2 Hz, 1H), 3.68 (d, J=13.2 Hz, 1H), 3.49-3.43 (m, 1H), 3.31 (d, J=5.6 Hz, 2H), 3.28 (m, 1H), 2.74-2.67 (m, 1H), LC/MS: m/e=372 (M+H)⁺.

### Compound I-35

### (±)-8,9-Dibenzyl-1-methyl-6,7,8,9-tetrahydropyrazino[1,2-a]pyrazolo[3,4-d]pyrimidin-4(1H)-one

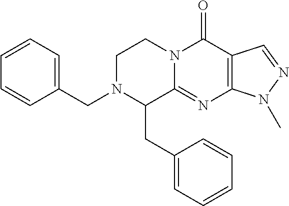 [SMILES: Cn1ncc2c(=O)n3c(nc21)C(Cc1ccccc1)N(Cc1ccccc1)CC3]

The title compound was prepared via the procedure used for I-19, substituting 5-amino-1-methyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, substituting (+/−)-ethyl 2-((tert-butoxycarbonyl)amino)-3-phenylpropanoate for ethyl 2-(tert-butoxycarbonylamino)acetate, and substituting benzaldehyde for paraformaldehyde. ¹H NMR (400 MHz, CDCl₃): δ 8.05 (s, 1H), 7.33-7.26 (m, 3H), 7.24-7.19 (m, 5H), 7.13 (m, 2H), 4.16-4.11 (m, 2H), 3.97 (d, J=13.6 Hz, 1H), 3.96 (s, 3H), 3.66 (d, J=13.6 Hz, 1H), 3.51-3.44 (m, 1H), 3.36-3.22 (m, 3H), 2.72-2.66 (m, 1H), LC/MS: m/e=386 (M+H)⁺.

### Compound I-36

### (±)-8,9-Dibenzyl-1-Isopropyl-6,7,8,9-tetrahydropyrazino[1,2-a]pyrazolo[3,4-d]pyrimidin-4(1H)-one

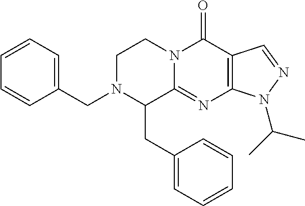 [SMILES: CC(C)n1ncc2c(=O)n3c(nc21)C(Cc1ccccc1)N(Cc1ccccc1)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett 2005, 15, 3900) for 5-amino-1-phenyl-1H-pyrazole-4-carboxamide, substituting (+/−)-ethyl 2-((tert-butoxycarbonyl)amino)-3-phenylpropanoate for ethyl 2-(tert-butoxycarbonylamino)acetate, and substituting benzaldehyde for paraformaldehyde. ¹H NMR (400 MHz, CDCl₃): δ 8.07 (s, 1H), 7.34-7.19 (m, 8H), 7.14 (m, 2H), 4.99 (sept, J 6.8 Hz, 1H), 4.17-4.11 (m, 2H), 3.98 (d, J=13.6 Hz, 1H), 3.67 (d, J=13.6 Hz, 1H), 3.52-3.46 (m, 1H), 3.37-3.22 (m, 3H), 2.72-2.66 (m, 1H), 1.55 (dd, J₁=6.8 Hz, J₂=3.2 Hz, 6H), LC/MS: m/e=414 (M+H)⁺.

### Compound I-37

### (±)-8,9-Dibenzyl-1-phenyl-6,7,8,9-tetrahydropyrazino[1,2-a]pyrazolo[3,4-d]pyrimidin-4(1H)-one

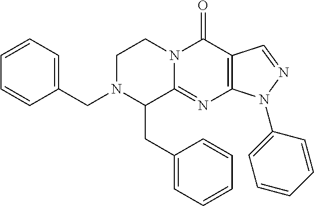 [SMILES: O=c1c2cnn(-c3ccccc3)c2nc2n1CCN(Cc1ccccc1)C2Cc1ccccc1]

The title compound was prepared via the procedure used for I-19, substituting (+/−)-ethyl 2-((tert-butoxycarbonyl)amino)-3-phenylpropanoate for ethyl 2-(tert-butoxycarbonylamino)acetate, and substituting benzaldehyde for paraformaldehyde. ¹H NMR (400 MHz, CDCl₃): δ 8.24 (s, 1H), 7.98 (d, J=8.0 Hz, 2H), 7.48 (t, J=8.0 Hz, 2H), 7.36-7.31 (m, 4H), 7.25 (m, 5H), 7.13 (m, 2H), 4.23-4.15 (m, 2H), 4.02 (d, J=13.2 Hz, 1H), 3.72 (d, J=13.2 Hz, 1H), 3.58 (m, 1H), 3.40 (m, 1H), 3.31 (m, 2H), 2.74 (m, 1H), LC/MS: m/e=448 (M+H)⁺.

### Compound I-56

### (±)-8-benzyl-9-isobutyl-1-isopropyl-6,7,8,9-tetrahydropyrazino[1,2-a]pyrazolo[3,4-d]pyrimidin-4(1H)-one

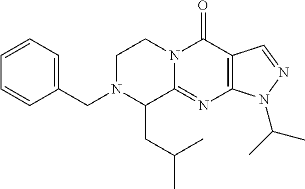 [SMILES: CC(C)CC1c2nc3c(cnn3C(C)C)c(=O)n2CCN1Cc1ccccc1]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, substituting (+/−)-ethyl 2-((tert-butoxycarbonyl)amino)-4-methylpentanoate for ethyl 2-(tert-butoxycarbonylamino)acetate, and substituting benzaldehyde for paraformaldehyde. ¹H NMR (400 MHz, d⁴-MeOH): δ 7.92 (s, 1H), 7.28-7.15 (m, 5H), 4.91 (sept, J=6.8 Hz, 1H), 3.99-3.89 (m, 1H), 3.83-3.77 (m, 1H), 3.74-3.65 (m, 3H), 3.32-3.22 (m, 1H), 2.89-2.84 (m, 1H), 1.87-1.81 (m, 2H), 1.60 (m, 1H), 1.41 (dd, J₁=7.6 Hz, J₂=6.8 Hz, 6H), 0.78 (d, J=6.8 Hz, 3H), 0.71 (d, J=6.4 Hz, 3H), LC/MS: m/e=380 (M+H)⁺.

### Compound I-58

### (±)-1-cyclopentyl-8,9-diisobutyl-6,7,8,9-tetrahydropyrazino[1,2-a]pyrazolo[3,4-d]pyrimidin-4(1H)-one

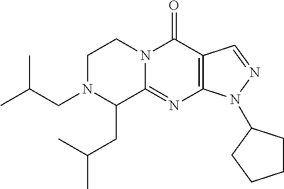 [SMILES: CC(C)CC1c2nc3c(cnn3C3CCCC3)c(=O)n2CCN1CC(C)C]

The title compound was prepared via the procedure used for I-19, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, substituting (+/−)-ethyl 2-((tert-butoxycarbonyl)amino)-4-methylpentanoate for ethyl 2-(tert-butoxycarbonylamino)acetate, and substituting isobutyraldehyde for paraformaldehyde. ¹H NMR (400 MHz, d⁴-MeOH): δ8.07 (s, 1H), 5.03 (quint, J=6.8 Hz, 1H), 4.46 (dd, J₁=8.8 Hz, J₂=4.4 Hz, 1H), 4.36 (dt, J₁=14.8 Hz, J₂=5.2 Hz, 1H), 4.30 (m, 1H), 3.83 (m, 1H), 3.67 (m, 1H), 3.08 (m, 2H), 2.23-2.12 (m, 2H), 2.00 (m, 1H), 1.87 (m, 1H), 1.54 (dd, J₁=6.8 Hz, J₂=2.4 Hz, 6H), 1.12-1.05 (m, 12H), LC/MS: m/e=346 (M+H)⁺.

### Compound I-60

### (±)-1-Isopropyl-8-methyl-9-phenyl-6,7,8,9-tetrahydropyrazino[1,2-a]pyrazolo[3,4-d]pyrimidin-4(1H)-one

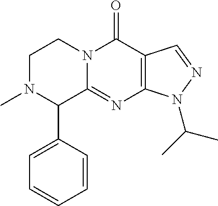 [SMILES: CC(C)n1ncc2c(=O)n3c(nc21)C(c1ccccc1)N(C)CC3]

The title compound was prepared via the procedure used for I-19, substituting 5-amino-1-isopropyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, and substituting (+/−)-ethyl 2-((tert-butoxycarbonyl)amino)-2-phenylacetate for ethyl 2-(tert-butoxycarbonylamino)acetate. ¹H NMR (400 MHz, CDCl₃): δ 8.01 (s, 1H), 7.40-7.28 (m, 5H), 4.71 (sept, J=6.4 Hz, 1H), 4.47 (dt, J₁=13.2 Hz, J₂=3.6 Hz, 1H), 4.41 (s, 1H), 3.88 (m, 1H), 3.30 (dt, J₁=12.0 Hz, J₂=4.0 Hz, 1H), 2.79-2.73 (m, 1H), 2.34 (s, 3H), 1.32 (dd, J₁=15.2 Hz, J₂=6.8 Hz, 6H), LC/MS: m/e=324 (M+H)⁺.

### Compound I-117

### (±)-8-(4-Chlorobenzyl)-1-cyclopentyl-9-methyl-6,7,8,9-tetrahydropyrazino[1,2-a]pyrazolo[3,4-d]pyrimidin-4(1H)-one

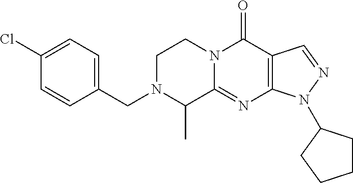 [SMILES: CC1c2nc3c(cnn3C3CCCC3)c(=O)n2CCN1Cc1ccc(Cl)cc1]

The title compound was prepared via the procedure used for I-19, substituting 5-amino-1-cyclopentyl-1H-pyrazole-4-carboxamide (prepared according to the procedure in Haning, H., et al., Bioorg. Med. Chem. Lett. 2005, 15, 3900) for 5-amino-1-phenyl-1H-pyrazole-4-carboxamide, substituting (+/−)-ethyl 2-((tert-butoxycarbonyl)amino)propanoate for ethyl 2-(tert-butoxycarbonylamino)acetate, and substituting 4-chlorobenzaldehyde for paraformaldehyde. ¹H NMR (400 MHz, CDCl₃): δ 8.05 (s, 1H), 7.31 (m, 4H), 5.11 (quint, J=7.6 Hz, 1H), 4.02 (m, 1H), 3.92 (m, 2H), 3.89 (d, J=13.6 Hz, 1H), 3.60 (d, J=13.6 Hz, 1H), 3.14 (m, 1H), 2.73 (m, 1H), 2.14-2.06 (m, 4H), 1.97 (m, 2H), 1.70 (m, 2H), 1.60 (d, J=6.8 Hz, 3H), LC/MS: m/e=398 (M+H)⁺.

### Compound I-118

### (±)-1-Cyclopentyl-8-(4-methoxybenzyl)-9-methyl-6,7,8,9-tetrahydropyrazino[1,2-a]pyrazolo[3,4-d]pyrimidin-4(1H)-one

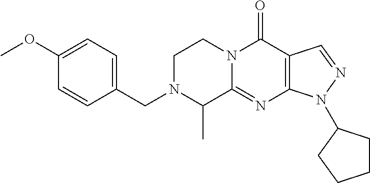 [SMILES: COc1ccc(CN2CCn3c(nc4c(cnn4C4CCCC4)c3=O)C2C)cc1]

The title compound was prepared via the procedure used for I-19, substituting 5-amino-1-cyclopentyl-1H-pyrazole-4-carboxamide (prepared according to the procedure in Hailing, H., et al., Bioorg. Med. Chem. Lett. 2005, 15, 3900) for 5-amino-1-phenyl-1H-pyrazole-4-carboxamide, substituting (+/−)-ethyl 2-((tert-butoxycarbonyl)amino)propanoate for ethyl 2-(tert-butoxycarbonylamino)acetate, and substituting 4-methoxybenzaldehyde for paraformaldehyde. ¹H NMR (400 MHz, CDCl₃): δ 8.04 (s, 1H), 7.27 (m, 2H), 6.88 (m, 2H), 5.12 (quint, J=7.6 Hz, 1H), 4.04 (m, 1H), 3.95-3.85 (m, 3H), 3.81 (s, 3H), 3.56 (d, J=13.2 Hz, 1H), 3.14 (m, 1H), 2.72 (m, 1H), 2.14-2.06 (m, 4H), 1.97 (m, 2H), 1.70 (m, 2H), 1.61 (d, J=6.8 Hz, 3H), LC/MS: m/e=394 (M+H)⁺.

### Example 4

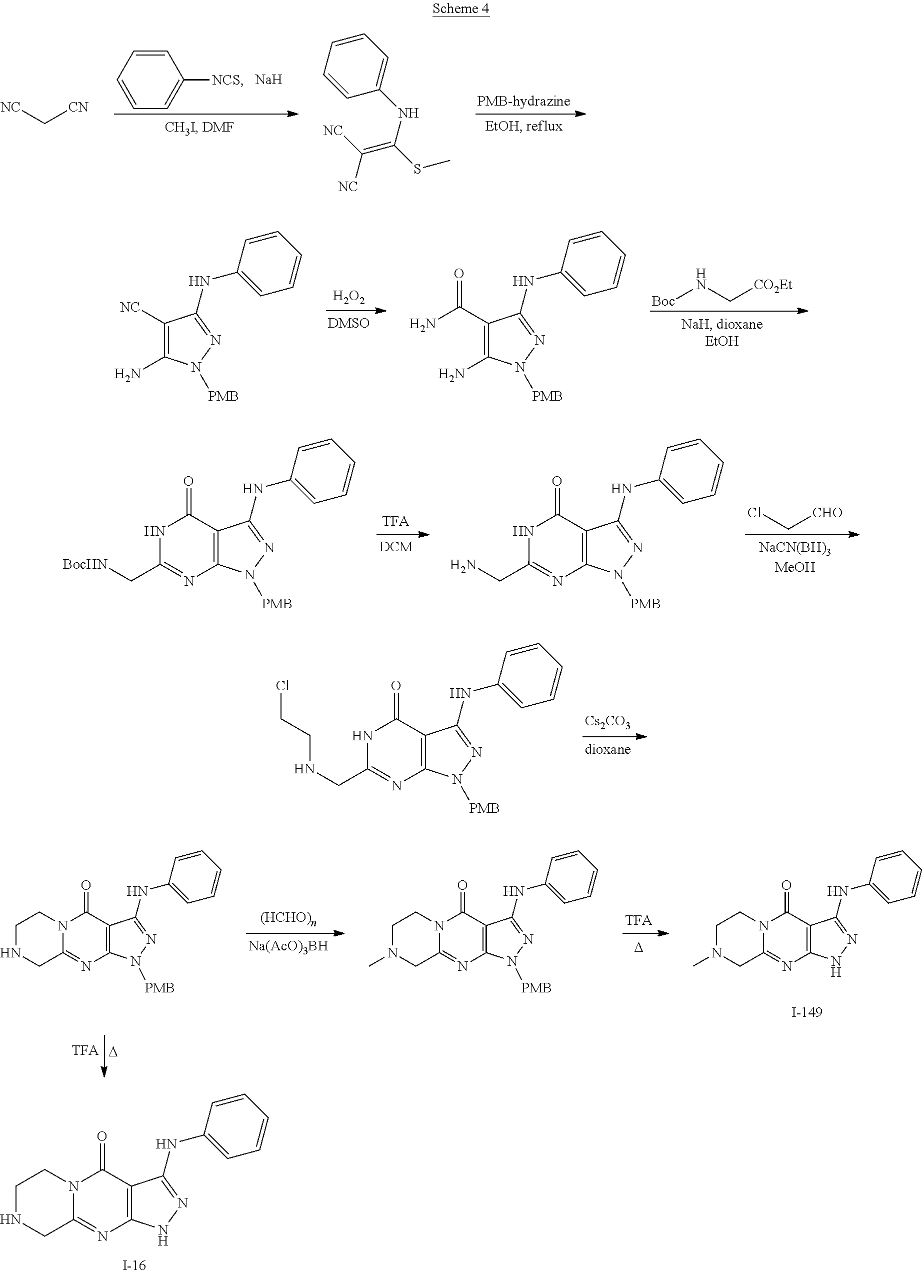

### Compound I-16

### 3-(Phenylamino)-6,7,8,9-tetrahydropyrazino[1,2-a]pyrazolo[3,4-d]pyrimidin-4(1H)-one

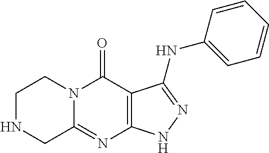 [SMILES: O=c1c2c(Nc3ccccc3)n[nH]c2nc2n1CCNC2]

To a solution of 1-(4-methoxybenzyl)-3-(phenylamino)-6,7,8,9-tetrahydropyrazino[1,2-a]pyrazolo[3,4-d]pyrimidin-4(1H)-one (I-198.7) (50 mg, 0.12 mmol) trifluoroacetic acid (1 mL) was added conc. sulfuric acid (cat.) in a microwave tube. The mixture was heated to 120° C. with stirring for 20 mins. Upon completion, the mixture was concentrated in vacuo, neutralized with 25% aqueous ammonia (0.5 mL), extracted with dichloromethane (20 mL×2), dried and concentrated under reduced pressure. Purification by prep. HPLC afforded the title compound (15 mg, 43%) as a white solid. ¹H NMR (400 MHz, d⁶-DMSO): δ 12.62 (bs, 1H), 7.88 (s, 1H), 7.65 (d, J=8.0 Hz, 2H), 7.24 (dd, J₁=8.4 Hz, J₂=7.2 Hz, 2H), 6.84 (t, J=7.2 Hz, 1H), 3.84 (s, 2H), 3.75 (t, J=6.0 Hz, 2H), 3.09 (t, J=5.6 Hz, 2H), 2.73 (bs, 1H), LC/MS: m/e=283 (M+H)⁺.

### 2-(Methylthio(phenylamino)methylene)malononitrile

A solution of malononitrile (6.6 g, 0.1 mol) in DMF (20 mL) was added slowly to an ice-cooled suspension of sodium hydride (60%, dispersion in mineral oil, 4 g, 0.1 mol) in DMF (30 mL). The rate of addition was such as to maintain the temperature of the reaction mixture below 5° C. After the addition was complete, the reaction continued to stir at 0° C. for 30 mins. Isothiocyanatobenzene (13.5 g, 0.1 mol) was added in one portion to give an orange mixture, which was allowed to warm to room temperature and stirred for 2 h. The mixture was cooled to 0° C. Iodomethane (14.2 g, 0.1 mol) was added slowly to the reaction mixture at a rate that maintained the temperature below 5° C. The resulting yellow mixture was allowed to warm to room temperature and stirred for 2 h. Upon completion, the reaction was poured into water and extracted with ethyl acetate. The organic phase was dried over sodium sulfate and evaporated to dryness under reduced pressure to give the crude product, which was recrystallized from ethyl acetate to give the title compound (15 g, 70%). LC/MS: m/e=216 (M+H)⁺.

### 3-Amino-1-(4-methoxybenzyl)-5-(phenylamino)-1H-pyrazole-4-carbonitrile

A mixture of 2-(methylthio(phenylamino)methylene)malononitrile (2.4 g, 11 mmol), (4-methoxybenzyl)hydrazine hydrochloride (2 g, 11 mmol) and triethylamine (2.3 g, 22 mmol) in ethanol (50 mL) was stirred in a microwave at 130° C. for 2 h. Upon completion, the reaction mixture was poured into sodium bicarbonate (100 mL) and extracted with dichloromethane (300 mL). The organic phase was dried over sodium sulfate and evaporated to dryness under reduced pressure to give the title compound which was suitable for use without further purification (2.8 g, 80%). LC/MS: m/e=320 (M+H)⁺.

### 3-Amino-1-(4-methoxybenzyl)-5-(phenylamino)-1H-pyrazole-4-carboxamide

To a solution of 3-amino-1-(4-methoxybenzyl)-5-(phenylamino)-1H-pyrazole-4-carbonitrile (2.8 g, 8.7 mmol) and potassium carbonate (3.6 g, 26.1 mmol) in DMSO (20 mL) was added hydrogen peroxide (30% in water, 9.8 g, 87 mmol). The resulting exotherm reached about 100° C. Upon completion, the reaction mixture was poured into ice water (150 mL). The precipitate was collected by filtration, and dried in vacuo to give the title compound as a yellow solid (1.9 g, 65%). LC/MS: m/e=338 (M+H)⁺.

### tert-Butyl (2-(4-methoxybenzyl)-4-oxo-3-(phenylamino)-4,5-dihydro-2H-Pyrazolo[3,4-d]pyrimidin-6-yl)methylcarbamate

To a solution of 3-amino-1-(4-methoxybenzyl)-5-(phenylamino)-1H-pyrazole-4-carboxamide (1.4 g, 4.3 mmol) and ethyl 2-(tert-butoxycarbonylamino)acetate (2.5 g, 12.8 mmol) in dioxane (30 mL) was added 60% NaH in mineral oil (1.7 g, 43.0 mmol) portionwise. The mixture was stirred at rt overnight. Ethanol (20 mL) was added and the reaction was heated to reflux for 2 h. Upon completion, the reaction was evaporated in vacuo, diluted with water (200 mL) and extracted with ethyl acetate (100 mL×2). The combined organic layers were dried (Na₂SO₄) and concentrated in vacuo to give the title compound which was suitable for use without further purification (2.0 g, 100%). LC/MS: m/e=477 (M+H)⁺.

### 6-(Aminomethyl)-2-(4-methoxybenzyl)-3-(phenylamino)-2H-pyrazolo[3,4-d]pyrimidin-4(5H)-one trifluoroacetic acid

To a solution of (2-(4-methoxybenzyl)-4-oxo-3-(phenylamino)-4,5-dihydro-2H-pyrazolo[3,4-d]pyrimidin-6-yl)methylcarbamate (2.0 g, 4.3 mmol) in dichloromethane (30 mL) was added trifluoroacetic acid (5 mL). The mixture was heated to 50° C. with stirring for 2 h. Upon completion, the mixture was concentrated in vacuo to give the title compound as the trifluoroacetate salt (3.1 g, 100%). LC/MS: m/e=377 (M+H)⁺.

### 6-((2-Chloroethylamino)methyl)-2-(4-methoxybenzyl)-3-(phenylamino)-2H-pyrazolo[3,4-d]pyrimidin-4(5H)-one

To a solution of 6-(aminomethyl)-2-(4-methoxybenzyl)-3-(phenylamino)-2H-pyrazolo[3,4-d]pyrimidin-4(5H)-one trifluoroacetate (3.1 g, 6 mmol) in methanol (20 mL) was added 50% aqueous 2-chloroacetaldehyde (0.34 g, 2.1 mmol) and NaCNBH₃ (1.1 g, 17 mmol). The mixture was heated to reflux for 4 hours. The reaction mixture was concentrated in vacuo and water (100 mL) was added. The resulting suspension was extracted with dichloromethane (50 mL). The organic layer was dried over sodium sulfate and filtered. The filtrate was concentrated in vacuo to afford crude material, which was purified by silica gel chromatography (eluting with dichloromethane:methanol=50:1) to give the title compound (0.47 g, 25%). LC/MS: m/e=439 (M+H)⁺.

### 1-(4-Methoxybenzyl)-3-(phenylamino)-6,7,8,9-tetrahydropyrazino[1,2-a]pyrazolo[3,4-d]pyrimidin-4(1H)-one

To a solution of compound 6-((2-chloroethylamino)methyl)-2-(4-methoxy benzyl)-3-(phenylamino)-2H-pyrazolo[3,4-d]pyrimidin-4(5H)-one (0.47 g, 1.1 mmol) in dioxane (15 mL) was added cesium carbonate (0.69 g, 2.2 mmol). The mixture was heated to reflux with stirring for 1 hour. Upon completion, the reaction mixture was filtered and the filtrate was concentrated in vacuo to give the title compound (0.26 g, 56%). LC/MS: m/e=403 (M+H)⁺.

### 1-(4-Methoxybenzyl)-8-methyl-3-(phenylamino)-6,7,8,9-tetrahydropyrazino[1,2-a]pyrazolo[3,4-d]pyrimidin-4(1H)-one

To a solution of 1-(4-methoxybenzyl)-3-(phenylamino)-6,7,8,9-tetrahydropyrazino[1,2-a]pyrazolo[3,4-d]pyrimidin-4(1H)-one (100 mg, 0.25 mmol) in dichloromethane (5 mL) was added paraformaldehyde (75 mg, 2.5 mmol) and sodium triacetoxyborohydride (270 mg, 1.25 mmol). The reaction mixture was stirred at rt overnight. Upon completion, the mixture was filtered and the filtrate concentrated in vacuo to give a residue that was purified by prep. TLC (eluting with dichloromethane:methanol=55:1) to give the title compound (40 mg, 39%) as a white solid. HPLC LC/MS: m/e=417 (M+H)⁺.

### Compound I-49

### 8-Methyl-3-(phenylamino)-6,7,8,9-tetrahydropyrazino[1,2-a]pyrazolo[3,4-d]pyrimidin-4(1H)-one

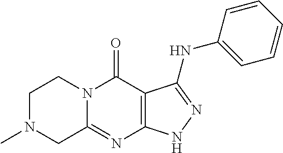 [SMILES: CN1CCn2c(nc3[nH]nc(Nc4ccccc4)c3c2=O)C1]

To a solution 1-(4-methoxybenzyl)-8-methyl-3-(phenylamino)-6,7,8,9-tetrahydropyrazino[1,2-a]pyrazolo[3,4-d]pyrimidin-4(1H)-one (40 mg, 0.1 mmol) in trifluoroacetic acid (0.5 mL) was added conc. sulfuric acid (cat) in a microwave tube. The mixture was heated to 120° C. with stirring for 20 mins. Upon completion, the mixture was concentrated in vacuo, neutralized with 25% aqueous ammonia (0.5 mL) and extracted with dichloromethane (20 mL×2). The combined organic layers were dried and concentrated under reduced pressure to give a residue that was purified by prep. HPLC to give the title compound (10 mg, 34%) as a white solid. ¹H NMR (400 MHz, d⁶-DMSO): δ 7.90 (s, 1H), 7.65 (d, J=7.6 Hz, 2H), 7.24 (t, J=8.4 Hz, 2H), 6.84 (t, J=7.2 Hz, 1H), 3.83 (t, J=5.2 Hz, 2H), 3.57 (s, 2H), 2.79 (t, J=5.6 Hz, 2H), 2.34 (s, 3H), LC/MS: m/e=297 (M+H)⁺.

### Compound I-50

### 8-Benzyl-3-phenylamino)-6,7,8,9-tetrahydropyrazino[1,2-a]pyrazolo[3,4-d]pyrimidin-4(1H)-one

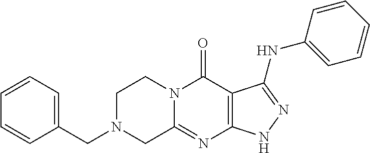 [SMILES: O=c1c2c(Nc3ccccc3)n[nH]c2nc2n1CCN(Cc1ccccc1)C2]

The title compound was prepared in a manner similar to I-49, substituting benzaldehyde for paraformaldehyde. ¹H NMR (400 MHz, d⁶-DMSO): δ 12.66 (s, 1H), 7.89 (s, 1H), 7.65 (d, J=8.0 Hz, 2H), 7.37 (m, 4H), 7.30 (m, 1H), 7.24 (t, J=7.6 Hz, 2H), 6.84 (t, J=7.6 Hz, 1H), 3.84 (t, J=5.6 Hz, 2H), 3.69 (s, 2H), 3.63 (s, 2H), 2.87 (t, J=5.6 Hz, 2H), LC/MS: m/e=373 (M+H)⁺.

### Example 5

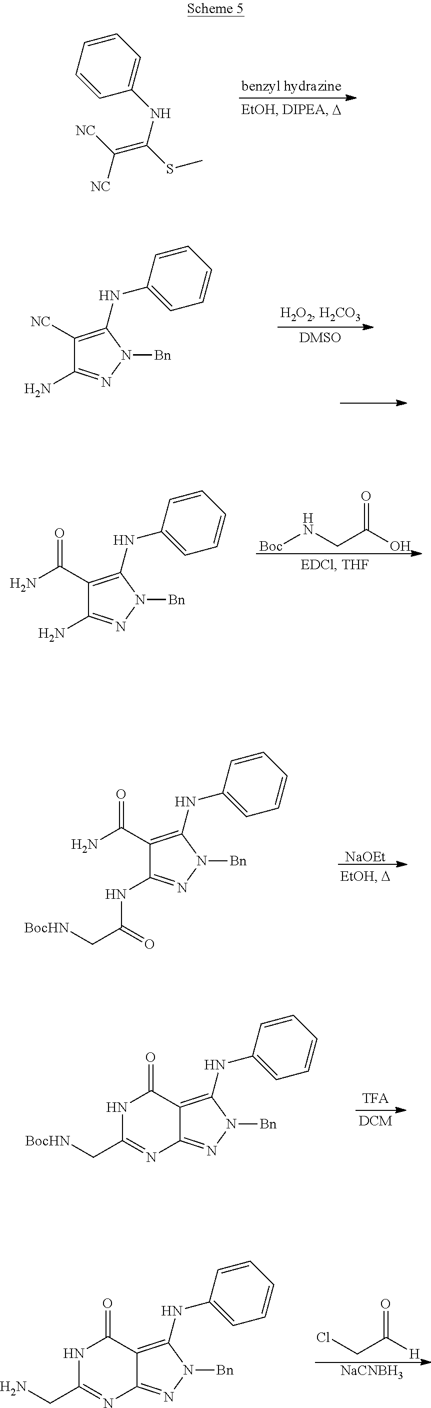

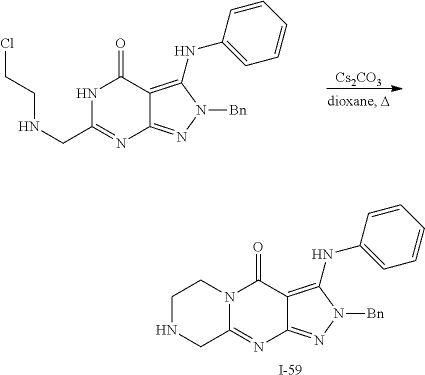

### Compound I-59

### 2-Benzyl-3-(phenylamino)-6,7,8,9-tetrahydropyrazino[1,2-a]pyrazolo[3,4-d]pyrimidin-4(2H)-one

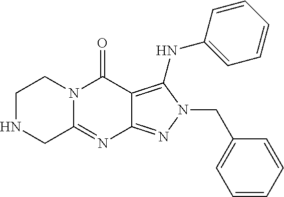 [SMILES: O=c1c2c(Nc3ccccc3)n(Cc3ccccc3)nc2nc2n1CCNC2]

To a solution of 2-benzyl-6-((2-chloroethylamino)methyl)-3-(phenylamino)-2H-pyrazolo[3,4-d]pyrimidin-4(5H)-one (I-201.6) (45 mg, 0.069 mmol) in dioxane (5 mL) was added cesium carbonate (45 mg, 0.14 mmol). The mixture was stirred at 100° C. for 1 h. The reaction mixture was evaporated, suspended in saturated brine and extracted with dichloromethane (10 mL×5). The combined organic layers were dried with sodium sulfate and evaporated in vacuo. The residue was purified via reverse phase HPLC to obtain the title compound (12 mg, 45%). ¹H NMR (400 MHz, CDCl₃): δ 7.28-7.23 (m, 5H), 7.11 (m, 2H), 7.03 (t, J=7.6 Hz, 1H), 6.80 (d, J=7.6 Hz, 2H), 6.49 (s, 1H), 5.16 (s, 2H), 4.07 (s, 2H), 3.86 (t, J=5.6 Hz, 2H), 3.23 (t, J=7.2 Hz, 2H), 1.91 (bs, 1H), LC/MS: m/e=373 (M+H)⁺.

### 3-Amino-1-benzyl-5-(phenylamino)-1H-pyrazole-4-carbonitrile

2-(Methylthio(phenylamino)methylene)malononitrile (prepared as described in the synthesis of compound I-16) (8.61 g, 40.0 mmol) was suspended in ethanol (200 mL). Benzyl hydrazine dichloride (11.72 g, 60.0 mmol) and diisopropylethylamine (10.32 g, 80.0 mmol) was added, and the reaction was stirred overnight at reflux. The resulting mixture was concentrated in vacuo to give the crude product. Purification by silica gel chromatography (eluting with petroleum ether:ethyl acetate=1:1) gave the title compound as a white solid (1.79 g, 15%), LC/MS: m/e=290 (M+H)⁺.

### 3-Amino-1-benzyl-5-(phenylamino)-1H-pyrazole-4-carboxamide

To a solution of 3-amino-1-benzyl-5-(phenylamino)-1H-pyrazole-4-carbonitrile (1.1 g, 3.8 mmol) in dimethylsulfoxide (6 mL) was added potassium carbonate (1.58 g, 11.42 mmol). Hydrogen peroxide (30%, 43.1 g, 38 mmol) was added dropwise with stirring at room temperature. The reaction produced an exotherm, then returned to room temperature. A residue precipitated that was collected and dried to afford the title compound as a light yellow solid (765 mg, 62%). LC/MS: m/e=308 (M+H)⁺.

### tert-Butyl 2-(1-benzyl-4-carbamoyl-5-(phenylamino)-1H-pyrazol-3-ylamino)-2-oxoethylcarbamate

3-Amino-1-benzyl-5-(phenylamino)-1H-pyrazole-4-carboxamide (83 mg, 0.27 mmol), EDCI (77 mg, 0.40 mmol) and 2-(tert-butoxycarbonylamino)acetic acid (70 mg, 0.40 mmol) were suspended in a mixture of tetrahydrofuran:dichloromethane (2:1, 3 mL). The mixture was stirred at room temperature for 6 hours. The reaction mixture was evaporated in vacuo. The residue was washed with saturated brine and extracted with dichloromethane (10 mL×3). The combined organic layers were dried with sodium sulfate then evaporated in vacuo to give the title compound as a white solid (125 mg, 68%). LC/MS: m/e=465 (M+H)⁺.

### tert-Butyl (2-benzyl-4-oxo-3-(phenylamino)-4,5-dihydro-2H-pyrazolo[3,4-d]pyrimidin-6-yl)methylcarbamate

A solution of sodium ethoxide (92 mg, 1.35 mmol) and tert-butyl-2-(1-benzyl-4-carbamoyl-5-(phenylamino)-1H-pyrazol-3-ylamino)-2-oxoethylcarbamate (125 mg, 0.27 mmol) in ethanol (8 mL) was stirred at reflux for 6 h. The reaction mixture was diluted with brine and extracted with ethyl acetate (10 mL×5). The combined organic layers were dried with sodium sulfate and evaporated to give the title compound as a white solid (84 mg, 97%). LC/MS: m/e=447 (M+H)⁺.

### 6-(Aminomethyl)-2-benzyl-3-(phenylamino)-2H-pyrazolo[3,4-d]pyrimidin-4(5H)-one

To a solution of test-butyl (2-benzyl-4-oxo-3-(phenylamino)-4,5-dihydro-2H-pyrazolo[3,4-d]pyrimidin-6-yl)methylcarbamate (84 mg, 0.1 mmol) in dichloromethane (10 mL) was added trifluoroacetic acid (2 mL). The mixture was stirred at reflux for 30 min. The reaction was evaporated to give the title compound as the trifluoroacetate salt (34.6 mg, 49%). LC/MS: m/e=347 (M+H)⁺.

### 2-Benzyl-6-((2-chloroethylamino)methyl)-3-(phenylamino)-2H-pyrazolo[3,4-d]pyrimidin-4(5H)-one

To a solution of 6-(aminomethyl)-2-benzyl-3-(phenylamino)-2H-pyrazolo[3,4-d]pyrimidin-4(5H)-one (34.6 mg, 0.1 mmol) in methanol (5 mL) was added sodium cyanoborohydride (13 mg, 0.2 mmol). 2-Chloroacetaldehyde (8.0 mg, 0.1 mmol) was added dropwise. Additional sodium cyanoborohydride (13 mg, 0.2 mmol) was added and stirring continued at room temperature for 2 h. The reaction was quenched with water and the mixture was extracted with dichloromethane (10 mL×5). The organic layer were combined and dried with sodium sulfate then evaporated in vacuo. The residue was purified, by prep-TLC to give the title compound (45 mg, 69%). LC/MS: m/e=409 (M+H)⁺.

### Compound I-115

### 8-(4-Chlorobenzoyl)-1-cyclopentyl-6,7,8,9-tetrahydropyrazino[1,2-a]pyrazolo[3,4-d]pyrimidin-4(1H)-one

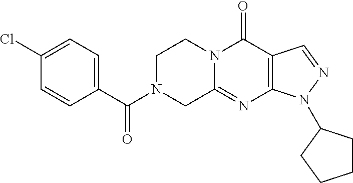 [SMILES: O=C(c1ccc(Cl)cc1)N1CCn2c(nc3c(cnn3C3CCCC3)c2=O)C1]

To a solution of 1-cyclopentyl-6,7,8,9-tetrahydropyrazino[1,2-a]pyrazolo[3,4-d]pyrimidin-4(1H)-one (I-1.6) (40 mg, 0.15 mmol) and triethylamine (30 mg, 0.3 mmol) in dichloromethane (5 mL) was added 4-chlorobenzoyl chloride (52 mg, 0.3 mmol) dropwise at 0° C. The reaction was stirred for 30 mins. Upon completion, the mixture was poured into water (20 mL) and extracted with dichloromethane (20 mL×2). The combined organic layers were dried over sodium sulfate and concentrated in vacuo to give a residue. Purification by prep TLC (eluting with petroleum ether:ethyl acetate 1:1) gave the title compound (45 mg, 73%) as a white solid. ¹H NMR (400 MHz, CDCl₃): δ 8.06 (s, 1H), 7.46 (m, 4H), 5.12 (m, 1H), 4.80 (m, 2H), 4.23 (m, 2H), 3.90 (m, 2H), 2.15-2.00 (m, 4H), 1.98 (m, 2H), 1.70 (m, 2H), LC/MS: m/e=398 (M+H)⁺.

### Compound I-116

### 1-Cyclopentyl-8-(4-methoxybenzoyl)-6,7,8,9-tetrahydropyrazino[1,2-a]pyrazolo[3,4-d]pyrimidin-4(1H)-one

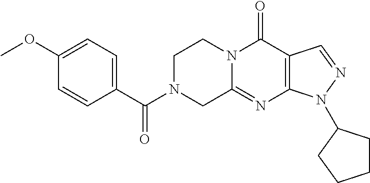 [SMILES: COc1ccc(C(=O)N2CCn3c(nc4c(cnn4C4CCCC4)c3=O)C2)cc1]

The title compound was prepared in a manner similar to I-115, substituting 4-methoxybenzoyl chloride for 4-chlorobenzoyl chloride. ¹H NMR (400 MHz, CDCl₃): δ 8.07 (s, 1H), 7.48 (d, J=8.8 Hz, 2H), 6.96 (d, J=8.4 Hz, 2H), 5.11 (m, 1H), 4.84 (s, 2H), 4.23 (t, J=6.0 Hz, 2H), 3.93 (m, 2H), 3.86 (s, 3H), 2.13-2.02 (m, 4H), 1.96 (m, 2H), 1.70 (m, 2H), LC/MS: m/e=394 (M+H)⁺.

### Example 6

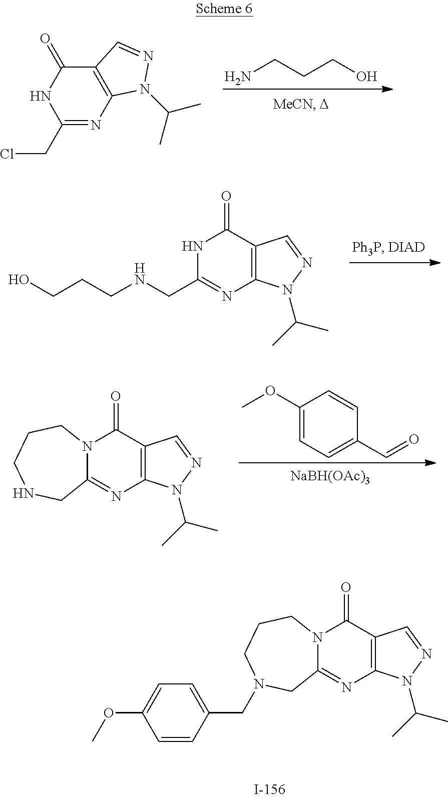

### Compound I-156

### 1-Isopropyl-9-(4-methoxybenzyl)-7,8,9,10-tetrahydro-1H-pyrazolo[3′,4′:4,5]pyrimido[1,2-a][1,4]diazepin-4(6H)-one

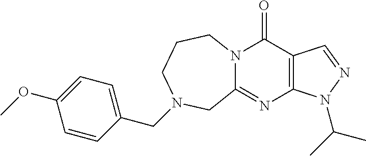 [SMILES: COc1ccc(CN2CCCn3c(nc4c(cnn4C(C)C)c3=O)C2)cc1]

A mixture of 1-isopropyl-7,8,9,10-tetrahydro-1H-pyrazolo[3′,4′:4,5]pyrimido[1,2-a][1,4]diazepin-4(6H)-one (53 mg, 0.21 mmol) (I-204.2), 4-methoxybenzaldehyde (57 mg, 0.42 mmol) and sodium triacetoxyborohydride (134 mg, 0.63 mmol) in dichloromethane (6 mL) was stirred at room temperature overnight. Upon completion, water (6 mL) was added to the mixture, and the aqueous layer was extracted with dichloromethane (3×30 mL). The combined organic layers were washed with brine (30 mL), dried over sodium sulfate and evaporated in vacuo to give the crude product. Purification by Prep-TLC (ethyl acetate/petroleum ether 3/1) gave the title compound (20.46 mg, 26%) as a light yellow solid. ¹H NMR (400 MHz, CDCl₃): δ 8.07 (s, 1H), 7.23 (d, J=8.4 Hz, 2H), 6.87 (d, J=8.8 Hz, 2H), 4.99 (sept, J=6.8 Hz, 1H), 4.42 (bs, 2H), 4.03 (s, 2H), 3.82 (s, 3H), 3.66 (s, 2H), 3.00 (m, 2H), 1.88 (m, 2H), 1.55 (d, J=6.8 Hz, 6H), LC/MS: m/e=368 (M+M)⁺.

### 6-(((3-Hydroxypropyl)amino)methyl)-1-isopropyl-1H-pyrazolo[3,4-d]pyrimidin-4(5H)-one

A mixture of 6-(chloromethyl)-1-isopropyl-1H-pyrazolo[3,4-d]pyrimidin-4(5H)-one (200 mg, 0.88 mmol) and 3-aminopropan-1-ol (133 mg, 1.77 mmol) in acetonitrile (2 mL) was heated overnight at reflux. The mixture was purified by flash chromatography to yield the title compound (150 mg, 64%) as a light yellow solid. LC/MS: m/e=266 (M+H)⁺.

### 1-Isopropyl-7,8,9,10-tetrahydro-1H-pyrazolo[3′,4′:4,5]pyrimido[1,2-a][1,4]diazepin-4(6R)-one

A mixture of 6-(((3-hydroxypropyl)amino)methyl)-1-isopropyl-1H-pyrazolo[3,4-d]pyrimidin-4(5H)-one (150 mg, 0.57 mmol) and triphenylphosphine (296 mg, 1.13 mmol) in dry toluene (6 mL) was stirred in a three-neck flask under argon atmosphere. Diisopropyl azodicarboxylate (228 mg, 1.13 mmol) was added to the mixture, and the reaction was stirred at room temperature overnight. The reaction was evaporated under reduced pressure to give crude material that was purified by flash chromatography to yield the title compound (53 mg, 37.6%) as a light yellow solid. LC/MS: m/e=248 (M+H)⁺.

### Compound I-157

### 1-Isopropyl-8-(4-methoxybenzyl)-7,8,9,10-tetrahydro-1H-pyrazolo[3′,4′:4,5]pyrimido[1,2-d][1,4]diazepin-4(6H)-one

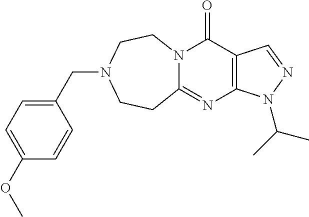 [SMILES: COc1ccc(CN2CCc3nc4c(cnn4C(C)C)c(=O)n3CC2)cc1]

The title compound was prepared in a manner similar to I-156. 6-(2-Chloroethyl)-1-isopropyl-1H-pyrazolo[3,4-d]pyrimidin-4(5H)-one was prepared from 3-chloropropanoyl chloride in a manner similar to 6-(chloromethyl)-1-isopropyl-1H-pyrazolo[3,4-d]pyrimidin-4(5H)-one, and ethanolamine was substituted for 3-propanol-1-amine. ¹H NMR (400 MHz, CDCl₃): δ 8.04 (s, 1H), 7.23 (d, J=8.8 Hz, 2H), 6.88 (dd, J₁=11.2 Hz, J₂=2.8 Hz, 2H), 5.00 (sept, J=6.4 Hz, 1H), 4.46 (m, 2H), 3.82 (s, 3H), 3.56 (s, 2H), 3.18 (m, 2H), 2.76 (m, 2H), 2.70 (m, 2H), 1.53 (d, J=6.8 Hz, 6H), LC/MS: m/e=368 (M+H)⁺.

### Compound I-112

### 1-Cyclopentyl-8-(4-methoxybenzyl)-3-methyl-6,7,8,9-tetrahydropyrazino[1,2-d]pyrazolo[3,4-d]pyrimidin-4(1H)-one

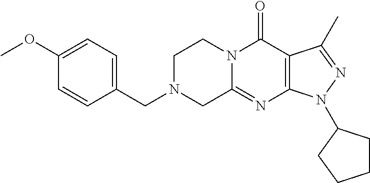 [SMILES: COc1ccc(CN2CCn3c(nc4c(c(C)nn4C4CCCC4)c3=O)C2)cc1]

The title compound was prepared in a manner similar to I-84 substituting 5-amino-1-cyclopentyl-3-methyl-1H-pyrazole-4-carboxamide (synthesized from 2-(ethoxymethlene) malononitrile using the procedure in Haning, H., et al., Bioorg. Med. Chem. Lett. 2005, 15, 3900) for 5-amino-1-cyclopentyl-1H-pyrazole-4-carboxamide. ¹H NMR (400 MHz, CDCl₃): δ 7.26 (m, 2H), 6.89 (m, 2H), 4.98 (quint, J=7.6 Hz, 1H), 3.99 (t, J=5.6 Hz, 2H), 3.82 (s, 3H), 3.66 (s, 2H), 3.62 (s, 2H), 2.86 (t, J=6.0 Hz, 2H), 2.58 (s, 3H), 2.07-1.98 (m, 4H), 1.88 (m, 2H), 1.65 (m, 2H), LC/MS: m/e=394 (M+H)⁺.

### Compound I-111

### 8-(4-Chlorobenzyl)-1-cyclopentyl-3-methyl-6,7,8,9-tetrahydropyrazino[1,2-d]pyrazolo[3,4-d]pyrimidin-4(1H)-one

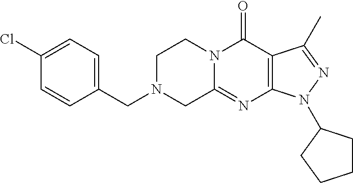 [SMILES: Cc1nn(C2CCCC2)c2nc3n(c(=O)c12)CCN(Cc1ccc(Cl)cc1)C3]

The title compound was prepared in a manner similar to I-112, substituting 4-chlorobenzaldehyde for 4-methoxybenzaldehyde. ¹H NMR (400 MHz, CDCl₃): δ 7.31 (m, 4H), 4.98 (quint, J=8.0 Hz, 1H), 4.00 (t, J=5.6 Hz, 2H), 3.66 (s, 2H), 3.65 (s, 2H), 2.88 (t, J=5.6 Hz, 2H), 2.58 (s, 3H), 2.07-1.98 (m, 4H), 1.88 (m, 2H), 1.65 (m, 2H), LC/MS: m/e=398 (M+H)⁺.

### Example 7

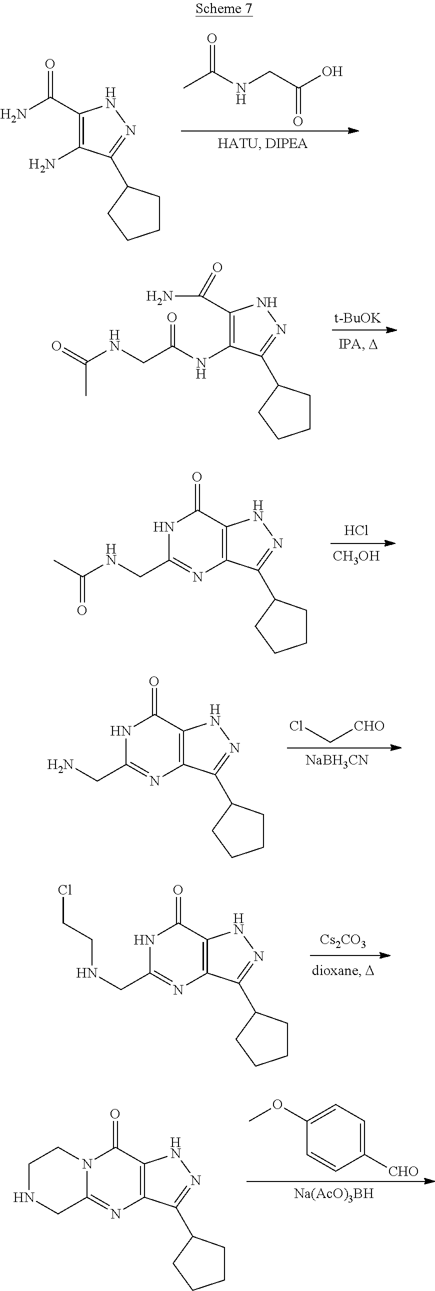

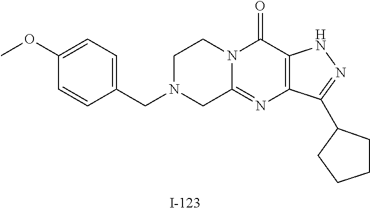 [SMILES: COc1ccc(CN2CCn3c(nc4c(C5CCCC5)n[nH]c4c3=O)C2)cc1]

### Compound I-123

### 3-Cyclopentyl-6-(4-methoxybenzyl)-5,6,7,8-tetrahydropyrazino[1,2-a]pyrazolo[4,3-d]pyrimidin-10(1H)-one

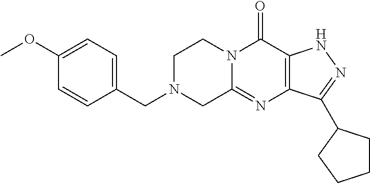 [SMILES: COc1ccc(CN2CCn3c(nc4c(C5CCCC5)n[nH]c4c3=O)C2)cc1]

To a solution of 3-cyclopentyl-5,6,7,8-tetrahydropyrazino[1,2-a]pyrazolo[4,3-d]pyrimidin-10(1H)-one (I-123) (22 mg, 0.085 mmol) in dichloromethane (3 mL) was added 4-methoxybenzaldehyde (12 mg, 0.085 mmol) and acetic acid (8 mg, 0.127 mmol). The mixture was stirred at room temperature for 1 h. Sodium triacetoxyborohydride (72 mg, 0.340 mmol) was added, and the reaction was stirred at room temperature for 2 h. Saturated sodium bicarbonate (3 mL) was added, and the mixture was extracted with dichloromethane (3×6 mL). The combined organic phases were dried over sodium sulfate, and the residue was purified by prep-TLC to give the product (16 mg, 50%) as a yellow solid. ¹H NMR (400 MHz, CDCl₃): δ 11.42 (bs, 1H), 7.27 (d, J=10.4 Hz, 2H), 6.89 (d, J=8.8 Hz, 2H), 4.10 (t, J=5.6 Hz, 2H), 3.82 (s, 3H), 3.74 (s, 2H), 3.64 (s, 2H), 3.41 (quint, J=8.0 Hz, 1H), 2.89 (t, J=5.6 Hz, 2H), 2.11 (m, 2H), 1.95-1.82 (m, 4H), 1.71 (m, 2H), LC/MS: m/e=380 (M+H)⁺.

### 4-(2-Acetamidoacetamido)-3-cyclopentyl-1H-pyrazole-5-carboxamide

A solution of 4-amino-3-cyclopentyl-1H-pyrazole-5-carboxamide (synthesized using the procedure in Haning, H., et al., Bioorg. Med. Chem. Lett. 2005, 15, 3900) (500 mg, 2.58 mmol), 2-acetamidoacetic acid (317 mg, 2.71 mmol), HATU (1.03 g, 2.71 mmol) and diisopropylethyl amine (665 mg, 5.15 mmol) in dichloromethane (12 mL) was stirred at reflux for 2 h. The reaction mixture was concentrated to dryness and was suitable for use without further purification. LC/MS: m/e=294 (M+H)⁺.

### N-((3-Cyclopentyl-7-oxo-6,7-dihydro-1H-pyrazolo[4,3-d]pyrimidin-5-yl)methyl)acetamide

A solution of 4-(2-acetamidoacetamido)-3-cyclopentyl-1H-pyrazole-5-carboxamide and potassium tert-butoxide (881 mg, 7.73 mmol) in isopropanol (10 mL) was stirred at reflux for 16 h. Then reaction was concentrated to dryness, quenched with water (6 mL) and extracted with dichloromethane (3×10 mL). The combined organic phases were dried over sodium sulfate and concentrated to dryness. The residue was purified by flash chromatography (dichloromethane/methanol:100/1-20/1) to give the product (680 mg, 96%) as a yellow solid. LC/MS: m/e=276 (M+H)⁺.

### 5-(Aminomethyl)-3-cyclopentyl-1H-pyrazolo[4,3-d]pyrimidin-7(6H)-one

N-((3-Cyclopentyl-7-oxo-6,7-dihydro-1H-pyrazolo[4,3-d]pyrimidin-5-yl)methyl)acetamide (680 mg, 2.47 mmol) was dissolved in a mixture of concentrated hydrochloric acid (2 mL) and methanol (6 mL) and stirred at reflux for 2 h. The reaction was concentrated to dryness to give the title compound as its hydrochloride salt (600 mg, 90%). LC/MS: m/e=234 (M+H)⁺.

### 5-((2-Chloroethylamino)methyl)-3-cyclopentyl-1H-pyrazolo[4,3-d]pyrimidin-7(6H)-one

To a solution of 5-(aminomethyl)-3-cyclopentyl-1H-pyrazolo[4,3-d]pyrimidin-7(6H)-one hydrochloride (450 mg, 1.67 mmol) in methanol (8 mL) was added chloroacetaldehyde (50% aqueous solution, 261 mg, 1.67 mmol) and sodium cyanoborohydride (843 mg, 13.4 mmol). The mixture was stirred at room temperature for 0.5 h. Saturated sodium bicarbonate (3 mL) was added, and the mixture was extracted with dichloromethane (3×8 mL). The combined organic phases were dried over sodium sulfate, and concentrated to dryness. The residue was purified by prep-TLC (dichloromethane/methanol=10/1) to give the product (120 mg, 24%) as a yellow solid. LC/MS: m/e=296 (M+H)⁺.

### 3-Cyclopentyl-5,6,7,8-tetrahydropyrazino[1,2-a]pyrazolo[4,3-d]pyrimidin-10(1H)-one

To a solution of 5-((2-chloroethylamino)methyl)-3-cyclopentyl-1H-pyrazolo[4,3-d]pyrimidin-7(6H)-one (120 mg, 0.41 mmol) in dioxane (5 mL) was added cesium carbonate (264 mg, 0.82 mmol). The reaction was stirred at reflux for 0.5 h. The mixture was concentrated to dryness, and the residue was purified by prep-TLC (dichloromethane/methanol=10/1) to give 60 mg (77%) of the product (60 mg, 77%) as a yellow solid. LC/MS: m/e=260 (M+H)⁺.

### Compound I-10

### 3-Isopropyl-5,6,7,8-tetrahydropyrazino[1,2-a]pyrazolo[4,3-a]pyrimidin-10(1H)-one

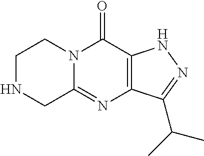 [SMILES: CC(C)c1n[nH]c2c(=O)n3c(nc12)CNCC3]

The title compound was prepared using the procedure for Compound I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide. ¹H NMR (400 MHz, CDCl₃): δ 4.15 (s, 2H), 4.08 (t, J=6.0 Hz, 2H), 3.39 (sept, J=6.8 Hz, 1H), 3.32 (t, J=5.6 Hz, 2H), 1.43 (d, J=7.2 Hz, 6H), LC/MS: m/e=234 (M+H)⁺.

### Compound I-11

### (±)-3-Isopropyl-5-methyl-5,6,7,8-tetrahydropyrazino[1,2-a]pyrazolo[4,3-d]pyrimidin-10(1H)-one

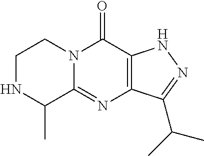 [SMILES: CC(C)c1n[nH]c2c(=O)n3c(nc12)C(C)NCC3]

The title compound was prepared using the procedure for Compound I-123, substituting 4-amino-3-isopropyl-1H-pyrazole-5-carboxamide (synthesized using the procedure in Haning, H., et al., Bioorg. Med. Chem. Lett 2005, 15, 3900) for 4-amino-3-cyclopentyl-1H-pyrazole-5-carboxamide, and substituting 2-acetamidopropanoic acid for 2-acetamidoacetic acid. ¹H NMR (400 MHz, CDCl₃): δ 4.23 (de, J₁=14.0 Hz, J₂=4.4 Hz, 1H), 4.13 (dd, J₁=13.2 Hz, J₂=6.8 Hz, 1H), 4.08-4.01 (m, 1H), 3.45-3.36 (m, 2H), 3.26-3.20 (m, 1H), 1.66 (d, J=6.0 Hz, 3H), 1.45 (d, J=7.2 Hz, 6H), LC/MS: m/e=248 (M+H)⁺.

### Compound I-112

### (±)-5-Benzyl-3-isopropyl-5,6,7,8-tetrahydropyrazino[1,2-a]pyrazolo[4,3-d]pyrimidin-10(1H)-one

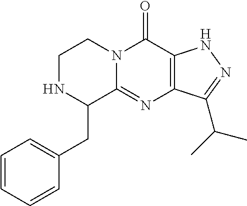 [SMILES: CC(C)c1n[nH]c2c(=O)n3c(nc12)C(Cc1ccccc1)NCC3]

The title compound was prepared using the procedure for Compound I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, and substituting 2-acetamido-3-phenylpropanoic acid for 2-acetamidoacetic acid. ¹H NMR (400 MHz, CDCl₃): δ 7.35-7.24 (m, 5H), 430-4.23 (m, 2H), 3.91-3.80 (m, 2H), 3.44 (sept, J=6.8 Hz, 1H), 3.40-3.34 (m, 1H), 3.11-3.03 (m, 2H), 1.49 (dd, J₁=6.8 Hz, J₂=2.0 Hz, 6H), LC/MS: m/e=324 (M+H)⁺.

### Compound I-13

### 3-Isopropyl-1-methyl-5,6,7,8-tetrahydropyrazino[1,2-a]pyrazolo[4,3-d]pyrimidin-10(1H)-one

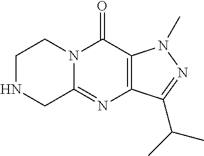 [SMILES: CC(C)c1nn(C)c2c(=O)n3c(nc12)CNCC3]

The title compound was prepared using the procedure for Compound I-123, substituting 4-amino-3-isopropyl-1-methyl-1H-pyrazole-5-carboxamide (synthesized using the procedure in Hailing, H., et al., Bioorg. Med. Chem. Lett. 2005, 15, 3900) for 4-amino-3-cyclopentyl-1H-pyrazole-5-carboxamide. ¹H NMR (400 MHz, CDCl₃): δ 4.24 (s, 3H), 4.09 (s, 2H), 4.00 (t, J=6.0 Hz, 2H), 3.35-3.28 (m, 3H), 1.39 (d, J=6.8 Hz, 6H), LC/MS: m/e=248 (M+H)⁺.

### Compound I-14

### (±)-3-Isopropyl-1,5-dimethyl-5,6,7,8-tetrahydropyrazino[1,2-a]pyrazolo[4,3-d]pyrimidin-10(1H)-one

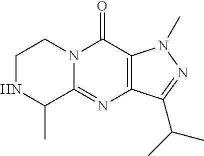 [SMILES: CC(C)c1nn(C)c2c(=O)n3c(nc12)C(C)NCC3]

The title compound was prepared using the procedure for Compound I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, and substituting 2-acetamidopropanoic acid for 2-acetamidoacetic acid. ¹H NMR (400 MHz, CDCl₃): δ 4.23 (s, 3H), 4.14 (m, 1H), 4.08 (dd, J₁=13.2 Hz, J₂=6.4 Hz, 1H), 3.98-3.91 (m, 1H), 3.43-3.36 (m, 1H), 3.33 (sept, J=6.8 Hz, 1H), 3.23-3.16 (m, 1H), 1.72 (bs, 1H) 1.63 (d, J=6.4 Hz, 3H), 1.41 (d, J=6.8 Hz, 6H), LC/MS: m/e=262 (M+H)⁺.

### Compound I-15

### (±)-5-Benzyl-3-isopropyl-1-methyl-5,6,7,8-tetrahydropyrazino[1,2-a]pyrazolo[4,3-d]pyrimidin-10(1H)-one

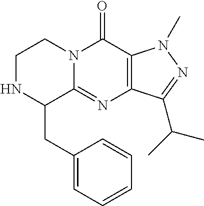 [SMILES: CC(C)c1nn(C)c2c(=O)n3c(nc12)C(Cc1ccccc1)NCC3]

The title compound was prepared using the procedure for Compound I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, and substituting 2-acetamido-3-phenylpropanoic acid for 2-acetamidoacetic acid. ¹H NMR (400 MHz, CDCl₃): δ 7.34-7.23 (m, 5H), 4.24 (s, 3H), 4.20-4.13 (m, 2H), 3.81-3.74 (m, 2H), 3.41-3.31 (m, 2H), 3.07-2.98 (m, 2H), 1.85 (bs, 1H), 1.43 (dd, J₁=6.8 Hz, J₂=1.6 Hz, 6H), LC/MS: m/e=338 (M+H)⁺.

### Compound I-38

### (±)-3-Isopropyl-5,6-dimethyl-5,6,7,8-tetrahydropyrazino[1,2-a]pyrazolo[4,3-d]pyrimidin-10(1H)-one

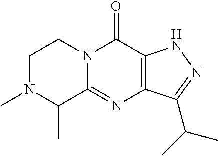 [SMILES: CC(C)c1n[nH]c2c(=O)n3c(nc12)C(C)N(C)CC3]

The title compound was prepared using the procedure for I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamidopropanoic acid for 2-acetamidoacetic acid, and substituting paraformaldehyde for 4-methoxybenzaldehyde. ¹H NMR (400 MHz, CDCl₃): δ 4.35 (dt, J₁=14.0 Hz, J₂=4.0 Hz, 1H), 3.97-3.90 (m, 1H), 3.58 (dd, J₁=13.2 Hz, J₂=6.4 Hz, 1H), 3.40 (sept, J=7.2 Hz, 1H), 3.21 (dt, J₁=12.8 Hz, J₂=4.0 Hz, 1H), 2.74 (m, 1H), 2.51 (s, 3H), 1.64 (d, J=6.4 Hz, 3H), 1.45 (dd, J₁=6.8 Hz, J₂=1.2 Hz, 6H), LC/MS: m/e=262 (M+H)⁺.

### Compound I-39

### (±)-5-Benzyl-3-isopropyl-6-methyl-5,6,7,8-tetrahydropyrazino[1,2-a]pyrazolo[4,3-d]pyrimidin-10(1H)-one

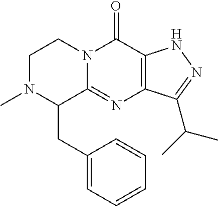 [SMILES: CC(C)c1n[nH]c2c(=O)n3c(nc12)C(Cc1ccccc1)N(C)CC3]

The title compound was prepared using the procedure for I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamido-3-phenylpropanoic acid for 2-acetamidoacetic acid, and substituting paraformaldehyde for 4-methoxybenzaldehyde. ¹H NMR (400 MHz, CDCl₃): δ 7.14 (m, 3H), 7.06 (m, 2H), 4.33 (dt, J₁=13.4 Hz, J₂=3.6 Hz, 1H), 3.93 (t, J=4.8 Hz, 1H), 3.45-3.36 (m, 3H), 3.29 (dd, J₁=14.0 Hz, J₂=4.0 Hz, 1H), 3.17 (dt, J₁=12.0 Hz, J₂=4.4 Hz, 1H), 2.67 (m, 1H), 2.56 (s, 3H), 1.47 (dd, J₁=6.8 Hz, J₂=2.8 Hz, 6H), LC/MS: m/e=338 (M+H)⁺.

### Compound I-40

### 3-Isopropyl-1,6-dimethyl-5,6,7,8-tetrahydropyrazino[1,2-a]pyrazolo[4,3-d]pyrimidin-10(1H)-one

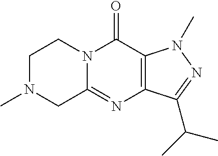 [SMILES: CC(C)c1nn(C)c2c(=O)n3c(nc12)CN(C)CC3]

The title compound was prepared using the procedure for I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, and substituting paraformaldehyde for 4-methoxybenzaldehyde. ¹H NMR (400 MHz, CDCl₃): δ 4.24 (s, 3H), 4.05 (t, J=6.4 Hz, 2H), 3.67 (s, 2H), 3.33 (sept, J=6.8 Hz, 1H), 2.83 (t, J=5.6 Hz, 2H), 2.46 (s, 3H), 1.39 (d, J=7.2 Hz, 6H), LC/MS: m/e=262 (M+H)⁺.

### Compound I-41

### (±)-3-Isopropyl-1,5,6-trimethyl-5,6,7,8-tetrahydropyrazino[1,2-a]pyrazolo[4,3-d]pyrimidin-10(1H)-one

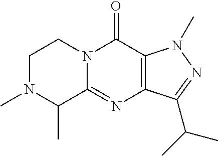 [SMILES: CC(C)c1nn(C)c2c(=O)n3c(nc12)C(C)N(C)CC3]

The title compound was prepared using the procedure for I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamidopropanoic acid for 2-acetamidoacetic acid, and substituting paraformaldehyde for 4-methoxybenzaldehyde. ¹H NMR (400 MHz, CDCl₃): δ 4.27 (m, 1H), 4.23 (s, 3H), 3.85 (m, 1H), 3.52 (m, 1H), 3.32 (sept, J=6.4 Hz, 1H), 3.18 (m, 1H), 2.70 (m, 1H), 2.50 (s, 3H), 1.62-1.56 (m, 3H), 1.41 (d, J=7.2 Hz, 6H), LC/MS: m/e=276 (M+H)⁺.

### Compound I-42

### (±)-5-Benzyl-3-isopropyl-1,6-dimethyl-5,6,7,8-tetrahydropyrazino[1,2-a]pyrazolo[4,3-d]pyrimidin-10(1H)-one

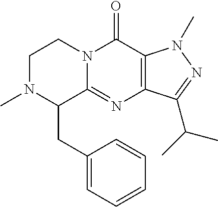 [SMILES: CC(C)c1nn(C)c2c(=O)n3c(nc12)C(Cc1ccccc1)N(C)CC3]

The title compound was prepared using the procedure for I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamido-3-phenylpropanoic acid for 2-acetamidoacetic acid, and substituting paraformaldehyde for 4-methoxybenzaldehyde. ¹H NMR (400 MHz, CDCl₃): δ 7.14 (m, 3H), 7.06 (m, 2H), 4.24 (m, 1H), 4.21 (s, 3H), 3.87 (t, J=4.8 Hz, 1H), 3.43-3.24 (m, 4H), 3.14 (dt, J₁=12.4 Hz, J₂=4.0 Hz, 1H), 2.73 (m, 1H), 2.53 (s, 3H), 1.43 (dd, J₁=6.0 Hz, J₂=3.6 Hz, 6H), LC/MS: m/e=352 (M+H)⁺.

### Compound I-43

### 6-Benzyl-3-isopropyl-5,6,7,8-tetrahydropyrazino[1,2-a]pyrazolo[4,3-d]pyrimidin-10(1H)-one

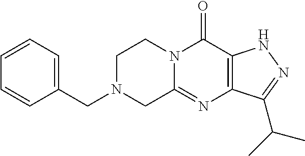 [SMILES: CC(C)c1n[nH]c2c(=O)n3c(nc12)CN(Cc1ccccc1)CC3]

The title compound was prepared using the procedure for I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, and substituting benzaldehyde for 4-methoxybenzaldehyde. ¹H NMR (400 MHz, CDCl₃): δ 7.37-7.30 (m, 5H), 4.09 (t, J=5.6 Hz, 2H), 3.76 (s, 2H), 3.70 (s, 2H), 3.35 (sept, J=6.8 Hz, 1H), 2.91 (t, J=6.0 Hz, 2H), 1.40 (d, J=6.8 Hz, 6H), LC/MS: m/e=324 (M+H)⁺.

### Compound I-44

### (±)-6-Benzyl-3-isopropyl-5-methyl-5,6,7,8-tetrahydropyrazino[1,2-a]pyrazolo[4,3-d]pyrimidin-10(1H)-one

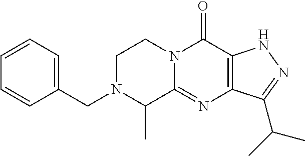 [SMILES: CC(C)c1n[nH]c2c(=O)n3c(nc12)C(C)N(Cc1ccccc1)CC3]

The title compound was prepared using the procedure for I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamidopropanoic acid for 2-acetamidoacetic acid, and substituting benzaldehyde for 4-methoxybenzaldehyde. ¹H NMR (400 MHz, CDCl₃): δ 7.40-7.26 (m, 5H), 4.18-4.12 (m, 1H), 4.02 (m, 2H), 3.96 (d, J=13.6 Hz, 1H), 3.63 (d, J=13.2 Hz, 1H), 3.40 (sept, J=6.8 Hz, 1H), 3.18 (m, 1H), 2.79-2.73 (m, 1H), 1.63 (d, J=6.8 Hz, 3H), 1.45 (d, J=7.2 Hz, 6H), LC/MS: m/e=338 (M+H)⁺.

### Compound I-45

### (±)-5,6-dibenzyl-3-isopropyl-5,6,7,8-tetrahydropyrazino[1,2-a]pyrazolo[4,3-d]pyrimidin-10(1H)-one

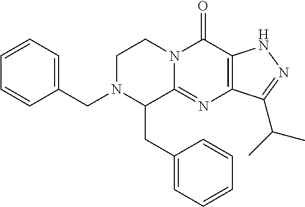 [SMILES: CC(C)c1n[nH]c2c(=O)n3c(nc12)C(Cc1ccccc1)N(Cc1ccccc1)CC3]

The title compound was prepared using the procedure for I-123, substituting 4-amino-3-isopropyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamido-3-phenylpropanoic for 2-acetamidoacetic acid, and substituting benzaldehyde for 4-methoxybenzaldehyde. ¹H NMR (400 MHz, CDCl₃): δ 7.26-7.09 (m, 8H), 7.02 (m, 2H), 4.19-4.13 (m, 2H), 3.95 (d, J=13.6 Hz, 1H), 3.58 (d, J=13.2 Hz, 1H), 3.43-3.10 (m, 5H), 2.60-2.54 (m, 1H), 1.39 (dd, J₁=7.2 Hz, J₂=4.8 Hz, 6H), LC/MS: m/e=414 (M+H)⁺.

### Compound I-46

### 6-Benzyl-3-isopropyl-1-methyl-5,6,7,8-tetrahydropyrazino[1,2-a]pyrazolo[4,3-d]pyrimidin-10(1H)-one

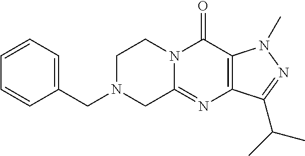 [SMILES: CC(C)c1nn(C)c2c(=O)n3c(nc12)CN(Cc1ccccc1)CC3]

The title compound was prepared using the procedure for I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, and substituting benzaldehyde for 4-methoxybenzaldehyde. ¹H NMR (400 MHz, CDCl₃): δ 7.36-7.27 (m, 5H), 4.23 (s, 3H), 4.03 (t, J=6.0 Hz, 2H), 3.72 (s, 2H), 3.68 (s, 2H), 3.30 (sept, J=7.2 Hz, 1H), 2.88 (t, J=5.6 Hz, 2H), 1.37 (d, J=7.2 Hz, 6H), LC/MS: m/e 338 (M+H)⁺.

### Compound I-47

### (±)-6-Benzyl-3-isopropyl-1,5-dimethyl-5,6,7,8-tetrahydropyrazino[1,2-a]pyrazolo[4,3-d]pyrimidin-10(1H)-one

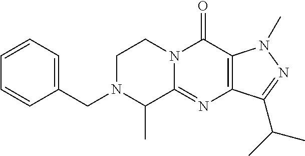 [SMILES: CC(C)c1nn(C)c2c(=O)n3c(nc12)C(C)N(Cc1ccccc1)CC3]

The title compound was prepared using the procedure for I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamidopropanoic acid for 2-acetamidoacetic acid, and substituting benzaldehyde for 4-methoxybenzaldehyde. ¹H NMR (400 MHz, CDCl₃): δ 7.35 (m, 3H), 7.28 (m, 2H), 4.23 (s, 3H), 4.06 (m, 1H), 3.95-3.87 (m, 3H), 3.60 (d, J=13.6 Hz, 1H), 3.34 (sept, J=6.8 Hz, 1H), 3.18 (m, 1H), 2.72 (m, 1H), 1.61 (d, J=7.2 Hz, 3H), 1.41 (d, J=7.2 Hz, 6H), LC/MS: m/e=352 (M+H)⁺.

### Compound I-48

### (±)-5,6-Dibenzyl-3-isopropyl-1-methyl-5,6,7,8-tetrahydropyrazino[1,2-a]pyrazolo[4,3-d]pyrimidin-10(1H)-one

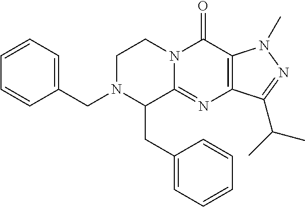 [SMILES: CC(C)c1nn(C)c2c(=O)n3c(nc12)C(Cc1ccccc1)N(Cc1ccccc1)CC3]

The title compound was prepared using the procedure for I-123, substituting 4-amino-3-isopropyl-1-methyl-1H-pyrazole-5-carboxamide (synthesized using the procedure in Haning, H., et al., Bioorg. Med. Chem. Lett. 2005, 15, 3900) for 4-amino-3-cyclopentyl-1H-pyrazole-5-carboxamide, substituting 2-acetamido-3-phenylpropanoic for 2-acetamidoacetic acid, and substituting benzaldehyde for 4-methoxybenzaldehyde. ¹H NMR (400 MHz, CDCl₃): δ 7.35-7.26 (m, 3H), 7.24-7.17 (m, 5H), 7.09 (m, 2H), 4.22 (s, 3H), 4.20-4.09 (m, 2H), 4.01 (d, J=13.6 Hz, 1H), 3.62 (d, J=13.6 Hz, 1H), 3.41-3.24 (m, 4H), 3.21-3.15 (m, 1H), 2.64-2.57 (m, 1H), 1.42 (dd, J₁=7.2 Hz, J₂=3.2 Hz, 6H), LC/MS: m/e=428 (M+H)⁺.

### Compound I-124

### 6-(4-Chlorobenzyl)-3-cyclopentyl-5,6,7,8-tetrahydropyrazino[1,2-a]pyrazolo[4,3-d]pyrimidin-10(1H)-one

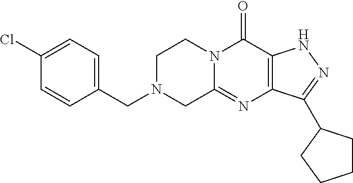 [SMILES: O=c1c2[nH]nc(C3CCCC3)c2nc2n1CCN(Cc1ccc(Cl)cc1)C2]

The title compound was prepared using the procedure for I-123, substituting 4-chlorobenzaldehyde for 4-methoxybenzaldehyde. ¹H NMR (400 MHz, CDCl₃): δ 7.35-7.27 (m, 4H), 4.12 (t, J=5.6 Hz, 2H), 3.74 (s, 2H), 3.67 (s, 2H), 3.41 (quint, J=7.6 Hz, 1H), 2.91 (t, J=5.6 Hz, 2H), 2.11 (m, 2H), 1.95-1.84 (m, 4H), 1.70 (m, 2H), LC/MS: m/e=384 (M+H)⁺.

### Example 8

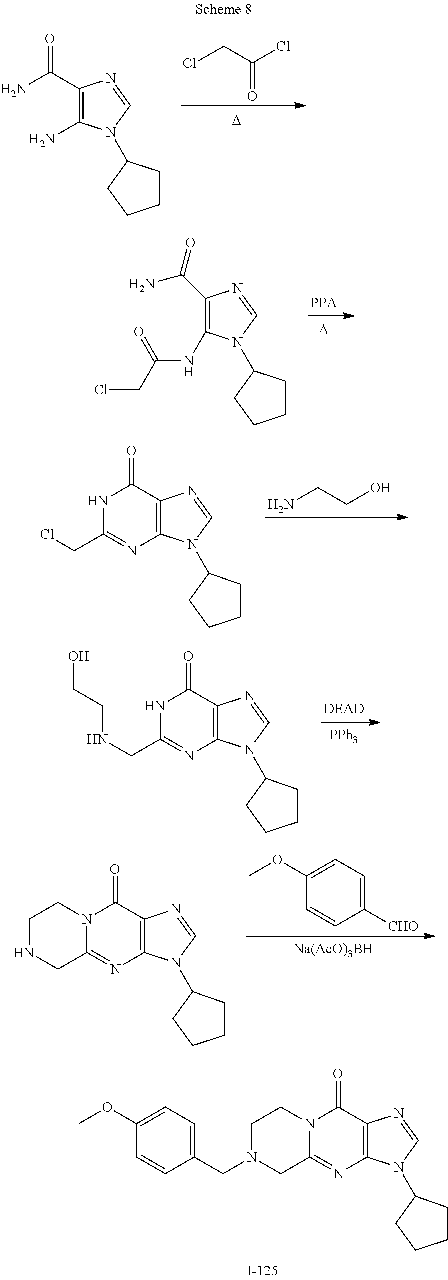

### Compound I-125

### 3-Cyclopentyl-6-(4-methoxybenzyl)-5,6,7,8-tetrahydropyrazino[1,2-a]purin-10(3H)-one

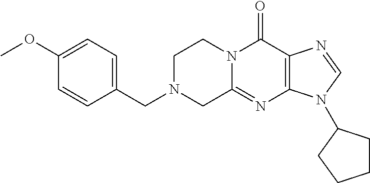 [SMILES: COc1ccc(CN2CCn3c(nc4c(ncn4C4CCCC4)c3=O)C2)cc1]

To a solution of 3-cyclopentyl-5,6,7,8-tetrahydropyrazino[1,2-f]purin-10(3H)-one (I-227.4) (18 mg, 0.069 mmol) in dichloromethane (2 mL) was added 4-methoxybenzaldehyde (11 mg, 0.083 mmol) and acetic acid (8 mg, 0.138 mmol). The mixture was stirred at room temperature for 1 h. Sodium triacetoxyborohydride (73 mg, 0.347 mmol) was added. The resulting mixture was stirred at room temperature for 2 h. Saturated sodium bicarbonate (3 mL) was added, and the mixture was extracted with dichloromethane (3×6 mL). The combined organic phases were dried over sodium sulfate and concentrated to dryness. The residue was purified by prep-HPLC to give the product (11 mg, 42%) as a yellow solid. ¹H NMR (400 MHz, CDCl₃): δ 7.74 (s, 1H), 7.28 (d, J=15.6 Hz, 2H), 6.90 (m, 2H), 4.77 (quint, J=6.8 Hz, 1H), 4.10 (t, J=6.0 Hz, 2H), 3.82 (s, 3H), 3.71 (s, 2H), 3.64 (s, 2H), 2.88 (t, J=6.4 Hz, 2H), 2.23 (m, 2H), 1.95-1.73 (m, 6H), LC/MS: m/e=380 (M+H)⁺.

### 5-(2-Chloroacetamido)-1-cyclopentyl-1H-imidazole-4-carboxamide

A mixture of 5-amino-1-cyclopentyl-1H-imidazole-4-carboxamide (synthesized using the procedure in Haning, H., et al., Bioorg. Med. Chem. Lett. 2005, 15, 3900) (800 mg, 4.1.2 mmol) and 2-chloroacetyl chloride (559 mg, 4.95 mmol) was stirred at 11.0° C. for 1 h. The mixture was purified by flash column chromatography (dichloromethane/methanol=100/1-20/1) to give the product as a yellow solid (500 mg, 45%): LC/MS: m/e=271 (M+H)⁺.

### 2-(Chloromethyl)-9-cyclopentyl-1H-purin-6(9H)-one

A mixture of 5-(2-chloroacetamido)-1-cyclopentyl-1H-imidazole-4-carboxamide (400 mg, 1.48 mmol) and polyphosphoric acid (3 mL) was stirred at 150° C. for 20 min. After cooling to room temperature, water (5 mL) was added. The pH of the aqueous layer was adjusted to 8 with sodium bicarbonate. The mixture was extracted with dichloromethane (3×15 mL). The combined organic phases were dried over sodium sulfate, and concentrated to give the product (200 mg, 54%) as a yellow solid. LC/MS: m/e=253 (M+H)⁺.

### 9-Cyclopentyl-2-((2-hydroxyethylamino)methyl)-1H-purin-6(9H)-one

A mixture of 2-(chloromethyl)-9-cyclopentyl-1H-purin-6(9H)-one (200 mg, 0.79 mmol) and 2-aminoethanol (3 mL) was stirred at room temperature for 10 min. The mixture was purified by reverse phase chromatography to give the product (120 mg, 54%) as a yellow solid. LC/MS: m/e=278 (M+H)⁺.

### 3-Cyclopentyl-5,6,7,8-tetrahydropyrazino[1,2-f]purin-10(3H)-one

A solution of 9-cyclopentyl-2-((2-hydroxyethylamino)methyl)-1H-purin-6(9H)-one (30 mg, 0.11 mmol) in toluene (1.5 mL) was added triphenylphosphine (43 mg, 0.16 mmol) and diethyl azodicarboxylate (28 mg, 0.16 mmol). The mixture was stirred at room temperature for 2 h. The reaction mixture was concentrated to dryness. The residue was redissolved in ethyl acetate (5 mL) and extracted with 1N hydrochloric acid (2×6 mL). The pH of the aqueous layer was adjusted to 8 with sodium bicarbonate aqueous solution. The aqueous layer was extracted with dichloromethane (3×6 mL). The combined organic phases were dried over sodium sulfate and concentrated to give the product (18 mg, 64%) as a yellow solid. LC/MS: m/e=260 (M+H)⁺.

### Compound I-126

### 6-(4-Chlorobenzyl)-3-cyclopentyl-5,6,7,8-tetrahydropyrazino[1,2-a]purin-10(3H)-one

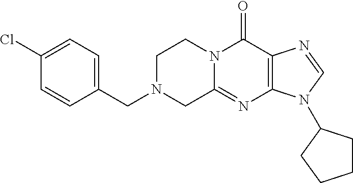 [SMILES: O=c1c2ncn(C3CCCC3)c2nc2n1CCN(Cc1ccc(Cl)cc1)C2]

The title compound was prepared in a manner similar to I-125, substituting 4-chlorobenzaldehyde for 4-methoxybenzaldehyde. ¹H NMR (400 MHz, CDCl₃): δ 7.74 (s, 1H), 7.35-7.30 (m, 4H), 4.77 (quint, J=6.8 Hz, 1H), 4.11 (t, J=6.0 Hz, 2H), 3.70 (s, 2H), 3.67 (s, 2H), 2.90 (t, J=5.6 Hz, 2H), 2.21 (m, 2H), 1.94-1.82 (m, 4H), 1.75 (m, 2H), LC/MS: m/e=384 (M+H)⁺.

### Example 9

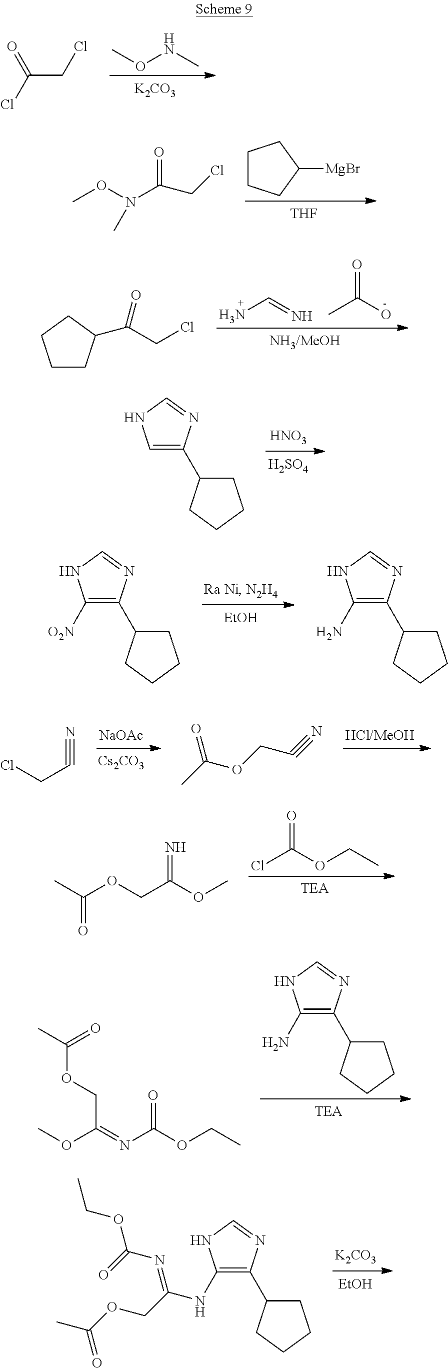

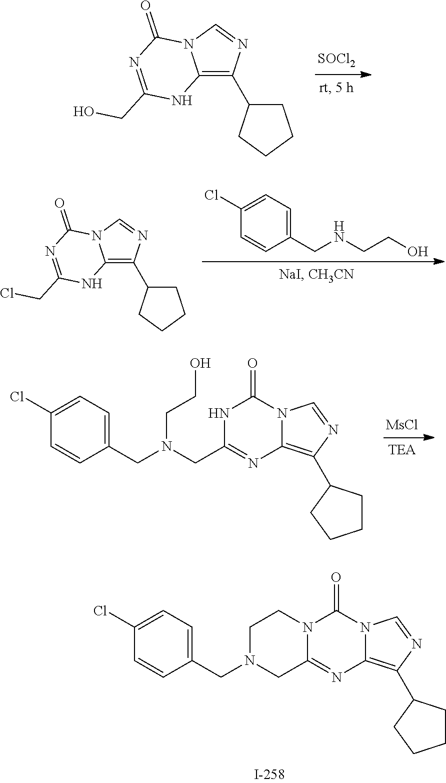

### Compound I-258

### 6-(4-Chlorobenzyl)-3-cyclopentyl-7,8-dihydro-5H-imidazo[1,5-a]pyrazino[2,1-d][1,3,5]triazin-10(6H)-one

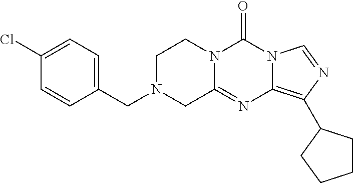 [SMILES: O=c1n2c(nc3c(C4CCCC4)ncn13)CN(Cc1ccc(Cl)cc1)CC2]

To a solution of 2-(((4-chlorobenzyl)(2-hydroxyethyl)amino)methyl)-8-cyclopentylimidazo[1,5-a][1,3,5]triazin-4(3H)-one (I-229.12) (12 mg, 0.03 mmol) in dichloroethane (2 mL) was added methanesulfonyl chloride (5 mg, 0.04 mmol). After stirring for 20 minutes, triethylamine (9 mg, 0.09 mmol) was added and the mixture was stirred at room temperature for 5 h. The mixture was concentrated, the residue was suspended in water (5 mL), and extracted with dichloromethane (3×15 mL). The combined organic phases were dried over sodium sulfate, concentrated and the residue purified by HPLC to give the product (7 mg, 61%) as a white solid. ¹H NMR (400 MHz, d⁴-MeOH): δ 8.27 (s, 1H), 7.40 (dd, J₁=13.6 Hz, 8.8 Hz, 4H), 3.99 (t, J=5.6 Hz, 2H), 3.73 (s, 2H), 3.64 (s, 2H), 3.33 (m, 1H), 2.95 (t, J=5.6 Hz, 2H), 1.98 (m, 2H), 1.85 (m, 4H), 1.72 (m, 2H), LC/MS: m/e=384 (M+H)⁺.

### 2-Chloro-N-methoxy-N-methylacetamide

To a solution of potassium carbonate (93 g, 750 mmol) in H₂O (300 mL) was added N,O-dimethylhydroxylamine hydrochloride (30 g, 300 mmol) at 0° C. The mixture was further cooled to −5° C., and 2-chloroacetyl chloride (40.6 g, 360 mmol) was added. The solution was stirred at room temperature for 2 h. The aqueous mixture was extracted with toluene (3×300 mL). The combined organic layers were washed with brine, dried over sodium sulfate, and concentrated to give the crude product (35 g, 85%) as a white solid. LC/MS: m/e=138 (M+H)⁺.

### 2-Chloro-1-cyclopentylethanone

To a solution of 2-chloro-N-methoxy-N-methylacetamide (27 g, 200 mmol) in THF (300 mL) was added 2N cyclopentylmagnesium bromide solution (100 mL, 200 mmol) while maintaining the temperature at 0° C. The reaction was stirred at room temperature for 1 h. After separation, the aqueous layer was extracted with tert-Butyl methyl ether (3×300 mL). The combined organic layer was washed with brine, dried over Na₂SO₄, and concentrated and the residue was purified by chromatography (dichloromethane/methanol=100/1-20/1) to give the product (13.7 g, 47%) as a yellow oil. ¹H NMR (400 MHz, CDCl₃) δ 4.17 (s, 2H), 3.08-3.17 (m, 1H), 1.60-1.90 (m, 8H), LC/MS: m/e=147 (M+H)⁺.

### 4-Cyclopentyl-1H-imidazole

To a solution of 2-chloro-1-cyclopentylethanone (13 g, 89 mmol) in methanol (120 mL) was added formamidine acetate (14 g, 134 mmol), ammonium hydroxide (100 mL) and THF (40 mL). The mixture was stirred at room temperature for 20 h. The solution was concentrated and the residue purified by flash chromatography (dichloromethane/methanol 20/1) to give the product (6.0 g, 50%) as a yellow oil. LC/MS: m/e=137 (M+H)⁺.

### 4-Cyclopentyl-5-nitro-1H-imidazole

A solution of 4-cyclopentyl-1H-imidazole (6.0 g, 44.1 mmol) in fuming sulfuric acid (20 mL) was added fuming nitric acid (20 mL). The mixture was stirred at room temperature for 2 h. then poured into ice (100 g). The pH of the mixture was adjusted to 7 with sodium hydroxide. The mixture was extracted with dichloromethane (3×100 mL). The combined organic phases were dried over sodium sulfate, concentrated and the residue purified by flash chromatography (dichloromethane/methanol 20/1) to give the product (6.2 g, 77%) as a yellow oil. LC/MS: m/e=182 (M+H)⁺.

### 4-Cyclopentyl-1H-imidazol-5-amine hydrochloride

To a solution of 4-cyclopentyl-5-nitro-1H-imidazole (6.2 g, 34.2 mmol) in ethanol (80 mL) was added hydrazine hydrate (5.1 g, 102.6 mmol) and Raney Ni (600 mg). The mixture was stirred at room temperature for 1 h. The reaction was filtered over celite and the solvent removed in vacuo. The crude material was dissolved in dioxane (10 mL) and a solution of HCl in 1,4-dioxane (8 mL) was added at 0° C. The mixture was stirred at rt for 30 min, then concentrated to give the product (5.5 g, 86%) as a white solid. LC/MS: m/e=152 (M+H)⁺.

### Cyanomethyl acetate

To a solution of 2-chloroacetonitrile (6.2 g, 34.2 mmol) in dichloroethane (80 mL) and H₂O (80 mL) was added sodium acetate (5.1 g, 102.6 mmol) and cesium carbonate (5.1 g, 102.6 mmol). The mixture was heated at reflux for 16 h. After filtration, the solution was extracted with dichloromethane (3×100 mL). The combined organic phases were washed with brine, dried over sodium sulfate, and concentrated to give the product (5.5 g, 86%) as a white solid. ¹H NMR (400 MHz, CDCl₃) δ: 4.72 (s, 2H), 2.17 (s, 3H).

### 2-Imino-2-methoxyethyl acetate hydrochloride

HCl gas was bubbled through methanol (100 mL) at 0° C. for 30 minutes to give a saturated solution. Cyanomethyl acetate (7.5 g, 75.8 mmol) was added, and the mixture was stirred at rt for 16 hours. Evaporation gave the product (7.0 g, 70%), which was used in the next reaction without further purification. LC/MS: m/e=132 (M+H)⁺.

### 2-(Ethoxycarbonylimino)-2-methoxyethyl acetate

To a solution of 2-imino-2-methoxyethyl acetate hydrochloride (7.0 g, 53.4 mmol) in dichloroethane (80 mL) was added ethyl carbonochloridate (6.96 g, 64.1 mmol) and triethylamine (16.2 g, 16.02 mmol) at 0° C. The mixture was stirred at room temperature for 2 h. Evaporation of the solvent gave the product (4.5 g, 42%) which was used without further purification. LC/MS: m/e=204 (M+H)⁺.

### 2-(4-Cyclopentyl-1H-imidazol-5-ylamino)-2-(ethoxycarbonylimino)ethyl acetate

To a solution of 4-cyclopentyl-1H-imidazol-5-amine hydrochloride (I-228.5) (1.2 g, 6.4 mmol) in dioxane (15 mL) was added 2-(ethoxycarbonylimino)-2-methoxyethyl acetate (1.3 g, 6.4 mmol) and triethylamine (1.9 g, 19.2 mmol) at 0° C. The reaction was stirred at room temperature for 2 h. The solution was extracted with ethyl acetate (3×30 mL). The combined organic layers were washed with brine, dried over sodium sulfate, and concentrated to dryness. The residue was purified by flash chromatography (dichloromethane/methanol=15/1) to give the product (160 mg, 8%) as a yellow solid. LC/MS: m/e=323 (M+H)⁺.

### 8-Cyclopentyl-2-(hydroxymethyl)imidazo[1,5-a][1,3,5]triazin-4(1H)-one

To a solution of 2-(4-cyclopentyl-1H-imidazol-5-ylamino)-2-(ethoxycarbonylimino)ethyl acetate (160 mg, 0.5 mmol) in ethanol (8 mL) was added potassium carbonate (207 mg, 1.5 mmol). The reaction was heated at reflux for 2 h. After filtration, the solution was extracted with ethyl acetate (3×10 mL). The combined organic layers were washed with brine, dried over sodium sulfate, and concentrated to dryness. The residue was purified by flash chromatography (dichloromethane/methanol=20/1) to give the product (60 mg, 51%) as a yellow oil. LC/MS: m/e=235 (M+H)⁺.

### 2-(Chloromethyl)-8-cyclopentylimidazo[1,5-a][1,3,5]triazin-4(1H)-one

To a 10 mL of flask was added 8-cyclopentyl-2-(hydroxymethyl)imidazo[1,5-a][1,3,5]triazin-4(1H)-one (60 mg, 0.26 mmol) and thionyl chloride (5 mL) at 0° C. The reaction was stirred at 0° C. for 5 hours. Upon completion, the mixture was concentrated to dryness while maintaining the vessel at room temperature to afford 2-(chloromethyl)-8-cyclopentylimidazo[1,5-a][1,3,5]triazin-4(1H)-one (54 mg, 51%) as a brown solid which was used directly in the next step. LC/MS: m/e=253 (M+H)⁺.

### 2-(((4-Chlorobenzyl)(2-hydroxyethyl)amino)methyl)-8-cyclopentylimidazo[1,5-a][1,3,5]triazin-4(3H)-one

To a solution of 2-(chloromethyl)-8-cyclopentylimidazo[1,5-a][1,3,5]triazin-4(1H)-one (25 mg, 0.1 mmol) in acetonitrile (3 mL) was added 2-(4-chlorobenzylamino)ethanol (28 mg, 0.15 mmol) and sodium iodide (15 mg, 0.1 mmol). The reaction was stirred at room temperature for 16 h. The mixture was concentrated to dryness, and the residue was suspended in water (5 mL) and extracted with dichloromethane (3×15 mL). The combined organic phases were dried over sodium sulfate, concentrated and the residue was purified by flash chromatography (dichloromethane/methanol=100/1-20/1) to give the product (12 mg, 30%) as a yellow solid. LC/MS: m/e=402 (M+H)⁺.

### Example 10

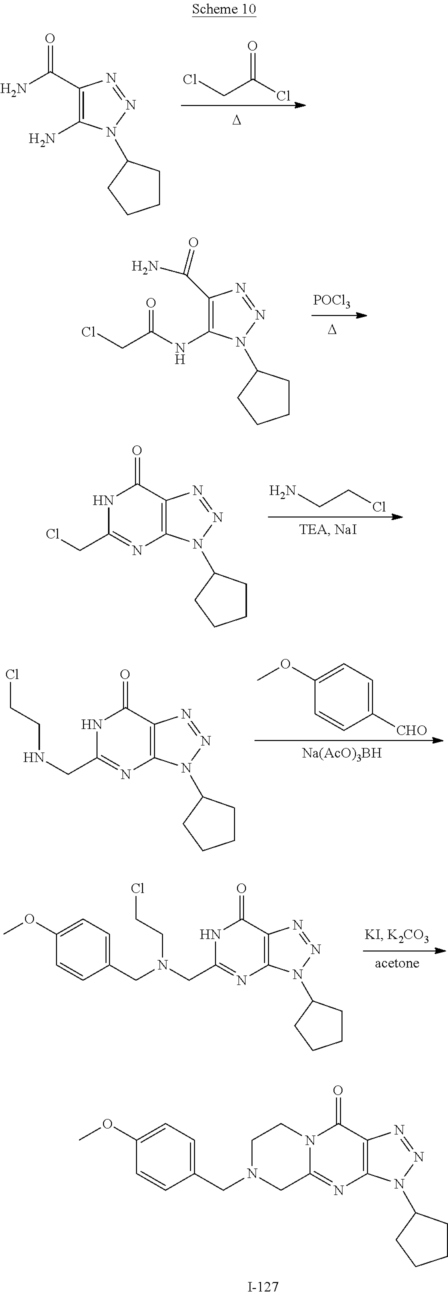

### Compound I-127

### 3-Cyclopentyl-6-(4-methoxybenzyl)-5,6,7,8-tetrahydropyrazino[1,2-a][1,2,3]triazolo[4,5-d]pyrimidin-10(3H)-one

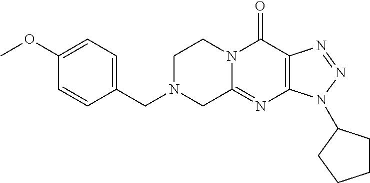 [SMILES: COc1ccc(CN2CCn3c(nc4c(nnn4C4CCCC4)c3=O)C2)cc1]

To a solution of 5-(((2-chloroethyl)(4-methoxybenzyl)amino)methyl)-3-cyclopentyl-3H-[1,2,3]triazolo[4,5-d]pyrimidin-7(6H)-one (I-230.4) (48 mg, 0.12 mmol) in acetone (2 mL) was added potassium iodide (19 mg, 0.12 mmol) and potassium carbonate (24 mg, 0.17 mmol). The mixture was stirred at room temperature for 8 h. The mixture was concentrated; the residue was suspended in water (3 mL), and extracted with dichloromethane (3×5 mL). The combined organic phases were dried over sodium sulfate, concentrated and the residue was purified by prep-HPLC to give the product (12 mg, 27%) as a yellow solid. ¹H NMR (400 MHz, d⁶-acetone): δ 7.24 (m, 2H), 6.85 (m, 2H), 4.69 (quint, J=6.0 Hz, 1H), 3.74 (t, J=5.6 Hz, 2H), 3.71 (s, 3H), 3.59 (s, 2H), 3.28 (s, 2H), 2.88 (t, J=5.2 Hz, 2H), 2.11 (m, 2H), 1.98 (m, 2H), 1.85 (m, 2H), 1.63 (m, 2H), LC/MS: m/e=381 (M+H)⁺.

### 5-(2-Chloroacetamido)-1-cyclopentyl-1H-1,2,3-triazole-4-carboxamide

A mixture of 5-amino-1-cyclopentyl-1H-1,2,3-triazole-4-carboxamide (synthesized using the procedure in Haning, H., et al., Bioorg. Med. Chem. Lett 2005, 15, 3900) (5.6 g, 28.7 mmol) and 2-chloroacetyl chloride (3.25 g, 28.7 mmol) was stirred at 110° C. for 1 h. The mixture was purified by flash chromatography (dichloromethane/methanol=100/1-20/1) to give the product (4.0 g, 51%) as a yellow solid. LC/MS: m/e=272 (M+H)⁺.

### 5-(Chloromethyl)-3-cyclopentyl-3H-[1,2,3]triazolo[4,5-d]pyrimidin-7(6H)-one

A solution of 5-(2-chloroacetamido)-1-cyclopentyl-1H-1,2,3-triazole-4-carboxamide (4.0 g, 14.8 mmol) in phosphorus oxychloride (12 mL) was stirred at 50° C. for 4 h. The mixture was concentrated to dryness in vacuo. The residue was dissolved in dichloromethane (30 mL) and washed with water (3×10 mL). The organic phase was dried over sodium sulfate, concentrated and the residue was purified by flash chromatography (dichloromethane/methanol=100/1-20/1) to give the product (2.0 g, 54%) as a yellow solid. ¹H NMR (400 MHz, DMSO-d₆) δ 11.17 (s, 1H), 4.86 (m, 1H), 4.50 (s, 2H), 2.17-2.14 (m, 2H), 1.99-1.86 (m, 4H), 1.70-1.67 (m, 2H), LC/MS: m/e=254 (M+H)⁺.

### 5-((2-Chloroethylamino)methyl)-3-cyclopentyl-3H-[1,2,3]triazolo[4,5-d]pyrimidin-7(6H)-one

A solution of 5-(chloromethyl)-3-cyclopentyl-3H-[1,2,3]triazolo[4,5-d]pyrimidin-7(6H)-one (400 mg, 1.58 mmol) in acetonitrile (8 mL) was added 2-chloroethanamine hydrochloride (364 mg, 3.16 mmol), triethylamine (319 mg, 3.16 mmol), sodium iodide (237 mg, 1.58 mmol). The mixture was stirred at room temperature for 16 h. The mixture was concentrated to dryness. The residue was suspended in water (5 mL) and extracted with dichloromethane (3×15 mL). The combined organic phases were dried over sodium sulfate, concentrated and the residue was purified by flash chromatography (dichloromethane/methanol=100/1-20/1) to give the product (360 mg, 77%) as a yellow solid. LC/MS: m/e=297 (M+H)⁺.

### 5-(((2-Chloroethyl)(4-methoxybenzyl)amino)methyl)-3-cyclopentyl-3H-[1,2,3]triazolo[4,5-d]pyrimidin-7(6H)-one

A solution of 5-((2-chloroethylamino)methyl)-3-cyclopentyl-3H-[1,2,3]triazolo[4,5-d]pyrimidin-7(6H)-one (140 mg, 0.47 mmol) in dichloromethane (4 mL) was added 4-methoxybenzaldehyde (96 mg, 0.71 mmol) and acetic acid (43 mg, 0.71 mmol). The mixture was stirred at room temperature for 1 h. Sodium triacetoxyborohydride (301 mg, 1.41 mmol) was added, and the reaction was stirred at room temperature for 5 h. Saturated sodium bicarbonate (3 mL) was added, and the mixture was extracted with dichloromethane (3×6 mL). The combined organic phases were dried over sodium sulfate, concentrated and the residue was purified by prep-HPLC (dichloromethane/methanol=10/1) to give the product (48 mg, 24%) as a yellow solid. LC/MS: m/e=417 (M+H)⁺.

### Compound I-128

### 6-(4-Chlorobenzyl)-3-cyclopentyl-5,6,7,8-tetrahydropyrazino[1,2-a][1,2,3]triazolo[4,5-d]pyrimidin-10(3H)-one

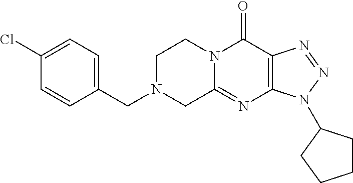 [SMILES: O=c1c2nnn(C3CCCC3)c2nc2n1CCN(Cc1ccc(Cl)cc1)C2]

The title compound was prepared in a manner similar to I-127, substituting 4-chlorobenzaldehyde for 4-methoxybenzaldehyde. ¹H NMR (400 MHz, d⁶-acetone): δ 7.28 (m, 4H), 4.65 (m, 1H), 3.71 (t, J=5.2 Hz, 2H), 3.61 (s, 2H), 3.28 (s, 2H), 2.89 (t, J=5.6 Hz, 2H), 2.08 (m, 2H), 1.95 (m, 2H), 1.80 (m, 2H), 1.58 (m, 2H), LC/MS: m/e=385 (M+H)⁺.

### Example 11

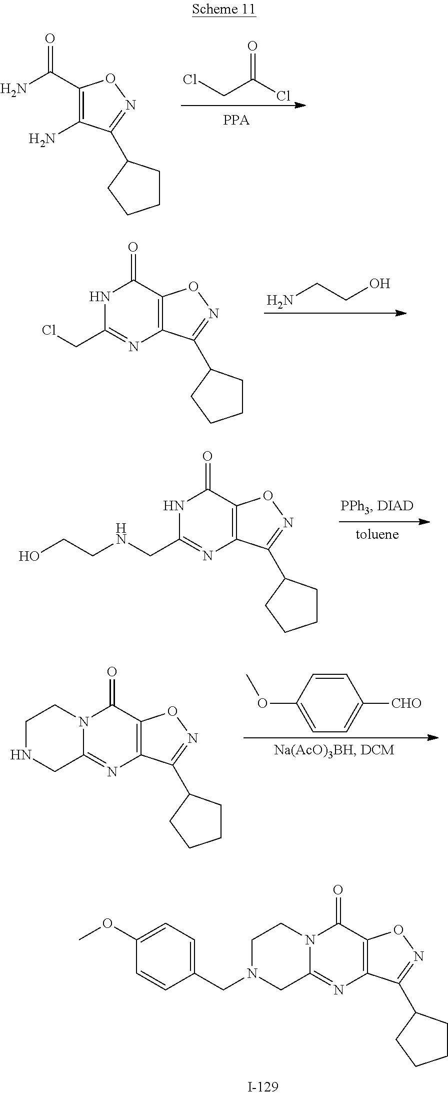

### Compound I-129

### 3-Cyclopentyl-6-(4-methoxybenzyl)-7,8-dihydro-5H-isoxazolo[4,5-d]pyrazino[1,2-a]pyrimidin-10(6H)-one

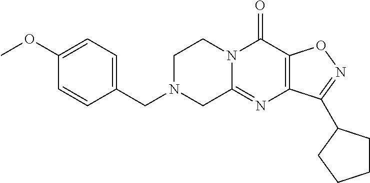 [SMILES: COc1ccc(CN2CCn3c(nc4c(C5CCCC5)noc4c3=O)C2)cc1]

A mixture of 3-cyclopentyl-7,8-dihydro-5H-isoxazolo[4,5-d]pyrazino[1,2-a]pyrimidin-10(6H)-one (I-232.3) (15 mg, 0.057 mmol), 4-methoxybenzaldehyde (16 mg, 0.11 mmol) and sodium triacetoxyborohydride (49 mg, 0.23 mmol) in dichloromethane (5 mL) was stirred at room temperature for 16 h. Upon completion, the mixture was filtered and the filtrate was concentrated to dryness. The crude was purified by prep-TLC (petroleum ether:ethyl acetate=3:1) to give the title compound (14.2 mg, 65%) as a colorless oil. ¹H NMR (400 MHz, CDCl₃): δ 7.28 (d, J=8.8 Hz, 2H), 6.90 (d, J=8.8 Hz, 2H), 4.10 (t, J=5.6 Hz, 2H), 3.83 (s, 3H), 3.74 (s, 2H), 3.66 (s, 2H), 3.39 (quint, J=8.0 Hz, 1H), 2.91 (t, J=5.6 Hz, 2H), 2.14 (m, 2H), 1.96 (m, 2H), 1.86 (m, 2H), 1.72 (m, 2H), LC/MS: m/e=381 (M+H)⁺.

### 5-(Chloromethyl)-3-cyclopentylisoxazolo[4,5-d]pyrimidin-7(6H)-one

A solution of 4-amino-3-cyclopentylisoxazole-5-carboxamide (synthesized using the procedure in Haning, H., et al., Bioorg. Med. Chem. Lett. 2005, 15, 3900) (200 mg, 1 mmol) and 2-chloroacetyl chloride (233 mg, 2 mmol) was heated to 120° C. for 30 min. The solvent was removed and the residue was resuspended in polyphosphosphoric acid (1 mL). The reaction was heated to 150° C. for 1 hour. Upon completion, the reaction was quenched into ice-water. Dichloromethane (15 mL) was added, the mixture was cooled to 0° C. and the pH was carefully adjusted to 8 with saturated sodium bicarbonate. The mixture was extracted with dichloromethane (20 mL×2), and the combined organic extracts were dried and concentrated to dryness. The crude material was purified by silica gel chromatography (dichloromethane:methanol=20:1) to give the title compound (140 mg, 54%) as a white solid. LC/MS: m/e=254 (M+H)⁺.

### 3-Cyclopentyl-5-((2-hydroxyethylamino)methyl)isoxazolo[4,5-d]pyrimidin-7(6H)-one

A mixture of 5-(chloromethyl)-3-cyclopentylisoxazolo[4,5-d]pyrimidin-7(6H)-one (140 mg, 0.55 mmol) and 2-aminoethanol (0.5 mL) was stirred at room temperature for 30 min. Upon completion, methanol (2 mL) was added, and the mixture was charged to a reverse-phase column (eluting from 100% water to water:acetonitrile=88:12) to give the title compound (100 mg, 65%) as white solid. LC/MS: m/e=279 (M+H)⁺.

### 3-Cyclopentyl-7,8-dihydro-5H-isoxazolo[4,5-d]pyrazino[1,2-a]pyrimidin-10(6H)-one

To a mixture of 3-cyclopentyl-5-((2-hydroxyethylamino)methyl)isoxazolo[4,5-d]pyrimidin-7(6H)-one (100 mg, 0.36 mmol) and triphenylphosphine (188 mg, 0.72 mmol) in dry toluene (5 mL) was added diisopropyl azodicarboxylate (145 mg, 0.72 mmol) slowly at 0° C. The reaction mixture was stirred for 2 hours at room temperature. Upon completion, the solvent was removed in vacuo. Purification by reverse-phase chromatography (eluting from 100% water to water:acetonitrile=100:30) gave the title compound (30 mg, 32%) as white solid. LC/MS: m/e=261 (M+H)⁺.

### Compound I-130

### 6-(4-Chlorobenzyl)-3-cyclopentyl-7,8-dihydro-5H-isoxazolo[4,5-d]pyrazino[1,2-a]pyrimidin-10(6H)-one

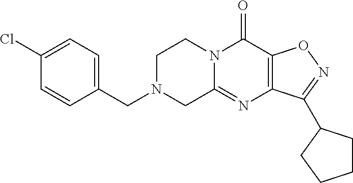 [SMILES: O=c1c2onc(C3CCCC3)c2nc2n1CCN(Cc1ccc(Cl)cc1)C2]

The title compound was prepared in a manner similar to I-129, substituting 4-chlorobenzaldehyde for 4-methoxybenzaldehyde. ¹H NMR (400 MHz, CDCl₃): δ 7.33 (m, 4H), 4.11 (t, J=6.0 Hz, 2H), 3.74 (s, 2H), 3.67 (s, 2H), 3.39 (quint, J=8.4 Hz, 1H), 2.92 (t, J=6.0 Hz, 2H), 2.13 (m, 2H), 1.98 (m, 2H), 1.86 (m, 2H), 1.72 (m, 2H), LC/MS: m/e=385 (M+H)⁺.

### Example 12

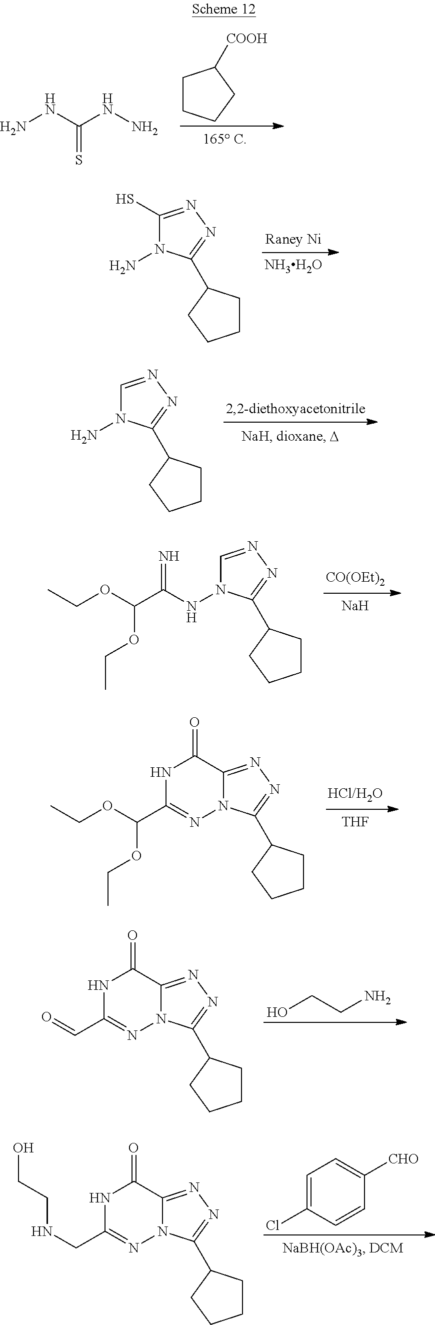

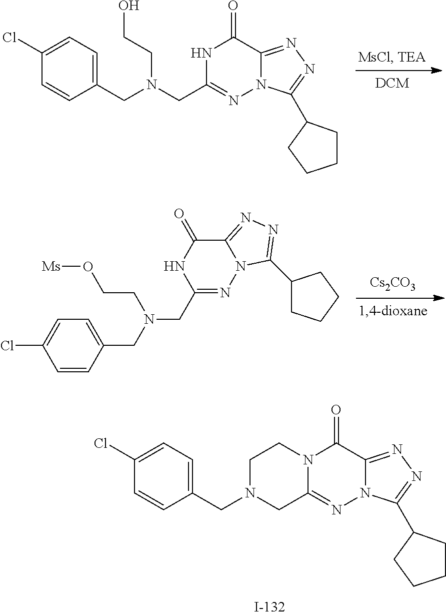

### Compound I-132

### 7-(4-Chlorobenzyl)-3-cyclopentyl-8,9-dihydro-6H-pyrazino[2,1-c][1,2,4]triazolo[3,4-f][1,2,4]triazin-11(7H)-one

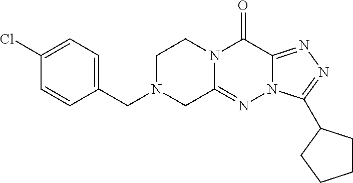 [SMILES: O=c1c2nnc(C3CCCC3)n2nc2n1CCN(Cc1ccc(Cl)cc1)C2]

2-((4-chlorobenzyl)((3-cyclopentyl-8-oxo-7,8-dihydro-[1,2,4]triazolo[4,3-f][1,2,4]triazin-6-yl)methyl)amino)ethyl methanesulfonate (62 mg, 0.13 mmol) and cesium carbonate (126 mg, 0.39 mmol) were dissolved in dioxane (5 mL) and heated to reflux with stirring for 1 hour. Upon completion, the mixture was concentrated to dryness and purified by Pre-HPLC to give the title compound (22.66 mg, 44%) as a white solid. ¹H NMR (400 MHz, CDCl₃): δ 7.38-7.29 (m, 4H), 4.05 (t, J=6.0 Hz, 2H), 3.70 (s, 2H), 3.64 (s, 2H), 3.48 (quint, J=8.4 Hz, 1H), 2.93 (t, J=6.0 Hz, 2H), 2.15 (m, 2H), 2.04 (m, 2H), 1.88 (m, 2H), 1.71 (m, 2H), LC/MS: m/e=385 (M+H)⁺.

### 4-Amino-5-cyclopentyl-4H-1,2,4-triazole-3-thiol

The title compound was synthesized based on the procedure in Haning, H., et al., Bioorg. Med. Chem. Lett. 2005, 15, 3900. A mixture of thiourea (14.3 g, 135.1 mmol), and cyclopentanecarboxylic acid (14.0 g, 122.8 mmol) was stirred at 165° C. for 30 min. The water produced as a byproduct of the reaction was distilled off until a yellowish condensate appeared. After cooling, a mixture of dichloromethane/methanol (95/5) (250 mL) was added to the suspension and the precipitate was filtered and discarded. The filtrate was concentrated and subjected to flash column chromatography (dichloromethane/methanol=100/1). The product (10.3 g, 42%) was obtained as a colorless solid. LC/MS: m/e=185 (M+H)⁺.

### 3-Cyclopentyl-4H-1,2,4-triazol-4-amine

A mixture of 4-amino-5-cyclopentyl-4H-1,2,4-triazole-3-thiol (7.0 g, 38.0 mmol) in aqueous ammonia (100 mL) was stirred at room temperature. Raney Ni (500 mg) in water (15 L) was added. The reaction was stirred at 90° C. for about 2 hrs. The mixture was filtered through a pad of celite and washed with methanol (100 mL). The filtrate was concentrated in vacuo to yield the crude product (3.1 g, 54%) which was used in the next step without further purification. LC/MS: m/e=153 (M+H)⁺.

### N-(3-cyclopentyl-4H-1,2,4-triazol-4-yl)-2,2-diethoxyacetimidamide

To a mixture of 3-cyclopentyl-4H-1,2,4-triazol-4-amine (1.18 g, 7.7 mmol) in dry dioxane (20 mL) was added sodium hydride (1.2 g, 31 mmol), and the reaction stirred for 30 min. 2,2-Diethoxyacetonitrile (2 g, 15.5 mmol) was added in portions, and the reaction was stirred overnight at 90° C. Upon completion, water (5 mL) was added, and the solution was extracted with dichloromethane (30 mL×3). The combined organic phases were evaporated in vacuo to get the crude product (1.13 g, 52%), which was suitable for use without further purification. LC/MS: m/e=282 (M+H)⁺.

### 3-Cyclopentyl-6-(diethoxymethyl)-[1,2,4]triazolo[4,3-f][1,2,4]triazin-8(7H)-one

To a mixture of N-(3-cyclopentyl-4H-1,2,4-triazol-4-yl)-2,2-diethoxyacetimidamide (1.13 g, 4 mmol) and diethyl carbonate (1.42 g, 12 mmol) in dry dioxane (20 mL) was added sodium hydride (480 mg, 12 mmol). The reaction was stirred at 100° C. for 2 days. Upon completion, water (5 nit) was added, and the solution was extracted with ethyl acetate (30 mL 3). The combined organic phases were dried and concentrated. The crude material was purified by silica gel chromatography (eluting with dichloromethane:methanol=100:1) to give the title compound (680 mg, 55%) as a yellow oil. LC/MS: m/e=308 (M+H)⁺.

### 3-Cyclopentyl-8-oxo-7,8-dihydro-[1,2,4]triazolo[4,3-f][1,2,4]triazine-6-carbaldehyde

A mixture of 3-cyclopentyl-6-(diethoxymethyl)-[1,2,4]triazolo[4,3-f][1,2,4]triazin-8(7H)-one (680 mg, 2.2 mmol) in hydrochloric acid (6N, 6 mL) and tetrahydrofuran (2 mL) was heated to reflux overnight. Upon completion, the solvent was removed in vacuo, affording the crude product (520 mg, 93%) which was used without further purification. LC/MS: m/e=252 (M+H)⁺.

### 3-Cyclopentyl-6-((2-hydroxyethylamino)methyl)-[1,2,4]triazolo[4,3-f][1,2,4]triazin-8(7H)-one

A mixture of 3-cyclopentyl-8-oxo-7,8-dihydro-[1,2,4]triazolo[4,3-f][1,2,4]triazine-6-carbaldehyde (516 mg, 2.2 mmol), 2-aminoethanol (540 mg, 8.8 mmol) and sodium triacetoxyborohydride (1.88 g, 8.8 mmol) in dichloromethane (20 mL) was stirred at room temperature overnight. Upon completion, the mixture was purified by reverse phase chromatography (eluted from water to water:acetonitrile=100:8) to give the title compound (186 mg, 30%) as a white solid. LC/MS: m/e=279 (M+H)¹.

### 6-(((4-Chlorobenzyl)(2-hydroxyethyl)amino)methyl)-3-cyclopentyl-[1,2,4]triazolo[4,3-f][1,2,4]triazin-8(7H)-one

A mixture of 3-cyclopentyl-6-((2-hydroxyethylamino)methyl)-[1,2,4]triazolo[4,3-f][1,2,4]triazin-8(7H)-one (90 mg, 0.32 mmol), 4-chlorobenzaldehyde (137 mg, 0.97 mmol) and sodium triacetoxyborohydride (412 mg, 1.94 mmol) in dichloromethane (5 mL) was stirred at room temperature for 16 h. Upon completion, the mixture was washed with water (10 mL×2), dried and concentrated in vacuo. The crude material was purified by reverse phase chromatography (eluting from water to water:acetonitrile=100:30) to give the title compound (54 mg, 41%) as a white solid. LC/MS: m/e=403 (M+H)⁺.

### 2-((4-Chlorobenzyl)((3-cyclopentyl-8-oxo-7,8-dihydro-[1,2,4]triazolo[4,3-f][1,2,4]triazin-6-yl)methyl)amino)ethyl methanesulfonate

To a solution of 6-(((4-chlorobenzyl)(2-hydroxyethyl)amino)methyl)-3-cyclopentyl-[1,2,4]triazolo[4,3-f][1,2,4]triazin-8(7H)-one (54 mg, 0.13 mmol) and triethylamine (41 mg, 0.4 mmol) in dichloromethane (5 mL) was added methanesulfonyl chloride (31 mg, 0.26 mmol) slowly at 0° C. The mixture was stirred for 1 hour at room temperature. Upon completion, the reaction mixture was washed with water (10 mL×2), dried and concentrated in vacuo to give the title compound (62 mg, 96%) which was used without further purification. LC/MS: m/e=481 (M+H)⁺.

### Compound I-131

### 3-Cyclopentyl-7-(4-methoxybenzyl)-8,9-dihydro-6H-pyrazino[2,1-c][1,2,4]triazolo[3,4-f][1,2,4]triazin-11(7H)-one

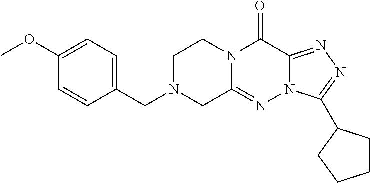 [SMILES: COc1ccc(CN2CCn3c(nn4c(C5CCCC5)nnc4c3=O)C2)cc1]

The title compound was prepared in a manner similar to I-132, substituting 4-methoxybenzaldehyde for 4-chlorobenzaldehyde. ¹H NMR (400 MHz, d⁴-MeOH): δ 7.29 (d, J=8.8 Hz, 2H), 6.90 (d, J=8.8 Hz, 2H), 3.97 (t, J=6.0 Hz, 2H), 3.79 (s, 3H), 3.68 (s, 2H), 3.64 (s, 2H), 3.54 (quint, J=8.4 Hz, 1H), 2.95 (t, J=6.0 Hz, 2H), 2.15 (m, 2H), 1.98 (m, 2H), 1.87 (m, 2H), 1.75 (m, 2H), LC/MS: m/e=381 (M+H)⁺.

### Compound I-213

### 3-Isobutyl-7-(4-methoxybenzyl)-8,9-dihydro-6H-pyrazino[2,1-c][1,2,4]triazolo[3,4-f][1,2,4]triazin-11(7H)-one

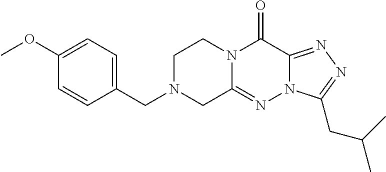 [SMILES: COc1ccc(CN2CCn3c(nn4c(CC(C)C)nnc4c3=O)C2)cc1]

The title compound was prepared in a manner similar to I-132, substituting 3-methylbutanoic acid for cyclopentanecarboxylic acid, and substituting 4-methoxybenzaldehyde for 4-chlorobenzaldehyde. ¹H NMR (400 MHz, CDCl₃): δ 7.30-7.25 (m, 2H), 6.93-6.90 (m, 2H), 4.06-4.03 (m, 2H), 3.84 (s, 3H), 3.65 (m, 4H), 2.94-2.88 (m, 4H), 2.31-2.24 (m, 1H), 0.99 (d, J=6.8 Hz, 6H), LC/MS: m/e=369 (M+H)⁺.

### Compound I-214

### 7-Benzyl-3-isobutyl-8,9-dihydro-6H-pyrazino[2,1-c][1,2,4]triazolo[3,4-f][1,2,4]triazin-11(7H)-one

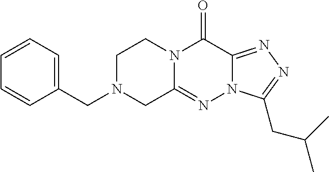 [SMILES: CC(C)Cc1nnc2c(=O)n3c(nn12)CN(Cc1ccccc1)CC3]

The title compound was prepared in a manner similar to I-132, substituting 3-methylbutanoic acid for cyclopentanecarboxylic acid, and substituting benzaldehyde for 4-chlorobenzaldehyde. ¹H NMR (400 MHz, CDCl₃): δ 7.43-7.32 (m, 5H), 4.06 (t, J=5.6 Hz, 2H), 3.73 (s, 2H), 3.67 (s, 2H), 2.96-2.89 (m, 4H), 2.34-2.24 (m, 1H), 0.99 (d, J=6.8 Hz, 6H), LC/MS: m/e=339 (M+H)⁺.

### Compound I-215

### 7-(4-Chlorobenzyl)-3-isobutyl-8,9-dihydro-6H-pyrazino[2,1-c][1,2,4]triazolo[3,4-f][1,2,4]triazin-11(7H)-one

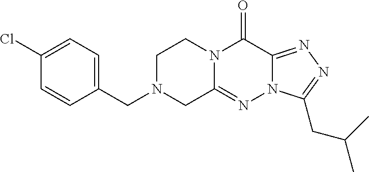 [SMILES: CC(C)Cc1nnc2c(=O)n3c(nn12)CN(Cc1ccc(Cl)cc1)CC3]

The title compound was prepared in a manner similar to I-132, substituting 3-methylbutanoic acid for cyclopentanecarboxylic acid. ¹H NMR (400 MHz, CDCl₃): δ 7.37 (d, J=8.4 Hz, 2H), 7.31 (d, J=8.4 Hz, 2H), 4.06 (t, J=5.6 Hz, 2H), 3.70 (s, 2H), 3.64 (s, 2H), 2.97-2.89 (m, 4H), 2.35-2.25 (m, 1H), 0.99 (d, J=6.8 Hz, 6H), LC/MS: m/e=373 (M+H)⁺.

### Compound I-221

### 7-(4-Chlorobenzyl)-3-isopropyl-8,9-dihydro-6H-pyrazino[2,1-c][1,2,4]triazolo[3,4-f][1,2,4]triazin-11(7H)-one

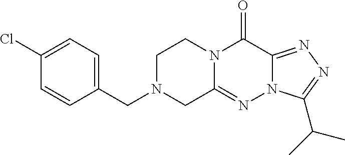 [SMILES: CC(C)c1nnc2c(=O)n3c(nn12)CN(Cc1ccc(Cl)cc1)CC3]

The title compound was prepared in a manner similar to I-132, substituting isobutyric acid for cyclopentanecarboxylic acid. ¹H NMR (400 MHz, CDCl₃): δ 7.36 (d, J=8.0 Hz, 2H), 7.29 (d, J=8.0 Hz, 2H), 4.05 (t, J=5.8 Hz, 2H), 3.67 (s, 2H), 3.64 (s, 2H), 3.48-3.44 (m, 1H), 2.92 (t, J=5.8 Hz, 2H), 1.46 (d, J=6.8 Hz, 6H), LC/MS: m/e=359 (M+H)⁺.

### Compound I-122

### 3-Isopropyl-7-(4-methoxybenzyl)-8,9-dihydro-6H-pyrazino[2,1-c][1,2,4]triazolo[3,4-f][1,2,4]triazin-11(7H)-one

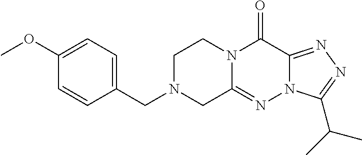 [SMILES: COc1ccc(CN2CCn3c(nn4c(C(C)C)nnc4c3=O)C2)cc1]

The title compound was prepared in a manner similar to I-132, substituting isobutyric acid for cyclopentanecarboxylic acid, and substituting 4-methoxybenzaldehyde for 4-chlorobenzaldehyde. ¹H NMR (400 MHz, CDCl₃): δ 7.27 (d, J=8.4 Hz, 2H), 6.92 (d, J=8.4 Hz, 2H), 4.04 (t, J=5.8 Hz, 2H), 3.84 (s, 3H), 3.67 (s, 2H), 3.64 (s, 2H), 3.47-3.40 (m, 1H), 2.91 (t, J=5.8 Hz, 2H), 1.46 (d, J=7.2 Hz, 6H), LC/MS: m/e=355 (M+H)⁺.

### Compound I-223

### 7-Benzyl-3-isopropyl-8,9-dihydro-6H-pyrazino[2,1-c][1,2,4]triazolo[3,4-f][1,2,4]triazin-11(7H)-one

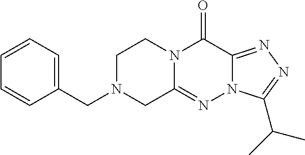 [SMILES: CC(C)c1nnc2c(=O)n3c(nn12)CN(Cc1ccccc1)CC3]

The title compound was prepared in a manner similar to I-132, substituting isobutyric acid for cyclopentanecarboxylic acid, and substituting benzaldehyde for 4-chlorobenzaldehyde. ¹H NMR (400 MHz, CDCl₃): δ 7.44-7.34 (m, 5H), 4.07 (t, J=5.8 Hz, 2H), 3.75 (s, 2H), 3.69 (s, 2H), 3.50-3.40 (m, 1H), 2.95 (t, J=5.8 Hz, 2H), 1.48 (d, J=6.8 Hz, 6H), LC/MS: m/e=325 (M+H)⁺.

### Compound I-224

### 3-Isobutyl-7-(pyridin-2-ylmethyl)-8,9-dihydro-6H-pyrazino[2,1-c][1,2,4]triazolo[3,4-f][1,2,4]triazin-11(7H)-one

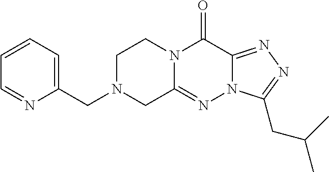 [SMILES: CC(C)Cc1nnc2c(=O)n3c(nn12)CN(Cc1ccccn1)CC3]

The title compound was prepared in a manner similar to I-132, substituting 3-methylbutanoic acid for cyclopentanecarboxylic acid, and substituting picolinaldehyde for 4-chlorobenzaldehyde. ¹H NMR (400 MHz, CDCl₃): δ 8.67-8.62 (m, 1H), 7.79-7.72 (m, 1H), 7.44 (d, J=8.0 Hz, 1H), 7.31-7.24 (m, 1H), 4.10 (t, J=5.6 Hz, 2H), 3.91 (s, 2H), 3.78 (s, 2H), 3.04 (t, J=5.6 Hz, 2H), 2.93 (d, J=7.2 Hz, 2H), 2.34-2.24 (m, 1H), 1.00 (d, J=6.8 Hz, 6H), LC/MS: m/e=340 (M+H)⁺.

### Compound I-225

### 3-Isopropyl-7-phenethyl-8,9-dihydro-6H-pyrazino[2,1-c][1,2,4]triazolo[3,4-f][1,2,4]triazin-11(7H)-one

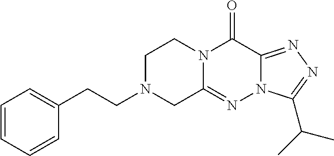 [SMILES: CC(C)c1nnc2c(=O)n3c(nn12)CN(CCc1ccccc1)CC3]

The title compound was prepared in a manner similar to I-132, substituting isobutyric acid for cyclopentanecarboxylic acid, and substituting phenylacetaldehyde for 4-chlorobenzaldehyde. ¹H NMR (400 MHz, CDCl₃): δ 7.34-7.30 (m, 2H), 7.25-7.19 (m, 3H), 4.05 (t, J=5.8 Hz, 2H), 3.74 (s, 2H), 3.52-3.42 (m, 1H), 2.97 (t, J=5.8 Hz, 2H), 2.92-2.36 (m, 2H), 2.35-2.29 (m, 2H), 1.48 (d, J=6.8 Hz, 6H), LC/MS: m/e=339 (M+H)⁺.

### Compound I-226

### 3-Isopropyl-7-(pyridin-2-ylmethyl)-8,9-dihydro-6H-pyrazino[2,1-c][1,2,4]triazolo[3,4-f][1,2,4]triazin-11(7H)-one

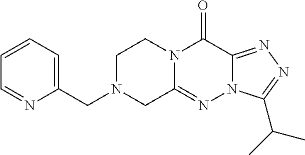 [SMILES: CC(C)c1nnc2c(=O)n3c(nn12)CN(Cc1ccccn1)CC3]

The title compound was prepared in a manner similar to I-132, substituting isobutyric acid for cyclopentanecarboxylic acid, and substituting picolinaldehyde for 4-chlorobenzaldehyde. ¹H NMR (400 MHz, CDCl₃): δ 8.68-8.63 (m, 1H), 7.76-7.74 (m, 1H), 7.42 (d, J=8.0 Hz, 1H), 7.29-7.22 (m, 1H), 4.08 (t, J=5.8 Hz, 2H), 3.89 (s, 2H), 3.76 (s, 2H), 3.46-3.39 (m, 1H), 3.02 (t, J=5.8 Hz, 2H), 1.45 (d, J=6.8 Hz, 6H), LC/MS: m/e=326 (M+H)⁺.

### Compound I-227

### 3-Isobutyl-7-phenethyl-8,9-dihydro-6H-pyrazino[2,1-c][1,2,4]triazolo[3,4-f][1,2,4]triazin-11(7H)-one

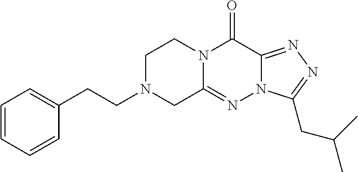 [SMILES: CC(C)Cc1nnc2c(=O)n3c(nn12)CN(CCc1ccccc1)CC3]

The title compound was prepared in a manner similar to I-132, substituting 3-methylbutanoic acid for cyclopentanecarboxylic acid, and substituting phenylacetaldehyde for 4-chlorobenzaldehyde. ¹H NMR (400 MHz, CDCl₃): δ 7.35-7.30 (m, 2H), 7.27-7.22 (m, 3H), 4.10 (br s, 2H), 3.83 (br s, 2H), 3.08 (br s, 2H), 2.97-2.86 (m, 6H), 2.34-2.24 (m, 1H), 1.00 (d, J=6.8 Hz, 6H), LC/MS: m/e=353 (M+H)⁺.

### Compound I-228

### 3-Isobutyl-7-(pyridin-4-ylmethyl)-8,9-dihydro-6H-pyrazino[2,1-c][1,2,4]triazolo[3,4-f][1,2,4]triazin-11(7H)-one

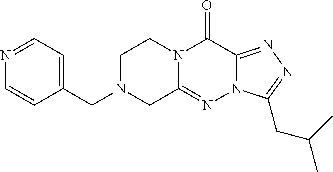 [SMILES: CC(C)Cc1nnc2c(=O)n3c(nn12)CN(Cc1ccncc1)CC3]

The title compound was prepared in a manner similar to I-132, substituting 3-methylbutanoic acid for cyclopentanecarboxylic acid, and substituting isonicotinaldehyde for 4-chlorobenzaldehyde. ¹H NMR (400 MHz, CDCl₃): δ 8.63 (d, J=6.0 Hz, 2H), 7.32 (d, J=6.0 Hz, 2H), 4.08 (t, J=5.8 Hz, 2H), 3.74 (s, 2H), 3.66 (s, 2H), 2.96 (t, J=5.8 Hz, 2H), 2.91 (d, J=7.6 Hz, 2H), 2.32-2.23 (m, 1H), 0.98 (d, J=6.4 Hz, 6H), LC/MS: m/e=340 (M+H)⁺.

### Compound I-245

### 3-Isopropyl-7-(pyridin-4-ylmethyl)-8,9-dihydro-6H-pyrazino[2,1-c][1,2,4]triazolo[3,4-f][1,2,4]triazin-11(7H)-one

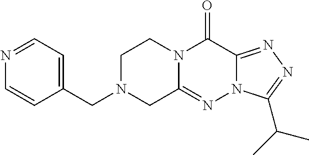 [SMILES: CC(C)c1nnc2c(=O)n3c(nn12)CN(Cc1ccncc1)CC3]

The title compound was prepared in a manner similar to I-132, substituting isobutyric acid for cyclopentanecarboxylic acid, and substituting isonicotinaldehyde for 4-chlorobenzaldehyde. ¹H NMR (400 MHz, CDCl₃): δ 8.66 (d, J=6.0 Hz, 2H), 7.34 (d, J=6.0 Hz, 2H), 4.11 (t, J=5.8 Hz, 2H), 3.77 (s, 2H), 3.70 (s, 2H), 3.50-3.40 (m, 1H), 2.98 (t, J=5.8 Hz, 2H), 1.48 (d, J=6.8 Hz, 6H), LC/MS: m/e=326 (M+H)⁺.

### Example 13

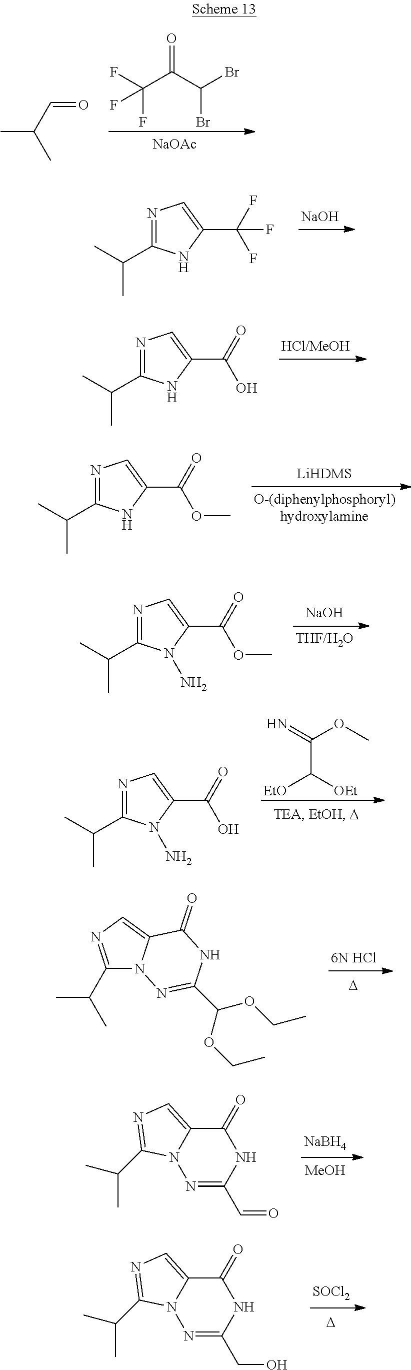

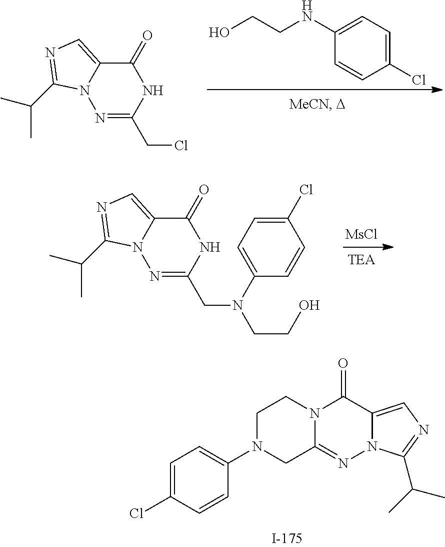

### Compound I-175

### 7-(4-Chlorophenyl)-3-isopropyl-8,9-dihydro-6H-imidazo[5,1-f]pyrazino[2,1-c][1,2,4]triazin-11(7H)-one

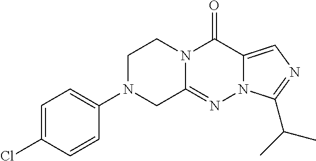 [SMILES: CC(C)c1ncc2c(=O)n3c(nn12)CN(c1ccc(Cl)cc1)CC3]

To a solution of 2-(((4-chlorophenyl)(2-hydroxyethyl)amino)methyl)-7-isopropylimidazo[1,5-f][1,2,4]triazin-4(3H)-one (I-248.11) (540 mg, 1.5 mmol) in dichloromethane (20 mL) was added methanesulfonyl chloride (400 mg, 3.0 mmol) and triethylamine (610 mg, 6.0 mmol) at 0° C. The reaction was allowed to warm to room temperature and stirred overnight. Upon completion, the mixture was quenched with water (100 mL) and extracted with dichloromethane (100 mL×2). The combined organic layers were dried and concentrated in vacuo to give a residue, that was recrystallized from acetonitrile (10 mL) to give the title compound (286 mg, 56%) as an off-white solid. ¹H NMR (400 MHz, CDCl₃): δ 7.87 (s, 1H), 7.30 (m, 2H), 6.85 (m, 2H), 4.40 (s, 2H), 4.21 (t, J=6.0 Hz, 2H), 3.66 (t, J=6.0 Hz, 2H), 3.52 (sept; J=7.2 Hz, 1H), 1.42 (d, J=6.8 Hz, 6H), LC/MS: m/e=344 (M+H)⁺.

### 2-Isopropyl-5-(trifluoromethyl)-1H-imidazole (I-248.1)

The title compound was synthesized based on the method of Jones, L. H. et al. (WO 2006 072833). A mixture of 3,3-dibromo-1,1,1-trifluoropropan-2-one (8.1 g, 30 mmol) and sodium acetate trihydrate (8.1 g, 60 mmol) in water (54 mL) was heated to reflux for 30 mins, then cooled to room temperature. After cooling, the mixture was slowly added to a solution of isobutyraldehyde (1.95 g, 27 mmol), ammonium aydroxide (33 mL) and methanol (135 mL). The reaction was stirred overnight at room temperature. Upon completion, methanol was removed in vacuo and the aqueous mixture was extracted with ethyl acetate (150 mL×2). The combined organic phases were dried over sodium sulfate, filtered and concentrated to dryness in vacuo. The crude product (4.3 g, 89%) was obtained and used in the next step without further purification. LC/MS: m/e=179 (M+H)⁺.

### 2-Isopropyl-1H-imidazole-5-carboxylic acid (I-248.2)

The title compound was synthesized based on the method of Adams, R. S., et al. (US 2008 0171754). A mixture of 2-isopropyl-5-(trifluoromethyl)-1H-imidazole (11.6 g, 65 mmol) and sodium hydroxide (7.0 g, 176 mmol) in water/methanol (80 mL/120 mL) were stirred at room temperature overnight. Upon completion, methanol was removed in vacuo and the pH adjusted to 2 with hydrochloric acid (1N). The solvent was removed in vacuo. The residue was dissolved in ethanol (60 mL), filtered and the filtrate was concentrated in vacuo. The crude product (12.0 g) was used directly in the next step. LC/MS: m/e=155 (M+H)⁺.

### Methyl 2-isopropyl-1H-imidazole-5-carboxylate (I-248.3)

Hydrogen chloride gas was bubbled into a solution of 2-isopropyl-1H-imidazole-5-carboxylic acid (12.0 g, 77 mmol) in methanol (100 mL) until saturated. The mixture was heated to reflux overnight. Upon completion, the mixture was washed with sodium bicarbonate solution (80 mL×3), dried and concentrated to dryness. Purification by silica gel (gradient elution from petroleum ether:ethyl acetate=50:1 to petroleum ether:ethyl acetate=10:1) gave the title compound (6.6 g, 46%) as a white solid. LC/MS: m/e=169 (M+H)⁺.

### Methyl 1-amino-2-isopropyl-1H-imidazole-5-carboxylate (I-248.4)

To a mixture of methyl 2-isopropyl-1H-imidazole-5-carboxylate (3.4 g, 20.2 mmol) in DMF (50 mL) was added lithium his (trimethylsilyl) amide (1N in tetrahydrofuran, 27 mL, 27 mmol) at −10° C. After stirring for 10 min, O-(diphenylphosphoryl)hydroxylamine (6.7 g, 25.3 mmol) was added at 0° C., and the reaction was warmed to room temperature and stirred for 3 hours. Upon completion, the mixture was quenched with water (150 mL), and extracted with ethyl acetate (100 mL×3). The combined organic phases were dried and concentrated to give crude product. Purification by silica gel chromatography (elution with petroleum ether:ethyl acetate=5:1 gave the title compound (2.9 g, 80%) as a colorless oil. LC/MS: m/e=184 (M+H)⁺.

### 1-Amino-2-isopropyl-1H-imidazole-5-carboxylic acid

A mixture of methyl 1-amino-2-isopropyl-1H-imidazole-5-carboxylate (4.0 g, 21.8 mmol) and sodium hydroxide (2.2 g, 54.5 mmol) in water/tetrahydrofuran (20 mL/20 mL) was stirred at room temperature overnight. Upon completion, tetrahydrofuran was removed in vacuo and the pH was adjusted to 3 with hydrochloric acid (1N). The solvent was removed in vacuo, and the residue was dissolved in ethanol (50 mL). The mixture was filtered and the filtrate was concentrated to dryness in vacuo to yield the title compound (2.7 g, 74%), which was used directly in the next step. LC/MS: m/e=170 (M+H)⁺.

### 2-(Diethoxymethyl)-7-isopropylimidazo[1,5-f][1,2,4]triazin-4(3H)-one

A microwave tube was charged with 1-amino-2-isopropyl-1H-imidazole-5-carboxylic acid (1.0 g, 5.9 mmol), methyl 2,2-diethoxyacetimidate (prepared according to the procedure in Zhang, L., et al., Bioorg. Med. Chem. Lett. 2008, 18, 5493) (1.14 g, 7.1 mmol), triethylamine (1.2 g, 11.8 mmol) and ethanol (9 mL). The mixture was heated to 120° C. using microwave with stirring for 2 hours. Upon completion, the mixture was concentrated in vacuo to dryness, diluted with water (100 mL) and extracted with dichloromethane (100 mL×2). The combined organic extracts were dried and concentrated to dryness in vacuo. Purification by silica gel chromatography (elution with petroleum ether:ethyl acetate=2:1) gave the title compound (1.1 g, 65%) as a yellow solid. LC/MS: m/e=281 (M+H)⁺.

### 7-Isopropyl-4-oxo-3,4-dihydroimidazo[1,5-f][1,2,4]triazine-2-carbaldehyde

To a solution of 2-(diethoxymethyl)-7-isopropylimidazo[1,5-f][1,2,4]triazin-4(3H)-one (2.92 g, 10.4 mmol) in tetrahydrofuran (6 mL) was added 6N hydrochloric acid (14 mL). The mixture was heated to reflux for 3 hours. Upon completion, the mixture was concentrated to dryness in vacuo to get the crude product (3.3 g), which was used without further purification. LC/MS: m/e=207 (M+H)⁺.

### 2-(Hydroxymethyl)-7-isopropylimidazo[1,5-f][1,2,4]triazin-4(3H)-one

To a solution of 7-isopropyl-4-oxo-3,4-dihydroimidazo[1,5-f][1,2,4]triazine-2-carbaldehyde (3.3 g, 15.9 mmol) in methanol (20 mL) was added sodium borohydride (1.3 g, 31.2 mmol) by portions at room temperature. The reaction was stirred at rt for 2 hours. The mixture was filtered to remove any solids. The filtrate was concentrated to dryness and purified by reverse phase chromatography (elution with acetonitrile:water=10:90, 0.1% TFA) to give the title compound (2.9 g, 88%) as a light brown solid. LC/MS: m/e=209 (M+H)⁺.

### 2-(Chloromethyl)-7-isopropylimidazo[1,5-f][1,2,4]triazin-4(3H)-one

A suspension of 2-(hydroxymethyl)-7-isopropylimidazo[1,5-f][1,2,4]triazin-4(3H)-one (1.9 g, 9.1 mmol) in thionyl chloride (14 mL) was heated to 70° C. with stirring for 5 hours. The mixture was evaporated to dryness in vacuo to obtain the title compound as its hydro (1.5 g, 73%) as a light yellow solid. LC/MS: m/e=227 (M+H)⁺.

### 2-(4-Chlorophenylamino)ethanol

To a mixture of 1-chloro-4-iodobenzene (3.3 g, 14 mmol) in DMSO (4 mL) and water (1 mL) was added 2-aminoethanol (710 mg, 1.1.6 mmol), sodium hydroxide (1.1 g, 28 mmol) and cuprous iodide (315 mg, 1.6 mmol). The reaction was purged with nitrogen and stirred overnight at 90° C. Upon completion, the reaction was charged directly to a reverse-phase column (elution with acetonitrile/water=34/66, 0.1% ammonia), which afforded the title compound as a white oil (1.4 g, 58%). LC/MS: m/e=172 (M+H)⁺.

### 2-(((4-Chlorophenyl)(2-hydroxyethyl)amino)methyl)-7-isopropylimidazo[1,5-f][1,2,4]triazin-4(3H)-one

A mixture of 2-(chloromethyl)-7-isopropylimidazo[1,5-f][1,2,4]triazin-4(3H)-one (888 mg, 3.4 mmol) and 2-(4-chlorophenylamino)ethanol (1.13 g, 6.8 mmol) in acetonitrile (10 mL) was heated to 90° C. with stirring for 48 hours. The mixture was purified by reverse phase chromatography (elution with acetonitrile:water=40:60, 0.1% TFA) to give the title compound (540 mg, 48%) as a light brown solid. LC/MS: m/e=362 (M+H)⁺.

### Compound I-173

### 7-(3-Ethoxyphenyl)-3-isopropyl-8,9-dihydro-6H-imidazo[5,1-f]pyrazino[2,1-c][1,2,4]triazin-11(7H)-one

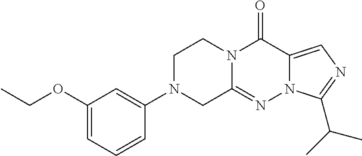 [SMILES: CCOc1cccc(N2CCn3c(nn4c(C(C)C)ncc4c3=O)C2)c1]

The title compound was prepared using the procedure for I-175, substituting 2-(3-ethoxyphenylamino)ethanol for 2-(4-chlorophenylamino)ethanol. ¹H NMR (400 MHz, CDCl₃): δ 7.86 (s, 1H), 7.24 (t, J=8.4 Hz, 1H), 6.52 (m, 2H), 6.46 (m, 1H), 4.42 (s, 2H), 4.19 (t, J=5.6 Hz, 2H), 4.06 (q, J=6.8 Hz, 2H), 3.67 (t, J=5.6 Hz, 2H), 3.52 (sept, J=6.8 Hz, 1H), 1.42 (m, 9H), LC/MS: m/e=354 (M+H)⁺.

### Compound I-174

### 3-Isopropyl-7-(3-methoxyphenyl)-8,9-dihydro-6H-imidazo[5,1-f]pyrazino[2,1-c][1,2,4]triazin-11(7H)-one

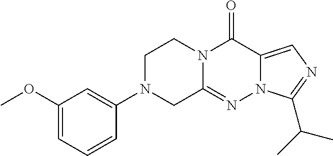 [SMILES: COc1cccc(N2CCn3c(nn4c(C(C)C)ncc4c3=O)C2)c1]

The title compound was prepared using the procedure for I-175, substituting 2-(3-methoxyphenylamino)ethanol for 2-(4-chlorophenylamino)ethanol. ¹H NMR (400 MHz, CDCl₃): δ 7.86 (s, 1H), 7.25 (t, J=8.4 Hz, 1H), 6.52 (m, 2H), 6.45 (m, 1H), 4.42 (s, 2H), 4.19 (t, J=5.6 Hz, 2H), 3.83 (s, 3H), 3.68 (t, J=6.0 Hz, 2H), 3.52 (sept, J=6.8 Hz, 1H), 1.41 (d, J=7.2 Hz, 6H), LC/MS: m/e=340 (M+H)⁺.

### Compound I-176

### 7-(4-Fluorophenyl)-3-isopropyl-8,9-dihydro-6H-imidazo[5,1-f]pyrazino[2,1-c][1,2,4]triazin-11(7H)-one

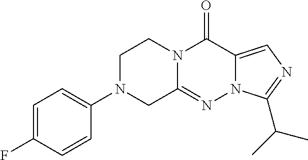 [SMILES: CC(C)c1ncc2c(=O)n3c(nn12)CN(c1ccc(F)cc1)CC3]

The title compound was prepared using the procedure for I-175, substituting 2-(4-fluorophenylamino)ethanol for 2-(4-chlorophenylamino)ethanol. ¹H NMR (400 MHz, CDCl₃): δ 7.87 (s, 1H), 7.06 (m, 2H), 6.92 (m, 2H), 4.34 (s, 2H), 4.16 (t, J=5.6 Hz, 2H), 3.62 (t, J=6.4 Hz, 2H), 3.52 (sept, J=6.8 Hz, 1H), 1.42 (d, J=6.8 Hz, 6H), LC/MS: m/e=328 (M+H)⁺.

### Compound I-177

### 7-(3-Ethoxy-4-fluorophenyl)-3-isopropyl-8,9-dihydro-6H-imidazo[5,1-f]pyrazino[2,1-c][1,2,4]triazin-11(7H)-one

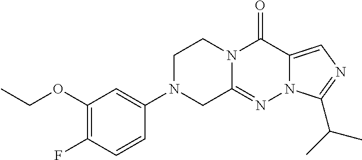 [SMILES: CCOc1cc(N2CCn3c(nn4c(C(C)C)ncc4c3=O)C2)ccc1F]

The title compound was prepared using the procedure for I-175, substituting 2-(3-ethoxy-4-fluorophenylamino)ethanol for 2-(4-chlorophenylamino)ethanol. ¹H NMR (400 MHz, CDCl₃): δ 7.86 (s, 1H), 7.04 (dd, J₁=10.8 Hz, J₂=8.8 Hz, 1H), 6.56 (dd, J₁=6.8 Hz, J₂=2.8 Hz, 1H), 6.44 (dt, J₁=8.8 Hz, J₂=3.2 Hz, 1H), 4.32 (s, 2H), 4.15 (m, 4H), 3.61 (t, J=5.6 Hz, 2H), 3.51 (sept, J=7.2 Hz, 1H), 1.48 (e, J=7.2 Hz, 3H), 1.41 (d, J=8.0 Hz, 6H), LC/MS: m/e=372 (M+H)⁺.

### Compound I-178

### 3-Isopropyl-7-(4-methoxybenzyl)-8,9-dihydro-6H-imidazo[5,1-f]pyrazino[2,1-c][1,2,4]triazin-11(7H)-one

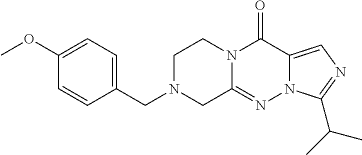 [SMILES: COc1ccc(CN2CCn3c(nn4c(C(C)C)ncc4c3=O)C2)cc1]

The title compound was prepared using the procedure for I-175, substituting 2-(4-methoxybenzylamino)ethanol for 2-(4-chlorophenylamino)ethanol. ¹H NMR (400 MHz, CDCl₃): δ 7.81 (s, 1H), 7.27 (d, J=8.8 Hz, 2H), 6.91 (d, J=8.8 Hz, 2H), 3.94 (t, 0.7=6.0 Hz, 2H), 3.83 (s, 3H), 3.64 (s, 2H), 3.57 (s, 2H), 3.44 (sept, J=7.2 Hz, 1H), 2.86 (t, J=5.6 Hz, 2H), 1.36 (d, J=7.2 Hz, 6H), LC/MS: m/e=354 (M+H)⁺.

### Compound I-179

### 7-(4-Chlorobenzyl)-3-isopropyl-8,9-dihydro-6H-imidazo[5,1-f]pyrazino[2,1-c][1,2,4]triazin-11(7H)-one

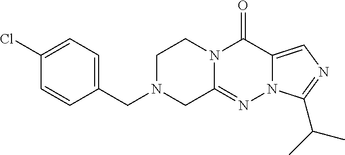 [SMILES: CC(C)c1ncc2c(=O)n3c(nn12)CN(Cc1ccc(Cl)cc1)CC3]

The title compound was prepared using the procedure for I-175, substituting 2-(4-chlorobenzylamino)ethanol for 2-(4-chlorophenylamino)ethanol. ¹H NMR (400 MHz, CDCl₃): δ 7.82 (s, 1H), 7.37-7.30 (m, 4H), 3.95 (t, J=5.6 Hz, 2H), 3.67 (s, 2H), 3.57 (s, 2H), 3.45 (sept, J=7.2 Hz, 1H), 2.88 (t, J=5.6 Hz, 2H), 1.37 (d, J=7.2 Hz, 6H), LC/MS: m/e=358 (M+H)⁺.

### Compound I-180

### 3-Cyclopentyl-7-(3-ethoxyphenyl)-8,9-dihydro-6H-imidazo[5,1-f]pyrazino[2,1-c][1,2,4]triazin-11(7H)-one

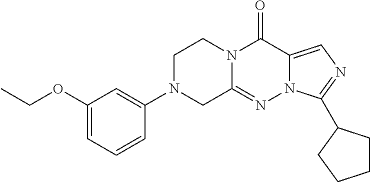 [SMILES: CCOc1cccc(N2CCn3c(nn4c(C5CCCC5)ncc4c3=O)C2)c1]

The title compound was prepared using the procedure for I-175, substituting cyclopentane-carbaldehyde for isobutyraldehyde, and substituting 2-(3-ethoxyphenylamino)ethanol for 2-(4-chlorophenylamino)ethanol. ¹H NMR (400 MHz, CDCl₃): δ 7.85 (s, 1H), 7.23 (t, J=8.0 Hz, 1H), 6.51 (m, 2H), 6.46 (m, 1H), 4.42 (s, 2H), 4.18 (t, J=5.6 Hz, 2H), 4.06 (q, J=6.8 Hz, 2H), 3.66 (t, J=5.6 Hz, 2H), 3.58 (quint, J=8.4 Hz, 1H), 2.14 (m, 2H), 2.00-1.83 (m, 4H), 1.72 (m, 2H), 1.48 (t, J=8.4 Hz, 3H), LC/MS: m/e=380 (M+H)⁺.

### Compound I-181

### 3-Cyclopentyl-7-(3-methoxyphenyl)-8,9-dihydro-6H-imidazo[5,1-f]pyrazino[2,1-c][1,2,4]triazin-11(7H)-one

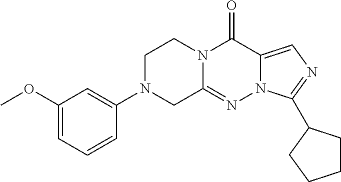 [SMILES: COc1cccc(N2CCn3c(nn4c(C5CCCC5)ncc4c3=O)C2)c1]

The title compound was prepared using the procedure for I-175, substituting cyclopentane-carbaldehyde for isobutyraldehyde, and substituting 2-(3-methoxyphenylamino)ethanol for 2-(4-chlorophenylamino)ethanol. ¹H NMR (400 MHz, CDCl₃): δ 7.85 (s, 1H), 7.25 (t, J=8.4 Hz, 1H), 6.52 (m, 2H), 6.45 (m, 1H), 4.42 (s, 2H), 4.19 (t, J=6.0 Hz, 2H), 3.83 (s, 3H), 3.68 (t, J=5.6 Hz, 2H), 3.58 (quint, J=8.4 Hz, 1H), 2.12 (m, 2H), 2.00-1.84 (m, 4H), 1.72 (m, 2H), LC/MS: m/e=366 (M+H)⁺.

### Compound I-183

### 3-Cyclopentyl-7-(4-fluorophenyl)-8,9-dihydro-6H-imidazo[5,1-f]pyrazino[2,1-c][1,2,4]triazin-11(7H)-one

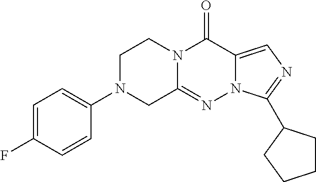 [SMILES: O=c1c2cnc(C3CCCC3)n2nc2n1CCN(c1ccc(F)cc1)C2]

The title compound was prepared using the procedure for I-175, substituting cyclopentane-carbaldehyde for isobutyraldehyde, and substituting 2-(4-fluorophenylamino)ethanol for 2-(4-chlorophenylamino)ethanol. ¹H NMR (400 MHz, CDCl₃): δ 7.86 (s, 1H), 7.05 (m, 2H), 6.92 (m, 2H), 4.34 (s, 2H), 4.15 (t, J=6.0 Hz, 2H), 3.62 (t, J=6.4 Hz, 2H), 3.58 (quint, J=8.0 Hz, 1H), 2.13 (m, 2H), 2.00-1.85 (m, 4H), 1.73 (m, 2H), LC/MS: m/e=354 (M+H)⁺.

### Compound I-184

### 3-Cyclopentyl-7-(3-ethoxy-4-fluorophenyl)-8,9-dihydro-6H-imidazo[5,1-f]pyrazino[2,1-c][1,2,4]triazin-11(7H)-one

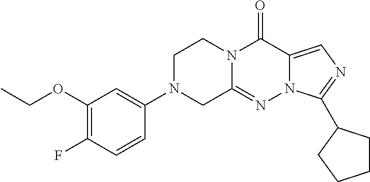 [SMILES: CCOc1cc(N2CCn3c(nn4c(C5CCCC5)ncc4c3=O)C2)ccc1F]

The title compound was prepared using the procedure for I-175, substituting cyclopentane-carbaldehyde for isobutyraldehyde, and substituting 2-(3-ethoxy-4-fluorophenylamino)ethanol for 2-(4-chlorophenylamino)ethanol. ¹H NMR (400 MHz, CDCl₃): δ 7.85 (s, 1H), 7.04 (t, J=9.2 Hz, 1H), 6.56 (d, J=4.8 Hz, 1H), 6.43 (d, J=4.8 Hz, 1H), 4.32 (s, 2H), 4.14 (m, 4H), 3.60 (m, 3H), 2.13 (m, 2H), 2.03-1.82 (m, 4H), 1.73 (m, 2H), 1.48 (t, J=7.6 Hz, 3H), LC/MS: m/e=398 (M+H)⁺.

### Compound I-229

### (±)-7-Phenyl-3-(tetrahydrofuran-3-yl)-8,9-dihydro-6H-imidazo[5,1-f]pyrazino[2,1-c][1,2,4]triazin-11(7H)-one

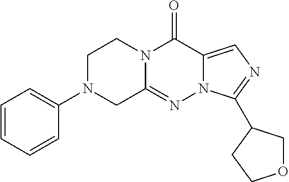 [SMILES: O=c1c2cnc(C3CCOC3)n2nc2n1CCN(c1ccccc1)C2]

The title compound was prepared using the procedure for I-175, substituting (±)-tetrahydrofuran-3-carbaldehyde for isobutyraldehyde, and substituting 2-phenylaminoethanol for 2-(4-chlorophenylamino)ethanol. ¹H NMR (400 MHz, CDCl₃): δ 7.88 (s, 1H), 7.37 (t, J=8.0 Hz, 2H), 6.99 (m, 3H), 4.44 (s, 2H), 4.27 (m, 1H), 4.22 (t, J=5.6 Hz, 2H), 4.13 (dd, J₁=14.0 Hz, J₂=8.0 Hz, 1H), 4.00 (m, 3H), 3.71 (t, J=6.0 Hz, 2H), 2.45 (m, 2H), LC/MS: m/e=338 (M+H)⁺.

### Compound I-230

### (±)-7-(4-Chlorophenyl)-3-(tetrahydrofuran-3-yl)-8,9-dihydro-6H-imidazo[5,1-f]pyrazino[2,1-c][1,2,4]triazin-11(7H)-one

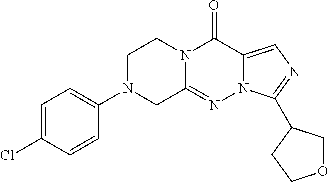 [SMILES: O=c1c2cnc(C3CCOC3)n2nc2n1CCN(c1ccc(Cl)cc1)C2]

The title compound was prepared using the procedure for I-175, substituting (±)-tetrahydrofuran-3-carbaldehyde for isobutyraldehyde. ¹H NMR (400 MHz, CDCl₃): δ 7.88 (s, 1H), 7.31 (d, J=9.2 Hz, 2H), 6.86 (d, J=9.2 Hz, 2H), 4.41 (s, 2H), 4.26 (t, J=7.2 Hz, 1H), 4.22 (t, J=6.0 Hz, 2H), 4.11 (dd, J₁=14.0 Hz, J₂=8.0 Hz, 1H), 3.98 (m, 3H), 3.67 (t, J=5.6 Hz, 2H), 2.45 (m, 2H), LC/MS: m/e=338 (M+H)⁺.

### Compound I-236

### 7-(4-Chlorophenyl)-3-(tetrahydro-2H-pyran-4-yl)-8,9-dihydro-6H-imidazo[5,1-f]pyrazino[2,1-c][1,2,4]triazin-11(7H)-one

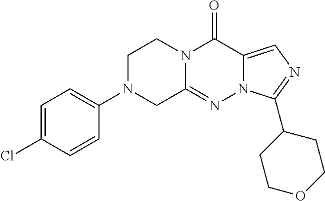 [SMILES: O=c1c2cnc(C3CCOCC3)n2nc2n1CCN(c1ccc(Cl)cc1)C2]

The title compound was prepared using the procedure for I-175, substituting tetrahydro-2H-pyran-4-carbaldehyde for isobutyraldehyde. ¹H NMR (400 MHz, d⁶-DMSO): δ 7.76 (s, 1H), 7.27 (d, J=8.8 Hz, 2H), 6.97 (d, J=9.2 Hz, 2H), 4.47 (s, 2H), 4.09 (t, J=5.6 Hz, 2H), 3.95 (d, J=11.2 Hz, 2H), 3.64 (d, J=5.6 Hz, 2H), 3.49 (m, 2H), 3.40 (m, 1H), 1.84 (m, 4H), LC/MS: m/e=386 (M+H)⁺.

### Compound I-238

### 4-(3-Isopropyl-11-oxo-8,9-dihydro-6H-imidazo[5,1-f]pyrazino[2,1-c][1,2,4]triazin-7(11H)-yl)benzonitrile

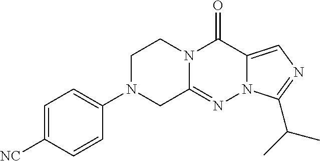 [SMILES: CC(C)c1ncc2c(=O)n3c(nn12)CN(c1ccc(C#N)cc1)CC3]

The title compound was prepared using the procedure for I-175, substituting 4-((2-hydroxyethyl)amino)benzonitrile for 2-(4-chlorophenylamino)ethanol. ¹H NMR (400 MHz, CDCl₃): δ 7.86 (s, 1H), 7.58 (d, J=8.4 Hz, 2H), 6.84 (d, J=8.8 Hz, 2H), 4.57 (s, 2H), 4.35 (t, J=5.6 Hz, 2H), 3.75 (t, J=5.2 Hz, 2H), 3.50 (sept, J=6.8 Hz, 1H), 1.40 (d, J=6.8 Hz, 6H), LC/MS: m/e=335 (M+H)⁺.

### Compound I-240

### 3-Isopropyl-7-(4-trifluoromethyl)phenyl)-8,9-dihydro-6H-imidazo[5,1-f]pyrazino[2,1-c][1,2,4]triazin-11(7H)-one

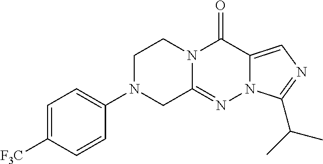 [SMILES: CC(C)c1ncc2c(=O)n3c(nn12)CN(c1ccc(C(F)(F)F)cc1)CC3]

The title compound was prepared using the procedure for I-175, substituting 2-(4-trifluoromethylphenylamino)ethanol for 2-(4-chlorophenylamino)ethanol. ¹H NMR (400 MHz, CDCl₃): δ 7.87 (s, 1H), 7.58 (d, J=8.4 Hz, 2H), 6.91 (d, J=8.4 Hz, 2H), 4.54 (s, 2H), 4.31 (t, J=6.0 Hz, 2H), 3.75 (t, J=6.0 Hz, 2H), 3.51 (m, 1H), 1.42 (d, J=6.8 Hz, 6H), LC/MS: m/e 378 (M+H)⁺.

### Compound I-249

### 7-(4-Chlorophenyl)-3-cyclobutyl-8,9-dihydro-6H-imidazo[5,1-f]pyrazino[2,1-c][1,2,4]triazin-11(7H)-one

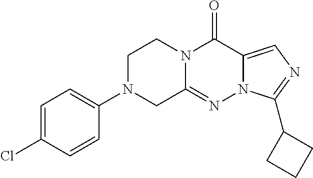 [SMILES: O=c1c2cnc(C3CCC3)n2nc2n1CCN(c1ccc(Cl)cc1)C2]

The title compound was prepared using the procedure for I-175, substituting cyclobutane-carbaldehyde for isobutyraldehyde. ¹H NMR (400 MHz, CDCl₃): δ 7.89 (s, 1H), 7.30 (m, 2H), 6.85 (m, 2H), 4.39 (s, 2H), 4.20 (t, J=5.6 Hz, 2H), 4.02 (quint, J=8.8 Hz, 1H), 3.65 (t, J=6.0 Hz, 2H), 2.56 (m, 2H), 2.43 (m, 2H), 2.13 (m, 1H), 2.03 (m, 1H), LC/MS: m/e 356 (M+H)⁺.

### Compound I-242

### 7-(5-Chloropyridin-2-yl)-3-isopropyl-8,9-dihydro-6H-imidazo[5,1-f]pyrazino[2,1-c][1,2,4]triazin-11(7H)-one

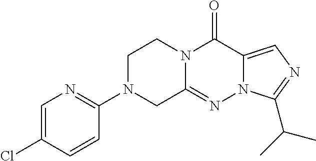 [SMILES: CC(C)c1ncc2c(=O)n3c(nn12)CN(c1ccc(Cl)cn1)CC3]

The title compound was prepared using the procedure for I-175, substituting 2-((6-chloropyridin-3-yl)amino)ethanol for 2-(4-chlorophenylamino)ethanol. ¹H NMR (400 MHz, CDCl₃): δ 8.21 (d, J=2.0 Hz, 1H), 7.88 (s, 1H), 7.55 (dd, J₁=8.8 Hz, J₂=2.4 Hz, 1H), 6.59 (d, J=8.8 Hz, 1H), 4.83 (s, 2H), 4.33 (t, J=6.0 Hz, 2H), 3.84 (t, J=5.6 Hz, 2H), 3.54 (m, 1H), 1.42 (d, J=6.8 Hz, 6H), LC/MS: m/e=345 (M+H)⁺.

### Compound I-251

### 7-(4-Chlorophenyl)-3-cyclopropyl-8,9-dihydro-6H-imidazo[5,1-f]pyrazino[2,1-c][1,2,4]triazin-11(7H)-one

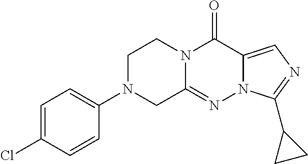 [SMILES: O=c1c2cnc(C3CC3)n2nc2n1CCN(c1ccc(Cl)cc1)C2]

The title compound was prepared using the procedure for I-175, substituting cyclopropane-carbaldehyde for isobutyraldehyde. ¹H NMR (400 MHz, CDCl₃): δ 7.79 (s, 1H), 7.31 (m, 2H), 6.86 (m, 2H), 4.42 (s, 2H), 4.20 (t, J=5.6 Hz, 2H), 3.66 (t, J=5.6 Hz, 2H), 2.41 (m, 1H), 1.21 (m, 2H), 1.12 (m, 2H), LC/MS: m/e=342 (M+H)⁺.

### Compound I-250

### 3-(tert-Butyl)-7-(4-chlorophenyl)-8,9-dihydro-6H-imidazo[5,1-f]pyrazino[2,1-c][1,2,4]triazin-11(7H)-one

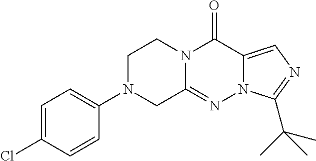 [SMILES: CC(C)(C)c1ncc2c(=O)n3c(nn12)CN(c1ccc(Cl)cc1)CC3]

The title compound was prepared using the procedure for I-175, substituting pivalaldehyde for isobutyraldehyde. ¹H NMR (400 MHz, CDCl₃): δ 7.84 (s, 1H), 7.31 (d, J=9.2 Hz, 2H), 6.86 (d, J=8.8 Hz, 2H), 4.41 (s, 2H), 4.22 (t, J=5.6 Hz, 2H), 3.67 (t, J=5.6 Hz, 2H), 1.55 (s, 9H), LC/MS: m/e=358 (M+H)⁺.

### Compound I-252

### 7-(4-Chlorophenyl)-3-(piperidin-4-yl)-8,9-dihydro-6H-imidazo[5,1-f]pyrazino[2,1-c][1,2,4]triazin-11(7H)-one

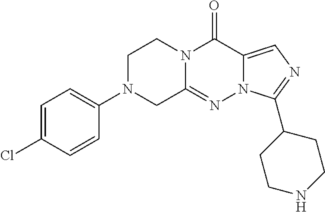 [SMILES: O=c1c2cnc(C3CCNCC3)n2nc2n1CCN(c1ccc(Cl)cc1)C2]

The title compound was prepared in a manner similar to I-175, substituting 1-benzylpiperidine-4-carbaldehyde for isobutyraldehyde. ¹H NMR (400 MHz, d⁴-MeOH): δ 7.77 (s, 1H), 7.29 (m, 2H), 7.00 (m, 2H), 4.48 (s, 2H), 4.19 (t, J=5.6 Hz, 2H), 3.73 (t, J=6.0 Hz, 2H), 3.47 (m, 1H), 3.22 (m, 2H), 2.83 (m, 2H), 2.04-1.91 (m, 4H), LC/MS: m/e=385 (M+H)⁺.

### Example 14

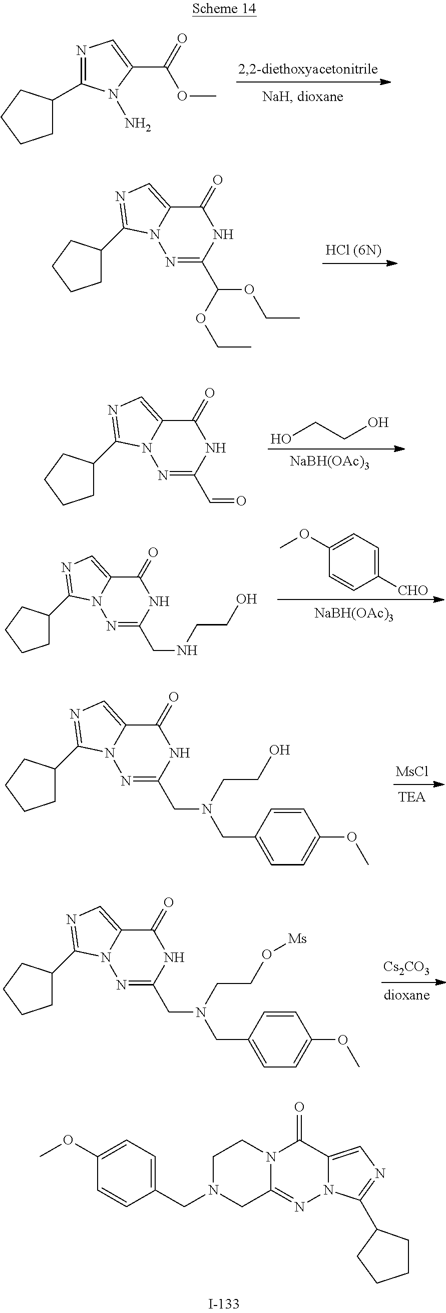

### Compound I-133

### 3-Cyclopentyl-7-(4-methoxybenzyl)-8,9-dihydro-6H-imidazo[5,1-f]pyrazino[2,1-c][1,2,4]triazin-11(7H)-one

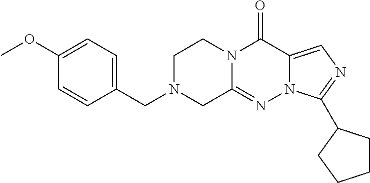 [SMILES: COc1ccc(CN2CCn3c(nn4c(C5CCCC5)ncc4c3=O)C2)cc1]

7-Cyclopentyl-2-(((2-hydroxyethyl)(4-methoxybenzyl)amino)methyl)imidazo[1,5-f][1,2,4]triazin-4(3H)-one (48 mg, 0.1 mmol) and cesium carbonate (98 mg, 0.3 mmol) were combined in dioxane (5 mL), and heated to reflux for 1 hour. Upon completion, the mixture was filtered and purified by prep-HPLC to give the title compound (16.7 mg, 44%) as a yellow solid. ¹H. ¹H NMR (400 MHz, CDCl₃): δ 7.80 (s, 1H), 7.27 (d, J=8.4 Hz, 2H), 6.91 (d, J=8.8 Hz, 2H), 3.93 (t, J=6.0 Hz, 2H), 3.83 (s, 3H), 3.64 (s, 2H), 3.56 (s, 2H), 3.51 (quint, J=8.0 Hz, 1H), 2.86 (t, 5.6 Hz, 2H), 2.08 (m, 2H), 1.96-1.82 (m, 4H), 1.69 (m, 2H), LC/MS: m/e=380 (M+H)⁺.

### 7-Cyclopentyl-2-(diethoxymethyl)imidazo[1,5-f][1,2,4]triazin-4(3H)-one

To a mixture of methyl 1-amino-2-cyclopentyl-1H-imidazole-5-carboxylate (1.5 g, 7 mmol) and 2,2-diethoxyacetonitrile (1.85 g, 14 mmol) in dry dioxane (30 mL) was added sodium hydride (1.14 g, 28.7 mmol) in small portions. The mixture was stirred at 60° C. for 3 hours. Upon completion, water (5 mL) was added, and the solvent was removed in vaeuo. The residue was diluted with water (15 mL), adjusted to pH 5 with hydrochloric acid, and extracted with ethyl acetate (30 mL×3). The combined organic extracts were dried and concentrated. Purification by silica gel chromatography (elution form dichloromethane to dichloromethane:methanol=100:1) gave the title compound (1.48 g, 69%) as a yellow oil. LC/MS: m/e=307 (M+H)⁺.

### 7-Cyclopentyl-4-oxo-3,4-dihydroimidazo[1,5-f][1,2,4]triazine-2-carbaldehyde

A mixture of 7-cyclopentyl-2-(diethoxymethyl)imidazo[1,5-f][1,2,4]triazin-4(3H)-one (1.48 g, 4.8 mmol) in hydrochloric acid (6N, 6 mL) and tetrahydrofuran (2 mL) was heated to reflux for 4 hours. Upon completion, the solvent was removed, and the crude material (1.12 g, 92%) used without further purification for next step. LC/MS: m/e=251 (M+H)⁺.

### 7-Cyclopentyl-2-((2-hydroxyethylamino)methyl)imidazo[1,5-f][1,2,4]triazin-4(3H)-one

A mixture of 7-cyclopentyl-4-oxo-3,4-dihydroimidazo[1,5-f][1,2,4]triazine-2-carbaldehyde (1.12 g, 4.8 mmol), 2-aminoethanol (1.77 g, 29 mmol) and sodium triacetoxyborohydride (6.14 g, 29 mmol) in dichloromethane (30 mL) was stirred at room temperature overnight. Upon completion, the mixture was purified by reverse phase chromatography (elution from water to water:acetonitrile=100:10) to give the title compound (322 mg, 24%) as a yellow oil. LC/MS: m/e=278 (M+H)⁺.

### 7-Cyclopentyl-2-(((2-hydroxyethyl)(4-methoxybenzyl)amino)methyl)imidazo[1,5-f][1,2,4]triazin-4(3H)-one

A mixture of 7-cyclopentyl-2-((2-hydroxyethylamino)methyl)imidazo[1,5-f][1,2,4]triazin-4(3H)-one (78 mg, 0.28 mmol), 4-methoxybenzaldehyde (115 mg, 0.84 mmol) and sodium triacetoxyborohydride (358 mg, 1.69 mmol) in dichloromethane (10 mL) was stirred at room temperature for 16 h. Upon completion, the mixture was washed with water (10 mL×2), dried and concentrated to dryness. The crude material was purified by reverse phase chromatography (elution from water to wateracetonitrile=100:30) to give the title compound (40 mg, 35%) as a white solid. LC/MS: m/e=398 (M+H)⁺.

### 7-Cyclopentyl-2-(((2-hydroxyethyl)(4-methoxybenzyl)amino)methyl)imidazo[1,5-f][1,2,4]triazin-4(3H)-one

To a solution of 7-cyclopentyl-2-(((2-hydroxyethyl)(4-methoxybenzyl)amino)methyl)imidazo[1,5-f][1,2,4]triazin-4(3H)-one (40 mg, 0.1 mmol) and triethylamine (31 mg, 0.3 mmol) in dichloromethane (5 mL) was added methanesulfonyl chloride (17 mg, 0.15 mmol) slowly at 0° C. The mixture was stirred for 1 hour at room temperature. Upon completion, the reaction mixture was washed with water (10 mL×2), dried and concentrated to give the title compound (48 mg, 100%) which was used without further purification. LC/MS: m/e=476 (M+H)⁺.

### Compound I-134

### 7-(4-Chlorobenzyl)-3-cyclopentyl-8,9-dihydro-6H-imdazo[5,1-f]pyrazino[2,1-c][1,2,4]triazin-11(7H)-one

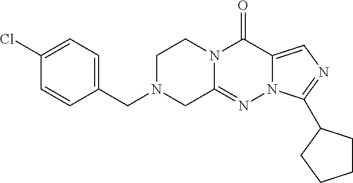 [SMILES: O=c1c2cnc(C3CCCC3)n2nc2n1CCN(Cc1ccc(Cl)cc1)C2]

The title compound was prepared using the procedure for I-133, substituting 4-chlorobenzaldehyde for 4-methoxybenzaldehyde. ¹H NMR (400 MHz, CDCl₃): δ 7.80 (s, 1H), 7.37-7.27 (m, 4H), 3.94 (t, J=6.0 Hz, 2H), 3.66 (s, 2H), 3.56 (s, 2H), 3.50 (quint, J=8.0 Hz, 1H), 2.88 (t, J=5.6 Hz, 2H), 2.08 (m, 2H), 1.96-1.80 (m, 4H), 1.69 (m, 2H), LC/MS: m/e=384 (M+H)⁺.

### Compound I-182

### 7-(4-Chlorophenyl)-3-cyclopentyl-8,9-dihydro-6H-imidazo[5,1-f]pyrazino[2,1-c][1,2,4]triazin-11(7H)-one

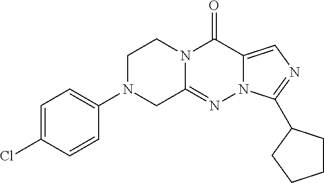 [SMILES: O=c1c2cnc(C3CCCC3)n2nc2n1CCN(c1ccc(Cl)cc1)C2]

The title compound was prepared in a manner similar to the procedure for I-133, substituting 4-chloroiodobenzene and cuprous iodide for 4-methoxybenzaldehyde and sodium triacetoxyborohydride. ¹H NMR (400 MHz, CDCl₃): δ 7.89 (s, 1H), 7.31 (m, 2H), 6.85 (m, 2H), 4.40 (s, 2H), 4.21 (t, J=6.0 Hz, 2H), 3.66 (t, J=6.0 Hz, 2H), 3.61 (quint, J=4.4 Hz, 1H), 2.15 (m, 2H), 2.10-1.85 (m, 4H), 1.74 (m, 2H), LC/MS: m/e=370 (M+H)⁺.

### Compound I-185

### 3-Cyclopentyl-7-(pyridin-4-yl)-8,9-dihydro-6H-imidazo[5,1-f]pyrazino[2,1-c][1,2,4]triazin-11(7H)-one

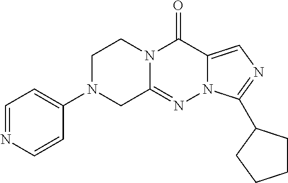 [SMILES: O=c1c2cnc(C3CCCC3)n2nc2n1CCN(c1ccncc1)C2]

The title compound was prepared in a manner similar to the procedure for I-133, substituting 4-iodopyridine and cuprous iodide for 4-methoxybenzaldehyde and sodium triacetoxyborohydride. ¹H NMR (400 MHz, CDCl₃): δ 8.38 (m, 2H), 7.89 (s, 1H), 6.74 (d, J=4.4 Hz, 2H), 4.60 (s, 2H), 4.40 (t, J=6.0 Hz, 2H), 3.77 (t, J=6.0 Hz, 2H), 3.57 (quint, J=4.4 Hz, 1H), 2.13 (m, 4H), 1.96 (m, 3H), 1.73 (m, 1H), LC/MS: m/e=337 (M+H)⁺.

### Compound I-188

### 3-Isopropyl-7-((6-methylpyridin-3-yl)methyl)-8,9-dihydro-6H-imidazo[5,1-f]pyrazino[2,1-c][1,2,4]triazin-11(7H)-one

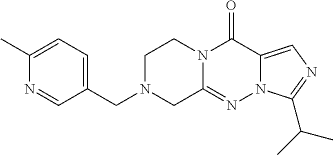 [SMILES: Cc1ccc(CN2CCn3c(nn4c(C(C)C)ncc4c3=O)C2)cn1]

The title compound was prepared using the procedure for I-133, substituting methyl 1-amino-2-isopropyl-1H-imidazole-5-carboxylate for methyl 1-amino-2-cyclopentyl-1H-imidazole-5-carboxylate, and substituting 6-methylnicotinaldehyde for 4-methoxybenzaldehyde. ¹H NMR (400 MHz, CDCl₃): δ 8.48 (s, 1H), 7.83 (s, 1H), 7.62 (dd, J₁=8.0 Hz, J₂=1.6 Hz, 1H), 7.20 (d, J=7.6 Hz, 1H), 3.96 (t, J=5.6 Hz, 2H), 3.69 (s, 2H), 3.60 (s, 2H), 3.45 (sept, J=6.8 Hz, 1H), 2.90 (t, J=5.6 Hz, 2H), 2.60 (s, 3H), 1.38 (d, J=6.8 Hz, 6H), LC/MS: m/e=339 (M+H)⁺.

### Compound I-189

### 3-Cyclopentyl-7-((6-methylpyridin-3-yl)methyl)-8,9-dihydro-6H-imidazo[5,1-f]pyrazino[2,1-c][1,2,4]triazin-11(7H)-one

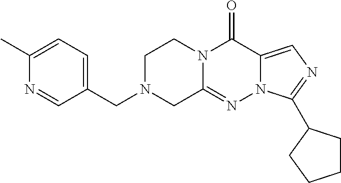 [SMILES: Cc1ccc(CN2CCn3c(nn4c(C5CCCC5)ncc4c3=O)C2)cn1]

The title compound was prepared using the procedure for I-133, substituting 6-methylnicotinaldehyde for 4-methoxybenzaldehyde. ¹H NMR (400 MHz, CDCl₃): δ 8.48 (s, 1H), 7.81 (s, 1H), 7.62 (dd, J₁=8.0 Hz, J₂=1.6 Hz, 1H), 7.20 (d, J=8.0 Hz, 1H), 3.95 (t, J=5.6 Hz, 2H), 3.68 (s, 2H), 3.59 (s, 2H), 3.50 (m, 1H), 2.89 (t, J=5.6 Hz, 2H), 2.60 (s, 3H), 2.08 (m, 2H), 1.97-1.82 (m, 4H), 1.70 (m, 2H), LC/MS: m/e=365 (M+H)⁺.

### Compound I-192

### 3-Isopropyl-7-(pyrimidin-5-ylmethyl)-8,9-dihydro-6H-imidazo[5,1-f]pyrazino[2,1-c][1,2,4]triazin-11(7H)-one

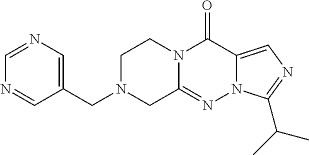 [SMILES: CC(C)c1ncc2c(=O)n3c(nn12)CN(Cc1cncnc1)CC3]

The title compound was prepared using the procedure for I-133, substituting methyl 1-amino-2-isopropyl-1H-imidazole-5-carboxylate for methyl 1-amino-2-cyclopentyl-1H-imidazole-5-carboxylate, and substituting pyrimidine-5-carbaldehyde for 4-methoxybenzaldehyde. ¹H NMR (400 MHz, CDCl₃): δ 9.23 (s, 1H), 8.79 (s, 2H), 7.84 (s, 1H), 3.99 (t, J=5.6 Hz, 2H), 3.75 (s, 2H), 3.64 (s, 2H), 3.45 (sept, J=6.8 Hz, 1H), 2.95 (t, J=5.6 Hz, 2H), 1.38 (d, J=6.8 Hz, 6H), LC/MS: m/e=326 (M+H)⁺.

### Compound I-193

### 3-Cyclopentyl-7-(pyrimidin-5-ylmethyl)-8,9-dihydro-6H-imidazo[5,1-f]pyrazino[2,1-c][1,2,4]triazin-11(7H)-one

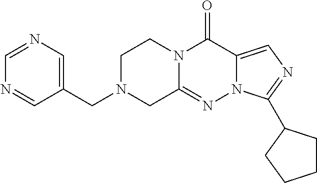 [SMILES: O=c1c2cnc(C3CCCC3)n2nc2n1CCN(Cc1cncnc1)C2]

The title compound was prepared using the procedure for I-133, substituting pyrimidine-5-carbaldehyde for 4-methoxybenzaldehyde. ¹H NMR (400 MHz, CDCl₃): δ 9.24 (s, 1H), 8.79 (s, 2H), 7.84 (s, 1H), 3.99 (t, J=5.6 Hz, 2H), 3.75 (s, 2H), 3.63 (s, 2H), 3.52 (quint, J=8.0 Hz, 1H), 2.95 (t, J=5.6 Hz, 2H), 2.10 (m, 2H), 1.97-1.73 (m, 4H), 1.71 (m, 2H), LC/MS: m/e=352 (M+H)⁺.

### Compound I-196

### 3-Isopropyl-7-((2-methylpyrimidin-5-yl)methyl)-8,9-dihydro-6H-imidazo[5,1-f]pyrazino[2,1-c][1,2,4]triazin-11(7H)-one

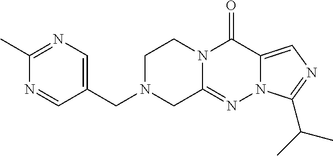 [SMILES: Cc1ncc(CN2CCn3c(nn4c(C(C)C)ncc4c3=O)C2)cn1]

The title compound was prepared using the procedure for I-133, substituting methyl 1-amino-2-isopropyl-1H-imidazole-5-carboxylate for methyl 1-amino-2-cyclopentyl-1H-imidazole-5-carboxylate, and substituting 2-methylpyrimidine-5-carbaldehyde for 4-methoxybenzaldehyde. ¹H NMR (400 MHz, CDCl₃): δ 8.66 (s, 2H), 7.83 (s, 1H), 3.97 (t, J=5.6 Hz, 2H), 3.70 (s, 2H), 3.62 (s, 2H), 3.45 (sept, J=6.8 Hz, 1H), 2.93 (t, J=5.6 Hz, 2H), 2.79 (s, 3H), 1.38 (d, J=7.2 Hz, 6H), LC/MS: m/e=340 (M+H)⁺.

### Compound I-197

### 3-Cyclopentyl-7-((2-methylpyrimidin-5-yl)methyl)-8,9-dihydro-6H-imidazo[5,1-f]pyrazino[2,1-c][1,2,4]triazin-11(7H)-one

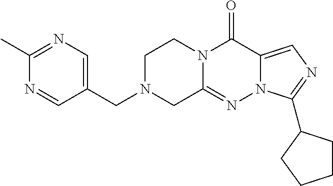 [SMILES: Cc1ncc(CN2CCn3c(nn4c(C5CCCC5)ncc4c3=O)C2)cn1]

The title compound was prepared using the procedure for I-133, substituting 2-methylpyrimidine-5-carbaldehyde for 4-methoxybenzaldehyde. ¹H NMR (400 MHz, CDCl₃): δ 8.66 (s, 2H), 7.82 (s, 1H), 3.97 (t, J=5.6 Hz, 2H), 3.69 (s, 2H), 3.61 (s, 2H), 3.51 (m, 1H), 2.92 (t, J=6.0 Hz, 2H), 2.78 (s, 3H), 2.08 (m, 2H), 1.95-1.75 (m, 4H), 1.70 (m, 2H), LC/MS: m/e 366 (M+H)⁺.

### Compound I-204

### 3-Isopropyl-7-(pyrazin-2-ylmethyl)-8,9-dihydro-6H-imidazo[5,1-f]pyrazino[2,1-c][1,2,4]triazin-11(7H)-one

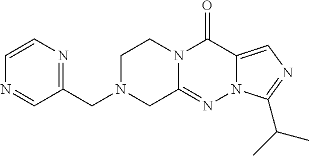 [SMILES: CC(C)c1ncc2c(=O)n3c(nn12)CN(Cc1cnccn1)CC3]

The title compound was prepared using the procedure for I-133, substituting methyl 1-amino-2-isopropyl-1H-imidazole-5-carboxylate for methyl 1-amino-2-cyclopentyl-1H-imidazole-5-carboxylate, and substituting pyrazine-2-carbaldehyde for 4-methoxybenzaldehyde. ¹H NMR (400 MHz, CDCl₃): δ 8.75 (s, 1H), 8.62 (m, 1H), 8.58 (m, 1H), 7.83 (s, 1H), 4.01 (t, J=5.6 Hz, 2H), 3.94 (s, 2H), 3.72 (s, 2H), 3.46 (sept, J=7.2 Hz, 1H), 3.02 (t, J=6.0 Hz, 2H), 1.38 (d, J=6.8 Hz, 6H), LC/MS: m/e=326 (M+H)⁺.

### Compound I-205

### 3-Cyclopentyl-7-(pyrazin-2-ylmethyl)-8,9-dihydro-6H-imidazo[5,1-f]pyrazino[2,1-c][1,2,4]triazin-11(7H)-one

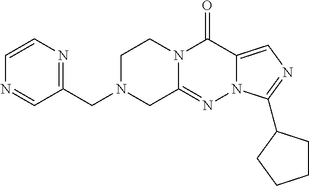 [SMILES: O=c1c2cnc(C3CCCC3)n2nc2n1CCN(Cc1cnccn1)C2]

The title compound was prepared using the procedure for I-133, substituting pyrazine-2-carbaldehyde for 4-methoxybenzaldehyde. ¹H NMR (400 MHz, CDCl₃): δ 8.75 (s, 1H), 8.61 (m, 1H), 8.58 (m, 1H), 7.82 (s, 1H), 4.00 (t, J=5.6 Hz, 2H), 3.93 (s, 2H), 3.71 (s, 2H), 3.45 (quint, J=8.0 Hz, 1H), 3.01 (t, J=5.6 Hz, 2H), 2.11 (m, 2H), 1.95-1.80 (m, 4H), 1.71 (m, 2H), LC/MS: m/e=352 (M+H)⁺.

### Compound I-208

### 3-Isopropyl-7-((5-methylpyrazin-2-yl)methyl)-8,9-dihydro-6H-imidazo[5,1-f]pyrazino[2,1-c][1,2,4]triazin-11(7H)-one

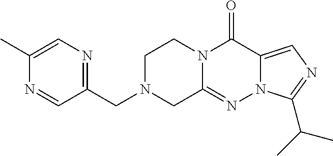 [SMILES: Cc1cnc(CN2CCn3c(nn4c(C(C)C)ncc4c3=O)C2)cn1]

The title compound was prepared using the procedure for I-133, substituting methyl 1-amino-2-isopropyl-1H-imidazole-5-carboxylate for methyl 1-amino-2-cyclopentyl-1H-imidazole-5-carboxylate, and substituting 5-methylpyrazine-2-carbaldehyde for 4-methoxybenzaldehyde. ¹H NMR (400 MHz, CDCl₃): δ 8.59 (s, 1H), 8.49 (s, 1H), 7.83 (s, 1H), 3.99 (t, J=6.0 Hz, 2H), 3.88 (s, 2H), 3.70 (s, 2H), 3.45 (sept, J=6.8 Hz, 1H), 3.00 (t, J=6.0 Hz, 2H), 2.62 (s, 3H), 1.38 (d, J=7.2 Hz, 6H), LC/MS: m/e=340 (M+H)⁺.

### Compound I-209

### 3-Cyclopentyl-7-((5-methylpyrazin-2-yl)methyl)-8,9-dihydro-6H-imidazo[5,1-f]pyrazino[2,1-c][1,2,4]triazin-11(7H)-one

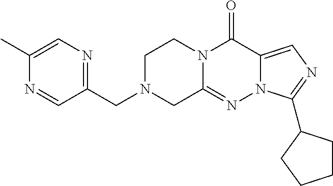 [SMILES: Cc1cnc(CN2CCn3c(nn4c(C5CCCC5)ncc4c3=O)C2)cn1]

The title compound was prepared using the procedure for I-133, substituting 5-methylpyrazine-2-carbaldehyde for 4-methoxybenzaldehyde. ¹H NMR (400 MHz, CDCl₃): δ 8.59 (s, 1H), 8.48 (s, 1H), 7.81 (s, 1H), 3.98 (t, J=5.6 Hz, 2H), 3.88 (s, 2H), 3.69 (s, 2H), 3.51 (quint, J=8.0 Hz, 1H), 2.99 (t, J=5.6 Hz, 2H), 2.61 (s, 3H), 2.08 (m, 2H), 1.97-1.71 (m, 4H), 1.69 (m, 2H), LC/MS: m/e=366 (M+H)⁺.

### Compound I-231

### (±)-7-Benzyl-3-(tetrahydrofuran-3-yl)-8,9-dihydro-6H-imidazo[5,1-f]pyrazino[2,1-c][1,2,4]triazin-11(7H)-one

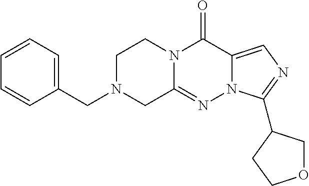 [SMILES: O=c1c2cnc(C3CCOC3)n2nc2n1CCN(Cc1ccccc1)C2]

The title compound was prepared using the procedure for I-133, substituting (±)-methyl 1-amino-2-(tetrahydrofuran-3-yl)-1H-imidazole-5-carboxylate for methyl 1-amino-2-cyclopentyl-1H-imidazole-5-carboxylate, and substituting benzaldehyde for 4-methoxybenzaldehyde. ¹H NMR (400 MHz, CDCl₃): δ 7.81 (s, 1H), 7.35 (m, 5H), 4.20 (t, J=8.0 Hz, 1H), 4.05 (m, 1H), 3.96 (m, 4H), 3.87 (n, 1H), 3.71 (s, 2H), 3.59 (s, 2H), 2.89 (t, J=6.0 Hz, 2H), 2.37 (m, 2H), LC/MS: m/e=352 (M+H)⁺.

### Compound I-232

### (±)-7-(4-Methoxybenzyl)-3-(tetrahydrofuran-3-yl)-8,9-dihydro-6H-imidazo[5,1-f]pyrazino[2,1-c][1,2,4]triazin-11(7H)-one

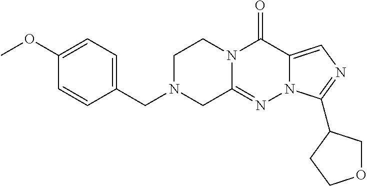 [SMILES: COc1ccc(CN2CCn3c(nn4c(C5CCOC5)ncc4c3=O)C2)cc1]

The title compound was prepared using the procedure for I-133, substituting (±)-methyl 1-amino-2-(tetrahydrofuran-3-yl)-1H-imidazole-5-carboxylate for methyl 1-amino-2-cyclopentyl-1H-imidazole-5-carboxylate. ¹H NMR (400 MHz, CDCl₃): δ 7.83 (s, 1H), 7.28 (d, J=8.4 Hz, 2H), 6.92 (d, J=8.4 Hz, 2H), 4.22 (t, J=7.6 Hz, 1H), 4.08 (m, 1H), 3.97 (m, 4H), 3.88 (m, 1H), 3.85 (s, 3H), 3.66 (s, 2H), 3.58 (s, 2H), 2.89 (t, J=5.6 Hz, 2H), 2.39 (m, 2H), LC/MS: m/e=382 (M+H)⁺.

### Compound I-233

### (±)-7-(4-Chlorobenzyl)-3-(tetrahydrofuran-3-yl)-8,9-dihydro-6H-imidazo[5,1-f]pyrazino[2,1-c][1,2,4]triazin-11(7H)-one

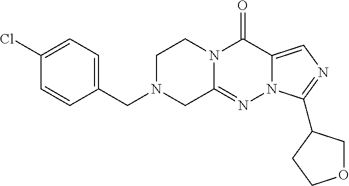 [SMILES: O=c1c2cnc(C3CCOC3)n2nc2n1CCN(Cc1ccc(Cl)cc1)C2]

The title compound was prepared using the procedure for I-133, substituting (1)-methyl 1-amino-2-(tetrahydrofuran-3-yl)-1H-imidazole-5-carboxylate for methyl 1-amino-2-cyclopentyl-1H-imidazole-5-carboxylate, and substituting 4-chlorobenzaldehyde for 4-methoxybenzaldehyde. ¹H NMR (400 MHz, CDCl₃): δ 7.83 (s, 1H), 7.37 (d, J=8.0 Hz, 2H), 7.31 (d, J=8.4 Hz, 2H), 4.21 (t, J=7.2 Hz, 1H), 4.09 (m, 1H), 3.97 (m, 4H), 3.89 (m, 1H), 3.69 (s, 2H), 3.58 (s, 2H), 2.90 (t, J=5.6 Hz, 2H), 2.39 (m, 2H), LC/MS: m/e=386 (M+H)⁺.

### Compound I-234

### 7-(4-Methoxybenzyl)-3-(tetrahydro-2H-pyran-4-yl)-8,9-dihydro-6H-imidazo[5,1-f]pyrazino[2,1-c][1,2,4]triazin-11(7H)-one

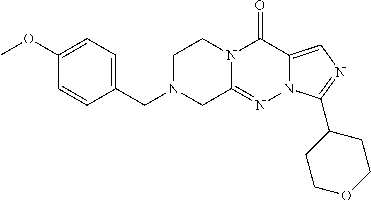 [SMILES: COc1ccc(CN2CCn3c(nn4c(C5CCOCC5)ncc4c3=O)C2)cc1]

The title compound was prepared using the procedure for I-133, substituting methyl 1-amino-2-(tetrahydro-2H-pyran-4-yl)-1H-imidazole-5-carboxylate for methyl 1-amino-2-cyclopentyl-1H-imidazole-5-carboxylate. ¹H NMR (400 MHz, d⁴-MeOH): δ 7.73 (s, 1H), 7.31 (d, J=8.0 Hz, 2H), 6.93 (d, J=8.0 Hz, 2H), 4.03 (d, J=10.0 Hz, 2H), 3.83 (m, 2H), 3.81 (s, 3H), 3.68 (s, 2H), 3.58 (m, 4H), 3.49 (m, 1H), 2.94 (m, 2H), 1.97 (m, 2H), 1.89 (m, 2H), LC/MS: m/e=396 (M+H)⁺.

### Compound I-244

### 3-Cyclopentyl-7-(4-methoxyphenethyl)-8,9-dihydro-6H-imidazo[5,1-f]pyrazino[2,1-c][1,2,4]triazin-11(7H)-one

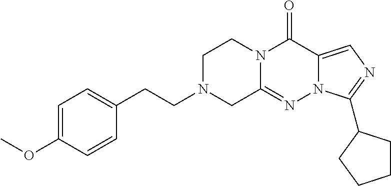 [SMILES: COc1ccc(CCN2CCn3c(nn4c(C5CCCC5)ncc4c3=O)C2)cc1]

The title compound was prepared using the procedure for I-133, substituting phenylacetaldehyde for 4-methoxybenzaldehyde. ¹H NMR (400 MHz, CDCl₃): δ 7.82 (s, 1H), 7.15 (d, J=8.4 Hz, 2H), 6.87 (d, J=8.8 Hz, 2H), 3.95 (t, J=5.6 Hz, 2H), 3.81 (s, 3H), 3.67 (s, 2H), 3.55 (m, 1H), 2.93 (t, J=5.6 Hz, 2H), 2.83 (m, 2H), 2.75 (m, 2H), 2.12 (m, 2H), 1.97-1.87 (m, 4H), 1.72 (m, 2H), LC/MS: m/e=394 (M+H)⁺.

### Compound I-235

### 7-(4-Chlorobenzyl)-3-(tetrahydro-2H-pyran-4-yl)-8,9-dihydro-6H-imidazo[5,1-f]pyrazino[2,1-c][1,2,4]triazin-11(7H)-one

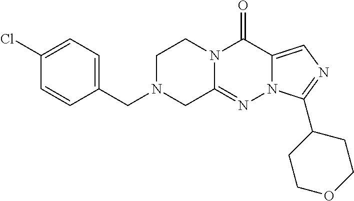 [SMILES: O=c1c2cnc(C3CCOCC3)n2nc2n1CCN(Cc1ccc(Cl)cc1)C2]

The title compound was prepared using the procedure for I-133, substituting methyl 1-amino-2-(tetrahydro-2H-pyran-4-yl)-1H-imidazole-5-carboxylate for methyl 1-amino-2-cyclopentyl-1H-imidazole-5-carboxylate, and substituting 4-chlorobenzaldehyde for 4-methoxybenzaldehyde. ¹H NMR (400 MHz, d⁴-MeOH): δ 7.74 (s, 1H), 7.40 (dd, J₁=14.2 Hz, J₂=8.4 Hz, 4H), 4.03 (dd, J₁=11.6 Hz, J₂=3.2 Hz, 2H), 3.93 (t, J=6.0 Hz, 2H), 3.74 (s, 2H), 3.62 (s, 2H), 3.58 (m, 2H), 3.50 (m, 1H), 2.94 (t, J=5.6 Hz, 2H), 1.98 (m, 2H), 1.87 (m, 2H), LC/MS: m/e=400 (M+H)⁺.

Additional compounds of formula I were prepared in a manner substantially similar to that described above.

In certain embodiments, compounds of the present invention are assayed as inhibitors of PDE1. In some embodiments compounds are assayed using Homogenous Time Resolved Fluorescence (HTRF).

### Example 15

### In Vitro HTRF PDE1 Inhibition Assay

An exemplary procedure for the in vitro Homogenous Time Resolved Fluorescence assay, which can be used to determine the inhibitory action of compounds of the invention toward. PDE1 or its isoforms, follows.

The HTRF PDE1 assay utilized the HTRF (Homogenous Time Resolved Fluorescence) technology, which is based on the competition between unlabeled cyclic nucleotide and cyclic nucleotide labeled with XL665 for the binding to cyclic nucleotide-specific antibody labeled with cryptate. The HTRF signal is thus inversely proportional to the concentration of cyclic nucleotide being measured. Since phosphodiesterases break down cyclic nucleotides the HTRF signal was used to determine PDE activity.

The Cisbio cGMP HTRF assay kit (Cat no: 62GM2PEC) was utilized. Cyclic GMP was diluted to 200 nM in HTRF assay buffer (1 mM CaCl₂, 10 mM MgCl₂, 10 mM Tris-HCl, 0.1% BSA, pH7.4). 10 μl of compound or DMSO was diluted in 200 nM cyclic GMP solution and added to wells of a 96 well white plate to give 100 nM cyclic GMP in 1% DMSO final concentration. PDE (1A3, 1B or 1C) was diluted to 2× working concentration in HTRF assay buffer with 2.1 g/ml Calmodulin, and 10 μl was added to initiate the reaction. The plate was then incubated for 45 minutes at 37° C. d2-Labelled cyclic GMP and anti-cGMP cryptate were diluted in 50 mM phosphate buffer, 0.8M KF, 1% Triton X100, 0.2% BSA, pH7.0. Following incubation 10 μl d2-cGMP, then 10 μl anti cGMP cryptate were added to each well and the plate was incubated for 45 minutes at room temperature. The plate was then read on Perkin Elmer Victor at 2 different FRET readings ex/emm 340 nm/665 nm and 340 nm/615 nm.

Data obtained from the HTRF assay for selected compounds of the invention are listed in Table 2 below. Compounds having an IC₅₀ of <1 μM, are denoted as +++. Compounds having an IC₅₀ of 1-10 μM, are denoted as ++. Compounds having an IC₅₀ of >10 μM, are denoted as +.

TABLE 2
Results of in vitro HTRF Assay

| Compound | |
|---|---|
| No. | IC₅₀ |
| I-1 | + |
| I-2 | + |
| I-3 | + |
| I-4 | + |
| I-5 | + |
| I-6 | + |
| I-7 | + |
| I-8 | ++ |
| I-9 | + |
| I-10 | + |
| I-11 | + |
| I-12 | + |
| I-13 | + |
| I-14 | + |
| I-15 | + |
| I-16 | + |
| I-17 | + |
| I-18 | + |
| I-19 | + |
| I-20 | + |
| I-21 | + |
| I-22 | + |
| I-23 | + |
| I-24 | + |
| I-25 | + |
| I-26 | + |
| I-27 | + |
| I-28 | + |
| I-29 | ++ |
| I-30 | + |
| I-31 | + |
| I-32 | + |
| I-33 | + |
| I-34 | + |
| I-35 | + |
| I-36 | + |
| I-37 | + |
| I-38 | + |
| I-39 | + |
| I-40 | + |
| I-41 | + |
| I-42 | + |
| I-43 | + |
| I-44 | + |
| I-45 | + |
| I-46 | + |
| I-47 | + |
| I-48 | + |
| I-49 | + |
| I-50 | + |
| I-51 | ++ |
| I-52 | ++ |
| I-53 | + |
| I-54 | ++ |
| I-55 | ++ |
| I-56 | ++ |
| I-57 | + |
| I-58 | + |
| I-59 | + |
| I-60 | + |
| I-61 | ++ |
| I-62 | ++ |
| I-63 | ++ |
| I-64 | ++ |
| I-65 | +++ |
| I-66 | ++ |
| I-67 | +++ |
| I-68 | ++ |
| I-69 | +++ |
| I-70 | +++ |
| I-71 | +++ |
| I-72 | +++ |
| I-73 | +++ |
| I-74 | +++ |
| I-75 | +++ |
| I-76 | ++ |
| I-77 | ++ |
| I-78 | +++ |
| I-79 | ++ |
| I-80 | ++ |
| I-81 | +++ |
| I-82 | +++ |
| I-83 | +++ |
| I-84 | +++ |
| I-85 | ++ |
| I-86 | ++ |
| I-87 | ++ |
| I-88 | +++ |
| I-89 | +++ |
| I-90 | ++ |
| I-91 | +++ |
| I-92 | +++ |
| I-93 | +++ |
| I-94 | ++ |
| I-95 | +++ |
| I-96 | ++ |
| I-97 | +++ |
| I-98 | +++ |
| I-99 | + |
| I-100 | + |
| I-101 | ++ |
| I-102 | + |
| I-103 | + |
| I-104 | + |
| I-105 | +++ |
| I-106 | + |
| I-107 | + |
| I-108 | + |
| I-109 | + |
| I-110 | + |
| I-111 | + |
| I-112 | + |
| I-113 | ++ |
| I-114 | ++ |
| I-115 | ++ |
| I-116 | +++ |
| I-117 | ++ |
| I-118 | ++ |
| I-119 | +++ |
| I-120 | +++ |
| I-121 | ++ |
| I-122 | ++ |
| I-123 | + |
| I-124 | ++ |
| I-125 | + |
| I-126 | + |
| I-127 | + |
| I-128 | + |
| I-129 | ++ |
| I-130 | ++ |
| I-131 | +++ |
| I-132 | +++ |
| I-133 | +++ |
| I-134 | +++ |
| I-135 | +++ |
| I-136 | ++ |
| I-137 | ++ |
| I-138 | + |
| I-139 | + |
| I-140 | +++ |
| I-141 | +++ |
| I-142 | +++ |
| I-143 | ++ |
| I-144 | +++ |
| I-145 | +++ |
| I-146 | ++ |
| I-147 | +++ |
| I-148 | +++ |
| I-149 | +++ |
| I-150 | ++ |
| I-151 | +++ |
| I-152 | +++ |
| I-153 | ++ |
| I-154 | ++ |
| I-155 | ++ |
| I-156 | ++ |
| I-157 | + |
| I-158 | ++ |
| I-159 | ++ |
| I-160 | + |
| I-161 | + |
| I-162 | ++ |
| I-163 | + |
| I-164 | ++ |
| I-165 | ++ |
| I-166 | ++ |
| I-167 | +++ |
| I-168 | ++ |
| I-169 | +++ |
| I-170 | ++ |
| I-171 | +++ |
| I-172 | +++ |
| I-173 | +++ |
| I-174 | +++ |
| I-175 | +++ |
| I-176 | +++ |
| I-177 | +++ |
| I-178 | +++ |
| I-179 | +++ |
| I-180 | +++ |
| I-181 | +++ |
| I-182 | +++ |
| I-183 | +++ |
| I-184 | +++ |
| I-185 | ++ |
| I-186 | ++ |
| I-187 | +++ |
| I-188 | ++ |
| I-189 | +++ |
| I-190 | + |
| I-191 | ++ |
| I-192 | + |
| I-193 | ++ |
| I-194 | + |
| I-195 | ++ |
| I-196 | ++ |
| I-197 | ++ |
| I-198 | + |
| I-199 | ++ |
| I-200 | ++ |
| I-201 | ++ |
| I-202 | ++ |
| I-203 | ++ |
| I-204 | ++ |
| I-205 | +++ |
| I-206 | ++ |
| I-207 | ++ |
| I-208 | ++ |
| I-209 | +++ |
| I-210 | ++ |
| I-211 | +++ |
| I-212 | +++ |
| I-213 | +++ |
| I-214 | ++ |
| I-215 | +++ |
| I-216 | ++ |
| I-217 | +++ |
| I-218 | ++ |
| I-219 | + |
| I-220 | ++ |
| I-221 | ++ |
| I-222 | ++ |
| I-223 | ++ |
| I-224 | ++ |
| I-225 | ++ |
| I-226 | + |
| I-227 | ++ |
| I-228 | ++ |
| I-229 | +++ |
| I-230 | +++ |
| I-231 | +++ |
| I-232 | ++ |
| I-233 | ++ |
| I-234 | +++ |
| I-235 | +++ |
| I-236 | +++ |
| I-237 | +++ |
| I-238 | +++ |
| I-239 | +++ |
| I-240 | +++ |
| I-241 | +++ |
| I-242 | +++ |
| I-243 | ++ |
| I-244 | ++ |
| I-245 | + |
| I-246 | ++ |
| I-247 | ++ |
| I-248 | +++ |
| I-249 | +++ |
| I-250 | ++ |
| I-251 | +++ |
| I-252 | ++ |
| I-253 | ++ |
| I-254 | ++ |
| I-255 | ++ |
| I-256 | ++ |
| I-257 | ++ |
| I-258 | +++ |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of formula I: 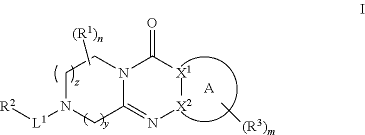
or a pharmaceutically acceptable salt thereof, wherein:
X¹ and X² are each independently C or N;
Ring A is a 5-membered heteroaryl ring having 1-4 heteroatoms independently selected from nitrogen, oxygen and sulfur;
L¹ is a covalent bond, or a C₁₋₄ bivalent straight or branched hydrocarbon chain, wherein one or more hydrogen atoms of the chain are optionally and independently replaced by halogen, and wherein one or two methylene units of the chain are optionally and independently replaced by —N(R)—, —N(R)C(O)—, —C(O)N(R)—, —N(R)C(O)N(R)—, —N(R)C(S)N(R)—, —N(R)S(O)₂—, —S(O)₂N(R)—, —C(O)—, —OC(O)—, —C(O)O—, —O—, —S—, —S(O)— or S(O)₂—;
each R¹ is independently halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)S(O)₂R, —S(O)₂N(R)₂, —OC(O)OR, —C(O)OR, —OC(O)R, —C(O)R, —S(O)R, or S(O)₂R;
R² is halogen, —R, —OR, —SR, —N(R)₂, —N(R)C(O)R, —C(O)N(R)₂, —N(R)C(O)N(R)₂, —N(R)C(S)N(R)₂, —N(R)C(O)OR, —OC(O)N(R)₂, —N(R)SO₂R, —S(O)₂N(R)₂, —C(O)R, —C(O)OR, —OC(O)R, —S(O)R, —S(O)₂R, or Cy;
Cy is a ring, substituted with p instances of R⁵; wherein said ring is a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; or an 8-10 membered bicyclic heteroaromatic ring having 1-5 heteroatoms independently selected from nitrogen, oxygen, and sulfur;
each R is independently hydrogen, or an optionally substituted group selected from C₁₋₆ aliphatic; a 3-8 membered saturated or partially unsaturated monocyclic carbocyclic ring; phenyl; an 8-10 membered bicyclic aromatic carbocyclic ring; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur; and an 8-10 membered bicyclic heteroaromatic ring having 1-5 heteroatoms independently selected from nitrogen, oxygen, and sulfur;
each R³ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂;
each R⁵ is independently halogen, —R, —CN, —OR, —SR, —N(R)₂, —N(R)C(O)R, —C(O)N(R)₂, —C(O)N(R)S(O)₂R, —N(R)C(O)N(R)₂, —N(R)C(S)N(R)₂, —N(R)C(O)OR, —OC(O)N(R)₂, —N(R)S(O)₂R, —S(O)₂N(R)₂, —C(O)R, —C(O)OR, —OC(O)R, —S(O)R, —S(O)₂R, or —B(OR)₂; or
two R⁵ are taken together with their intervening atoms to form a 5-6 membered saturated, partially unsaturated, or aromatic ring having 0-3 heteroatoms independently selected from nitrogen, oxygen, and sulfur;
wherein one or more of {two instances of R¹}, {R² and an R¹}, and {two instances of R³} may be taken together with their intervening atoms to form a ring, substituted with q instances of R⁵; wherein said ring is a 3-8 membered saturated or partially unsaturated monocyclic carbocyclic ring; phenyl; a 4-8 membered saturated or partially unsaturated monocyclic heterocyclic ring having 1-2 heteroatoms independently selected from nitrogen, oxygen, and sulfur; a 5-6 membered monocyclic heteroaromatic ring having 1-4 heteroatoms independently selected from nitrogen, oxygen, and sulfur;
y is 1-2;
z is 1-2;
m is 0-3;
n is 0-3;
p is 0-5; and
q is 0-5.

2. The compound of claim 1, wherein the compound is a compound of formula I-a, I-b, or II-c: 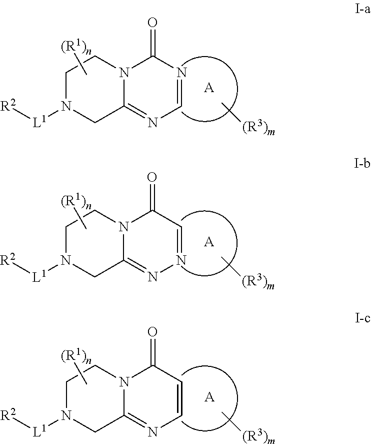
or a pharmaceutically acceptable salt thereof.

3. The compound of claim 1, wherein the compound is a compound of formula II-a, II-b, II-c, II-d, II-e, II-f, II-g, II-h, II-i, II-j or II-k: 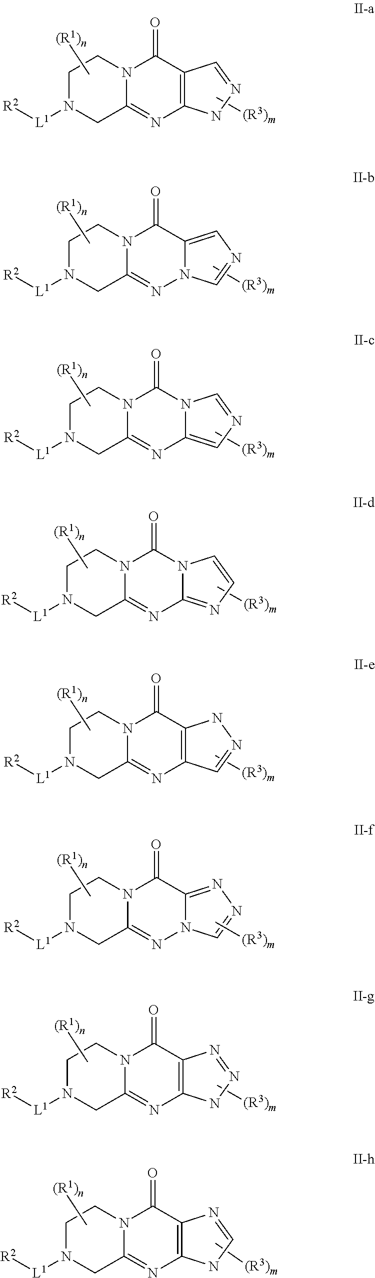 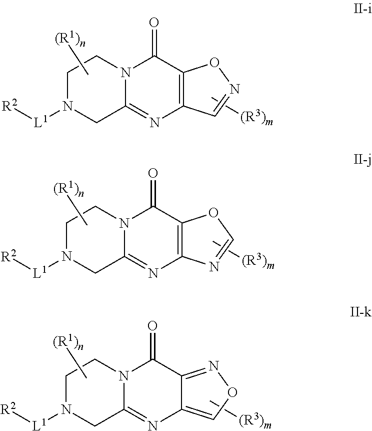
or a pharmaceutically acceptable salt thereof.

4. The compound of claim 1, wherein the compound is a compound of formula III-a, III-b, III-c, III-d, III-e, III-f, III-g, III-h, III-i, III-j or III-k: 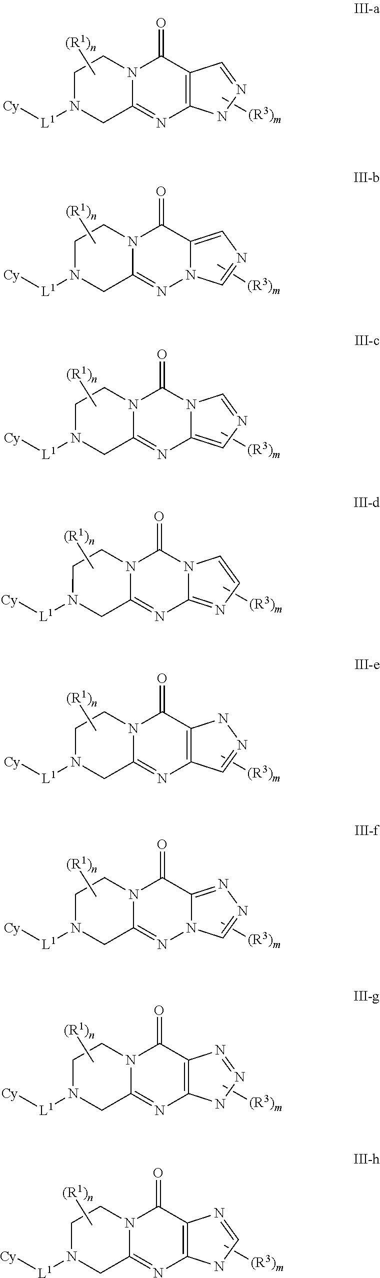 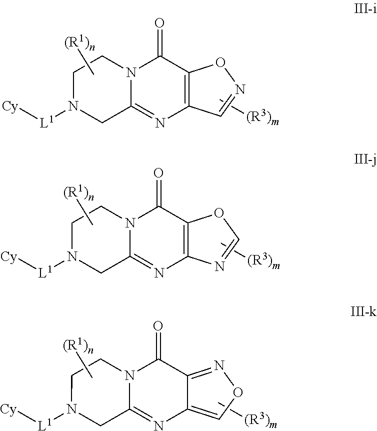
or a pharmaceutically acceptable salt thereof.

5. The compound according to claim 1 of formulas VI-a or VI-b: 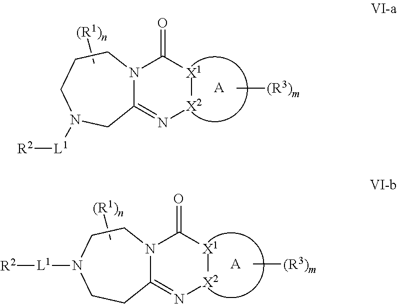
or a pharmaceutically acceptable salt thereof.

6. The compound of claim 1, wherein m is 1.

7. The compound of claim 1, wherein L¹ is a covalent bond.

8. The compound of claim 1, wherein L¹ is methylene.

9. The compound of claim 8, wherein R² is Cy.

10. A composition comprising the compound according to claim 1 and a pharmaceutically acceptable carrier, adjuvant, or vehicle.

11. A method of inhibiting PDE1 in a patient in need thereof, comprising administering to said patient the composition according to claim 10.

12. A method of inhibiting PDE1 in a biological sample in vitro, comprising contacting the biological sample with the compound according to claim 1.

13. A method for treating a neurological or psychiatric disorder in a patient in need thereof, comprising administering to said patient the composition according to claim 10.

14. The method according to claim 13, wherein the neurological or psychiatric disorder is Alzheimer's Disease, Parkinson's Disease, depression, cognitive impairment, stroke, schizophrenia, Down Syndrome, or Fetal Alcohol Syndrome.

15. The method according to claim 13, wherein the neurological or psychiatric disorder involves a deficit in one or more cognitive domains as defined by DSM-5.
